(19) United States
(12) Patent Application Publication
(10) Pub. No.: US 2009/0275538 A1
(43) Pub. Date: Nov. 5, 2009

(54) ARYLMETHYLIDENE HETEROCYCLES AS NOVEL ANALGESICS

(76) Inventors: Giorgio Attardo, Laval (CA); Sasmita Tripathy, Pierre Fonds (CA); Martin Gagnon, Montreal (CA)

Correspondence Address: CLARK & ELBING LLP, 101 FEDERAL STREET, BOSTON, MA, 02110, US

(21) Appl. No.: 12/368,058

(22) Filed: Feb. 9, 2009

Related U.S. Application Data
(60) Provisional application No. 61/027,329, filed on Feb. 8, 2008.
(60) Provisional application No. 61/135,253, filed on Jul. 17, 2008.

(51) Int. Cl. A61K 31/675; C07D 417/04; A61K 31/454; A61K 31/501; C07D 417/14; A61K 31/541; C07F 9/06; A61P 25/00

(52) U.S. Cl. 514/85; 546/209; 514/326; 514/252.05; 544/238; 544/58.4; 514/227.8; 544/232

(57) ABSTRACT

The present invention relates to Arylmethylidene heterocycles, compositions comprising an Arylmethylidene heterocycle, and methods useful for treating or preventing pain comprising administering an effective amount of an Arylmethylidene heterocycle. The compounds, compositions, and methods of the invention are also useful for treating or preventing inflammation.

### CROSS-REFERENCE TO RELATED APPLICATIONS

This application claims benefit of U.S. Provisional Application Nos. 61/027,329, filed Feb. 8, 2008, and 61/135,253, filed Jul. 17, 2008, each of which is hereby incorporated by reference.

### BACKGROUND OF THE INVENTION

The present invention relates pharmaceutical compositions and methods for treating or preventing pain and inflammation.

Pain is a common form of physical suffering and distress and is one of the most common reasons patients report to physicians. It may be categorized in terms of form (nociceptive or neuropathic), duration (chronic or acute), and degree (mild, moderate or severe). Typically, nociceptive pain is acute, and results from injury, such as burns, sprains, burns, fractures, or inflammation (inflammatory pain, including from osteo- and rheumatoid arthritis). Neuropathic pain, on the other hand, is defined by the International Association for the Study of Pain as a form of chronic pain that is caused by a lesion or dysfunction of the nervous system. Commonly, neuropathic pain results from diabetic neuropathy, HIV infections, and post-herpetic neuralgia. Other disorders that are associated with neuropathic pain include complex regional pain syndromes, trigeminal neuralgia, low back pain, sciatica, phantom limb pain, blast pain, fibromyalgia, and other conditions that result in chronic pain. Few therapeutics are approved by the US Food and Drug Administration and other regulatory agencies for the treatment of neuropathic pain. Those that are approved exhibit a modest efficacy in terms of pain reduction—at best (see Jensen, European Journal of Pain, 2002).

### SUMMARY OF THE INVENTION

The present invention features compounds having the Formula (Ia):

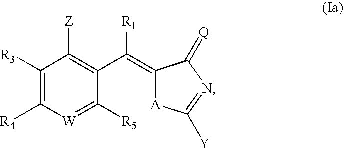

including stereoisomers, E/Z stereoisomers, prodrugs, and pharmaceutically acceptable salts thereof, wherein:

A is —O—, —S—, —SO—, —SO₂—, >NR₆, or >NC(O)R₆;

Q is O, S, or NR₆;

Z is —F, —Cl, —NO₂, —OR₂, —C(O)R₆, —C(O)(CR₆R₆)ₒNH₂, —N(R₆)₂, or —NHC(O)R₆;

W is CX or N;

X is —H, —F, —Cl, —CN, —OH, —C₂-C₈ alkyl, —C₂-C₈ alkenyl, —C₂-C₈ alkynyl, —C₃-C₁₂ cycloalkyl, —OC₂-C₄alkyl, —OC₂-C₄ alkenyl, —OC₂-C₄ alkynyl, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —S(O)R₆, —S(O)₂R₆, —S(O)₂N(R₆)₂, —NHC(O)R₆, —NHS(O)₂R₆, —NHC(NH)N(R₆)₂, —NR₆C(NH)N(R₆)₂, —NR₆C(NCN)N(R₆)₂, N-terminal linked amino acid, or C-terminal linked amino acid;

Y is —C₃-C₈ cycloalkyl, 3 to 8-membered aromatic or non aromatic heterocycle, —SR₆, —S(O)R₆, —S(O)₂R₆, —N(R₆)₂, —NHC(O)R₆, —NHS(O)₂R₆, —NHC(NH)N(R₆)₂, —NR₆C(NH)N(R₆)₂, or —NR₆C(NCN)N(R₆)₂;

R₁ is —H, halogen, —C₁-C₈ alkyl, —C₂-C₈ alkenyl, or —C₂-C₈ alkynyl;

R₂ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, —(CH₂)ₙOR₆, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid;

each R_{2A} and R_{2B} is, independently, H or C₁₋₅ alkyl;

R₃, R₄, and R₅ are each, independently, —H, —OH, halogen, —CN, —NO₂, —SH, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3 to 9-membered aromatic or non aromatic heterocycle, —OR₆, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —SOR₆, —S(O)₂R₆, —NHC(O)R₆, —NHS(O)₂R₆, —NHC(NH)N(R₆)₂, —NR₆C(NH)N(R₆)₂, —NHC(NCN)N(R₆)₂, —NR₆C(NCN)N(R₆)₂, or —PO(OR₆)₂, or R₃ and R₄, together with the carbon atoms to which each is attached, join to form a 5- to 6-membered aromatic or non aromatic carbocycle or heterocycle;

each R₆ is, independently, —H, —C₁-C₈ alkyl, alkcycloalkyl, alkheterocyclyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic carbocycle or heterocycle;

n is 1 or 2;

o is an integer between 0-3;

each r2 is an integer between 1-3;

each r3 is an integer between 0-2;

wherein R₃ is not —Br, when R₅ is —OH; and

wherein one of X and R₄ is not —H.

In some embodiments, Formula (Ia) excludes any compounds having the structure

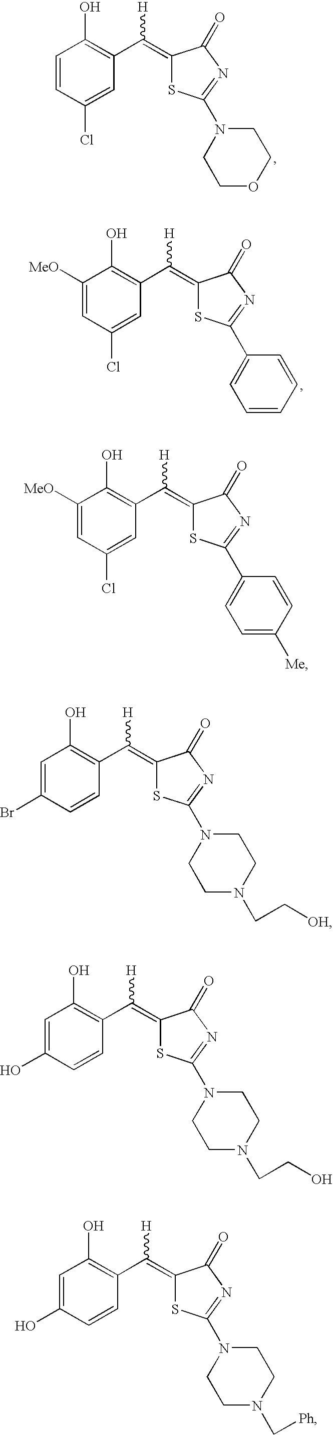

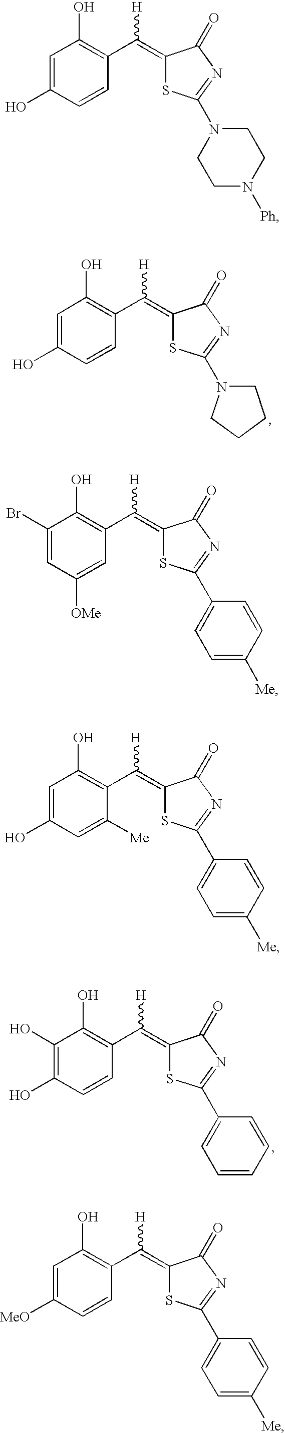

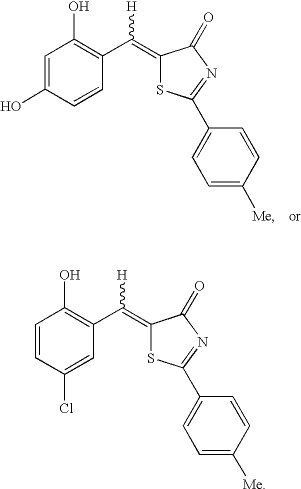

In some embodiments, when W is CX, one of X and R₄ is not —H.

In other embodiments, when Z is —H and R₅ is —OH, —OR₆, —O(CH₂)ₙOR₆, —OC(O)R₆, —OC(O)OR₆, or —OC(O)N(R₆)₂, one of R₃ and R₄ is not —H.

In some embodiments, Z is —F, —Cl, —NO₂, —OR₂, —N(R₆)₂, —NHC(O)R₆; X is —H, —F, —Cl, —CN, —OH, —C₂-C₈ alkyl, —C₂-C₈ alkenyl, —C₂-C₈ alkynyl, —C₃-C₁₂ cycloalkyl, —OC₂-C₄alkyl, —OC₂-C₄ alkenyl, —OC₂-C₄ alkynyl, —N(R₆)₂, —C(NH)N(R₆)₂, —O(CH₂)OR₆, —C(O)R₆, —OC(O)R₆, —OC(O)OR₆, —OC(O)N(R₆)₂, —C(O)N(R₆)₂, —C(O)OR₆, —SR₆, —S(O)R₆, —S(O)₂R₆, —S(O)₂N(R₆)₂, —NHC(O)R₆, —NHS(O)₂R₆, —NHC(NH)N(R₆)₂, —NR₆C(NH)N(R₆)₂, or —NR₆C(NCN)N(R₆)₂; R₂ is —H, —C₁-C₈ alkyl, —C₂-C₈ alkenyl, —C₂-C₈ alkynyl, —C₃-C₁₂ cycloalkyl, —C₆-C₁₂aryl, —C₇-C₁₄ arylalkyl, —(CH₂)ₙOR₆, —C(O)R₆, —C(O)OR₆, —C(O)NHR₆, —C(O)N(R₆)₂, or —PO(OR₆)₂; and each R₆ is, independently, —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic carbocycle or heterocycle.

In other embodiments, A is —O—, —S—, or >NR₆; Q is O, S, or NR₆; Z is —OR₂, —N(R₆)₂, —C(O)R₆, or —C(O)(C(R₆)₂)ₒNH₂; W is CX or N; X is —H, —F, —Cl, —CN, —C₂-C₅ alkyl, —C₂-C₅ alkenyl, —C₂-C₅ alkynyl, —OC₂-C₅ alkyl, —OC₂-C₅ alkenyl, —OC₂-C₅ alkynyl, —N(R₆)₂, —C(NH)N(R₆)₂, —C(O)R₆, —OC(O)R₆, —OC(O)OR₆, —OC(O)N(R₆)₂, —C(O)N(R₆)₂, —C(O)OR₆, —SR₆, —S(O)R₆, —S(O)₂R₆, —S(O)₂N(R₆)₂, —NHC(O)R₆, —NHS(O)₂R₆, —NHC(NH)N(R₆)₂, —NR₆C(NH)N(R₆)₂, —NHC(NCN)N(R₆)₂, —NR₆C(NCN)N(R₆)₂, N-terminal linked amino acid, or C-terminal linked amino acid; Y is —C₃-C₆ cycloalkyl, 5 to 9-membered aromatic or non aromatic heterocycle, —N(R₆)₂, —NHC(O)R₆, —NHS(O)₂R₆, —NHC(NH)N(R₆)₂, —NR₆C(NH)N(R₆)₂, —NHC(NCN)N(R₆)₂, or —NR₆C(NCN)N(R₆)₂; R₁ is —H, halogen, —C₁-C₄ alkyl, —C₂-C₄ alkenyl, or —C₂-C₄ alkynyl; R₂ is —H, —C₁-C₅ alkyl, —C₂-C₅ alkenyl, —C₂-C₅ alkynyl, —C₃-C₆ cycloalkyl, phenyl, —C₇-C₈ arylalkyl, —(CH₂)ₙOR₆, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid; R₃, R₄, and R₅ are each, independently, —H, —OH, —F, —Cl, —CN, —NO₂, —SH, —C₁-C₅ alkyl, —C₂-C₅ alkenyl, —C₂-C₅ alkynyl, —C₃-C₆ cycloalkyl, phenyl, —C₇-C₈ arylalkyl, 5 to 6-membered aromatic or non aromatic heterocycle, —OR₆, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —SOR₆, —S(O)₂R₆, —NHC(O)R₆, —NHS(O)₂R₆, —NHC(NH)N(R₆)₂, —NR₆C(NH)N(R₆)₂, —NHC(NCN)N(R₆)₂, —NR₆C(NCN)N(R₆)₂, or —PO(OR₆)₂, or R₃ and R₄, together with the carbon atoms to which each is attached, join to form a 5- to 6-membered aromatic or non aromatic carbocycle or heterocycle; and each R₆ is, independently, —H, —C₁-C₅ alkyl, —C₃-C₆ cycloalkyl, phenyl, —C₇-C₈ arylalkyl, 5 to 6-membered aromatic or non aromatic heterocycle, —C₂-C₅ alkenyl, or —C₂-C₅ alkynyl, or two R₆, together with the atom to which each is attached, join to form a 3- to 7-membered aromatic or non aromatic carbocycle or heterocycle; and wherein o is 1 or 2; and r2 is 1 or 2.

In still other embodiments, A is —O—, —S—, >NH, or >NCH₃; Q is O; Z is OR₂, —N(R₆)₂, —C(O)R₆, or —C(O)(C(R₆)₂)ₙNH₂; W is CX or N; X is —H, —F, —Cl, —CN, —C₂-C₅ alkyl, —C₂-C₅ alkenyl, —OC₂-C₅ alkyl, —OC₂-C₅ alkenyl, —N(R₆)₂, —C(NH)N(R₆)₂, —C(O)R₆, —O—C(O)N(R₆)₂, —C(O)N(R₆)₂, —C(O)OR₆, —SR₆, —S(O)₂R₆, —S(O)₂N(R₆)₂, —NHC(O)R₆, —NHS(O)₂R₆, —NHC(NH)N(R₆)₂, —NR₆C(NH)N(R₆)₂, —NHC(NCN)N(R₆)₂, —NR₆C(NCN)N(R₆)₂, N-terminal linked amino acid, or C-terminal linked amino acid; Y is 5 to 6-membered aromatic or non aromatic heterocycle, —N(R₆)₂, —NHC(O)R₆, or —NHS(O)₂R₆; R₁ is —H, halogen, —C₁-C₅ alkyl, or —C₂-C₅ alkenyl; R₂ is —H, —C₁-C₅ alkyl, —C₂-C₅ alkenyl, —C₂-C₅ alkynyl, —C₃-C₆ cycloalkyl, —(CH₂)ₙOR₆, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid; R₃, R₄, and R₅ are each, independently, —H, —OH, —F, —Cl, —CN, —NO₂, —SH, —C₁-C₅ alkyl, —C₂-C₅ alkenyl, —C₂-C₅ alkynyl, —OR₆, —N(R₆)₂, —C(NH)N(R₆)₂, —C(O)R₆, —OC(O)N(R₆)₂, —C(O)N(R₆)₂, —C(O)OR₆, —SR₆, —SOR₆, —S(O)₂R₆, —NHC(O)R₆, —NHS(O)₂R₆, or —PO(OR₆)₂, or R₃ and R₄, together with the carbon atoms to which each is attached, join to form a 5- to 6-membered aromatic or non aromatic carbocycle or heterocycle; each R₆ is, independently, —H, —C₁-C₅ alkyl, —C₃-C₆ cycloalkyl, —C₂-C₅ alkenyl, or —C₂-C₅ alkynyl, or two R₆, together with the atom to which each is attached, join to form a 5- to 6-membered aromatic or non aromatic carbocycle or heterocycle; and wherein o is 1 or 2; r2 is 1 or 2; and r3 is 0 or 2.

In some embodiments, A is —O— or —S—; Q is O or S; Z is —OR₂; W is CX; X is —H or —F; Y is —C₃-C₈ cycloalkyl, 3 to 8-membered aromatic or non aromatic heterocycle, or —N(R₆)₂; R₁ is —H; R₂ is —H, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid; each R_{2A} and R_{2B} is, independently, H or C₁₋₄ alkyl; R₃, R₄, and R₅ are each, independently, —H or halogen; and each R₆ is, independently, —H, —C₁-C₈ alkyl, alkcycloalkyl, alkheterocyclyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic carbocycle or heterocycle.

In some embodiments, the compound of Formula (Ia) has the following structure

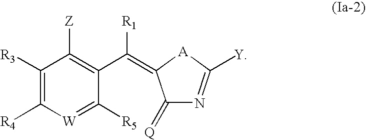

In other embodiments, the compound of Formula (Ia) has the following structure:

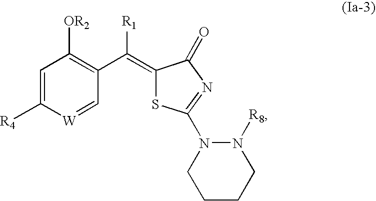

wherein R₈ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3 to 9-membered aromatic or non aromatic heterocycle, —(CH₂)ₙOR₆, —C(O)R₆, —C(O)OR₆, —C(O)NHR₆, —C(O)N(R₆)₂, —C(O)N(R₆)₂, —(CR_{Y1}R_{Y2})_{y2}PO(OR_{Y3})(OR_{Y4}); —C(NH)N(R₆)₂, or —S(O)₂R₆; each R_{Y1}, R_{Y2}, R_{Y3}, and R_{Y4} is, independently, H or C₁₋₅ alkyl; and y2 is 0 or 2.

In still other embodiments, the compound of Formula (Ia) has the following structure:

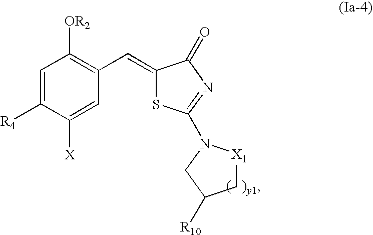

wherein

X is H or F; R₂ is —H, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid; R₄ is H or F; R₁₀ is H or N(CH₃)₂; XI is CH₂ or NR₈; R₈ is H or —(CR_{Y1}R_{Y2})_{y2}PO(OR_{Y3})(OR_{Y4}); each R_{Y1}, R_{Y2}, R_{Y3}, and R_{Y4} is, independently, H, C₁₋₅ alkyl, or R_{Y3} and R_{Y4} combine to form a 5 to 7 membered ring; and each y1 and y2 is, independently, 0, 1, or 2.

In some embodiments, A is —O—, —S—, —SO₂—, >NH, or >NCH₃.

In some embodiments, Q is O.

In some embodiments, W is CX. In certain embodiments, W is CF.

In other embodiments, R₄ is —F.

In still other embodiments, R₁ and R₂ are both H.

In some embodiments, Y is a 5 to 6-membered non aromatic heterocycle.

In certain embodiments, Y is

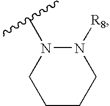

wherein R₈ is H, —(CR_{Y1}R_{Y2})_{y2}PO(OR_{Y3})(OR_{Y4}), or —C(O)R_{Y5}; each R_{Y1}, R_{Y2}, R_{Y3}, and R_{Y4} is, independently, H, C₁₋₅ alkyl, or R_{Y3} and R_{Y4} combine to form a 5 to 7 membered ring; each R_{Y5} is aryl; and Y₂ is 0, 1, or 2. In further embodiments, R₈ is H. In other embodiments, R₈ is —(CH₂)_{y2}PO(ORy₄)(OR_{Y5}).

In some embodiments, Y is optionally substituted azetidinyl, optionally substituted pyrrolidinyl, optionally substituted piperidinyl, optionally substituted piperazinyl, optionally substituted morpholinyl, optionally substituted tetrahydropyridinyl, or optionally substituted hexamethyleneiminyl. In further embodiments, Y has 0, 1, 2, 3, 4, 5, 6, or 7 substituents as defined herein. In some embodiments, Y is

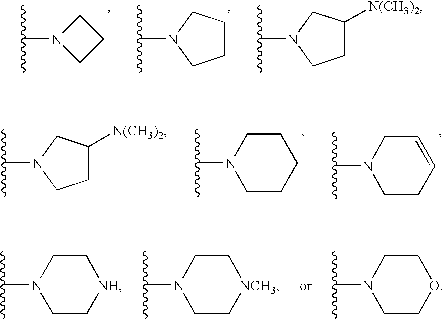

In some embodiments, R₆ is either H or CH₃.

In other embodiments, two R₆, together with the atom to which each is attached, join to form a 5-, 6-, or 7-membered non aromatic heterocycle.

In some embodiments where two R₆, together with the atom to which each is attached, join to form a 3- to 7-membered aromatic or non aromatic carbocycle or heterocycle, the carbocycle or the heterocycle is substituted with any of the substituent groups described herein. In some embodiments, the carbocycle or the heterocycle is substituted with, for example, 1, 2, 3, 4, 5, 6, or 7 substituents. In some embodiments, the carbocycle or the heterocycle is substituted with an amino group.

In certain embodiments, R₃ and R₄, together with the atom to which each is attached, join to form a 5- or 6-membered aromatic or non aromatic carbocycle or heterocycle.

In some embodiments, R₆ is H and Z is OR₂.

In some embodiments, R₂ is H, —C(O)N(R₆)₂, —C(O)R₆, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid. In some embodiments R₂ is —C(O)N(R₆)₂, and each R₆ is, independently, H, —C₁-C₄ alkyl, —C₆-C₁₂ aryl, —C₇-C₁₄ arylalkyl, or two R₆, together with the atom to which each is attached, join to form a 5- or 6-membered non aromatic heterocycle. In some embodiments, R₂ is —C(O)NHCH₃, —C(O)NHCH₂CH₃, —C(O)N(CH₃)₂, —C(O)N(CH₂CH₃)₂, —C(O)N(CH₃)(CH₂CH₃),

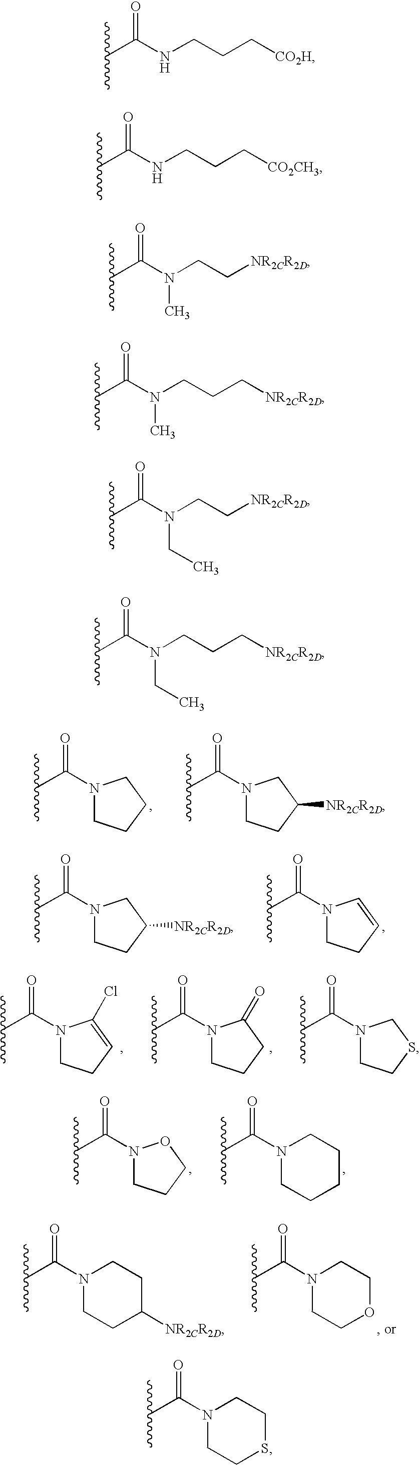

wherein R_{2C} and R_{2D} are, independently, H, C₁₋₃ alkyl, or R_{2C} and R_{2D} combine to form a 5- or 6-membered non aromatic heterocycle.

In some embodiments, R₂ is an N-terminal linked amino acid or a C-terminal linked amino acid. In certain embodiments, R₂ is an N-terminal linked natural amino acid or a C-terminal linked natural amino acid. In other embodiments, R₂ is an N-terminal linked unnatural amino acid or a C-terminal linked unnatural amino acid. In some embodiments, the unnatural amino acid is gabapentin or pregabalin. In other embodiments, R₂ is

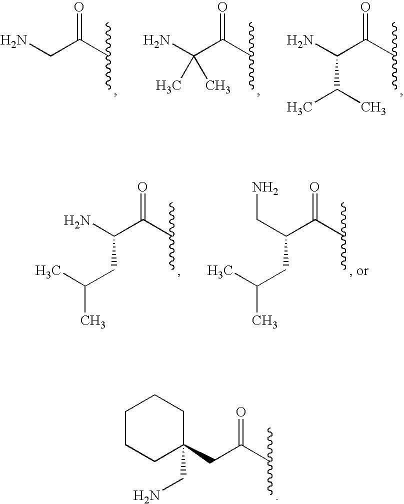

In some embodiments, R_{2C} and R_{2D} are, independently, —CH₃, —CH₂CH₃, or R_{2C} and R_{2D} combine to form unsubstituted pyrrolidinyl or unsubstituted piperidinyl.

In certain embodiments, R₂ is —PO(OR₆)₂, —CH₂PO(OR₆)₂, —C(CH₃)₂PO(OR₆)₂, or —CH₂CH₂PO(OR₆)₂.

In still other embodiments, each R₆ is, independently, H, C₁₋₃ alkyl, or two R₆ combine to form a 5-, 6-, or 7-membered ring. In some embodiments, each R₆ is, independently, H, CH₃, or CH₂CH₃.

In certain embodiments, R₂ is —C(O)R₆, wherein R₆ is —C₆-C₁₂ aryl or —C₇-C₁₄ arylalkyl.

In some embodiments, R₆ has the structure

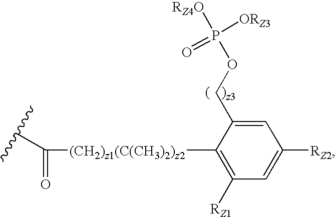

wherein R_{Z1} and R_{Z2} are, independently, H or CH₃; R_{Z3} and R_{Z4} are, independently, H, C₁₋₃ alkyl, or two R₆ combine to form a 5-, 6-, or 7-membered ring; and each z1, z2, and z3 is, independently, 0, 1, or 2.

In some embodiments, A is —S—, Z is OR₂, W is CX, and each of X and R₄ is —H or —F in any of the compounds, compositions, and methods of the invention.

In some embodiments, both R₆ are either H or CH₃ in any of the compounds, compositions, and methods of the invention.

In some embodiments, two R₆, together with the atom to which each is attached, join to form a 5-, 6-, or 7-membered non aromatic heterocycle in any of the compounds, compositions, and methods of the invention.

In some embodiments, R₃ and R₄, together with the atom to which each is attached, join to form a 5- or 6-membered aromatic or non aromatic carbocycle or heterocycle in any of the compounds, compositions, and methods of the invention.

In some embodiments, W is CX in any of the compounds, compositions, and methods of the invention.

In some embodiments, A is —O—, —S—, —SO₂—, >NH, or >NCH₃ in any of the compounds, compositions, and methods of the invention.

In some embodiments, Q is O in any of the compounds, compositions, and methods of the invention.

In some embodiments, W is CF in any of the compounds, compositions, and methods of the invention.

In some embodiments, R₄ is —F in any of the compounds, compositions, and methods of the invention.

In some embodiments, R₁ and R₂ are both H in any of the compounds, compositions, and methods of the invention.

In certain embodiments, A is —O—, and Q is O in any of the compounds, compositions, and methods of the invention.

In certain embodiments, A is —S—, and Q is O in any of the compounds, compositions, and methods of the invention.

In certain embodiments, A is —SO₂—, and Q is O in any of the compounds, compositions, and methods of the invention.

In certain embodiments, A is >NH, and Q is O in any of the compounds, compositions, and methods of the invention.

In certain embodiments, A is >NCH₃, and Q is O in any of the compounds, compositions, and methods of the invention.

In certain embodiments, X or R₄ is —F in any of the compounds, compositions, and methods of the invention.

In certain embodiments, Y is a 5- to 6-membered non aromatic heterocycle in any of the compounds, compositions, and methods of the invention.

In certain embodiments, R₁ and R₂ are both H in any of the compounds, compositions, and methods of the invention.

In certain embodiments, R₁ and R₂ are both H, A is —O—, and Q is O in any of the compounds, compositions, and methods of the invention.

In certain embodiments, R₁ and R₂ are both H, A is —S—, and Q is O in any of the compounds, compositions, and methods of the invention.

In certain embodiments, R₁ and R₂ are both H, A is —SO₂—, and Q is O in any of the compounds, compositions, and methods of the invention.

In certain embodiments, R₁ and R₂ are both H, A is >NH, and Q is O in any of the compounds, compositions, and methods of the invention.

In certain embodiments, R₁ and R₂ are both H, A is >NCH₃, and Q is O in any of the compounds, compositions, and methods of the invention.

In certain embodiments, X is —F, R₁ and R₂ are both H, A is —O—, and Q is O in any of the compounds, compositions, and methods of the invention.

In certain embodiments, X is —F, R₁ and R₂ are both H, A is —S—, and Q is O in any of the compounds, compositions, and methods of the invention.

In certain embodiments, X is —F, R₁ and R₂ are both H, A is —SO₂—, and Q is O in any of the compounds, compositions, and methods of the invention.

In certain embodiments, X is —F, R₁ and R₂ are both H, A is >NH, and Q is O in any of the compounds, compositions, and methods of the invention.

In certain embodiments, X is —F, R₁ and R₂ are both H, A is >NCH₃, and Q is O in any of the compounds, compositions, and methods of the invention.

In certain preferred embodiments, Y is a 5- to 6-membered non aromatic heterocycle, R₁ and R₂ are both H, A is —O—, and Q is O in any of the compounds, compositions, and methods of the invention.

In certain preferred embodiments, Y is a 5- to 6-membered non aromatic heterocycle, R₁ and R₂ are both H, A is —S—, and Q is O in any of the compounds, compositions, and methods of the invention.

In certain preferred embodiments, Y is a 5- to 6-membered non aromatic heterocycle, R₁ and R₂ are both H, A is —SO₂—, and Q is O in any of the compounds, compositions, and methods of the invention.

In certain preferred embodiments, Y is a 5- to 6-membered non aromatic heterocycle, R₁ and R₂ are both H, A is >NH, and Q is O in any of the compounds, compositions, and methods of the invention.

In certain preferred embodiments, Y is a 5- to 6-membered non aromatic heterocycle, R₁ and R₂ are both H, A is >NCH₃, and Q is O in any of the compounds, compositions, and methods of the invention.

In certain preferred embodiments, X is —F, Y is a 5- to 6-membered non aromatic heterocycle, R₁ and R₂ are both H, A is —O—, and Q is O in any of the compounds, compositions, and methods of the invention.

In certain preferred embodiments, X is —F, Y is a 5- to 6-membered non aromatic heterocycle, R₁ and R₂ are both H, A is —S—, and Q is O in any of the compounds, compositions, and methods of the invention.

In certain preferred embodiments, X is —F, Y is a 5- to 6-membered non aromatic heterocycle, R₁ and R₂ are both H, A is —SO₂—, and Q is O in any of the compounds, compositions, and methods of the invention.

In certain preferred embodiments, X is —F, Y is a 5- to 6-membered non aromatic heterocycle, R₁ and R₂ are both H, A is >NH, and Q is O in any of the compounds, compositions, and methods of the invention.

In certain preferred embodiments, X is —F, Y is a 5- to 6-membered non aromatic heterocycle, R₁ and R₂ are both H, A is >NCH₃, and Q is O in any of the compounds, compositions, and methods of the invention.

In some embodiments, the compound of Formula (Ia) has the following structure:

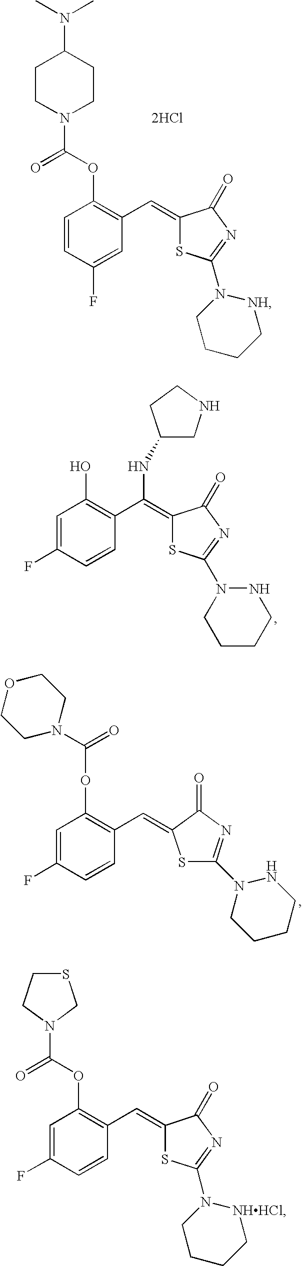

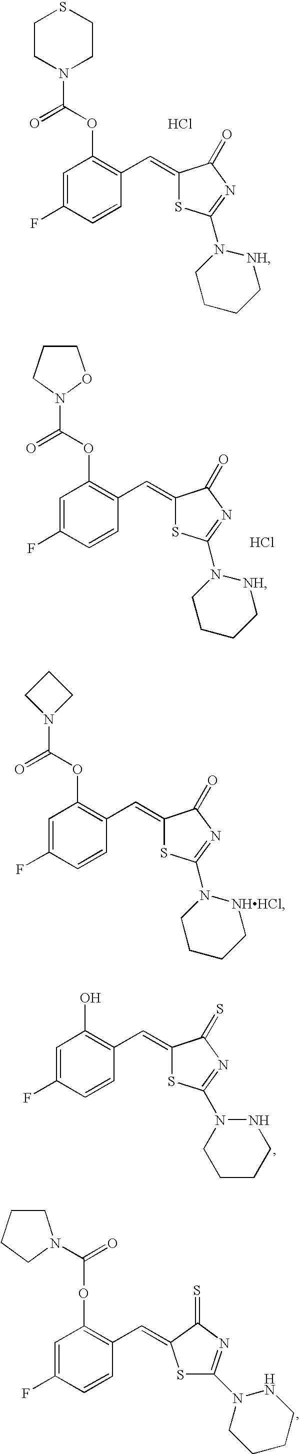

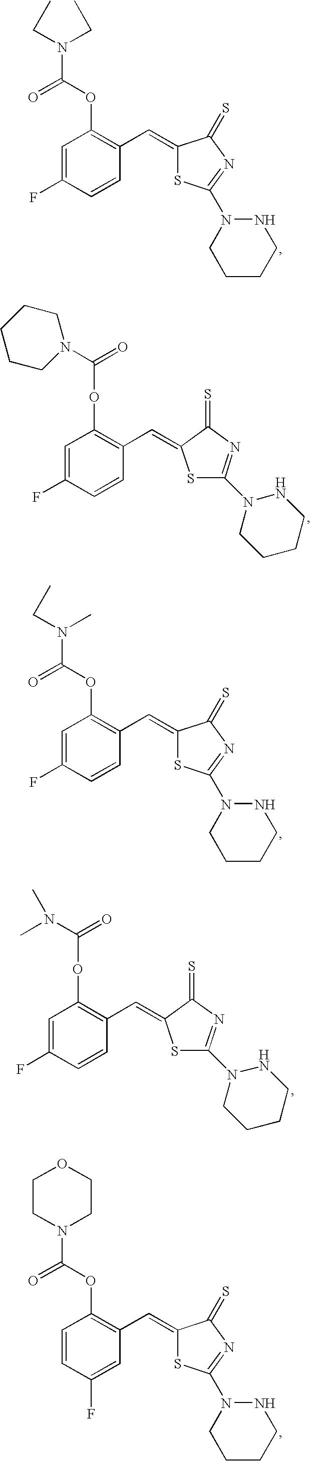

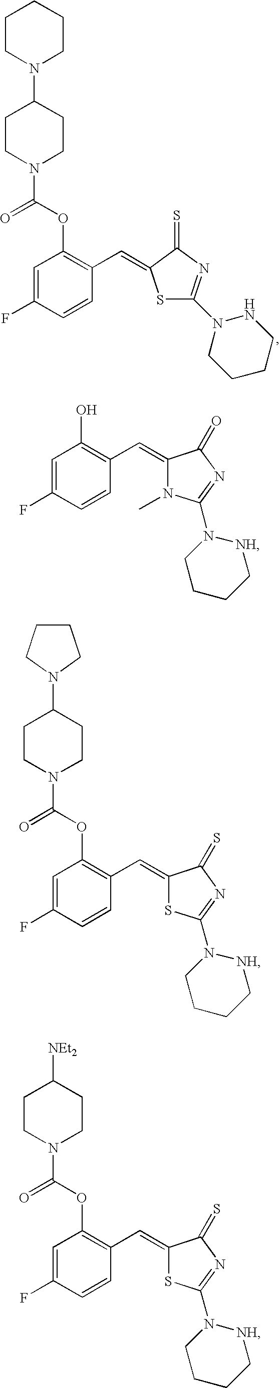

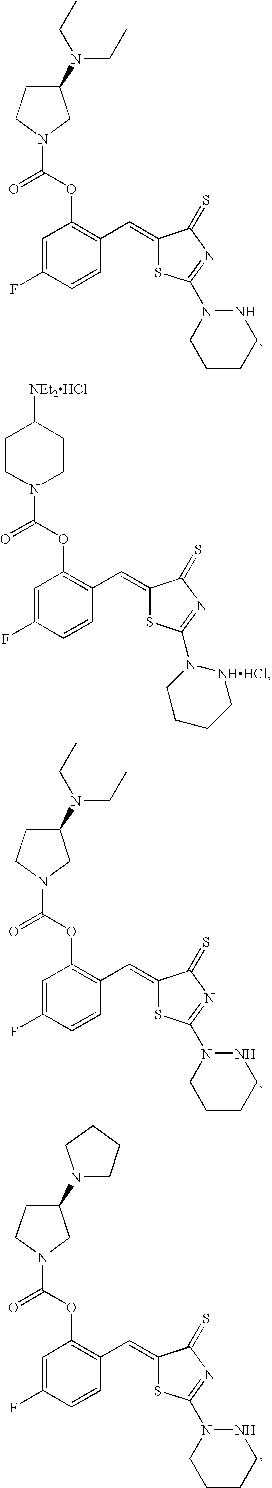

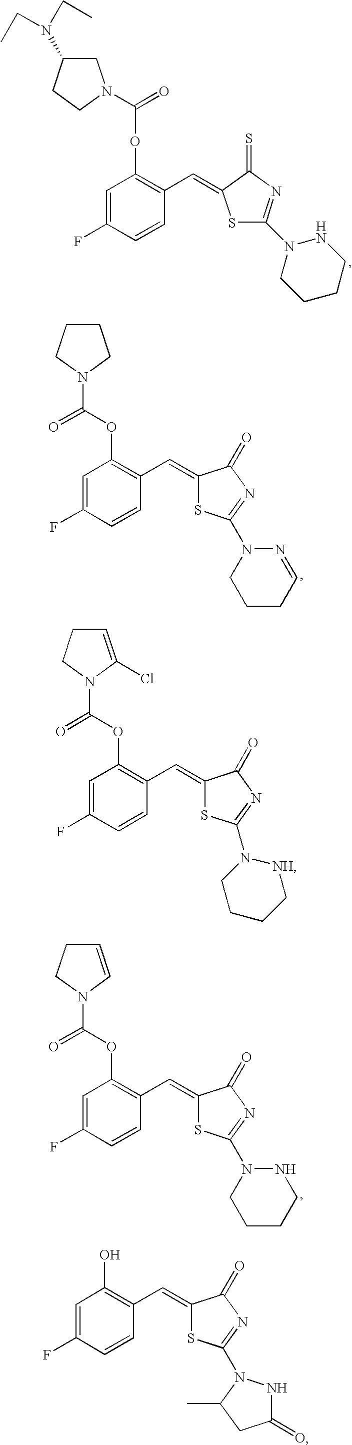

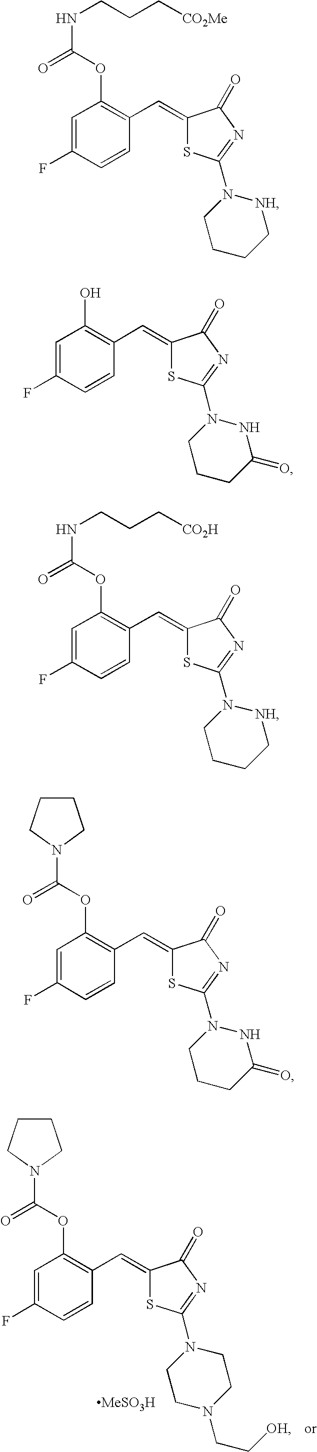

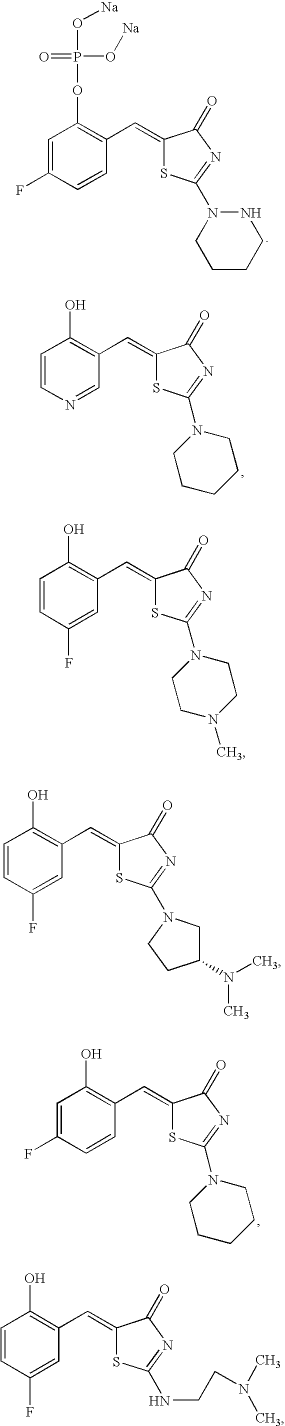

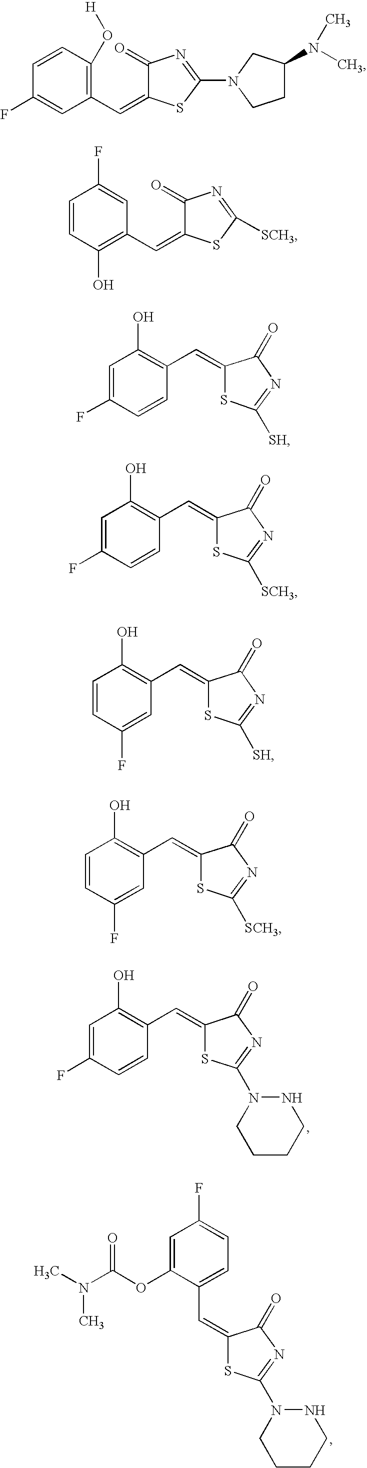

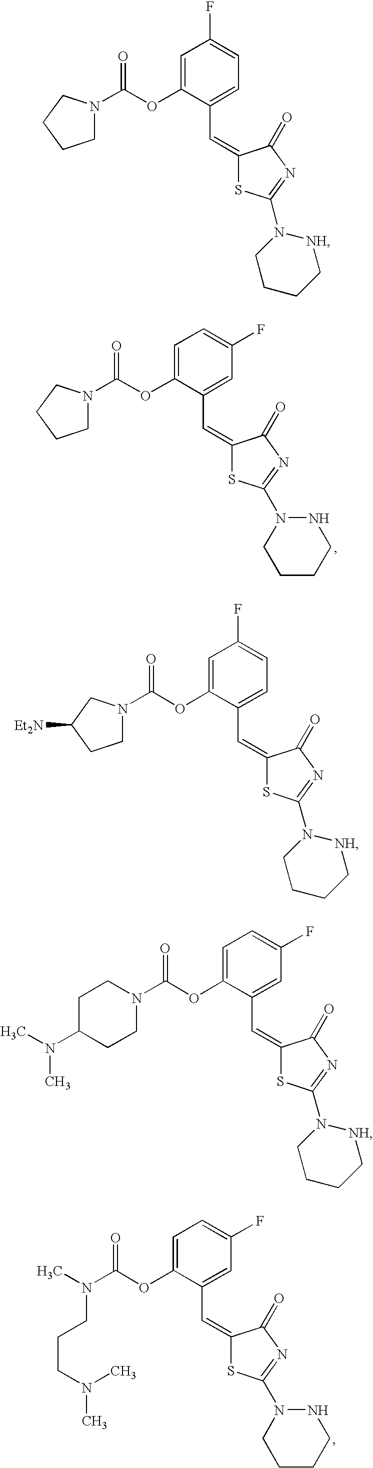

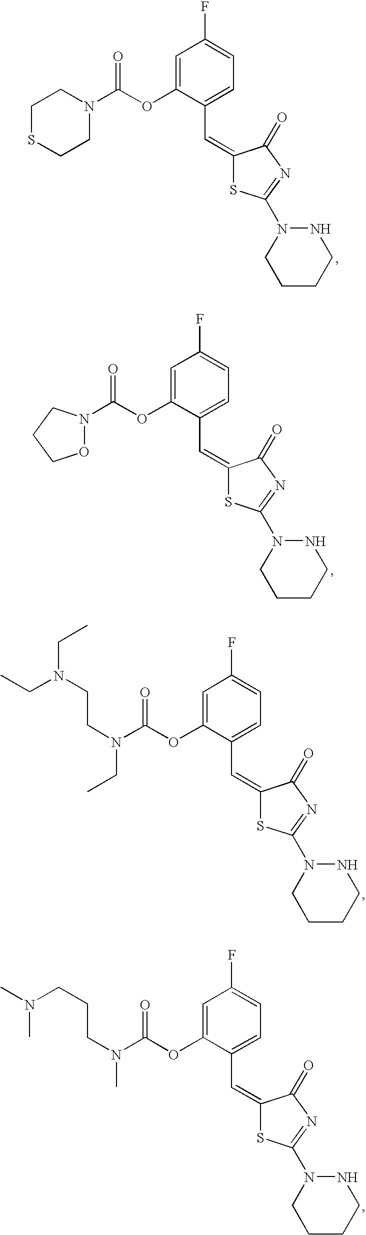

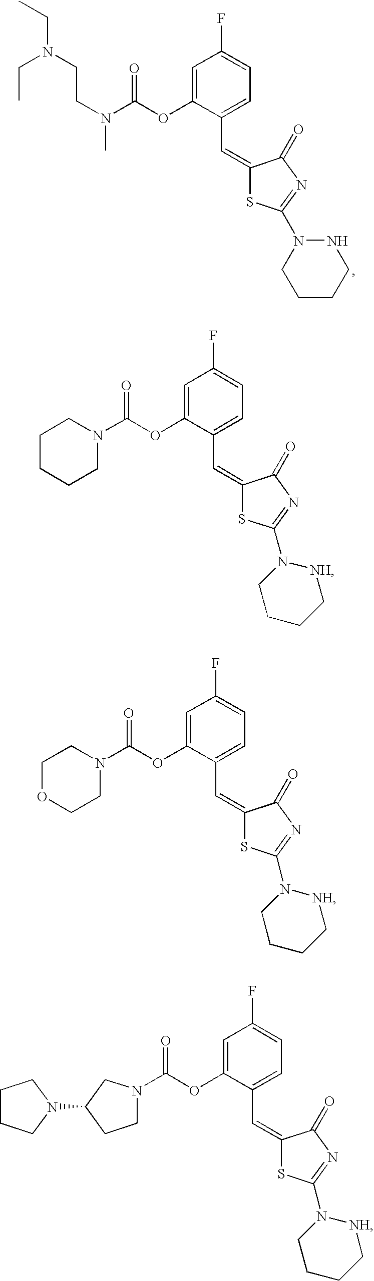

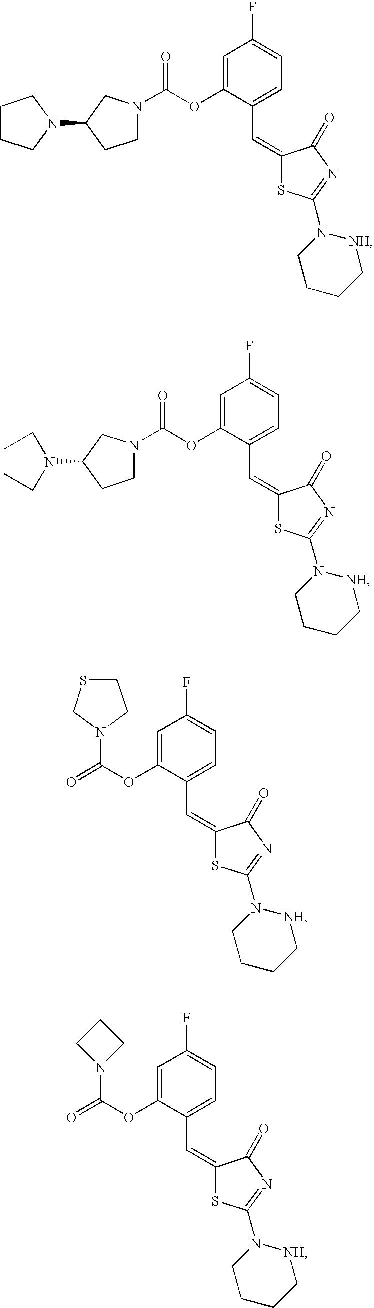

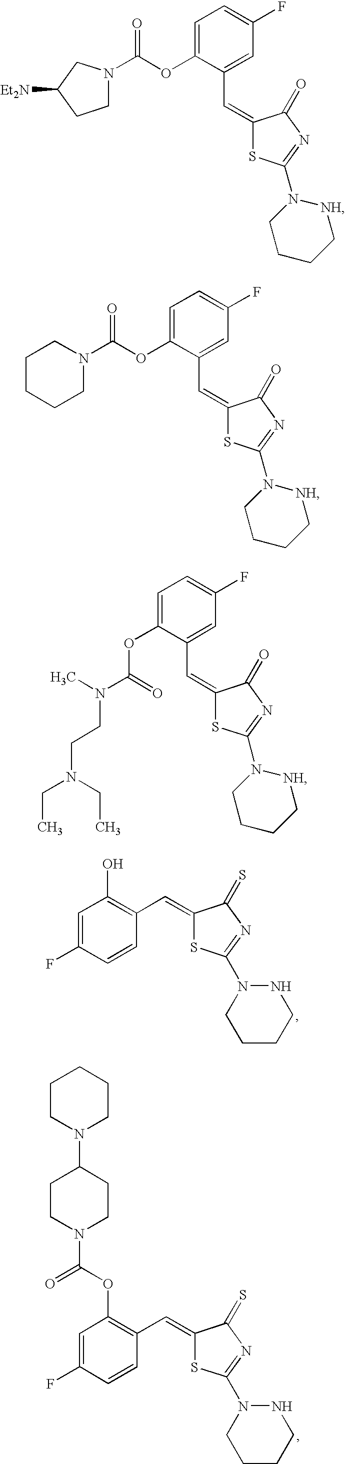

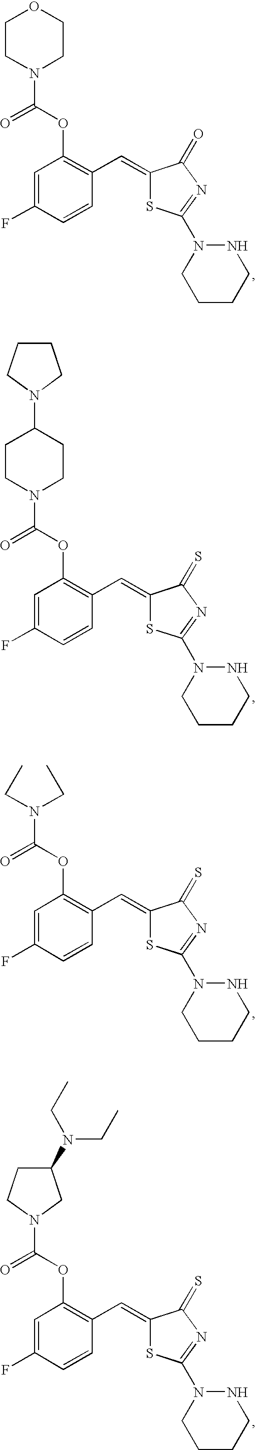

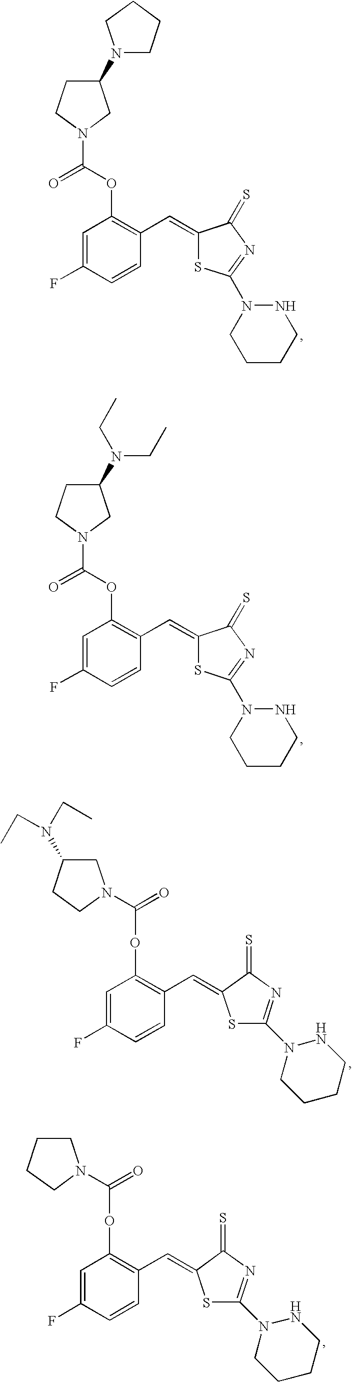

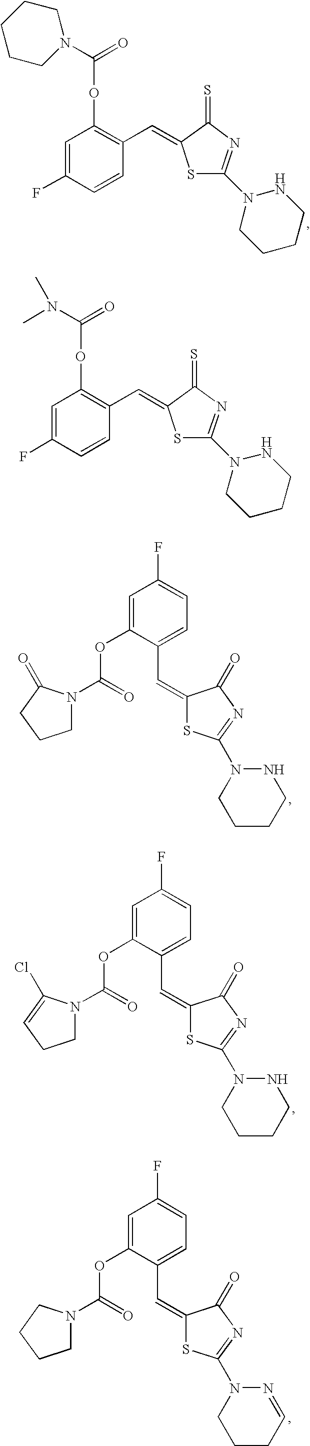

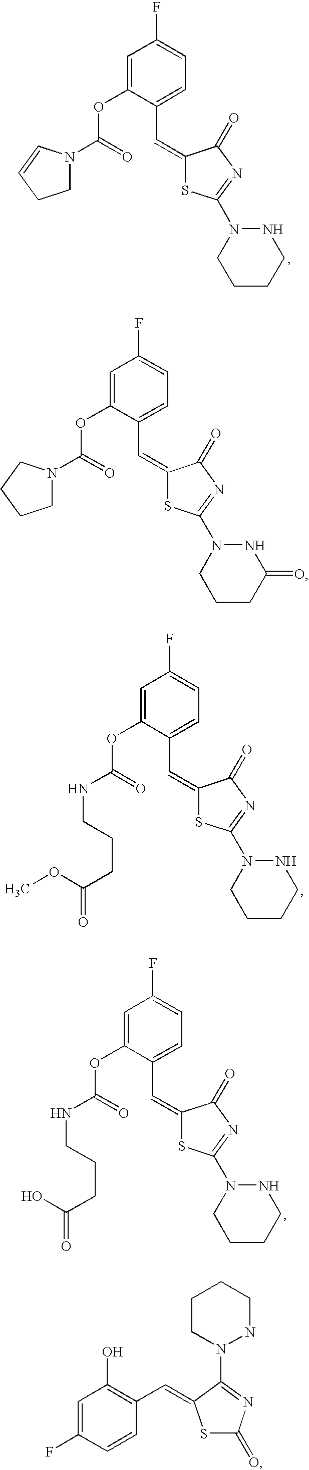

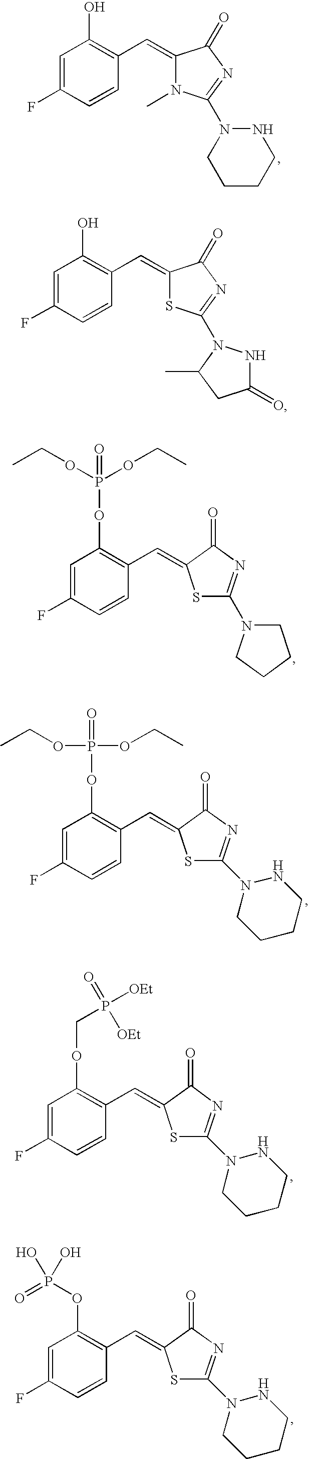

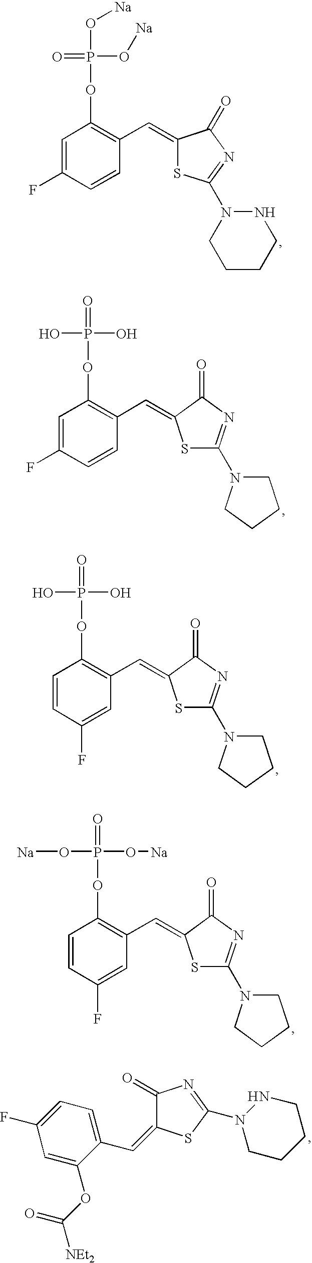

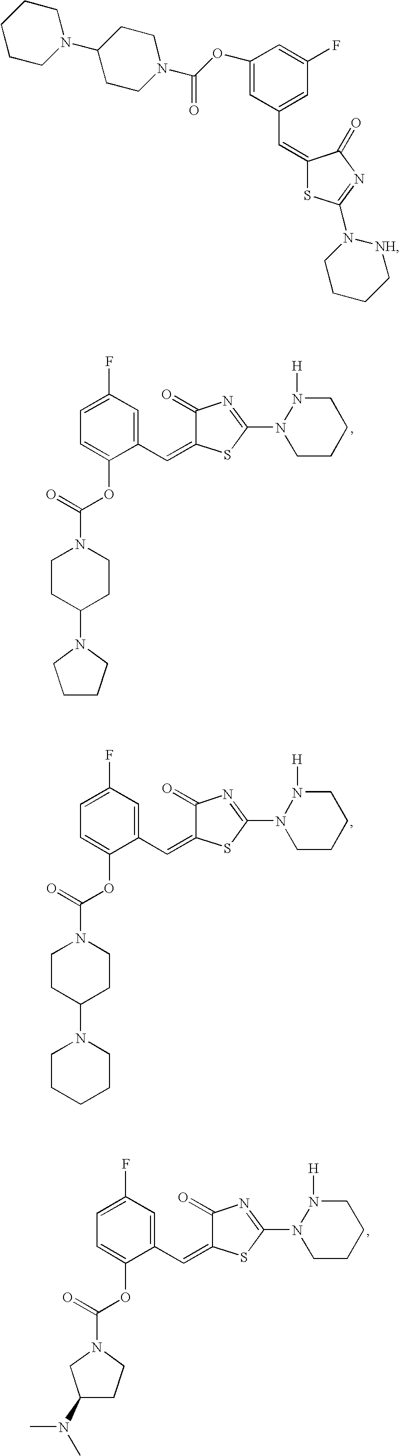

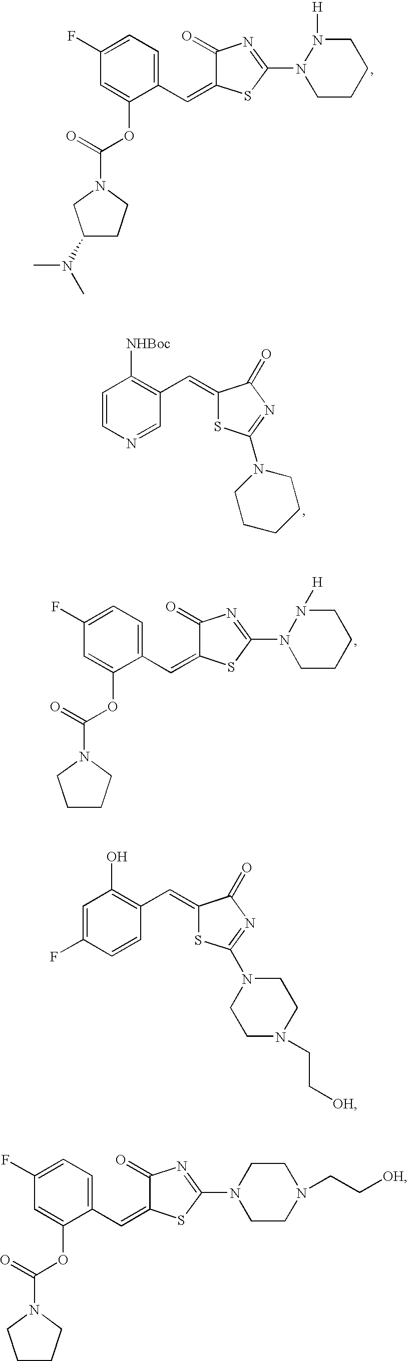

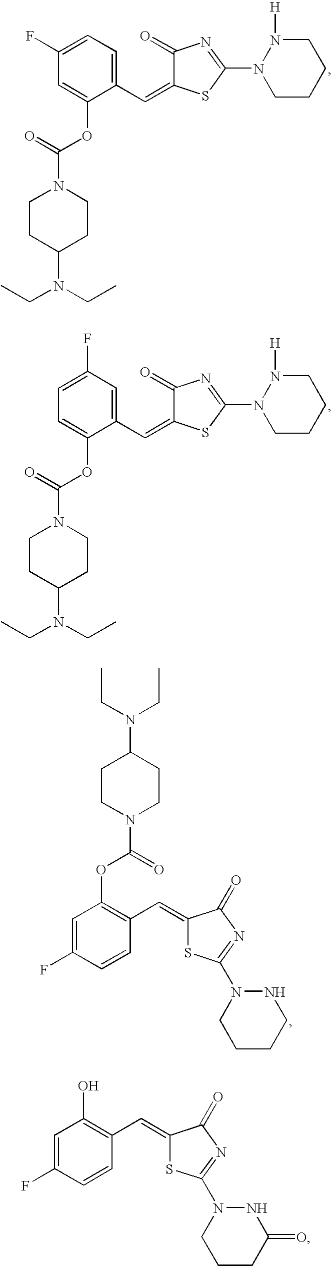

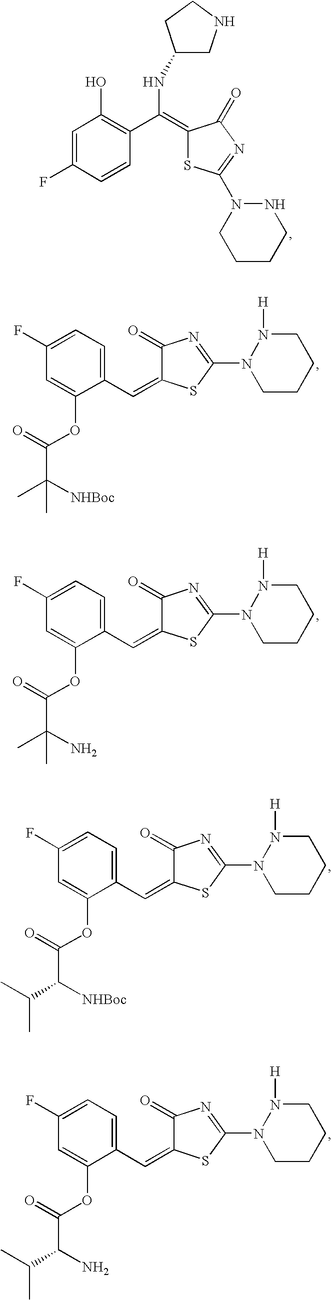

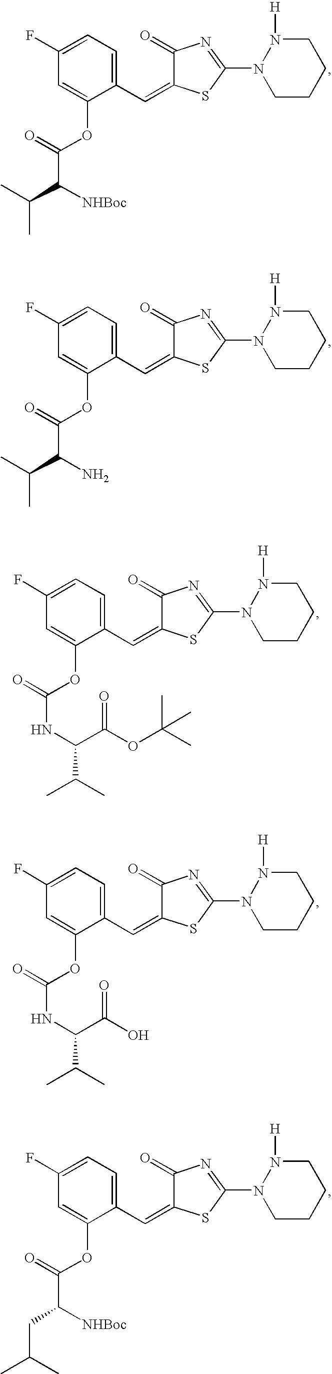

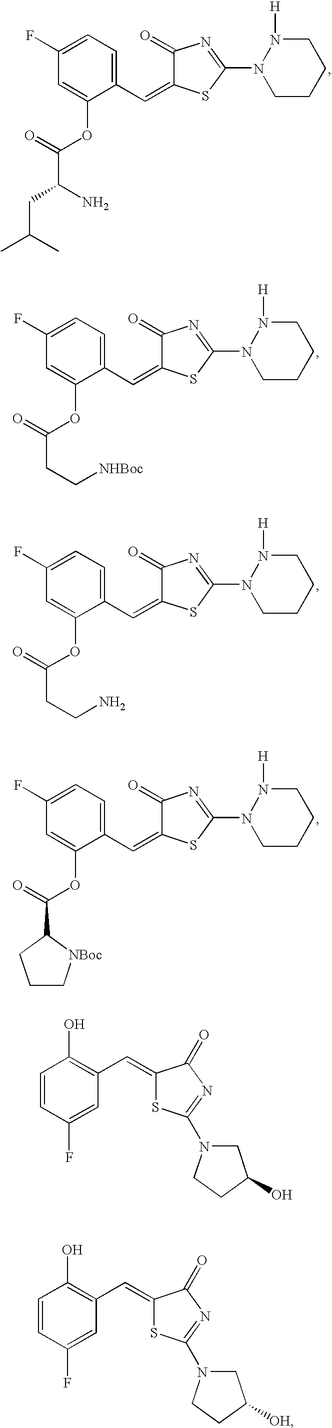

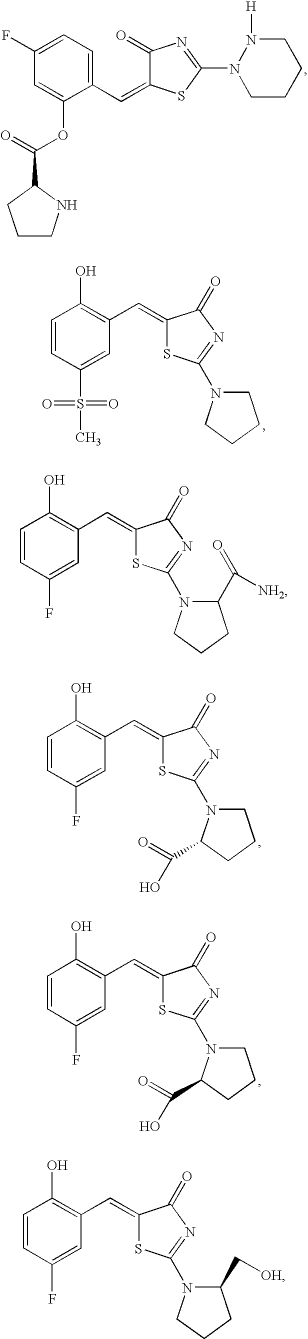

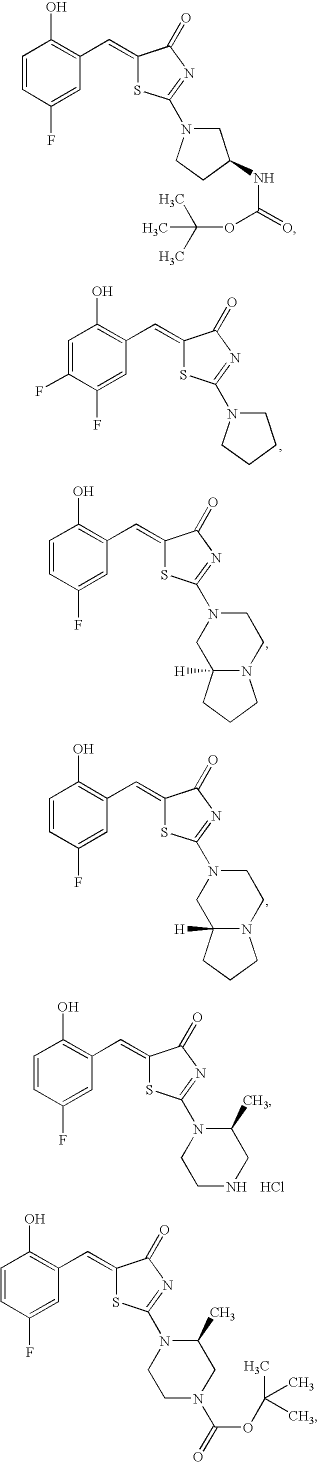

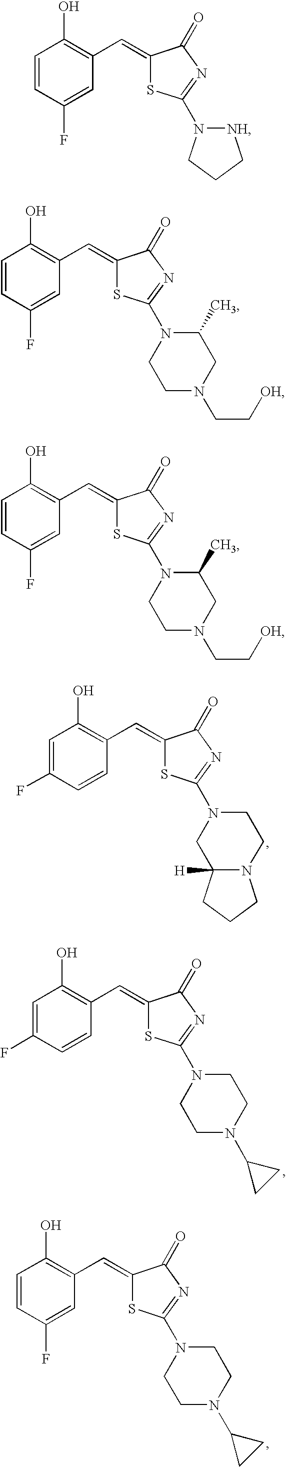

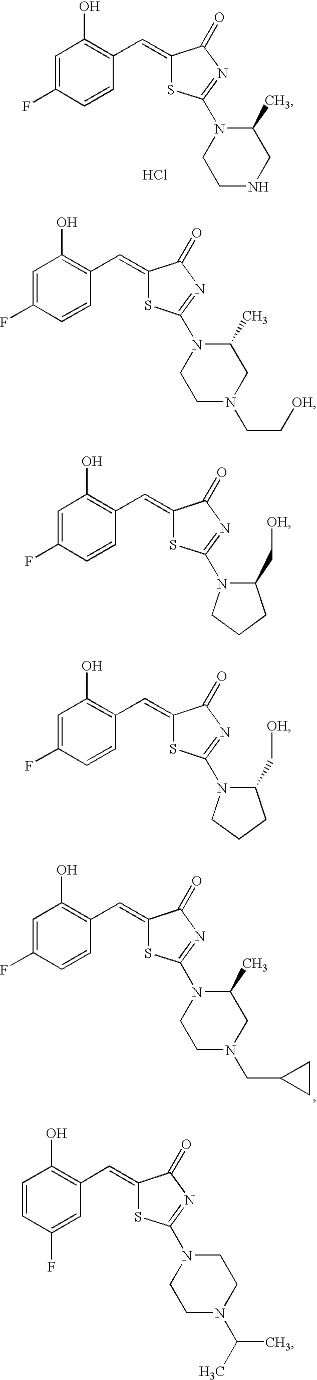

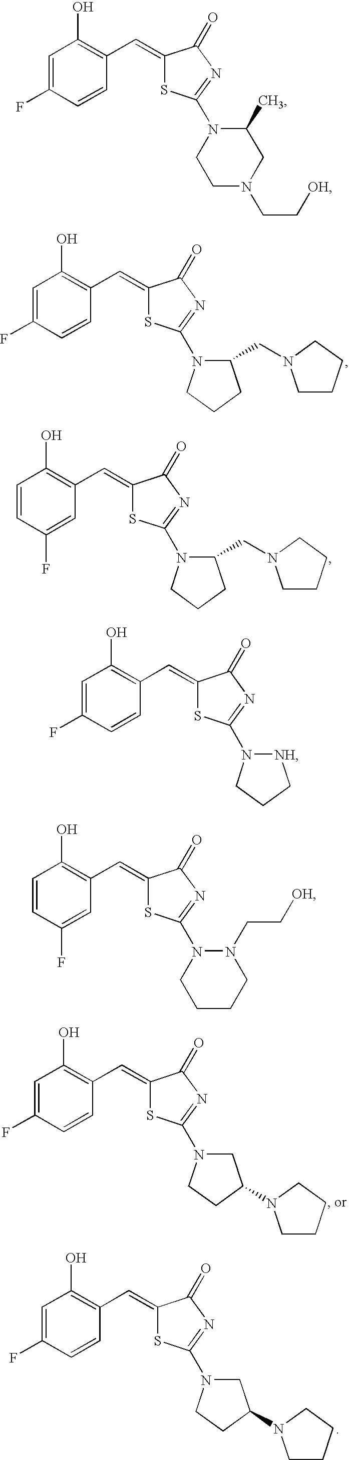

In some embodiments, the compound is

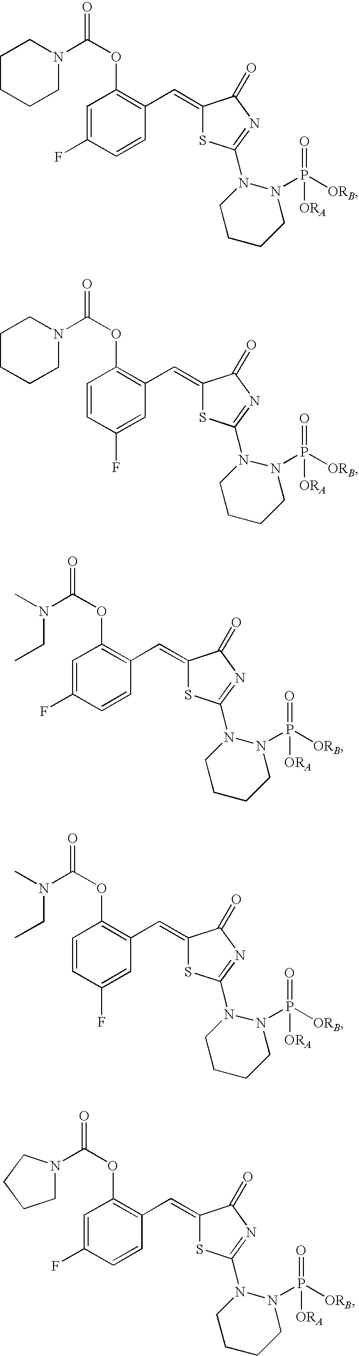

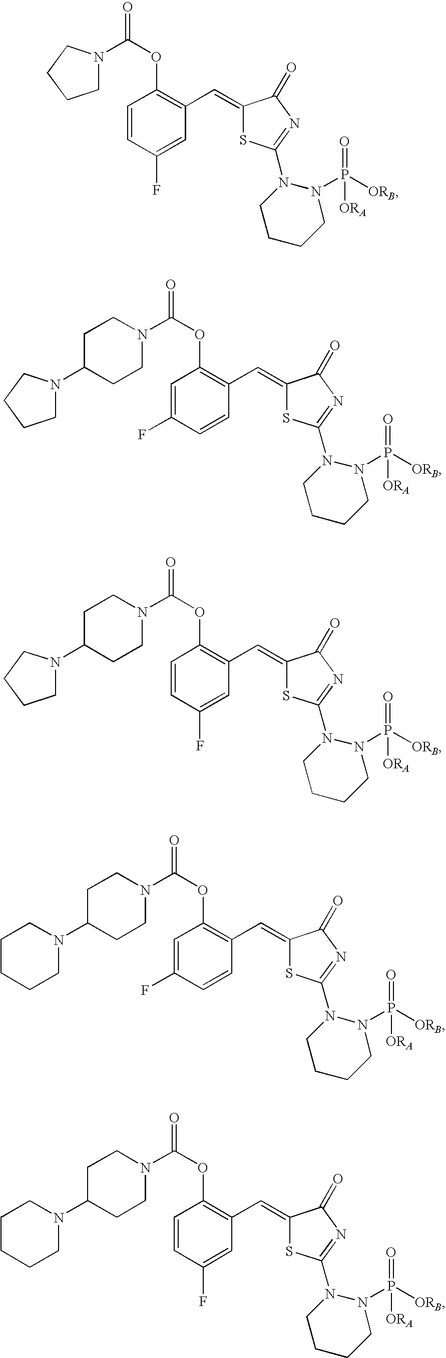

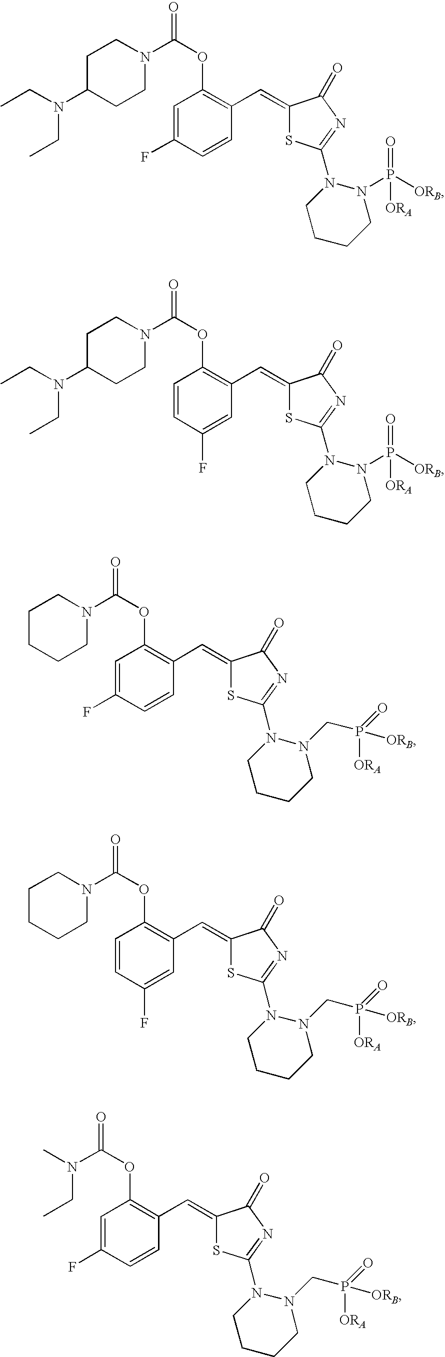

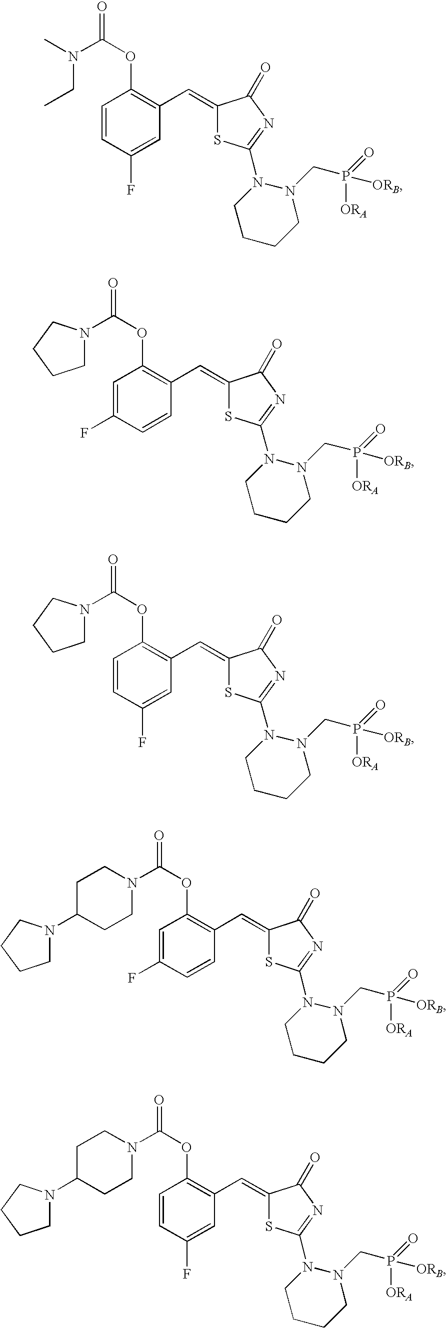

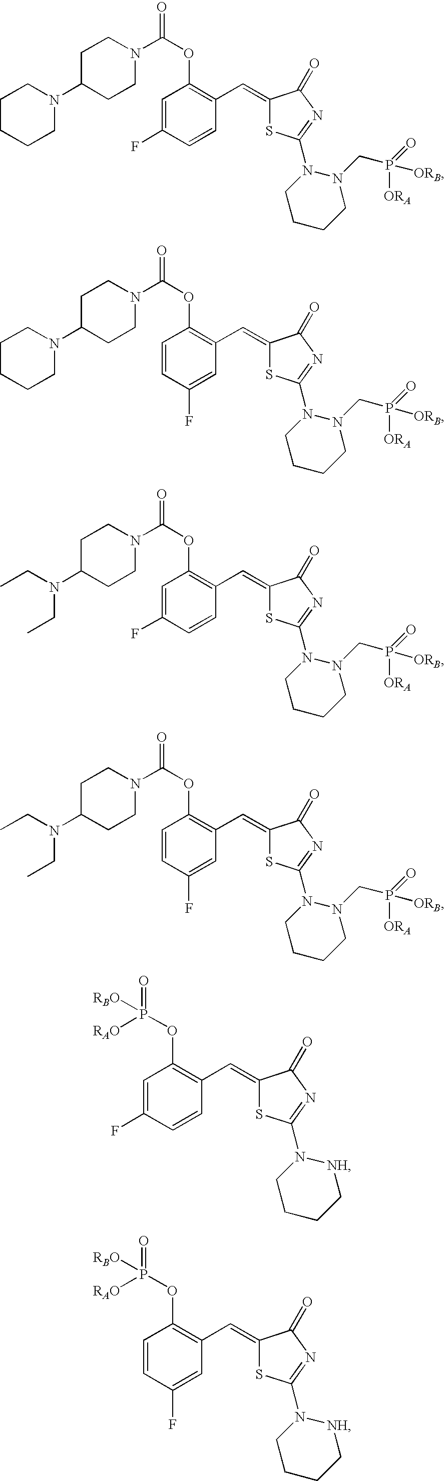

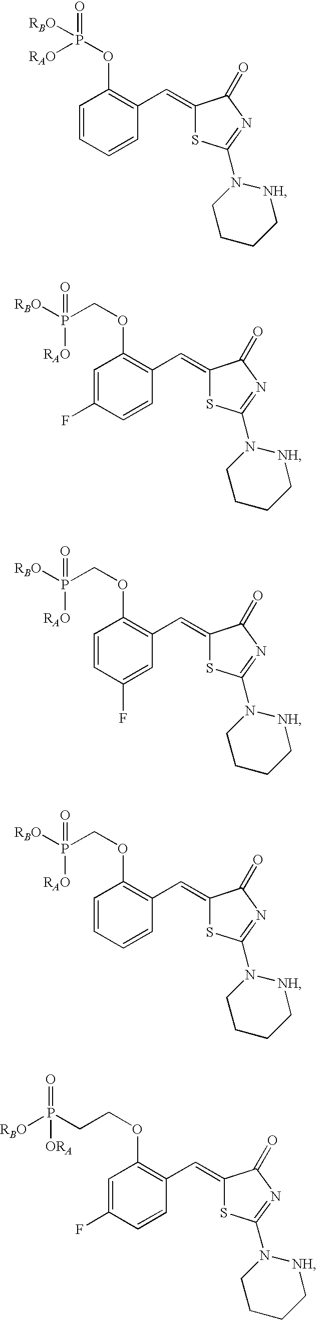

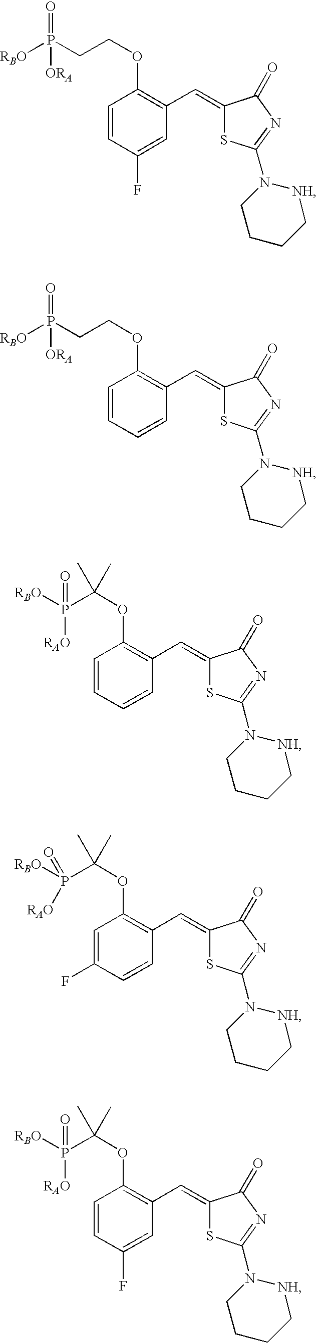

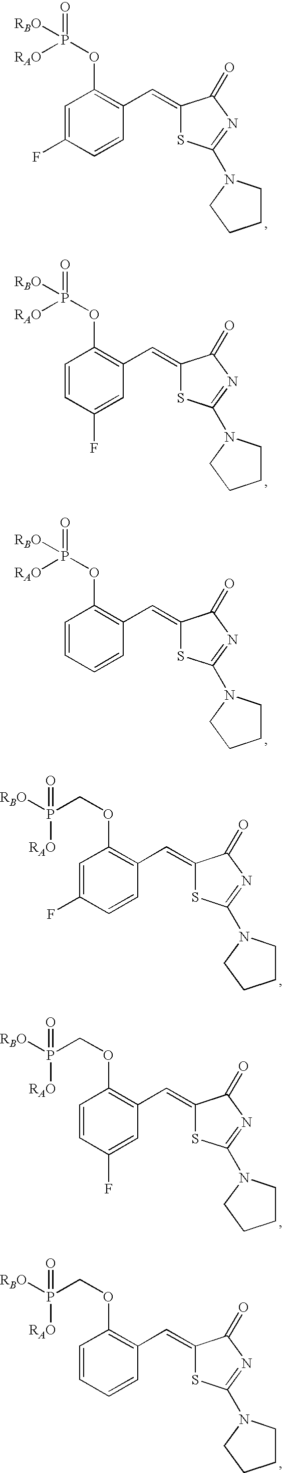

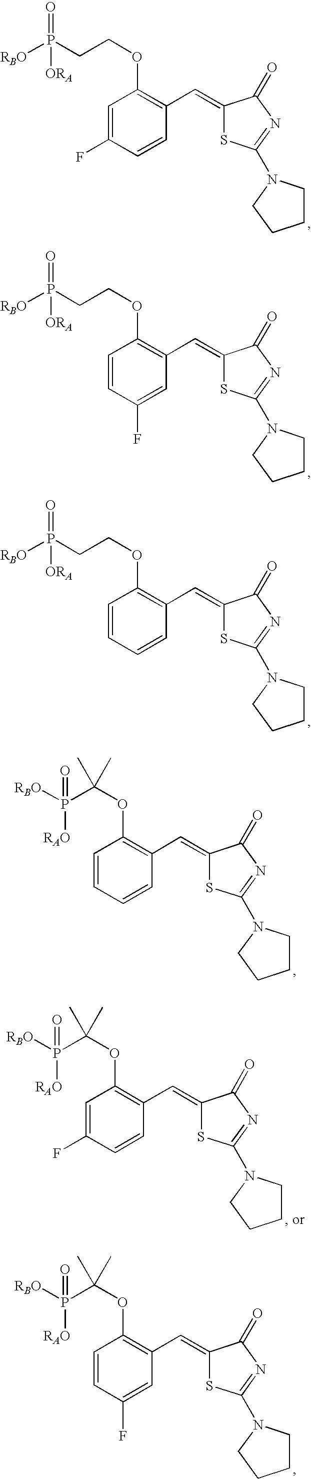

where each R_{A} and K_{B} is selected, independently, from H or optionally substituted C₁₋₅ alkyl, or R_{A} and R_{B} combine to form an optionally substituted 5-7 membered ring.

In other embodiments, the compound has a structure selected from:

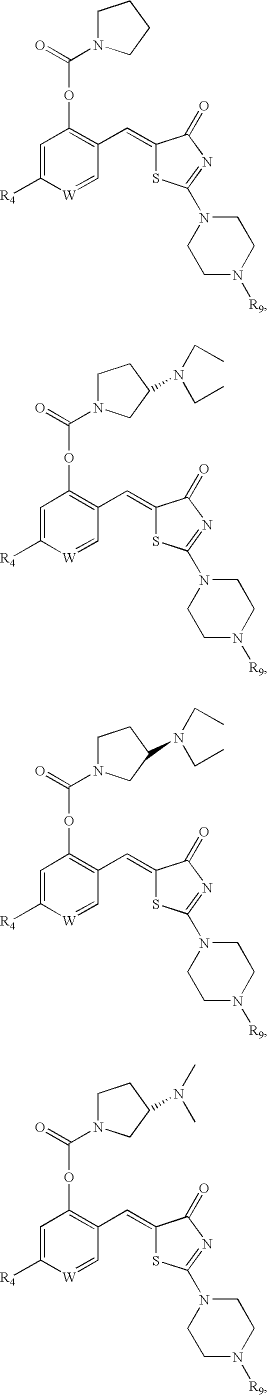

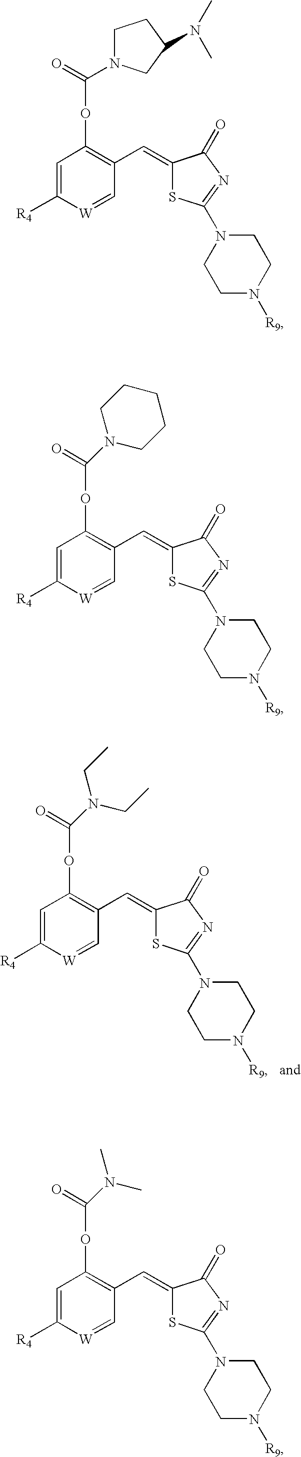

wherein, independently, W is CH or CF, R₄ is —H or —F, and R₉ is —C₁-C₃ alkyl that is optionally substituted with one —OH group.

In another aspect, the invention provides compositions including a pharmaceutically acceptable carrier or vehicle and an effective amount of a compound having the Formula (Ia).

In another aspect, the invention provides methods for treating or preventing pain (e.g., neuropathic pain) in a patient by administering to the patient in need thereof an effective amount of a compound of Formula (Ia).

In still another aspect, the invention provides methods for treating or preventing inflammation in a patient by administering to the patient in need thereof an effective amount of a compound of Formula (Ia).

In all of the compositions and methods of the invention, it is understood that stereoisomers and prodrugs of the structures of Formula (Ia), and pharmaceutically acceptable salts thereof, are encompassed by the invention. In some embodiments, the compound of Formula (Ia) has the Z-configuration. In other embodiments, the compound of Formula (Ia) has the E-configuration. In still other embodiments, the compound includes a mixture of EIZ isomers.

In another aspect, the invention features a method for treating or preventing pain (e.g., neuropathic pain) in a patient, comprising administering to a patient in need thereof by administering to the patient in need thereof an effective amount of a compound of Formula (Ib),

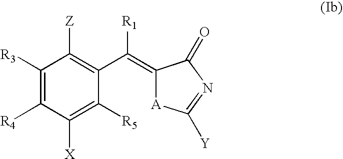

including stereoisomers, E/Z stereoisomers, prodrugs and pharmaceutically acceptable salts thereof, wherein:

A is —O—, —S—, —SO—, —SO₂—, >NR₆, or >NC(O)R₆;

Q is O, S, or NR₆;

Z is halogen, —NO₂, —OR₂, —N(R₆)₂, —C(O)R₆, or —C(O)(C(R₆)₂)ₒNH₂;

X is H, Br, I, OCH₃, NO₂, —C₆-C₁₂ aryl, —C₇-C₁₄ arylalkyl, N-terminal linked amino acid, or C-terminal linked amino acid;

Y is —C₃-C₈ cycloalkyl, —C₆-C₁₂ aryl, —C₇-C₁₄ arylalkyl, 3 to 9-membered heterocycle, —N(R₆)₂, —NHC(O)R₆, —NHS(O)₂R₆, —NHC(NH)N(R₆)₂, —NR₆C(NH)N(R₆)₂, —NHC(NCN)N(R₆)₂, or —NR₆C(NCN)N(R₆)₂;

R₁ is —H, halogen, —C₁-C₈ alkyl, —C₂-C₈ alkenyl, or —C₂-C₈ alkynyl;

R₂ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, —(CH₂)ₙOR₆, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid;

R₃, R₄, and R₅ are each, independently, —H, —OH, halogen, —CN, —NO₂, —SH, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3 to 9-membered aromatic or non aromatic heterocycle, —OR₆, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —SOR₆, —S(O)₂R₆, —NHC(O)R₆, —NHS(O)₂R₆, —NHC(NH)N(R₆)₂, —NR₆C(NH)N(R₆)₂, —NHC(NCN)N(R₆)₂, —NR₆C(NCN)N(R₆)₂, or —PO(OR₆)₂, or R₃ and R₄, together with the carbon atom to which each is attached, join to form a 5- to 6-membered aromatic or non aromatic carbocycle or heterocycle;

each R₆ is, independently, —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic carbocycle or heterocycle;

n is 1 or 2;

o is an integer between 0-3;

r2 is an integer between 1-3; and

r3 is an integer between 0-2.

In some embodiments, Z is halogen, —NO₂, —OR₂, or —N(R₆)₂; X is H, Br, I, OCH₃, NO₂, —C₆-C₁₂ aryl, or —C₇-C₁₄ arylalkyl; and R₂ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, —(CH₂)ₙOR₆, —C(O)R₆, —C(O)OR₆, —C(O)NHR₆, —C(O)N(R₆)₂, or —PO(OR₆)₂.

In some embodiments, the compound of Formula (Ib) has the following structure

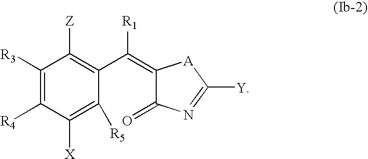

In another aspect, the invention features a method for treating inflammation in a patient, by administering to the patient in need thereof an effective amount of a compound of Formula (Ib) as described herein, including stereoisomers, E/Z stereoisomers, prodrugs and pharmaceutically acceptable salts thereof.

In any of the methods described herein, the compound of Formula (Ib) has the structure selected from the group consisting of:

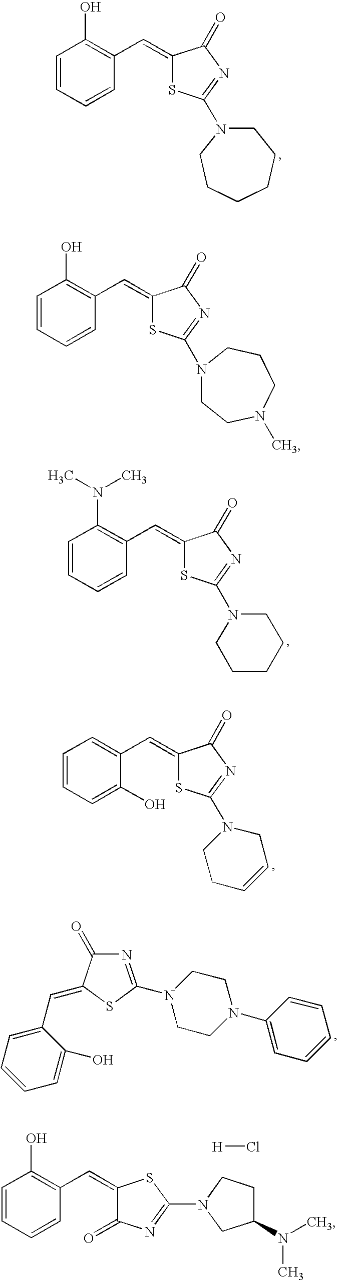

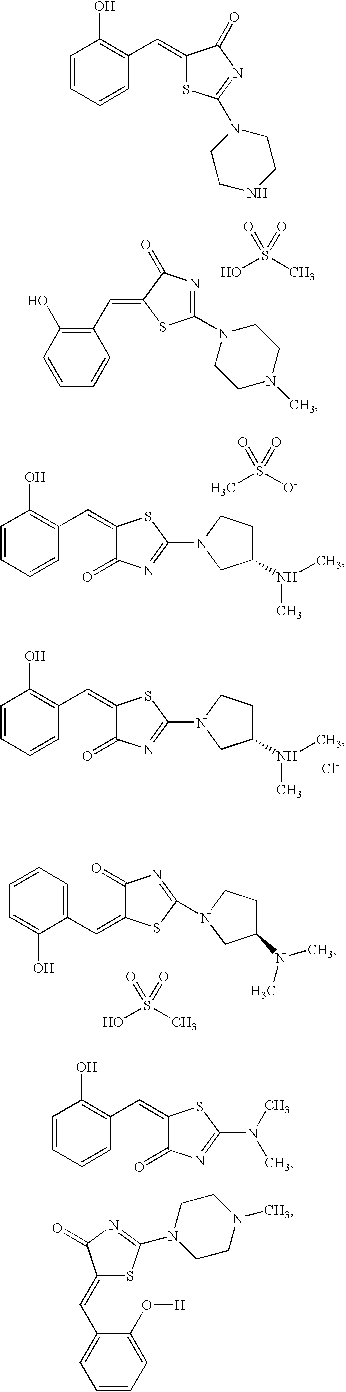

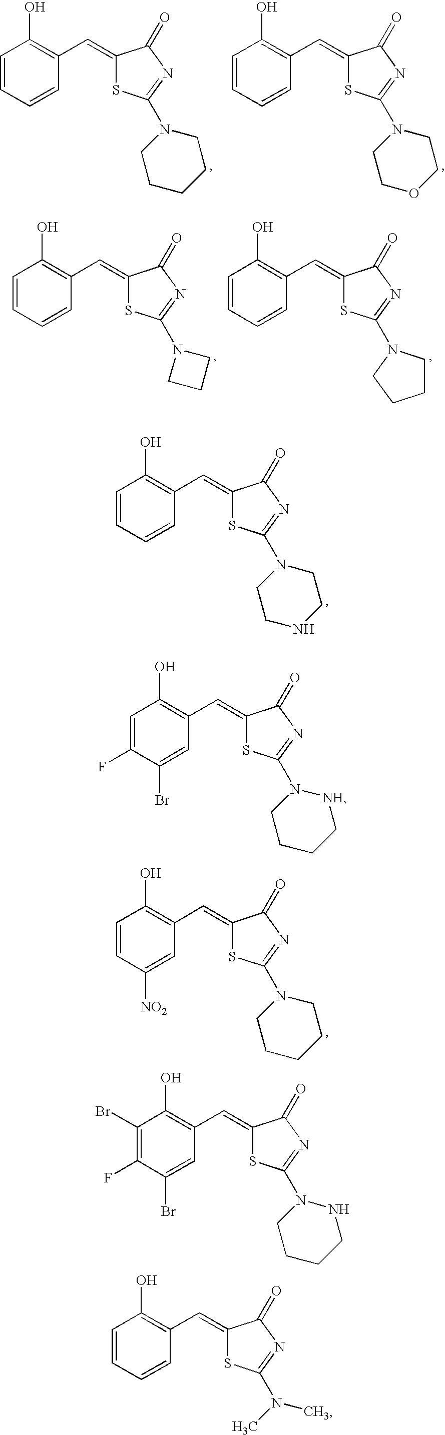

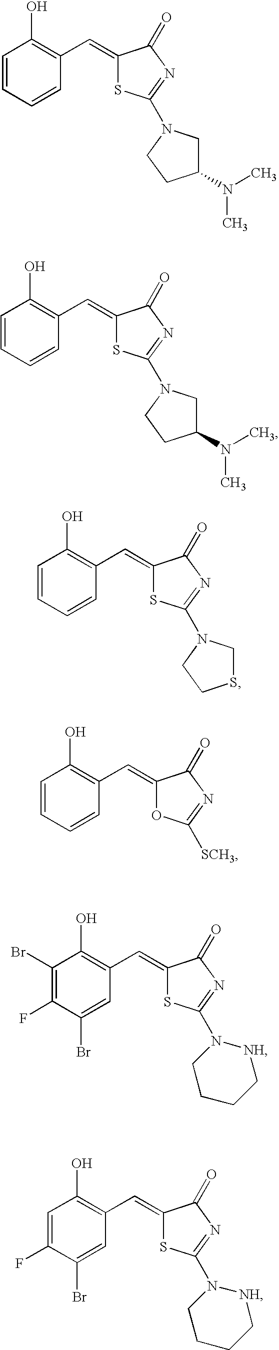

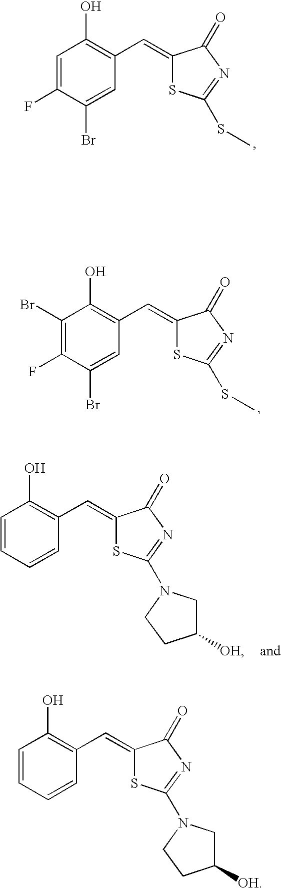

As used herein, it is understood that stereoisomers and prodrugs of the structures of Formula (Ib), and pharmaceutically acceptable salts thereof, are encompassed by the invention. In some embodiments, the compound of Formula (Ib) has the Z-configuration. In other embodiments, the compound of Formula (Ib) has the E-configuration. In still other embodiments, the compound includes a mixture of E/Z isomers.

In any of the compounds, compositions, and methods of the invention, where a compound, e.g., a compound of Formula (Ia) or (Ib) is depicted as a salt, the invention also includes the free acid or base, and vice versa.

### DEFINITIONS AND ABBREVIATIONS

As used herein, “aldehyde” refers to a carboxyl group having the structure represented by —CH(O).

As used herein, “Cₓ-alkyl” refers to an optionally substituted alkyl group containing x carbons where x is an integer ranging between 1 and 8. Exemplary values of x are 1, 2, 3, 4, 5, 6, 7, and 8.

As used herein, “Cₓ-C_{y} alkyl” refers to an optionally substituted straight or branched chain saturated hydrocarbon group containing x-y carbon atoms, wherein x is an integer 1 and 8 and y is an integer less than or equal to 8.

As used herein, “C₁-C₈ alkyl” or “alkyl” refers to a straight or branched chain saturated hydrocarbon group containing 1-8 carbon atoms, which can be unsubstituted or optionally substituted with one or more -halogen, —NH₂, NH(C₁-C₈ alkyl), N(C₁-C₈ alkyl)₂, —OH, —O—(C₁-C₈ alkyl), or C₆-C₁₀ aryl groups such as phenyl or naphthyl groups. As used herein, “C₂-C₈ alkyl” refers to a straight or branched chain saturated hydrocarbon group containing 2-8 carbon atoms, which can be unsubstituted or optionally substituted with one or more -halogen, —NH₂, —OH, —O—(C₁-C₈ alkyl), phenyl or naphthyl groups. Examples of C₁-C₈ or C₂-C₈ straight or branched chain alkyl groups include, but are not limited to, methyl, trifluoromethyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, 1-heptyl and 1-octyl.

As used herein, “C₁-C₅ alkyl” refers to an optionally substituted straight or branched chain saturated hydrocarbon group containing 1-5 carbon atoms. As used herein, “C₂-C₅ alkyl” refers to an optionally substituted straight or branched chain saturated hydrocarbon group containing 2-5 carbon atoms. Examples of C₁-C₅ or C₂-C₅ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, and 1-pentyl.

As used herein, “C₁-C₈ alkylene” refers to an optionally substituted C₁-C₈ alkyl group in which one of the C₁-C₈ alkyl group's hydrogen atoms has been replaced with a bond.

As used herein, “Cₓ-alkenyl” refers to an optionally substituted alkenyl group containing x carbons where x is an integer ranging between 2 and 8. Exemplary values of x are 2, 3, 4, 5, 6, 7, and 8.

As used herein, “alkenyl” or “C₂-C₈ alkenyl” refers to an optionally substituted unsaturated, straight or branched chain hydrocarbon group containing 2-8 carbon atoms and at least one carbon-carbon double bond that can be optionally substituted with a phenyl or naphthyl group.

As used herein, “C₂-C₅ alkenyl” refers to an optionally substituted unsaturated, straight or branched chain hydrocarbon group containing 2-5 carbon atoms and at least one carbon-carbon double bond that can be optionally substituted with a phenyl or naphthyl group.

As used herein, “C₂-C₈ alkenylene” refers to an optionally substituted C₂-C₈ alkenyl group in which one of the C₂-C₈ alkenyl group's hydrogen atoms has been replaced with a bond.

As used herein, “alkoxy” refers to a group having the structure OR², wherein R² is selected from —C₁-C₈ alkyl, —C₂-C₈ alkenyl, —C₂-C₈ alkynyl, —C₃-C₁₂ cycloalkyl, —C₆-C₁₂ aryl, or —C₇-C₁₄ arylalkyl.

As used herein, “Cₓ-alkynyl” refers to an optionally substituted alkynyl group containing x carbons where x is an integer ranging between 2 and 8. Exemplary values of x are 2, 3, 4, 5, 6, 7, and 8.

As used herein, “alkynyl” or “C₂-C₈ alkynyl” refers to an optionally substituted unsaturated, straight or branched chain hydrocarbon group containing 2-8 carbon atoms and at least one carbon-carbon triple bond that can be unsubstituted or optionally substituted. Exemplary substituents on the carbon-carbon triple bond are phenyl or naphthyl.

As used herein, “C₂-C₅ alkynyl” refers to an optionally substituted unsaturated, straight or branched chain hydrocarbon group containing 2-5 carbon atoms and at least one carbon-carbon triple bond that can be unsubstituted or optionally substituted with a phenyl or naphthyl group.

As used herein, “C₂-C₈ alkynylene” refers to an optionally substituted C₂-C₈ alkynyl group in which one of the C₂-C₈ alkynyl group's hydrogen atoms has been replaced with a bond.

As used herein, “amido” refers to a group having a structure selected from —N(R₆)₂, wherein each R₆ is selected from —C(O)R₆ₐ, —C(O)NR₆ₐR₇ₐ, —C(O)OR₆ₐ and, independently, R₆ₐ, R₇, and R₇ₐ are selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, —C₂-C₈ alkynyl, or two R₆ or R₆ₐ and R₇ₐ, together with the atom to which each is attached, join to form a 3- to 7-membered aromatic or non aromatic carbocycle or heterocycle

As used herein, “amino” refers to a group having the structure —NR₆R₇ wherein R₆ and R₇ are selected, independently, from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl.

As used herein, “amino acid” refers to a molecular fragment comprising an amino functional group and a carboxylic functional group. Amino acids include natural amino acids and unnatural amino acids, as defined herein. Types of amino acids include “α-amino acids,” wherein the amino and carboxylic groups are attached to the same carbon. In “β-amino acids,” the carbon to which the amino group is attached is adjacent to the carbon to which the carboxylic group is attached, and in “γ-amino acids,” there is an additional intervening carbon. Amino acids can have the L-configuration (for example, natural amino acids have the L-configuration) or the D-configuration. An amino acid can be attached to a compound of the invention through a covalent attachment to, for example, the carboxylic functional group (“C-linked”) or through the amino functional group (“N-linked”).

As used herein, “aromatic” refers to a cyclic ring system having (4n+2) π electrons in conjugation where n is 1, 2, or 3.

As used herein, “aromatic carbocyclic” refers to an aryl group.

As used herein, “Cₓ aryl” refers to an optionally substituted aryl group having x carbons wherein x is an integer between 6-12. Exemplary values for x are 6, 7, 8, 9, 10, 11, and 12.

As used herein, “aryl” or “C₆-C₁₂ aryl” refers to an optionally substituted monocyclic or bicyclic structure wherein all rings are aromatic and the rings are formed by carbon atoms. Exemplary aryl groups include phenyl and naphthyl. Where an aryl group is substituted, substituents can include, for example, one or more C₁₋₈ alkyl groups or a phosphorus (V) containing group. Exemplary phosphorus (V) containing groups include —(CH₂)ₙPO(OR₆R₇), wherein n is 0 to 3, —(CHR′)ₙPO(OR₆R₇), wherein n is 0 to 3, and —(C(R′)₂)ₙPO(OR₆R₇), wherein n is 0 to 3.

As used herein, “arylalkyl” or “C₇-C₁₄ arylalkyl” refers to an optionally substituted group having the formula —(Cₓ-alkyl)-(C_{y}-aryl) wherein (x+y) is an integer between 7 and 14 and x is at least 1. Exemplary arylalkyls include benzyl and phenethyl.

Where an arylalkyl group is substituted, substituents can include, for example, one or more C₁₋₈ alkyl groups or a phosphorus (V) containing group. Exemplary phosphorus (V) containing groups include —(CH₂)ₙPO(OR₆R₇), wherein n is 0 to 3, —(CHR′)ₙPO(OR₆R₇), wherein n is 0 to 3, and —(C(R′)₂)ₙPO(OR₆R₇), wherein n is 0 to 3.

As used herein, “carbocycle” refers to an optionally substituted C₃-C₁₂ monocyclic, bicyclic, or tricyclic structure in which the rings are formed by carbon atoms. Carbocycles may be aromatic or may be non-aromatic.

As used herein, “carboxyl” refers to a group having a structure selected from —C(O)R₆, —O—C(O)R₆, —O—C(O)OR₆, —O—C(O)NR₆R₇, —C(O)NR₆R₇, —C(O)OR₆, wherein R₆ and R₇ are independently selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, —C₂-C₈ alkynyl or two R₆, together with the atom to which each is attached, join to form a 3- to 7-membered aromatic or non aromatic carbocycle or heterocycle;

As used herein, “carrier” or “pharmaceutical carrier” refers to a diluent, adjuvant, excipient, or vehicle with which a compound of the invention is administered. Such pharmaceutical carriers can be liquids, such as water and oils, including those of petroleum, animal, vegetable or synthetic origin, such as peanut oil, soybean oil, mineral oil, sesame oil, and the like. Saline solutions and aqueous dextrose and glycerol solutions can also be employed as liquid carriers, particularly for injectable solutions. The pharmaceutical carriers can be gum acacia, gelatin, starch paste, talc, keratin, colloidal silica, urea, and the like. In addition, auxiliary, stabilizing, thickening, lubricating, and coloring agents may be used. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, wetting or emulsifying agents, or pH buffering agents.

As used herein, “cyano” refers to a group having the structure —CN.

As used herein, “cycloalkyl” or “C₃-C₁₂ cycloalkyl” refers to an optionally substituted, non-aromatic, saturated monocyclic, bicyclic or tricyclic hydrocarbon ring system containing 3-12 carbon atoms. Examples of C₃-C₁₂ cycloalkyl groups include but are not limited to cyclopropyl, cyclobutyl, cyclopentyl, cyclohexyl, cycloheptyl, cyclooctyl, norbornyl, adamantyl, bicyclo[2.2.2]oct-2-enyl, and bicyclo[2.2.2]octyl.

An “effective amount” is an amount of a compound of the invention that is effective for treating or preventing pain (e.g., neuropathic pain) or inflammation.

As used herein, “ester” refers to a group having the structure —C(O)OR₆, wherein R₆ is selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl.

As used herein, “haloalkyl” refers to an alkyl group wherein at least one substituent is a halogen. Haloalkyls may also be perhalogenated as exemplified by trifluoromethyl.

As used herein, “halogen” refers to —F, —Cl, —Br, or —I.

As used herein, a “heterocycle” or “−3- to 9-membered heterocycle” is an optionally substituted 3- to 9-membered aromatic or nonaromatic monocyclic or bicyclic ring of carbon atoms and from 1 to 4 heteroatoms selected from oxygen, nitrogen and sulfur. Non-aromatic heterocycles may have one or more double bonds. Examples of double bonds include carbon-carbon double bonds (C═C), carbon-nitrogen double bonds (C═N), and nitrogen-nitrogen double bonds (N═N). Examples of 3- to 9-membered heterocycles include, but are not limited to, aziridinyl, oxiranyl, thiiranyl, azirinyl, diaziridinyl, diazirinyl, oxaziridinyl, azetidinyl, azetidinonyl, oxetanyl, thietanyl, diazinanyl, piperidinyl, tetrahydropyridinyl, piperazinyl, morpholinyl, azepinyl or any partially or fully saturated derivatives thereof, diazepinyl or any partially or fully saturated derivatives thereof, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl and indazolyl. Where an heterocycle group is substituted, substituents include, for example, one or more alkyl groups or a phosphorus (V) containing group.

As used herein, “heteroaryl” or “heteroaromatic” refers to a 3-9 membered heterocycle that is aromatic.

A “5- to 6-membered ring” is an optionally substituted 5- to 6-membered aromatic or nonaromatic monocyclic or bicyclic ring of carbon atoms only, or of carbon atoms and from 1 to 4 heteroatoms selected from oxygen, nitrogen and sulfur. Examples of 5- to 6-membered rings include, but are not limited to, cyclopentyl, cyclohexyl or cycloheptyl, which may be saturated or unsaturated, diazinanyl, piperidinyl, piperazinyl, morpholinyl, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl and indazolyl.

As used herein, “hydroxy” refers to a group having the structure —OH.

As used herein, “imine” refers to a group having the structure —C(NR⁶) wherein R⁶ is selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, —C₂-C₈ alkynyl or

As used herein, “isolated” means that the compounds of the invention are separated from other components of either (a) a natural source, such as a plant or cell, preferably bacterial culture, or (b) a synthetic organic chemical reaction mixture. An isolated compound can be, for example, 10%, 20%, 30%, 40%, 50%, 60%, 70%, 80%, 85%, 90%, 95%, 97%, 98%, or 99% pure.

By “isomer” is meant any stereoisomer, enantiomer, or diastereomer of any compound of the invention. Representative stereoisomers include geometric isomers such as double bond isomers. Exemplary double bond isomers that are encompassed by the invention are the compounds of formulas (Ia-2) and (Ib-2)

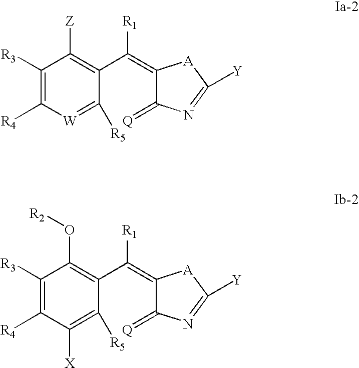

It is recognized that the compounds of the invention can have one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures, e.g., racemates.

Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, “ketone” refers to a carboxyl group that has the structure —C(O)R₆, wherein R₆ is selected from —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl.

As used herein, “natural amino acid” refers to an amino acid that is naturally produced or found in a mammal. Natural amino acids can be encoded by the standard genetic code or may result from, for example, post-translational modifications. Natural amino acids include the twenty proteinogenic L-amino acids (Alanine (A), Cysteine (C), Serine (S), Threonine (T), Aspartic Acid (D), Glutamic Acid (E), Asparagine (N), Glutamine (Q), Histidine (H), Arginine (R), Lysine (K), Isoleucine (I), Leucine (L), Methionine (M), Valine (V), Phenylalanine (F), Tyrosine (Y), Tryptophan (W), Glycine (G), and Proline (P)). Other natural amino acids include Gamma-aminobutyric acid (GABA; a γ-amino acid), 3,4-dihydroxy-L-phenylalanine (L-DOPA), camitine, ornithine, citrulline, homoserine, lanthionine, 2-aminoisobutyric acid, or dehydroalanine.

As used herein, “nitro” refers to a group having the structure —NO₂.

As used herein, “non-aromatic carbocycle” refers to an optionally substituted monocyclic, bicyclic, or tricyclic structure wherein the atoms that form the ring are all carbons and at least one ring does not have 4n+2 π electrons. Carbocycles contain 3-12 carbon atoms. Carbocycles include cycloalkyls, partially unsaturated cycloalkyls, or an aromatic ring fused to a cycloalkyl or partially unsaturated cycloalkyl. In addition to cycloalkyls and partially unsaturated cycloalkyls, exemplary non-aromatic carbocycles include tetrahydronaphthyl.

By “oxo” is meant a group having a structure ═O, wherein an oxygen atom makes a double bond to another element such as C, S, or P.

As used herein, “partially unsaturated cycloalkyl” refers to an optionally substituted C₃-C₁₂ cycloalkyl that has at least one carbon-carbon double bond. Exemplary partially unsaturated cycloalkyls include cyclopropenyl, cyclobutenyl, cyclopentenyl, cyclopentadienyl, cyclohexenyl, cyclohexadienyl, cycloheptenyl, cycloheptadienyl, cycloheptatrienyl, cyclooctenyl, and cyclooctadienyl.

As used herein, “pharmaceutically acceptable” means approved by a regulatory agency of the Federal or a state government or listed in the U.S. Pharmacopeia or other generally recognized pharmacopeia for use in animals, and more particularly in humans.

As used herein, “pharmaceutically acceptable salt(s),” includes but are not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, mesylate, hydroxymethylsulfonate, hydroxyethyl sulfonate, and pamoate (i.e., 1,1′-methylene-bis-(2-hydroxy-3-naphthoate)) salts. Similar, compounds of the invention that include ionizable hydrogens can be combined with various inorganic and organic bases to form salts.

As used herein, “phosphine” refers to a group having the structure —P(R₆ₐ)₃, wherein each R₆ₐ is selected, independently, from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or any two R₆ₐ, together with the atom to which each is attached, join to form a 3- to 7-membered aromatic or non aromatic heterocycle

As used herein, “phosphonato” refers to a group having the structure —P(═O)(OR₆)₂, wherein each R₆ is, independently, —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, —C₂-C₈ alkynyl, or two R₆, together with the atom to which each is attached, join to form a 3- to 7-membered aromatic or non aromatic heterocycle.

As used herein, a “phosphorus (V) containing group” refers to a group having the structure —(CR′R″)ₙOP(═O)(OR₆)(OR₇) or —(CR′R″)ₙP(═O)(OR₆)(OR₇), where each R′ and R″ is, independently, H or C₁₋₅ alkyl, R₆ and R₇ are independently —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, —C₂-C₈ alkynyl or two R₆, together with the atom to which each is attached, join to form a 3- to 7-membered aromatic or non aromatic heterocycle, and n is 0, 1, 2, or 3. An exemplary phosphorus (V) containing group is a phosphonato group as described herein. Still other exemplary phosphorus (V) containing groups include —(CH₂)ₙPO(OR₆R₇), wherein n is 0 to 3, —(CHR′)ₙPO(OR₆R₇), wherein n is 0 to 3, and —(C(R′)₂)ₙPO(OR₆R₇), wherein n is 0 to 3.

As used herein, the term “prevent” refers to prophylactic treatment or treatment that prevents one or more symptoms or conditions of a disease, disorder, or conditions described herein (e.g., pain such as neuropathic pain). Preventative treatment can be initiated, for example, prior to (“pre-exposure prophylaxis”) or following (“post-exposure prophylaxis”) an event that precedes the onset of the disease, disorder, or conditions (e.g., exposure to a headache trigger, to another cause of pain, or to a pathogen). Preventive treatment that includes administration of a compound of the invention, or a pharmaceutical composition thereof, can be acute, short-term, or chronic. The doses administered may be varied during the course of preventative treatment. See also: Kaniecki et al., “Treatment of Primary Headache: Preventive Treatment of Migraine.” In: Standards of Care for Headache Diagnosis and Treatment. Chicago (IL): National Headache Foundation; 2004. p. 40-52.

As used herein, a “prodrug” is a compound that is rapidly transformed in vivo to the parent compound of the compounds of the invention, for example, by hydrolysis in blood. Prodrugs of the compounds of the invention may be esters, carbamates, phosphorus (III) esters, or phosphorus (V) esters. Some common esters that have been utilized as prodrugs are phenyl esters, aliphatic (C₇-C₈ or C₈-C₂₄) esters, cholesterol esters, acyloxymethyl esters, and amino acid esters. Compounds of the invention (e.g., compounds of Formula (Ia) or (Ib)) can be converted to their corresponding prodrugs according to methods known in the art. For example, the phenol group of (Ia) or (Ib) can be treated with an electrophile (e.g., an acid chloride, an anhydride, a carboxylic ester, a carbonate, a carbamyl chloride, or a phosphorus (III) or (V) electrophile) to prepare the corresponding prodrug. Exemplary methods for the preparation of prodrugs are described herein. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

As used herein, “purified” means that when isolated, the isolate contains at least 95%, preferably at least 98%, of a single compound by weight of the isolate.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of stereoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

When the groups described herein are said to be “substituted or unsubstituted” or “optionally substituted,” when substituted, they may be substituted with any desired substituent or substituents selected from the following group: halogen (chloro, iodo, bromo, or fluoro); C₁₋₆ alkyl; C₂₋₆ alkenyl; C₂₋₆ alkynyl; hydroxyl; C₁₋₆ alkoxyl; amino; nitro; thiol; thioether; imine; cyano; amido; carbamoyl; phosphonato; phosphine; a phosphorus (V) containing group; carboxyl; thiocarbonyl; sulfonyl; sulfonamide; ketone; aldehyde; ester; oxo; haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocyclic, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic carbocylic (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl); benzyloxy; amino (primary, secondary, or tertiary); —N(CH₃)₂; O-alkyl; O-aryl; aryl; aryl-lower alkyl; CO₂CH₃; —OCH₂CH₃; methoxy; CONH₂; OCH₂CONH₂; SO₂NH₂; OCHF₂; CF₃; OCF₃; and such moieties may also be optionally substituted by a fused-ring structure or bridge, for example —OCH₂O—. These substituents may optionally be further substituted with a substituent listed herein. In other embodiments, these substituents are not further substituted.

The phrase “substantially anhydrous,” as used herein in connection with a reaction mixture or an organic solvent, means that the reaction mixture or organic solvent comprises less than about 1 percent of water by weight; in one embodiment, less than about 0.5 percent of water by weight; and in another embodiment, less than about 0.25 percent of water by weight of the reaction mixture or organic solvent.

As used herein, “sulfonamide” refers to a group having a structure selected from —S(O)N(R₆)₂ or —S(O)₂N(R₆)₂, wherein each R₆ is, independently, —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic heterocycle.

As used herein, “sulfonyl” refers to a group having a structure selected from —S(O)R₆, and —S(O)₂R₆, wherein R₆ is selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl.

As used herein, “thiocarbonyl” refers to a group having a structure selected from —C(S)R₆, —O—C(S)R₆, —O—C(S)OR₆, —O—C(S)N(R₆)₂, —C(S)N(R₆)₂, —C(S)OR₆, wherein each R₆ is, independently, selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic heterocycle;

As used herein, “thioether” refers to a group having the structure —SR₆, wherein R₆ is selected from —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl.

As used herein, “thiol” refers to a group having the structure SH.

As used herein, “unnatural amino acid” is an amino acid that is not naturally produced (e.g., encoded by the genetic code or resulting from a posttranslational modification) or naturally found in a mammal. Unnatural amino acids include amino acids that normally do not occur in proteins (e.g., an α-amino acid having the D-configuration, or a (D,L)-isomeric mixture thereof), homologues of naturally occurring amino acids (e.g., a β- or γ-amino acid analogue), an α,α-disubstituted analogue of a naturally occurring amino acid, or an α-amino acid wherein the amino acid side chain has been shortened by one or two methylene groups or lengthened to up to 10 carbon atoms. Other unnatural amino acids include γ-amino acids that are GABA analogues, such as (S)-3-(aminomethyl)-5-methylhexanoic acid (pregabalin), 2-[1-(aminomethyl)cyclohexyl]acetic acid (gabapentin), or those described in Yogeeswari et al., Recent Patents on CNS Drug Discovery, 1:113-118, 2006, herein incorporated by reference.

In one embodiment, when administered to a patient, e.g., a mammal for veterinary use or a human for clinical use, the compounds are administered in isolated form. In another embodiment, via conventional techniques, the compounds are purified.

It should be noted that if there is a discrepancy between a depicted structure and a name given that structure, the depicted structure controls. In addition, if the stereochemistry of a structure or a portion of a structure is not indicated with, for example, bold or dashed lines, the structure or portion of the structure is to be interpreted as encompassing all stereoisomers of it.

The following abbreviations and their definitions, unless defined otherwise, are used in this specification:

| Abbreviation | Definition |
|---|---|
| ACN | acetonitrile |
| BOC | —C(O)OC(CH₃)₃ |
| dba | dibenzylideneacetone |
| DBU | 1,8-diazabicyclo[5.4.0]undec-7-ene |
| DCM | dichloromethane |
| DEF | N,N-diethylformamide |
| DIPEA | N,N-diisopropylethylamine |
| DMAP | 4-dimethylaminopyridine |
| DMF | N,N-dimethylformamide |
| DMSO | dimethylsulfoxide |
| EtOAc | ethyl acetate |
| EtOH | ethanol |
| MTBE | methyl tert-butyl ether |
| MeOH | methanol |
| Ph | phenyl |
| TBDMSCl | tert-butyldimethylsilyl chloride |
| TEA | triethylamine |
| TFA | trifluoroacetic acid |
| THF | tetrahydrofuran |
| Tf | —SO₂CF₃ |

### DETAILED DESCRIPTION OF THE INVENTION

The present invention features compounds having the Formula (Ia) and use of these compounds in pharmaceutical compositions and methods of treatment or prevention of disease:

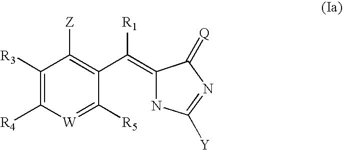

including stereoisomers, E/Z isomers, prodrugs and pharmaceutically acceptable salts thereof.

In some embodiments, the compounds of (Ia) have structures according to the following formulas

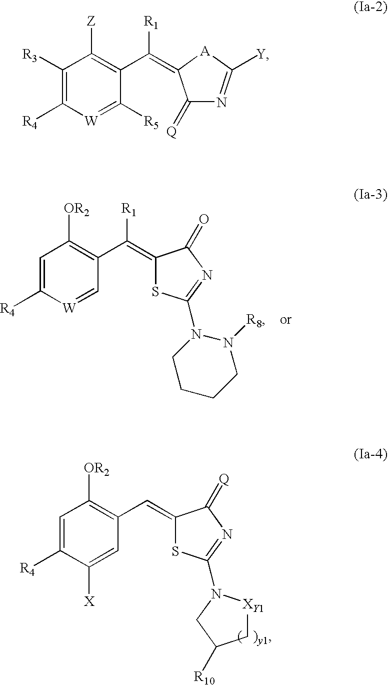

including stereoisomers, E/Z isomers, prodrugs and pharmaceutically acceptable salts thereof.

The invention further provides methods for treating disease by administering a compound having the Formula (Ib), depicted below,

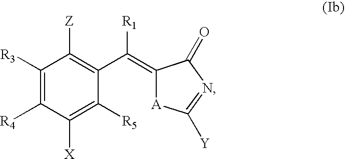

including stereoisomers, E/Z isomers, prodrugs and pharmaceutically acceptable salts thereof.

In some embodiments, the compound of Formula (Ib) has a structure according to the following formula

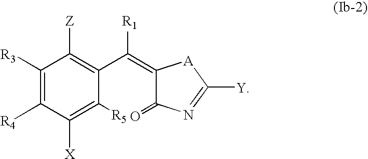

Exemplary compounds of the invention are shown herein.

Methods for making the compounds of Formula (Ia) and (Ib)

In general, the compounds of the invention can be obtained via standard, well-known synthetic methodology, see e.g. March, J. Advanced Organic Chemistry; Reactions Mechanisms, and Structure, 4^{th} ed., 1992. Illustrative methods are described below. Starting materials useful for preparing the compounds of the invention and intermediates therefore, are commercially available or can be prepared from commercially available materials using known synthetic methods and reagents. It is understood that the methods of synthesis provided below also encompass the synthesis of isomers (e.g. compounds having structures according to formulas (Ia-2) and (Ib-2).

An example of a synthetic pathway useful for making the compounds is set forth below and generalized in Scheme 1. The compounds of Formula (Ia) or (Ib) can be obtained via conventional organic synthesis, e.g., as described below. Scheme 1 indicates a general method by which the compounds can be obtained, wherein Q, Z, W, A, Y, X, n, and R₁-R₆ are defined above for the compounds of Formula (Ia) and wherein Q, A, Y, X, n, and R₁-R₆ are defined above for the compounds of Formula (Ib).

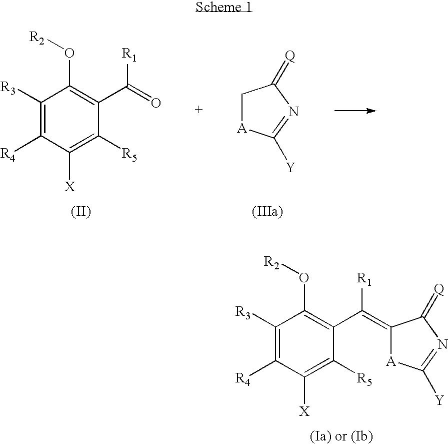

For example, a commercially available or synthetically prepared compound of Formula (II) is subjected to condensation reaction with a commercially available or synthetically prepared compound of Formula (IIa) under acidic or basic conditions in a polar solvent.

A second example of a synthetic pathway useful for making the compounds is set forth below and generalized in Scheme 2. The compounds of Formula (Ia) or (Ib) can be obtained via conventional organic synthesis, e.g., as described below. Scheme 2 provides a second general method by which the compounds can be obtained, wherein Q, Z, W, A, Y, X, n, and R₁—R₆ are defined above for the compounds of Formula (Ia) and wherein Q, A, Y, X, n, and R₁—R₆ are defined above for the compounds of Formula (Ib).

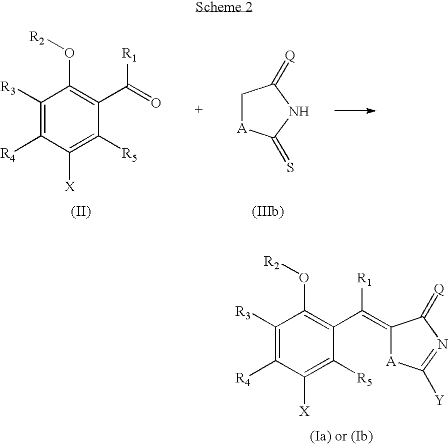

For example, a commercially available or synthetically prepared compound of Formula (II) is subjected to condensation reaction with a compound of Formula (IIIa), which itself may undergo nucleophilic substitution of the sulfur moiety by suitably basic nucleophile: Y such as pyrrolidine, piperidine, or piperazine, in a polar solvent such as ethanol. Alternatively, the reactions are conducted sequentially when the synthetic process from scheme 2 is not suitable or low yielding.

Scheme 3 provides a two step approach for the synthesis of compounds of Formula (Ia) or (Ib). In this case, the compound of Formula (IIIa) is first prepared by reacting a compound of Formula (IIIb) with a nucleophile: Y to yield a compound of Formula (IIa), which is then condensed under acidic or basic condition in a polar solvent with a commercially available or synthetically prepared compound of Formula (II).

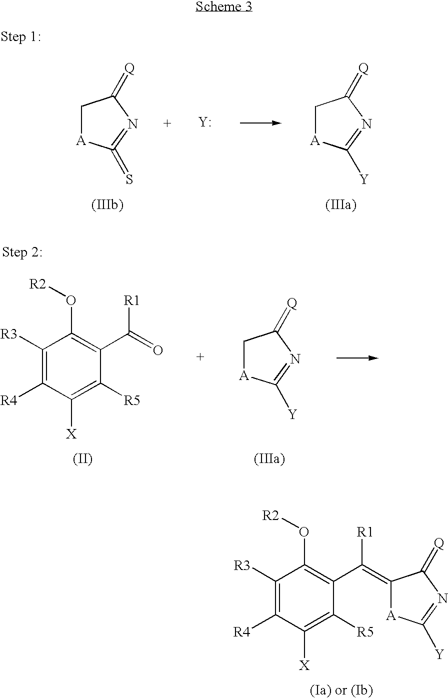

Scheme 4 shows another alternative for preparing the compounds of Formula (Ia) or (Ib).

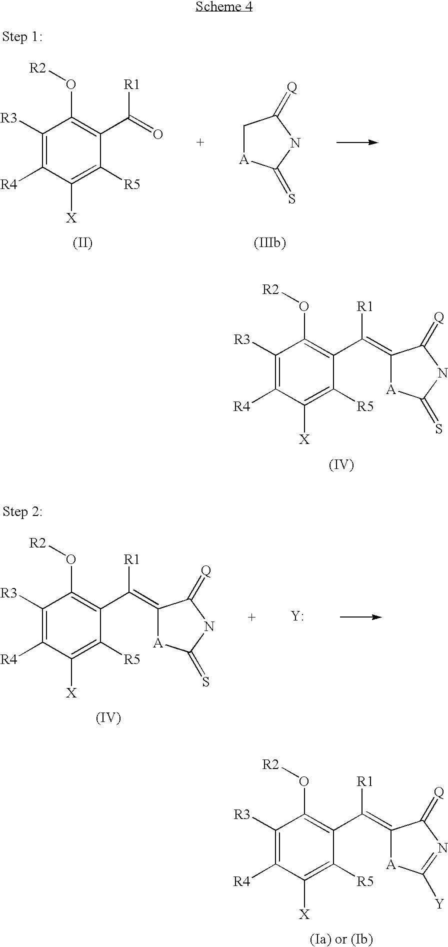

In Scheme 4, a commercially available or synthetically prepared compound of Formula (II) is subjected to condensation reaction under acidic or basic condition in a polar solvent with a compound of Formula (IIIb) to give compound (IV). Compounds of Formula (Ia) or (Ib) are then obtained from nucleophilic substitution of the sulfur moiety from compound (IV) by a suitable nucleophile: Y.

The formation of a compound of Formula (Ia) or (Ib) can be monitored using conventional analytical techniques, including, but not limited to, thin-layer chromatography, high-performance liquid chromatography, gas chromatography, and nuclear magnetic resonance spectroscopy such as ¹H or ¹³C NMR.

### Therapeutic/Prophylactic Use

Because of their activity, the compounds of the invention are advantageously useful in veterinary and human medicine. For example, the compounds described herein are useful for the treatment or prevention of pain.

The invention provides methods of treatment and prophylaxis by administration to a patient of an effective amount of a compound described herein. The patient is an animal, including, but not limited to, a human, mammal (e.g., cow, horse, sheep, pig, cat, dog, mouse, rat, rabbit, mouse, or guinea pig), or other animal, such as a chicken, turkey, or quail.

The present compositions, which include an effective amount of a compound of the invention, can be administered by any convenient route, for example by infusion or bolus injection, by absorption through epithelial or mucocutaneous linings (e.g., oral mucosa, rectal and intestinal mucosa, etc.) and can be administered alone or together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically to the ears, nose, eyes, or skin. The preferred mode of administration is left to the discretion of the practitioner.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an injury. In another embodiment, administration can be by direct injection at the site (or former site) of an infection, tissue or organ transplant, or autoimmune response.

In certain embodiments, it may be desirable to introduce one or more compounds of the invention into the central nervous system by any suitable route, including intraventricular and intrathecal injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulating with an aerosolizing agent, or via perfusion in a fluorocarbon or synthetic pulmonary surfactant. In certain embodiments, the compounds of the invention can be formulated as a suppository, with traditional binders and carriers such as triglycerides.

In another embodiment, the compounds of the invention can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compounds of the invention can be delivered in a controlled-release system. In one embodiment, a pump may be used (see Langer, supra; Sefton, CRC Crit. Ref. Biomed. Eng. 9:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the compounds of the invention, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) may be used.

Pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable carriers can be sterile. In one embodiment, water is a carrier when the compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, can also contain minor amounts of wetting or emulsifying agents, or pH buffering agents.

In one embodiment, compounds of the invention (e.g., a compound of Formula (Ia) or (Ib)) are formulated in 10 to 40% of a sulfobutylether β-cyclodextrin (Captisol®) or in 10 to 40% hydroxypropyl-α-cyclodextrin, optionally with precipitation inhibitors such as hydroxypropylmethylcellulose.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” by E. W. Martin.

Compounds of the invention included in the present compositions that include an amino moiety may form pharmaceutically acceptable salts with various amino acids, in addition to the acids mentioned above. Compounds, included in the present compositions, that are acidic in nature are capable of forming base salts with various pharmacologically or cosmetically acceptable cations. Examples of such salts include alkali metal or alkaline earth metal salts and, particularly, calcium, magnesium, sodium, lithium, zinc, potassium, and iron salts.

In another embodiment, the compounds of the invention are formulated in accordance with routine procedures as a pharmaceutical composition adapted for intravenous administration to human beings. Typically, compounds for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate may also be used. Oral compositions can include standard carriers such as mannitol, lactose, starch, magnesium stearate, sodium saccharine, cellulose, or magnesium carbonate. Such carriers can be of pharmaceutical grade.

The amount of the compound of the invention that will be effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable effective dosage ranges for intravenous administration are generally about 0.01 to about 5 g, preferably about 0.01 to about 1 g of the compound per kilogram body weight. In specific embodiments, the i.v. dose is about 0.005 to about 0.5 g/kg, about 0.01 to about 0.3 g/kg, about 0.025 to about 0.25 g/kg, about 0.04 to about 0.20 g/kg, or about 0.05 to about 0.20 g/kg (or the equivalent doses expressed per square meter of body surface area). Alternatively, a suitable dose range for i.v. administration may be obtained using doses of about 1 to about 2000 mg, without adjustment for a patient's body weight or body surface area. Suitable dosage ranges for intranasal administration are generally about 0.01 pg/kg body weight to 10 mg/kg body weight. Suppositories generally contain 0.5% to 20% by weight of one or more compounds of the invention alone or in combination with another therapeutic agent. Oral compositions can contain about 10% to about 95% by weight of one or more compounds alone or in combination with another therapeutic agent. In specific embodiments of the invention, suitable dose ranges for oral administration are generally about 0.1 to about 200 mg, preferably about 0.5 to about 100 mg, and more preferably about 1 to about 50 mg of arylmethylidene heterocycle per kilogram body weight or their equivalent doses expressed per square meter of body surface area. In specific embodiments the oral dose is about 0.25 to about 75 mg/kg, about 1.0 to about 50 mg/kg, about 2.0 to about 25 mg/kg, about 2.5 to about 15 mg/kg, or about 5.0 to about 20 mg/kg (or the equivalent doses expressed per square meter of body surface area). In another embodiment, a suitable dose range for oral administration, from about 10 to about 4000 mg, without adjustment for a patient's body weight or body surface area. Other effective doses may be extrapolated from dose-response curves derived from in vitro or animal model test systems. Such animal models and systems are well known in the art.

The invention also provides pharmaceutical packs or kits comprising one or more containers containing one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In certain embodiments, e.g., when administered for the treatment or prevention of pain, the kit may also contain one or more analgesic agents useful for treating pain to be administered in combination with an arylmethylidene heterocycle.

The compounds of the invention are preferably assayed in vivo, for the desired therapeutic or prophylactic activity, prior to use in humans. For example, in vivo assays can be used to determine whether administration of a specific compound or combination of compounds is preferred.

### Inhibition of Pain

Pain can be treated or prevented by administration of an effective amount of a compound of the invention. The compounds may be demonstrated to inhibit pain by using the procedure described by Bennett & Xie (Pain, 1988). Experimental details are provided in the Examples section.

Exemplary pain conditions that can be treated or prevented include, but are not limited to: musculoskeletal pain (e.g., back and leg pain, neck, shoulder and arm pain, whiplash injuries, motor vehicle, work-related and sports injuries, pre- or postoperative pain syndromes, cervicogenic headache, pain due to arthritis, myofascial pain, or fibromyalgia), cancer pain (e.g., primary or metastatic cancer pain or medication side effect management), vascular pain, Raynaud's disease, psychogenic pain, trigeminal neuralgia, spinal cord injury, spasticity, post dural puncture headache, pelvic pain, or neuropathic pain (e.g., Complex Regional Pain Syndrome (RSD), postherpetic neuralgia (shingles), peripheral neuralgia, nerve injuries, phantom limb pain, or AIDS-related pain).

Pain can be acute or chronic. The compounds of the invention can be used to treat or prevent acute or chronic pain associated with any of the following conditions: musculoskeletal disorders (e.g., osteoarthritis/degenerative joint disease/spondylosis, rheumatoid arthritis, lyme disease, Reiter syndrome, disk herniation/facet osteoarthropathy, fractures/compression fracture of lumbar vertebrae, faulty or poor posture, fibromyalgia, polymyalgia rheumatica, mechanical low back pain, chronic coccygeal pain, muscular strains and sprains, pelvic floor myalgia (levator ani spasm), Piriformis syndrome, rectus tendon strain, hernias (e.g., obturator, sciatic, inguinal, femoral, spigelian, perineal, or umbilical), abdominal wall myofascial pain (trigger points), chronic overuse syndromes (e.g., tendinitis, bursitis)), neurological disorders (e.g., brachial plexus traction injury, cervical radiculopathy, thoracic outlet syndrome, spinal stenosis, arachnoiditis, metabolic deficiency myalgias, polymyositis, neoplasia of spinal cord or sacral nerve, cutaneous nerve entrapment in surgical scar, postherpetic neuralgia (shingles), neuralgia (e.g., iliohypogastric, ilioinguinal, or genitofemoral nerves), polyneuropathies, polyradiculoneuropathies, mononeuritis multiplex, chronic daily headaches, muscle tension headaches, migraine headaches, temporomandibular joint dysfunction, temporalis tendonitis, sinusitis, atypical facial pain, trigeminal neuralgia, glossopharyngeal neuralgia, nervus intermedius neuralgia, sphenopalatine neuralgia, referred dental or temporomandibular joint pain, abdominal epilepsy, or abdominal migraine), urologic disorders (e.g., bladder neoplasm, chronic urinary tract infection, interstitial cystitis, radiation cystitis, recurrent cystitis, recurrent urethritis, urolithiasis, uninhibited bladder contractions (detrusor-sphincter dyssynergia), urethral diverticulum, chronic urethral syndrome, urethral carbuncle, prostatitis, urethral stricture, testicular torsion, or Peyronie disease)), gastrointestinal disorders (e.g., chronic visceral pain syndrome, gastroesophageal reflux, peptic ulcer disease, pancreatitis, chronic intermittent bowel obstruction, colitis, chronic constipation, diverticular disease, inflammatory bowel disease, or irritable bowel syndrome), reproductive disorders (e.g., adenomyosis, endometriosis, adhesions, adnexal cysts, atypical dysmenorrhea or ovulatory pain, cervical stenosis, chlamydial endometritis or salpingitis, chronic ectopic pregnancy, chronic endometritis, endometrial or cervical polyps, endosalpingiosis, from a intrauterine contraceptive device, leiomyomata, ovarian retention syndrome (residual ovary syndrome), ovarian remnant syndrome, ovarian dystrophy or ovulatory pain, pelvic congestion syndrome, postoperative peritoneal cysts, residual accessory ovary, subacute salpingo-oophoritis, symptomatic pelvic relaxation (genital prolapse), or tuberculous salpingitis), psychological disorders (e.g., bipolar personality disorders, depression, porphyria, or sleep disturbances), cardiovascular disease (e.g., angina), peripheral vascular disease, or from chemotherapeutic, radiation, or surgical complications.

### Treatment or Prevention of Pain Further Comprising Administering Other Pain Control Agents

Methods may include the administration of one or more additional pain control agent, including, but not limited to, gababentin, morphine, oxycodone, fentanyl, pethidine, methadone, propoxyphene, hydromorphone, hydrocodone, codeine, meperidine, gabapentin, pregabalin, lidocaine, ketamine, capsaicin, anticonvulsants such as valproate, oxcarbazepine or carbamazepine, tricyclic antidepressants such as amitriptyline, duloxetine, venlafaxine, and milnacipran, or serotonin-norepinephrine reuptake inhibitors (SNR^{1s}) such as bicifadine, desipramine, desvenlafaxine, duloxetine, milnacipran, nefazodone, sibutramine, or venlafaxine.

### Treatment or Prevention of Inflammation

Inflammation can be treated or prevented by administration of an effective amount of a compound of the invention. The compounds of the invention can also be used to treat or prevent pain that results from inflammation. Inflammatory pain can be acute or chronic. Exemplary conditions associated with inflammatory pain include, but are not limited to: osteoarthritis, rheumatoid arthritis, autoimmune conditions, burns, extreme cold, excessive stretching, fractures, infections, pancreatitis, penetration wounds, and vasoconstriction.

### Prodrugs

The present invention also provides prodrugs of the compounds of the invention. Prodrugs include derivatives of compounds that can hydrolyze, oxidize, or otherwise react under biological conditions (in vitro or in vivo) to provide an active compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, and biohydrolyzable phosphate analogues. In certain embodiments, prodrugs of the compounds of the invention with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh). Biohydrolyzable moieties of a compound of the invention either do not interfere with the biological activity of the compound but can confer upon that compound advantageous properties in vivo, such as uptake, duration of action, or onset of action or are biologically inactive but are converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

### EXAMPLES

### Synthesis of Representative Compounds of Formula (Ia) And (Ib)

Compounds of Formula (Ia) and (Ib) can be prepared by using the general procedures described earlier in Scheme 1-4 and further exemplified below.

### Prodrugs

Scheme 5 shows a method for the preparation of carbamate prodrugs.

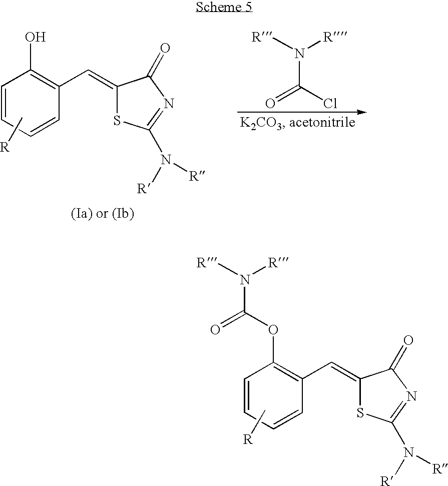

Compound (Ia) or (Ib) (10 mmol, 1.0 eq) and potassium carbonate (20 mmol, 2.0 eq) were stirred in acetonitrile (0.5 M). A solution of carbamoyl chloride 2 (14 mmol, 1.4 eq) in acetonitrile was then added at room temperature. The reaction mixture was heated at 80° C. overnight. The mixture was cooled to room temperature, filtered, and the solid was washed with CH₂Cl₂/MeOH (2:1). The filtrates were combined and concentrated to afford the crude solid, which was then washed with ethyl acetate to provide compound (Ic) as off-white solid. Other carbonyl-containing prodrugs can be obtained using analogous procedures.

Phosphorus-containing prodrugs can also be prepared according to methods known in the art. Exemplary methods are described herein.

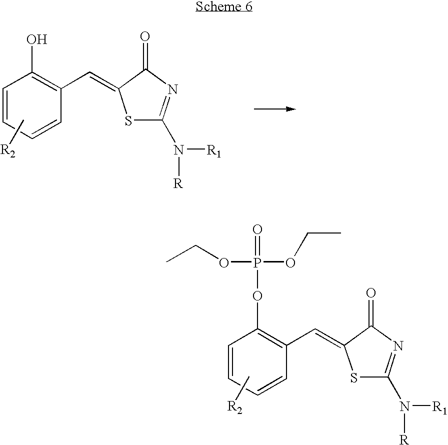

Method A is shown in Scheme 6. To a suspension of the phenol (1 equivalent) in acetonitrile at room temperature was added triethylamine (1.3 equiv) and diethylchlorophosphate (1.1 equiv), followed by catalytic DMAP. The reaction mixture clarified and then was stirred at room temperature overnight. The solvent was evaporated, and the residue was purified by combiflash to provide the phosphate ester.

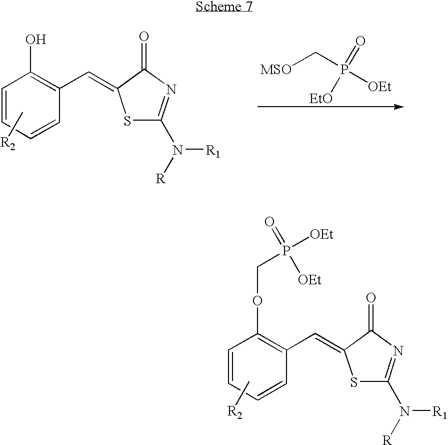

Method B is shown in Scheme 7. To a suspension of the phenol in anhydrous acetonitrile K₂CO₃ (1.5 equiv), trifluoromethanesulfonic acid diethoxy-phosphorylmethyl ester (1.2 equiv; prepared according to literature procedure (J. Org. Chem., 61:7697 (1996)) was heated at reflux overnight. The reaction mixture was filtered and evaporated to provide the product as a semi solid.

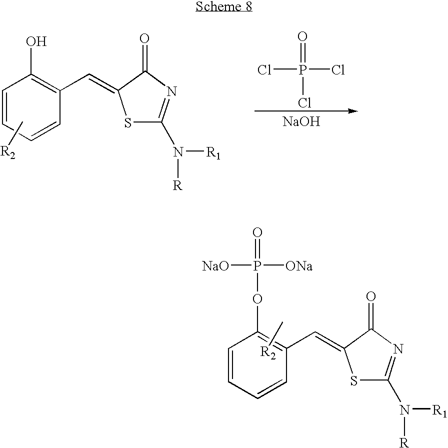

Method C is shown in Scheme 8. In a 250 mL round bottom flask, the phenol analogue (10 mmol) and triethylamine (3.08 mL, 22 mmol) were mixed in THF (100 mL). POCl₃ (1.0 mL, 11 mmol) was added slowly at 0° C. After 2 hours, the resulting mixture was stirred at room temperature for another 5 hours. The mixture was filtered to remove triethylamine salts and unreacted phenols. To the clear filtrate, water (0.72 mL, 40 mmol) was added. After another 3 hours, a yellow solid was collected and washed with THF to provide the phosphate product.

Where the phosphorus group includes one or more ionizable hydrogens, salts of the phosphorus-containing prodrugs (e.g., sodium salts) can be obtained in the following manner. To the slurry of 10% weight phosphoric prodrug in water, NaOH aq (1.0 eq, 2N) was added. The mixture became a clear solution, and the solution was then lyophilized to provide the dry sodium salt.

### Example 1

### (5Z)-5-[(2-Hydroxyphenyl)methylidene]-2-(pyrrolidin-1-yl)-4,5-dihydro-1,3-thiazol-4-one

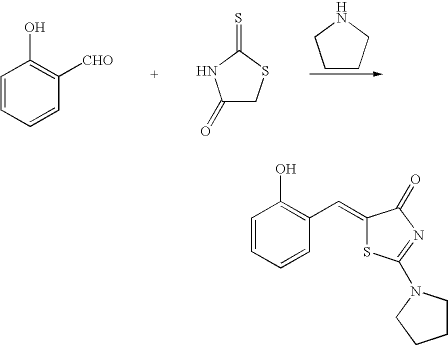

To a solution of rhodanine (500 mg, 3.8 mmol) in absolute ethanol (30 mL) was added dropwise a solution of salicaldehyde (419 μL, 4.0 mmol) and pyrrolidine (629 μL, 7.6 mmol) in absolute ethanol (5 mL). The reaction mixture was stirred at reflux for 2 hours. After cooling to room temperature, the solid material was recovered by filtration, washed with EtOH (2×15 mL) and acetone (1×15 ml), and dried in vacuo, affording the title compound (825 mg; 79%). ¹H NMR (400 MHz, DMSO-d₆) δ 1.97 (m, 4H), 3.58 (t, 2H, J=6.5 Hz), 3.67 (t, 2H, J=6.7 Hz), 6.92 (m, 2H), 7.24 (td, 1H, J=1.7 Hz, 7.2 Hz), 7.39 (dd, 1H, J=1.6 Hz, 8.0 Hz), 7.88 (s, 1H), 10.33 (s, 1H); M⁺ 275.

### Example 2

### (5Z)-5-(2-Hydroxybenzylidene)-2-(4-methylpiperazin-1-yl)-1,3-thiazol-4(5H)-one

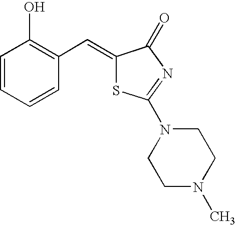

To a solution of rhodanine (500 mg, 3.8 mmol) in absolute ethanol (15 mL) was added salicaldehyde (419 μL, 4.0 mmol), followed by N-methyl piperazine (500 μL, 4.5 mmol). The reaction mixture was stirred at reflux overnight. After cooling to room temperature, the solid material was recovered by filtration, washed with EtOH (2×15 mL) and diethyl ether (1×15 ml), and dried in vacuo, affording the compound (434 mg; 38%). ¹H NMR (400 MHz, DMSO-d₆) δ 2.24 (s, 3H), 2.45 (m, 4H), 3.63 (t, 2H, J=5.0 Hz), 3.90 (t, 2H, J=5.0 Hz), 6.94 (m, 2H), 7.27 (td, 1H, J=1.6 Hz, 8.5 Hz), 7.44 (dd, 1H, J=1.6 Hz, 7.8 Hz), 7.92 (s, 1H), 10.36 (s, 1H); M⁺ 304.

### Example 3

### (5Z)-5-[(2-Hydroxy-5-methylphenyl)methylidene]-2-(piperidin-1-yl)-4,5-dihydro-1,3-thiazol-4-one

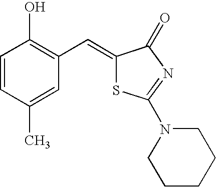

Example 3 was prepared following the procedure described for Example 1 using 2-hydroxy-5-methyl-benzaldehyde, piperidine, and rhodanine. The crude product was purified by flash chromatography (reverse phase C₁₈ column, 0-50% ACN/5 mM NH₄OH_{(aq)}), affording the compound (183 mg; 16%). ¹H NMR (400 MHz, DMSO-d₆) δ 1.62 (m, 6H), 2.21 (s, 3H), 3.58 (m, 2H), 3.86 (t, 2H, J=5.9 Hz), 6.78 (d, 1H, J=8.4 Hz), 7.16 (d, 1H, J=1.0 Hz), 7.91 (s, 1H); M⁺ 303.

### Example 4

### (5Z)-5-[(2-Hydroxy-5-nitrophenyl)methylidene]-2-(piperidin-1-yl)-4,5-dihydro-1,3-thiazol-4-one

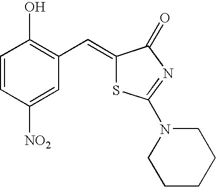

Example 4 was prepared following the procedure described for example 1 using 2-hydroxy-5-nitro-benzaldehyde, piperidine, and rhodanine. The solid material was recovered by filtration and dried in vacuo, affording the title compound (471 mg; 37%). ¹H NMR (400 MHz, DMSO-d₆) δ 1.64 (m, 6H), 3.60 (m, 2H), 3.89 (t, 2H, J=5.0 Hz), 7.07 (dd, 1H, J=1.6 Hz, 9.0 Hz), 8.15 (d, 1H, J=1.7 Hz), 8.15 (dd, 1H, J=3.0 Hz, 9.2 Hz), 8.24 (d, 1H, J=2.7 Hz); M+334.

### Example 5

### (5Z)-5-[(2-Hydroxy-5-methoxyphenyl)methylidene]-2-(piperidin-1-yl)-4,5-dihydro-1,3-thiazol-4-one

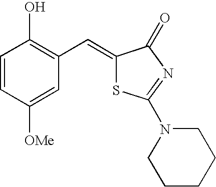

Example 5 was prepared following the procedure described for Example 1 using 2-hydroxy-5-methoxy-benzaldehyde, piperidine, and rhodanine. The crude product was purified by flash chromatography (reverse phase C₁₈ column, 0-30% ACN/5 mM NH₄OH_{(aq)}) twice, affording the compound (21 mg; 2%). ¹H NMR (400 MHz, DMSO-d₆) δ 1.65 (m, 6H), 3.61 (m, 2H), 3.73 (s, 2H), 3.89 (t, 1H, J=5.5 Hz), 6.90 (m, 4H), 7.87 (s, 1H), M⁺ 319.

### Example 6

### (5Z)-2-(Dimethylamino)-5-[(2-hydroxyphenyl)methylidene]-4,5-dihydro-1,3-thiazol-4-one

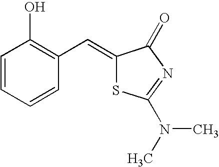

Example 6 was prepared following the procedure described for Example 1 using salicylaldehyde, dimethylamine, and rhodanine. After cooling to room temperature, the solid material was recovered by filtration, washed with EtOH (2×15 mL), and dried in vacuo, affording the compound (586 mg; 62%). ¹H NMR (400 MHz, DMSO-d₆) δ 3.21 (s, 3H), 3.27 (s, 3H), 6.92 (m, 2H), 7.24 (td, 1H, J=1.7 Hz, 7.2 Hz), 7.41 (dd, 1H, J=1.6 Hz, 8.0 Hz), 7.88 (s, 1H), 10.33 (s, 1H); M⁺ 249.

### Example 7

### (5Z)-5-[(2-Hydroxyphenyl)methylidene]-2-(methylamino)-4,5-dihydro-1,3-thiazol-4-one

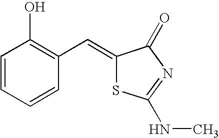

Example 7 was prepared following the procedure described for Example 1 using salicylaldehyde, methylamine, and rhodanine. The crude product was purified by flash chromatography (reverse phase C₁₈ column, 0-30% ACN/5 mM NH₄OH_{(aq)} and 0-10% ACN/5 mM NH₄OH_{(aq)}), affording the title compound (110 mg; 12%). ¹H NMR (400 MHz, DMSO-d₆) δ 3.04 (s, 3H), 6.94 (m, 2H), 7.24 (t, 1H, J=7.6 Hz), 7.33 (d, 1H, J=7.6 Hz), 7.9 (s, 1H); M⁺ 235.

### Example 8

### (5Z)-5-[(5-Fluoro-2-hydroxyphenyl)methylidene]-2-(4-methylpiperazin-1-yl)-4,5-dihydro-1,3-thiazol-4-one

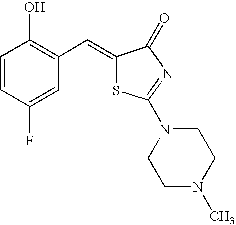

Example 8 was prepared following the procedure described for Example 1 using 5-fluoro-2-hydroxy benzaldehyde, N-methylpiperazine, and rhodanine. The product was obtained in 887 mg (73%). ¹H NMR (400 MHz, DMSO-d₆) δ 2.24 (s, 3H), 2.45 (m, 4H), 3.66 (t, 2H, J=5.0 Hz), 3.91 (t, 2H, J=5.0 Hz), 6.95 (m, 2H), 7.15 (m, 2H), 7.84 (m, 1H), 10.40 (s(br), 1H); M⁺ 322.

### Example 9

### (5Z)-5-[(4-Fluoro-2-hydroxyphenyl)methylidene]-2-(piperidin-1-yl)-4,5-dihydro-1,3-thiazol-4-one

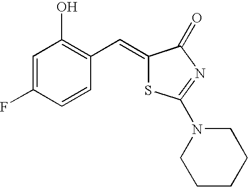

Example 9 was prepared following the procedure described for Example 1 using 4-fluoro-2-hydroxy benzaldehyde, piperidine, and rhodanine. ¹H NMR (400 MHz, DMSO-d₆) δ 1.65 (m, 6H), 3.60 (m, 2H), 3.89 (t, 2H, J=5.4 Hz), 6.73 (dd, 1H, J=2.7 Hz, 10.6 Hz), 6.80 (td, 1H, J=2.5 Hz, 8.6 Hz), 7.47 (m, 1H), 7.84 (s, 1H), 10.38 (s, 1H); M⁺ 307.

### Example 10

### Preparation of (5Z)-5-[(2-Hydroxyphenyl)methylidene]-2-(1,2,3,6-tetrahydropyridin-1-yl)-4,5-dihydro-1,3-thiazol-4-one

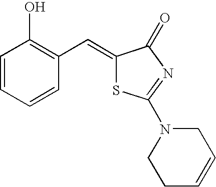

Example 10 was prepared following the procedure described for Example 1 using salicylaldehyde, 1,2,3,6-tetrahydropyridine, and rhodanine. The product was obtained in 715 mg (66%). ¹H NMR (400 MHz, DMSO-d₆) δ 2.28 (m, 2H), 3.73 (t, 2H, J=5.8 Hz), 4.01 (t, 2H, J=5.9 Hz), 4.16 (t, 2H, J=2.4 Hz), 4.37 (t, 2H, J=2.5 Hz), 5.79 (m, 1H), 5.93 (m, 1H), 6.94 (m, 2H), 7.26 (t, 1H, J=7.0 Hz), 7.45 (m, 1H), 7.93 (d, 1H, J=5.3 Hz), 10.37 (s, 1H); M⁺ 287.

### Example 11

### (5Z)-5-[(5-Fluoro-2-hydroxyphenyl)methylidene]-2-(1,2,3,6-tetrahydropyridin-1-yl)-4,5-dihydro-1,3-thiazol-4-one

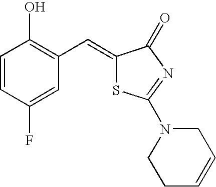

Example 11 was prepared following the procedure described for Example 1 using 5-fluoro-2-hydroxy benzaldehyde, 1,2,3,6-tetrahydropyridine, and rhodanine. The product was obtained in 715 mg (66%). ¹H NMR (400 MHz, DMSO-d₆) δ 2.29 (m, 2H), 3.76 (t, 2H, J=5.8 Hz), 4.01 (t, 2H, J=5.9 Hz), 4.19 (t, 2H, J=2.4 Hz), 4.38 (t, 2H, J=2.5 Hz), 5.79 (m, 1H), 5.93 (m, 1H), 6.95 (m, 2H), 7.16 (m, 2H), 7.45 (m, 1H), 7.84 (dd, 1H, J=1.4 Hz, 5.7 Hz), 10.40 (s, 1H); M⁺ 287.

### Example 12

### (5Z)-5-[(4-Hydroxypyridin-3-yl)methylidene]-2-(piperidin-1-yl)-4,5-dihydro-1,3-thiazol-4-one

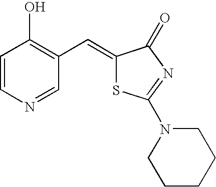

Example 11 was prepared following the procedure described for Example 1 using 4-hydroxypyridine-3-carbaldehyde, piperidine, and rhodanine. The crude product was purified by flash chromatography (reverse phase (C₁₈ column), 0-20% ACN/5 mM NH₄OH_{(aq)} and 0-10% ACN/0.05% TFA_{(aq)}), affording the compound (115 mg; 10%). ¹H NMR (400 MHz, DMSO-d₆) δ 1.65 (m, 6H), 3.35 (m, 2H), 3.55 (t, 2H, J=5.1 Hz), 6.18 (d, 1H, J=6.9 Hz), 7.48 (s, 1H), 7.67 (d, 1H, J=6.5 Hz), 8.03 (s, 1H), 11.84 (s(br), 1H); M⁺ 290.

### Example 13

### (5Z)-5-[(5-Chloro-2-hydroxyphenyl)methylidene]-2-(piperidin-1-yl)-4,5-dihydro-1,3-thiazol-4-one

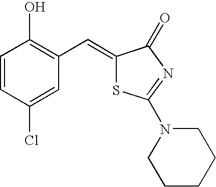

Example 13 was prepared following the procedure described for Example 1 using 2-hydroxy-5-chloro-benzaldehyde, piperidine, and rhodanine. The crude product was purified by flash chromatography using CH₂Cl₂-MeOH using 5-10% to provide 115 mg (10%) of the compound. ¹H NMR (400 MHz, DMSO-d₆) δ 1.65 (m, 6H), 3.65 (m, 2H), 3.85 (t, 2H, J=5.1 Hz), 6.97 (d, 1H, J=8.6 Hz), 7.32 (d, 1H, J=8.6 Hz), 7.36 (s, 1H), 7.80 (s, 1H), 10.69 (bs 1H).

### Example 14

### (3R)-1-[(5Z)-5-[(2-Hydroxyphenyl)methylidene]-4-oxo-4,5-dihydro-1,3-thiazol-2-yl]-N,N-dimethylpyrrolidin-3-aminium chloride

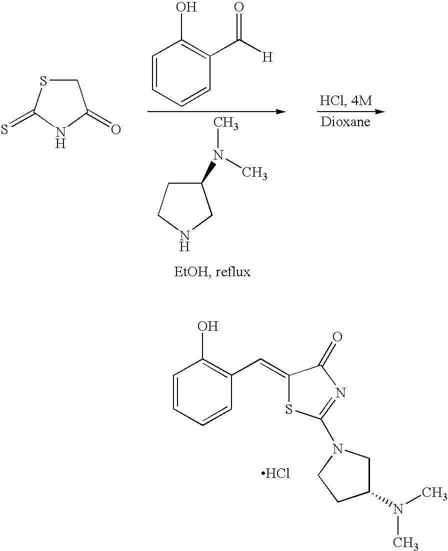

To a solution of rhodanine (500 mg, 3.8 mmol) in absolute ethanol (15 mL) was added salicylaldehyde (419 μL, 4.0 mmol) followed by (3R)-(+)-3-(dimethylamino)pyrrolidine (500 mg, 4.4 mmol). The reaction mixture was stirred at reflux overnight. After cooling to room temperature, the solid material was recovered by filtration, washed with EtOH (2×15 mL) and diethyl ether (2×15 ml), and dried in vacuo, affording the free base (886 mg; 73%). The solid material (2.6 mmol) was suspended in tert-butanol (10 mL) and water (10 mL) before 4M HCl in dioxane (4 mL, 16.0 mmol) was added. The solid material completely dissolved. The solution was then filtered, and the filtrate was lyophilized, affording the final product (920 mg; 93%). ¹H NMR (400 MHz, D₂O) δ 2.16 (m, 1H), 2.46 (m, 1H), 2.78 (m, 6H), 3.43 (m, 1H), 3.75 (m, 4H), 6.73 (m, 2H), 7.09 (m, 2H), 7.67 (d, 1H, J=5.1 Hz); M⁺ 318.

### Example 15

### (3R)-1-[(5Z)-5-[(5-Fluoro-2-hydroxyphenyl)methylidene]-4-oxo-4,5-dihydro-1,3-thiazol-2-yl]-N,N-dimethylpyrrolidin-3-aminium chloride

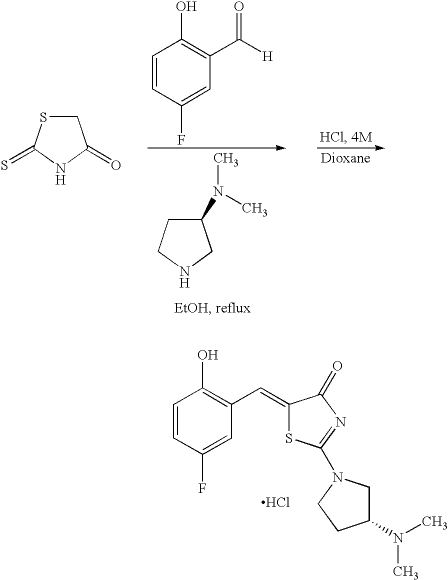

To a solution of rhodanine (500 mg, 3.8 mmol) in absolute ethanol (15 mL) was added 5-fluorosalicylaldehyde (560 mg, 4.0 mmol), followed by (3R)-(+)-3-(dimethylamino)pyrrolidine (500 mg, 4.4 mmol). The reaction mixture was stirred at reflux overnight. After cooling to room temperature, the solid material was recovered by filtration, washed with EtOH (2×15 mL) and diethyl ether (2×15 ml), and dried in vacuo, affording the free base, 900 mg (71%). The solid material (2.6 mmol) was suspended in tert-butanol (10 mL) and water (20 mL) before 4M HCl in dioxane (4 mL, 16.0 mmol) was added. The resulting mixture was lyophilized, affording the final product (920 mg; 93%). ¹H NMR (400 MHz, DMSO-d₆) δ 2.38 (m, 2H), 2.82 (m, 6H), 3.71 (m, 1H), 4.06 (m, 4H), 7.02 (m, 1H), 7.16 (m, 2H), 7.86 (dd, 1H, J=1.4 Hz, 11.67 (s(br), 0.5H), 11.76 (s(br), 0.5H); M⁺ 322.

### Example 16

### (3R)-1-[(5Z)-5-[(2-Hydroxyphenyl)methylidene]-4-oxo-4,5-dihydro-1,3-thiazol-2-yl]-N,N-dimethylpyrrolidin-3-aminium; methanesulfonate

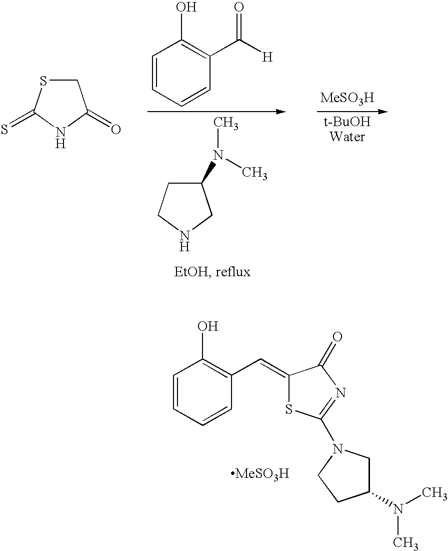

To a solution of rhodanine (500 mg, 3.8 mmol) in absolute ethanol (15 mL) was added salicylaldehyde (419 μL, 4.0 mmol), followed by (3R)-(+)-3-(dimethylamino)pyrrolidine (500 mg, 4.4 mmol). The reaction mixture was stirred at reflux overnight. After cooling to room temperature, the solid material was recovered by filtration, washed with EtOH (2×15 mL) and diethyl ether (2×15 ml), and dried in vacuo, affording the free base (684 mg; 57%). The solid material (2.1 mmol) was suspended in tert-butanol (25 mL) and water (25 mL) before methanesulfonic acid (162 μL, 2.5 mmol) was added. The solid material completely dissolved. The solution was filtered, and the filtrate was lyophilized, affording the product (845 mg; 97%). ¹H NMR (400 MHz, D₂O) δ 2.17 (m, 1H), 2.48 (m, 1H), 2.62 (s, 3H), 2.79 (m, 6H), 3.46 (m, 1H), 3.78 (m, 4H), 6.76 (m, 2H), 7.13 (m, 2H), 7.71 (d, 1H, J=6.5 Hz); M⁺ 318.

### Example 17

### (5Z)-2-(Azepan-1-yl)-5-[(2-hydroxyphenyl)methylidene]-4,5-dihydro-1,3-thiazol-4-one

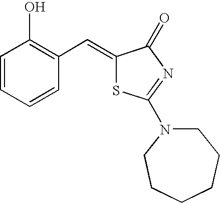

Example 17 was prepared following the procedure described for Example 1 using salicylaldehyde, azepane, and rhodanine. The product was obtained in 24% yield. ¹H NMR (400 MHz, DMSO-d₆) δ 1.54 (m, 4H), 1.90 (m, 4H), 3.67 (m, 2H), 3.87 (m, 2H), 6.95 (m, 2H), 7.25 (t, 1H), 7.45 (d, 1H), 7.92 (s, 1H), 10.35 (s, 1H).

### Example 18

### (5Z)-5-[(2-Hydroxyphenyl)methylidene]-2-(4-methyl-1,4-diazepan-1-yl)-4,5-dihydro-1,3-thiazol-4-one

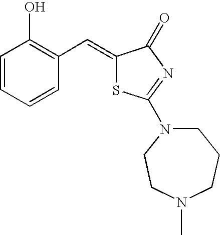

Example 18 was prepared following the procedure described for Example 1 using salicylaldehyde, 1-methyl-[1,4]diazepane, and rhodanine. The product was obtained in 39% yield. ¹H NMR (400 MHz, DMSO-d₆) δ 1.91 (m, 2H), 2.33 (s, 3H), 2.50-2.70 (m, 4H), 3.70 (m, 2H), 3.95 (m, 2H), 6.96 (m, 2H), 7.27 (t, 1H), 7.45 (d, 1H), 7.92 (s, 1H), 10.35 (s, 1H).

### Example 19

### (5Z)-5-[(5-Fluoro-2-hydroxyphenyl)methylidene]-2-(piperidin-1-yl)-4,5-dihydro-1,3-thiazol-4-one

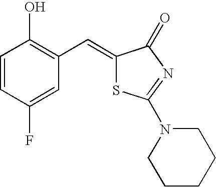

Example 19 was prepared following the procedure described for Example 1 using 5-fluoro-2-hydroxybenzaldehyde, piperidine, and rhodanine. The product was obtained in 50% yield. ¹H NMR (400 MHz, DMSO-d₆) δ 1.63 (m, 6H), 3.63 (m, 2H), 3.94 (m, 2H), 6.95 (m, 1H), 7.18 (m, 2H), 7.83 (s, 1H), 10.38 (s, 1H).

### Example 20

### (5Z)-2-Amino-5-[(2-hydroxyphenyl)methylidene]-4,5-dihydro-1,3-thiazol-4-one

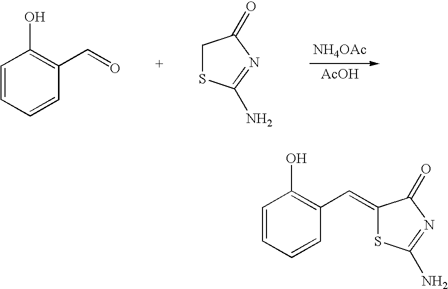

To 30 mL of acidic acid in a 100 mL round bottom flask, 2-amino-4-oxo-thiazole (1.16 g, 10 mmol), 2-hydroxybenzaldehyde (1.22 g, 10 mmol), and ammonium acetate (0.77 g, 10 mmol) were added. The resulting mixture was stirred at 100° C. overnight. After cooling down to 0° C., the solid was filtered, washed with water and ethanol, dried under vacuum, and 200 mg of yellow solid was collected in pure form. ¹H NMR (400 MHz, DMSO-d₆) δ 7.32 (dd, 1H), 7.45 (d, 1H), 7.58 (dd, 1H), 7.72 (d, 1H), 8.13 (s, 1H).

### Example 21

### (5Z)-5-[(3-Fluoro-2-hydroxyphenyl)methylidene]-2-(4-methylpiperazin-1-yl)-4,5-dihydro-1,3-thiazol-4-one

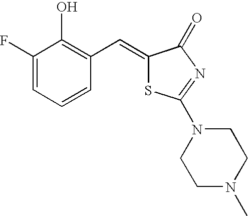

Example 21 was prepared following the procedure described for Example 1 from 3-fluoro-2-hydroxybenzaldehyde, N-methylpiperazine, and rhodanine. The product was obtained in 11% yield. ¹H NMR (400 MHz, DMSO-d₆) δ 2.25 (m, 4H), 3.60, 3.60 and 3.90 (2 Br, 4H), 7.50 (m, 1H), 7.76 (m, 1H), 8.17 (d, 1H).

### Example 22

### (5Z)-5-[(5-Chloro-2-hydroxyphenyl)methylidene]-2-(4-methylpiperazin-1-yl)-4,5-dihydro-1,3-thiazol-4-one

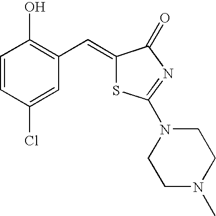

Example 22 was prepared following the procedure described for Example 1 from 5-chloro-2-hydroxybenzaldehyde, N-methylpiperazine, and rhodanine. The compound was obtained in 45% yield. ¹H NMR (400 MHz, DMSO-d₆) δ 2.26 (m, 4H), 3.62 and 3.90 (2 br, 4H), 7.78-7.88 (m, 3H), 8.35 (s, 1H).

### Example 23

### (5Z)-5-[(3-Fluoro-2-hydroxyphenyl)methylidene]-2-(piperidin-1-yl)-4,5-dihydro-1,3-thiazol-4-one

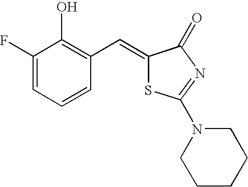

Example 23 was obtained following the procedure described for Example 16 using 3-Fluoro-2-hydroxybenzaldehyde, piperidine, and rhodanine. The compound was obtained in 9% yield. ¹H NMR (400 MHz, DMSO-d₆) δ 1.60 (m, 6H), 2.78 (m, 1H), 3.52 (dd, 2H), 3.88 (m, 2H), 4.66 (dd, 1H), 6.76 (1H), 6.93 (d, 1H), 7.07 (m, 1H), 9.71 (s, 1H).

### Example 24

### (3S)-1-[(5E and Z)-5-[(5-Fluoro-2-hydroxyphenyl)methylidene]-4-oxo-4,5-dihydro-1,3-thiazol-2-yl]-N,N-dimethylpyrrolidin-3-aminium; chloride

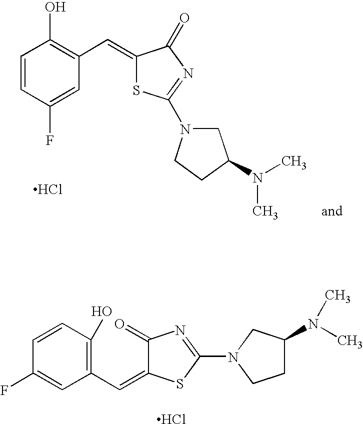

Example 24 was synthesized following the procedure described for Example 15 using 5-fluoro-2-hydroxybenzaldehyde, (3S)-(+)-3-(dimethylamino)pyrrolidine, and rhodanine. The product was obtained in 72% yield. ¹H NMR (400 MHz, DMSO-d₆) δ 1.98-2.39 (m, 1H), 2.48 (s, 6H), 3.26-4.07 (m, 6H), 6.91-7.189 (m, 3H), 7.61, 7.84 (2s, 1H), 10.45 (s, 1H).

### Example 25

### (3S)-1-[(5Z)-5-[(2-Hydroxyphenyl)methylidene]-4-oxo-4,5-dihydro-1,3-thiazol-2-yl]-N,N-dimethylpyrrolidin-3-aminium; methanesulfonate

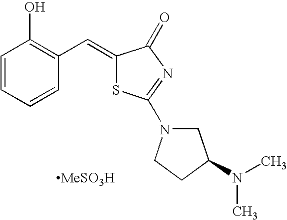

Example 25 was synthesized following the procedure described for Examplel 6 using 2-hydroxybenzaldehyde, (3S)-(+)-3-(dimethylamino)pyrrolidine, and rhodanine. The product was obtained in 60% yield. ¹H NMR (400 MHz, DMSO-d₆) δ 1.85-1.96 (m, 1H), 2.19 (s, 6H), 2.88-4.00 (m, 6H), 6.95 (d, 2H), 7.27 (t, 1H), 7.40 (t, 1H), 7.91 (s, 1H), 10.36 (br, 1H).

### Example 26

### (5Z)-2-{[2-(Dimethylamino)ethyl]amino}-5-(5-fluoro-2hydroxybenzylidene)-1,3-thiazol-4(5H)-one

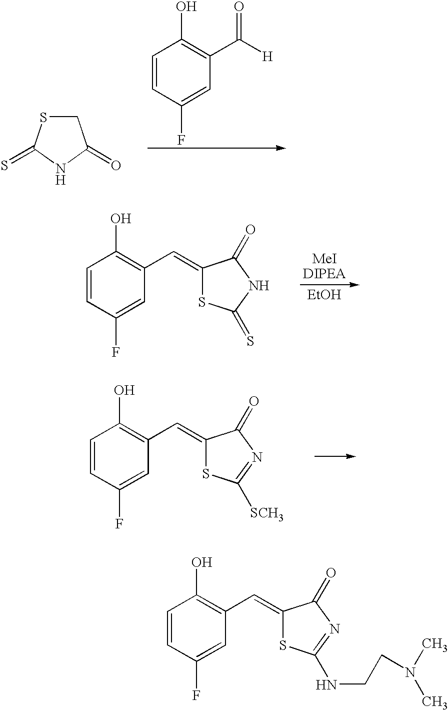

A solution of rhodanine (2.01 g, 15.1 mmol), 5-fluorosalicylaldehyde (2.00 g, 14.0 mmol), and ammonium acetate (430 mg, 5.6 mmol) in acetic acid (60 mL) was stirred at reflux for 60 hours. After cooling to room temperature, the solid material was recovered by filtration, washed with water (2×50 mL), and air-dried for 1 hour. The solid material was dissolved in diethyl ether (500 ml). This solution was dried over MgSO₄, filtered, evaporated, and dried in vacuo, affording (5Z)-5-[(5-fluoro-2-hydroxyphenyl)methylidene]-2-sulfanylidene-1,3-thiazolidin-4-one (2.59 g, 71%). The product was used without further purification.

To a suspension of (5Z)-5-[(5-fluoro-2-hydroxyphenyl)methylidene]-2-sulfanylidene-1,3-thiazolidin-4-one (1.2 g, 4.7 mmol) in absolute ethanol (20 mL) was added diisopropylethylamine (1.0 mL, 5.7 mmol) followed by iodomethane (475 μL, 7.6 mmol). The reaction mixture was stirred at room temperature overnight. The solid material was recovered by filtration, washed with EtOH (1×15 mL) and diethyl ether (2×15 ml), and dried in vacuo, affording (5Z)-5-[(5-fluoro-2-hydroxyphenyl)methylidene]-2-(methylsulfanyl)-4,5-dihydro-1,3-thiazol-4-one (769 mg; 61%). The product was used without further purification.

To a solution of (5Z)-5-[(5-fluoro-2-hydroxyphenyl)methylidene]-2-(methylsulfanyl)-4,5-dihydro-1,3-thiazol-4-one (300 mg, 1.1 mmol) in absolute ethanol (15 mL) was added N,N-dimethylethylenediamine (893 μL, 3.6 mmol). The reaction mixture was stirred at reflux overnight. The solvent was evaporated under reduced pressure. The crude product was purified by flash chromatography (reverse phase (C₁₈ column), 0-50% ACN/5 mM NH₄OH_{(aq)}). The solid residue was suspended in diethyl ether (100 mL), collected by filtration, and dried in vacuo, affording the final product (65 mg; 20%). ¹H NMR (400 MHz, DMSO-d₆) δ 2.19 (s, 6H), 2.46 (t, 2H, J=6.3 Hz), 3.61 (t, 2H, J=6.3 Hz), 6.94 (m, 1H), 7.11 (m, 2H), 7.79 (d, 2H, J=1.1 Hz); M⁺ 310.

### Example 27

### 4-Hydroxy-3-{[(5Z)-4-oxo-2-(piperidin-1-yl)-4,5-dihydro-1,3-thiazol-5-ylidene]methyl}benzonitrile

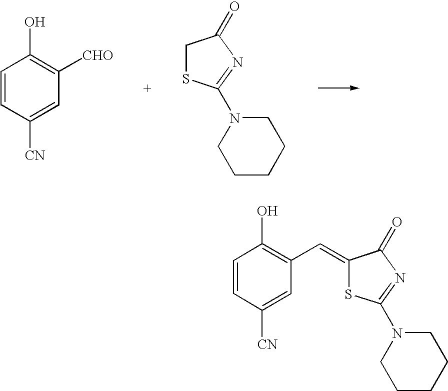

To a solution of 3-formyl-4-hydroxy-benzonitrile (0.367 g, 2.49 mmol) and 2-piperidin-1-yl-1,3-thiazol-4-one (0.46 g, 2.49 mmol) in acetic acid (20 mL) was added ammonium acetate (0.192 mg, 2.49 mmol). The mixture was heated at 100° C. overnight. A solid precipitated, and this solid was filtered and washed with water and ether to provide pure product (450 mg; 72%). ¹H NMR (400 MHz, DMSO-d₆) δ 1.67 (m, 6H), 3.89 (m, 2H), 3.90 (m, 2H), 7.09 (d, J=8.4 Hz, 1H), 7.70 (m, 2H), 7.77 (s, 1H), 11.6 (s, 1H).

### Example 28

### 4-Hydroxy-3-{[(5Z)-4-oxo-2-(piperidin-1-yl)-4,5-dihydro-1,3-thiazol-5-ylidene]methyl}benzoic acid

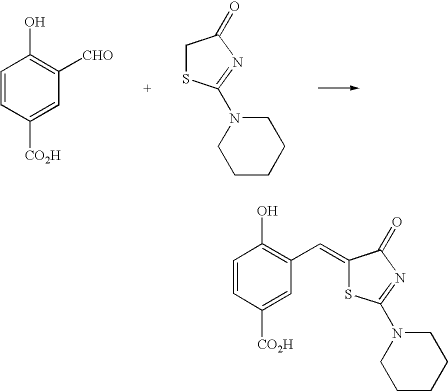

Example 28 was synthesized using the procedure described for Example 27 using 3-formyl-4-hydroxy-benzoic acid. 0.592 mg (60%) of product was obtained. ¹H NMR (400 MHz, DMSO-d₆) δ 1.67 (m, 6H), 3.60 (m, 2H), 3.90 (m, 2H), 7.09 (d, J=8.7 Hz, 1H), 7.84 (d, J=8.7 Hz, 1H), 7.88 (s, 1H), 8.07 (s, 1H), 11.24 (s, 1H).

### Example 29

### (5Z)-5-[(2-Hydroxyphenyl)methylidene]-2-(4-phenylpiperazin-1-yl)-4,5-dihydro-1,3-thiazol-4-one

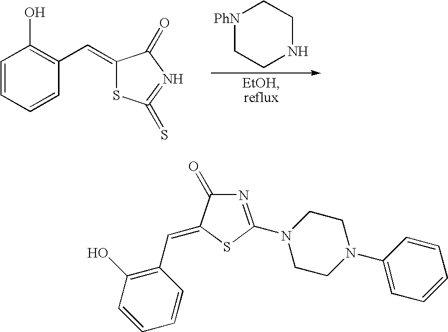

A solution of (5Z)-5-[(2-hydroxyphenyl)methylidene]-2-sulfanylidene-1,3-thiazolidin-4-one (500 mg, 2.1 mmol) and 1-phenylpiperazine (442 μL, 2.9 mmol) in absolute ethanol (30 mL) was stirred at reflux overnight. After cooling to room temperature, half of the solvent was removed under reduced pressure. The solid precipitate was recovered by filtration, washed with EtOH (2×15 mL), dried in vacuo, and air-dried in the oven (100° C.), affording 275 mg (36%) of product. ¹H NMR (400 MHz, DMSO-d₆) δ 3.32 (m, 4H), 3.79 (t, 2H, J=5.0 Hz), 4.06 (t, 2H, J=5.0 Hz), 6.85 (t, 1H, J=7.2 Hz), 6.97 (m, 4H), 7.27 (m, 3H), 7.46 (d, 1H, J=7.4 Hz), 7.95 (s, 1H); M⁺ 366.

### Example 30

### (5Z)-5-[(2-Hydroxyphenyl)methylidene]-2-(piperazin-1-yl)-4,5-dihydro-1,3-thiazol-4-one

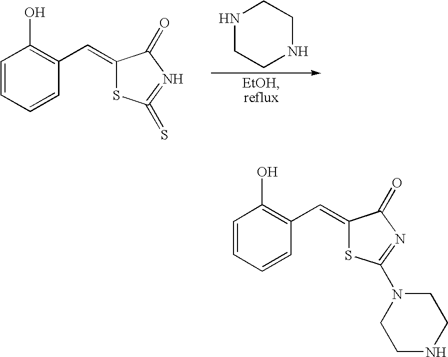

Example 30 was synthesized following the procedure described for Example 29 using (5Z)-5-[(2-hydroxyphenyl)methylidene]-2-sulfanylidene-1,3-thiazolidin-4-one and piperazine as starting materials. The product was purified by flash chromatography (reverse phase C₁₋₈ column, 0-30% ACN/5 mM NH₄OH_{(aq)} and 0-50% ACN/0.05% TFA_{(aq)}), affording 288 mg (26%) of product. ¹H NMR (400 MHz, DMSO-d₆) δ 3.30 (m, 4H), 3.85 (t, 2H, J=5.1 Hz), 4.09 (t, 2H, J=5.1 Hz), 6.95 (m, 2H), 7.30 (td, 1H, J=1.6 Hz, 8.5 Hz), 7.43 (dd, 1H, J=1.6 Hz, 7.7 Hz), 8.98 (s(br), 1H), 10.46 (s, 1H); M⁺ 290.

### Example 31

### (5Z)-2-(1,4-Diazepan-1-yl)-5-[(2-hydroxyphenyl)methylidene]-4,5-dihydro-1,3-thiazol-4-one

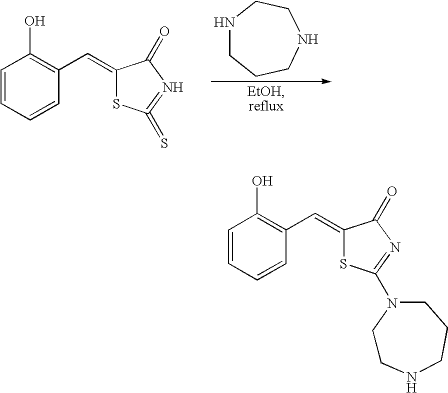

Example 31 was synthesized following the procedure described for Example 30 using (5Z)-5-[(2-hydroxyphenyl)methylidene]-2-sulfanylidene-1,3-thiazolidin-4-one and azepane as starting materials. The crude product was purified by flash chromatography (reverse phase C₁₈ column, 0-30% ACN/5 mM NH₄OH_{(aq)}), affording the product (52 mg; 16%). ¹H NMR (400 MHz, DMSO-d₆) δ 1.79 (m, 2H), 2.75 (m, 2H), 2.94 (m, 2H), 3.65 (t, 1H, J=5.5 Hz), 3.73 (t, 1H, J=6.1 Hz), 3.87 (t, 1H, J=5.3 Hz), 3.92 (t, 1H, J=5.7 Hz), 6.94 (m, 2H), 7.27 (td, 1H, J=1.6 Hz, 8.5 Hz), 7.43 (d, 1H, J=8.2 Hz), 7.92 (s, 1H); M⁺ 304.

### Example 32

### (5Z)-2-(Azetidin-1-yl)-5-[(2-hydroxyphenyl)methylidene]-4,5-dihydro-1,3-thiazol-4-one

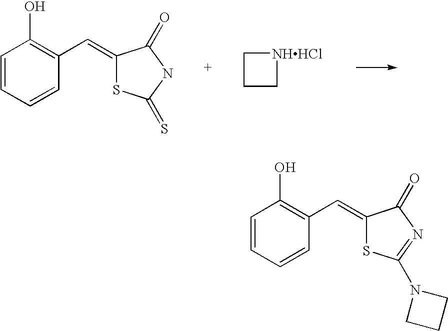

To a solution of azetidine hydrochloride (0.413 g, 4.42 mmol) in ethanol (10 mL) at room temperature was added triethylamine (0.467 g, 4.63 mmol). (5Z)-5-[(2-hydroxyphenyl)methylidene]-2-sulfanylidene-1,3-thiazolidin-4-one (0.5 g, 2.1 mmol) was then added to this solution. The reaction was then heated at reflux overnight. The precipitated solid was filtered off, and the solid was washed with ether to provide the product (100 mg; 18.2%). ¹H NMR (400 MHz, DMSO-d₆) δ2.55 (m, 4H), 4.31 (m, 4H), 6.93 (m, 2H), 7.24 (t, J=8.7, 7.2 Hz, 1H), 7.38 (d, J=7.2 Hz, 1H), 7.89 (s, 1H).

### Multistep Synthesis

Scheme 9 provides an example of a multistep synthetic approach for compounds of Formula (Ia) and (Ib).

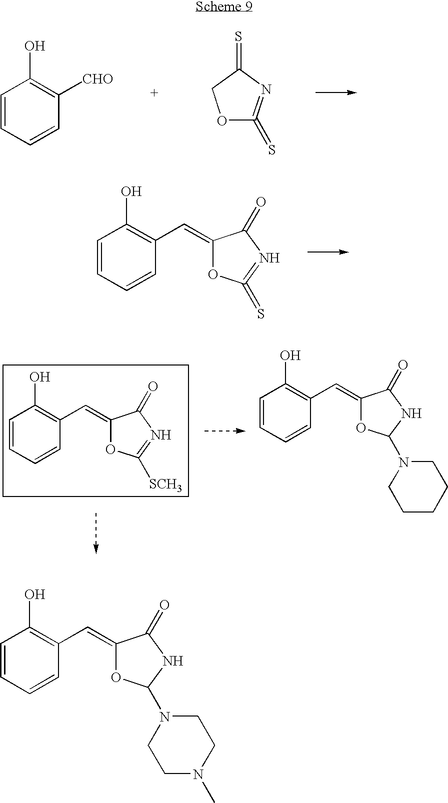

### Example 33

### (5Z)-5-[(2-Hydroxyphenyl)methylidene]-2-sulfanylidene-1,3-oxazolidin-4-one

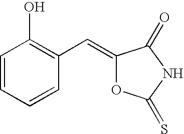

A mixture of 2-thioxo-4-oxazolidinone (5.6 g, 47.86 mmol), salicylaldehyde (5.25 g, 43.07 mmol), and sodium acetate (15.7 g, 191.45 mmol) in 30 mL of acetic acid was heated to reflux overnight. The reaction mixture was cooled to room temperature, and a thick solid appeared. The reaction mixture was poured into 300 mL of ice water and was stirred for 30 minutes. The solid was filtered, washed with water, hexane, and finally with dichloromethane to provide 7.61 g of brown solid. (yield 72%). ¹H NMR (400 MHz, DMSO-d₆) δ 6.96 (m, 3H), 7.32 (t, 1H, J=8.2 Hz, 17.2 Hz), 7.87 (d, J=8.2 Hz, 1H), 10.50 (s, 1H), M+221.

### Example 34

### (5Z)-5-[(2-Hydroxyphenyl)methylidene]-2-(methylsulfanyl)-4,5-dihydro-1,3-oxazol-4-one

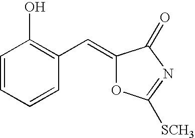

To a solution of (5Z)-5-(2-hydroxy-benzylidine)-2-thioxo-oxazolidin-4-one (0.85 g, 3.86 mmol) in anhydrous THF (15 mL) was added triethylamine (0.429 g, 4.25 mmol) at 0° C. The reaction mixture was stirred at 0° C. for 30 minutes, and then methyl iodide (2.74 g, 6 mL) was added. The reaction was allowed to stir overnight at room temperature. The precipitated solid was then filtered off, and the solid was washed several times with ethyl acetate. The filtrate was then evaporated to provide the solid, which was then washed with EtOH, EtOAc, and hexane to provide 0.86 g of product (95%). ¹H NMR (400 MHz, DMSO-d₆) δ 2.51 (s, 3H), 6.94 (m, 2H), 7.31 (t, J=7.5, 13.9 Hz, 1H), 7.80 (d, J=7.5 Hz, 1H), 10.53 (bs, 1H); M⁺ 235.

### Example 35

### (5Z)-5-[(5-Fluoro-2-hydroxyphenyl)methylidene]-2-sulfanylidene-1,3-thiazolidin-4-one

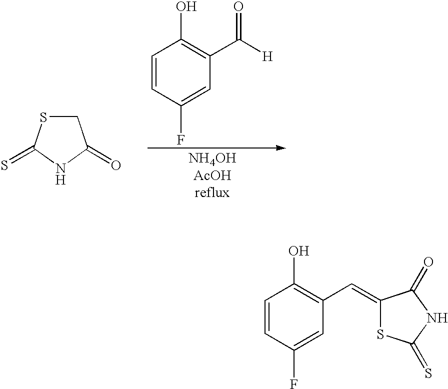

A solution of rhodanine (2.01, 15.1 mmol) and 5-fluorosalicylaldehyde (2.00 mg, 14.3 mmol) in acetic acid (60 mL) was stirred at reflux for 60 hours. After cooling to room temperature, the solid material was recovered by filtration, washed with water (3×50 mL), and the solid material was dissolved in diethyl ether (500 ml). The solution was dried over MgSO₄, filtered, evaporated, and dried in vacuo, affording the (5Z)-5-(5-fluoro-2-hydroxybenzylidene)-2-thioxo-1,3-thiazolidin-4-one (2.59 g, 71%). ¹H NMR (400 MHz, DMSO-d₆) δ 6.96 (dd, 1H, J=4.9 Hz, 9.0 Hz), 7.08 (dd, 1H, J=2.9 Hz, 6.6 Hz), 7.22 (td, 1H, J=2.9 Hz, 6.6 Hz), 7.74 (s, 1H), 10.71 (s, 1H); M⁺ 256.

### Example 36

### (5Z)-5-[(5-Fluoro-2-hydroxyphenyl)methylidene]-2-(methylsulfanyl)-4,5-dihydro-1,3-thiazol-4-one

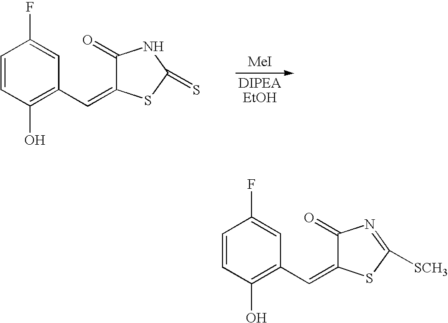

To a suspension of (5Z)-5-[(5-fluoro-2-hydroxyphenyl)methylidene]-2-sulfanylidene-1,3-thiazolidin-4-one (1.20, 4.7 mmol) in ethanol (20 mL) was added diisopropylethylamine (1.0 mL, 5.7 mmol), followed by iodomethane (475 μL, 7.6 mmol). The mixture was stirred at room temperature overnight. The solid material was recovered by filtration, washed with ethanol (1×15 mL) and diethyl ether (2×15 ml), and dried in vacuo, affording the product (769 mg; 61%). ¹H NMR (400 MHz, DMSO-d₆) δ 2.83 (s, 3H), 6.98 (dd, 1H, J=4.9 Hz, 9.0 Hz), 7.15 (dd, 1H, J=2.9 Hz, 6.6 Hz), 7.23 (td, 1H, J=2.9 Hz, 6.6 Hz), 7.99 (s, 1H), 10.71 (s, 1H); M⁺ 270.

Compounds of the invention having the Formula (Ia-3) can be prepared by analogous methods as exemplified by the following procedures.

### Example 37

### (5Z)-5-[(4-Fluoro-2-hydroxyphenyl)methylidene]-2-(methylsulfanyl)-4,5-dihydro-1,3-thiazol-4-one

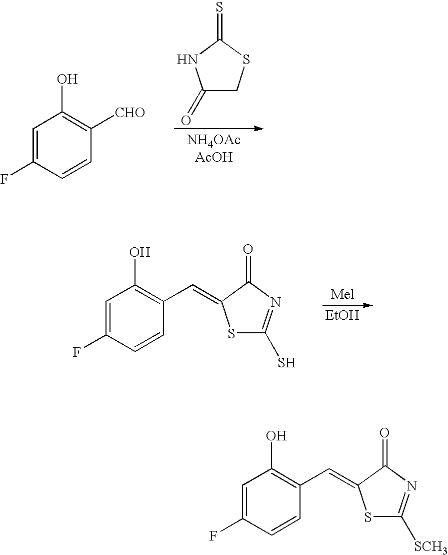

To 1.4 L of acetic acid in a 3 L round bottom flask were added 4-fluoro-2-hydroxybenzaldehyde (70.5 g, 500 mmol), rhodanine (66.6 g, 500 mmol), and NH₄OAc (19.5 g, 255 mmol). The resulting reaction mixture was stirred and heated at 100° C. overnight. After cooling to room temperature, the solid was filtered under vacuum. The solid was then washed thoroughly with water to remove acetic acid and ammonium salt until the filtrate became pale-yellow color. Hexane (500 mL) was used to remove excess water. The solid was air dried in the filter funnel for 15 minutes and kept under pump vacuum overnight to obtain the orange thiol intermediate.

To the mixture of the thiol intermediate (123 g, 480 mmol) and iodomethane (45.0 mL, 720 mmol) in 1.4 L of ethanol was added DIPEA (86 mL, 490 mmol) slowly. The resulting mixture was stirred overnight at room temperature. The slurry was filtered, and the filtrate was washed with water (600 mL) and ethanol thoroughly to remove DIPEA salt until the filtrate became colorless or a pale-yellow color. The solid was collected and dried under vacuum to provide the product (110 g, 82%). ¹H NMR (400 MHz, DMSO-d₆) δ 2.82 (s, 3H), 6.75 (dd, 1H, J=2.5 Hz, 10.6 Hz), 6.84 (td, 1H, J=2.5 Hz, 8.6 Hz), 7.45 (td, 1H, J=1.0 Hz, 8.6 Hz), 7.99 (s, 1H), 11.27 (s, 1H).

### Example 38

### Preparation of (5Z)-5-[(5-Fluoro-2-hydroxyphenyl)methylidene]-2-(methylsulfanyl)-4,5-dihydro-1,3-thiazol-4-one

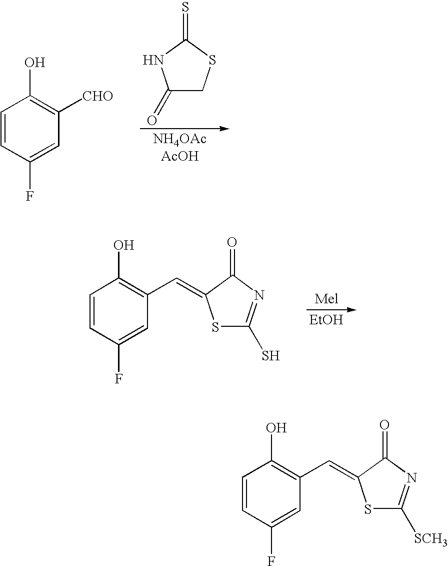

5-(5-Fluoro-2-hydroxybenzylidene)-2-(methylthio)-1,3-thiazol-4(5H)-one) was obtained from 5-fluoro-2-hydroxybenzaldehyde using the same procedure as described in Example 37. The product yield was also 82%. ¹H NMR (400 MHz, DMSO-d₆) δ 2.83 (s, 3H), 6.98 (dd, 1H, J=4.9 Hz, 9.0 Hz), 7.15 (dd, 1H, J=2.9 Hz, 6.6 Hz), 7.23 (td, 1H, J=2.9 Hz, 6.6 Hz), 7.99 (s, 1H), 10.71 (s, 1H); M⁺ 270.

### Example 39

### (5Z)-2-(1,2-diazinan-1-yl)-5-[(4-fluoro-2-hydroxyphenyl)methylidene]-4,5-dihydro-1,3-thiazol-4-one

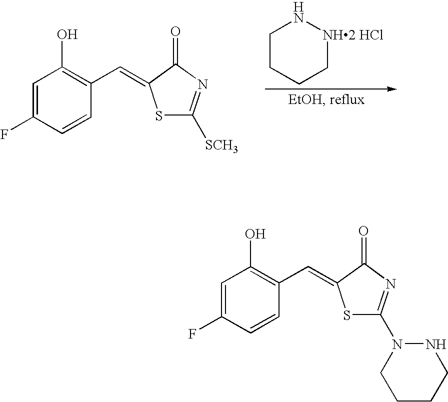

To a mixture of (5Z)-5-(4-fluoro-2-hydroxy-3-methylbenzylidene)-2-(methylthio)-1,3-thiazol-4(5H)-one (13.5 g, 50.0 mmol mmol) in absolute ethanol (100 mL) was added hexahydropyrazidine dihydrochloride (11.1 g, 70.0 mmol) and triethylamine (18.0 mL, 129 mmol). The reaction mixture was stirred at reflux overnight. After cooling to room temperature, the solid product was recovered by filtration and washed with EtOH (1×20 mL). A suspension of this solid in EtOH (50 mL) was stirred at reflux for 0.5 hours. After cooling to room temperature, the solid material was recovered by filtration, washed with EtOH (1×25 mL) and diethyl ether (2×25 mL), and was dried in vacuo, affording (5Z)-5-(4-fluoro-2-hydroxybenzylidene)-2-(hexahydropyridazin-1(2H)-yl)-1,3-thiazol-4(5H)-one (8.2 g; 53%). The product was used without further purification. ¹H NMR (400 MHz, DMSO-d₆) δ 1.64 (m, 2H), 1.74 (m, 2H), 2.93 (m, 2H), 3.86 (m, 2H), 6.01 (t, 1H, J=7.2 Hz), 6.71 (dd, 1H, J=2.6 Hz, 10.7 Hz), 6.81 (td, 1H, J=2.5 Hz, 8.6 Hz), 7.46 (td, 1H, J=2.2 Hz, 6.7 Hz), 7.80 (s, 1H), 10.85 (s, 1H).

### Example 40

### 2-{[(5E)-2-(1,2-Diazinan-1-yl)-4-oxo-4,5-dihydro-1,3-thiazol-5-ylidene]methyl}-5-fluorophenyl N,N-dimethylcarbamate hydrochloride

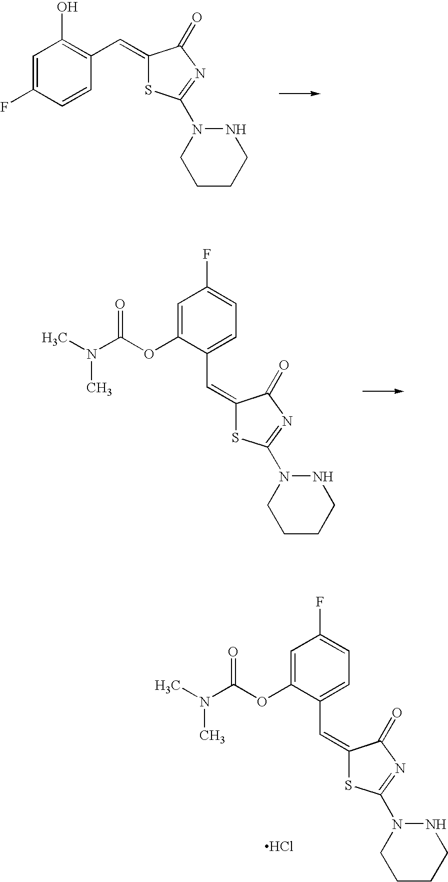

To a mixture of (5Z)-5-(4-fluoro-2-hydroxybenzylidene)-2-(hexahydropyridazin-1(2H)-yl)-1,3-thiazol-4(5H)-one (8.22 g, 26.7 mmol) in anhydrous acetonitrile (120 mL) was added potassium carbonate (7.48 g, 53.4 mmol), followed by dimethylcarbamoyl chloride (3.7 mL, 40.3 mmol). The reaction mixture was stirred and refluxed overnight. After cooling the mixture to room temperature, the solid material was recovered by filtration, washed with water (4×150 mL) and diethyl ether (2×50 mL), and dried in vacuo, affording the neutral product (8.18 g, 81%). ¹H NMR (400 MHz, DMSO-d₆) δ 1.64 (m, 2H), 1.74 (m, 2H), 2.94 (m, 5H), 3.11 (s, 3H), 3.87 (m, 2H), 6.10 (t, 1H, J=7.2 Hz), 7.29 (m, 2H), 7.51 (s, 1H), 7.66 (t, 1H, J=6.5 Hz); M+379. HPLC purity: 99.4%.

Additional product was obtained by recovery of the filtrate of the crude mixture. The filtrate was evaporated to dryness. The residue thus obtained was triturated with 50% acetone/diethyl ether (50 mL), filtered, washed with diethyl ether (2×10 mL), and dried in vacuo. 1.52 g of product (15%) was obtained and ¹H NMR (400 MHz, DMSO-d₆) data were identical. HPLC purity: 98%.

The corresponding dihydrochloride salt was prepared by treating 23.5 g (62 mmol) of the neutral compound in anhydrous methanol (100 mL) at 0° C. To this mixture was slowly added a 4M HCl (38.2 mL, 124 mmol) solution in dioxane. A clear solution was obtained, and this solution was then evaporated, washed with ether, and dried under vacuum to provide 24.5 g (96%) of the dihydrochloride salt as a beige powder. ¹H NMR (400 MHz, DMSO-d₆) δ 1.62 (m, 2H), 1.72 (m, 2H), 2.94 (m, 5H), 3.11 (s, 3H), 3.85 (m, 2H), 6.14 (bs, 1H), 7.29 (m, 2H), 7.48 (s, 1H), 7.67 (t, 1H, J=6.5 Hz); M⁺ 379. HPLC purity: 99%.

### Example 41

### 2-{[(5E)-2-(1,2-Diazinan-1-yl)-4-oxo-4,5-dihydro-1,3-thiazol-5-ylidene]methyl}-5-fluorophenyl pyrrolidine-1-carboxylate hydrochloride

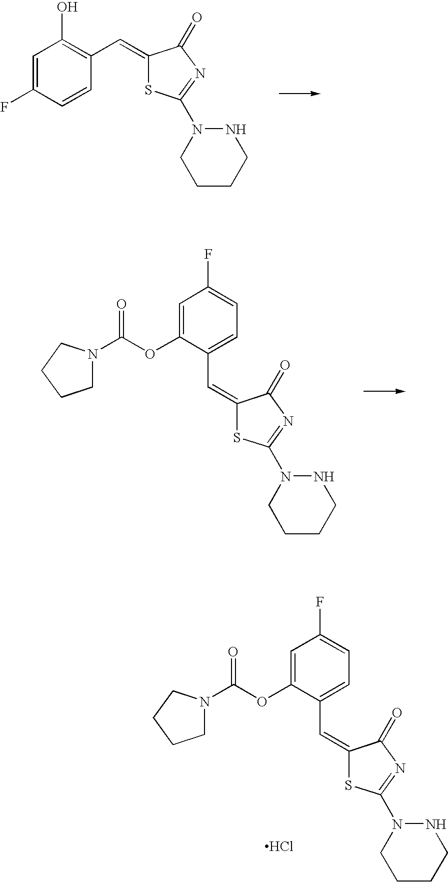

In a 500 mL round bottom flask, (5Z)-2-(1,2-diazinan-1-yl)-5-[(4-fluoro-2-hydroxyphenyl)methylidene]-4,5-dihydro-1,3-thiazol-4-one (17.8 g, 57.5 mmol), K₂CO₃ (86.25 mmol), and pyrrolidine carbonyl chloride (15.4 g, 115 mmol) were mixed in 300 mL of acetonitrile. The reaction was heated at 80° C. overnight. After cooling, the mixture was filtered and washed with MTBE. The solid was collected and dissolved in 7:3 DCM/MeOH (100 mL×2). The mixture was filtered to remove K₂CO₃, and the clear filtrate was evaporated to give the carbamate product as the free base.

To prepare the hydrochloride salt, the carbamate product was dissolved in 3N HCl methanol solution (50 mL). After a few minutes, the solvent was evaporated. The solid was washed with ethyl acetate and dried under vacuum to provide the corresponding salt (24.0 g, 95%). ¹H NMR (400 MHz, DMSO-d₆) δ 1.75 (m, 2H), 1.85 (m, 2H), 1.91 (m, 2H), 1.98 (m, 2H), 2.95 (m, 2H), 3.34 (m, 2H), 3.56 (m, 2H), 3.88 (bs, 2H), 6.13 (t, 1H), 7.33 (m, 2H), 7.52 (s, 1H).

### Example 42

### 2-{[(5E)-2-(1,2-Diazinan-1-yl)-4-oxo-4,5-dihydro-1,3-thiazol-5-ylidene]methyl}-4-fluorophenyl pyrrolidine-1-carboxylate hydrochloride

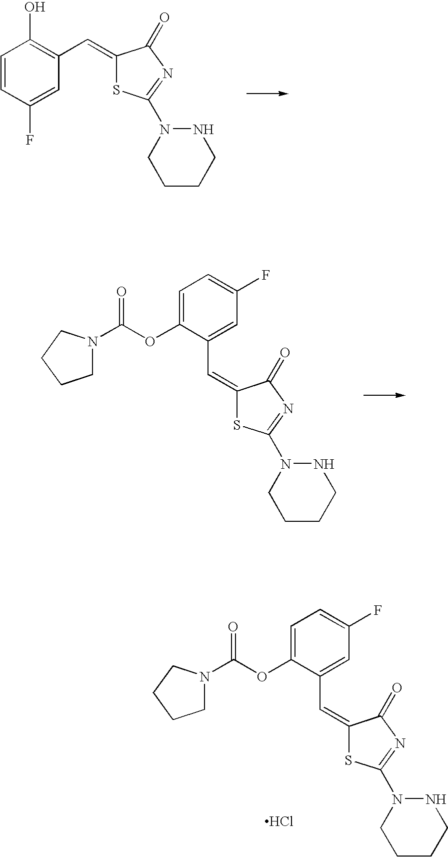

(5Z)-2-(1,2-diazinan-1-yl)-5-[(5-fluoro-2-hydroxyphenyl)methylidene]-4,5-dihydro-1,3-thiazol-4-one (13.7 g; 44.5 mmol) and potassium carbonate (8.0 g, 57.9 mmol) were stirred in acetone (200 mL). A solution of 1-pyrrolidine carbonyl chloride (7.2 mL, 80.2 mmol) in acetone (50 mL) was added portionwise at room temperature. The reaction mixture was heated with additional acetone (2×150 mL) to help solubility. The reaction was then stirred at 60° C. overnight. The mixture was cooled to room temperature and filtered. The solid was washed sequentially with acetone, DCM, and water to remove potassium carbonate. This provided 5.2 g of the neutral carbamate compound, with an HPLC purity of 99.3%. The filtrate from the initial reaction mixture was concentrated under vacuum, and the residue was crystallized in acetone/DCM to afford 12.4 g of carbamate intermediate as a pale yellow solid (69% yield), with an HPLC-purity of 99.5%. The combined yield of this reaction was 97%. ¹H NMR (400 MHz, DMSO) δ 1.65 (m, 2H), 1.75 (m, 2H), 1.85-1.98 (m, 4H), 2.94 (m, 2H), 3.31-3.39 (m, 2H), 3.56 (t, J=6.7 Hz, 2H), 3.87 (bs, 2H), 6.09 (t, J=7.2 Hz, 1H), 7.26-7.31 (m, 2H), 7.55 (s, 1H), 7.64-7.68 (m, 1H); LRMS (ES⁺) m/z 405 (M⁺, 100).

In order to obtain the corresponding hydrochloride salt, the neutral carbamate (12.4 g, 30.6 mmol) was stirred in methanol (80 mL), and a solution of HCl 4N in dioxane was added dropwise at 0° C. The reaction mixture was sonicated to obtain a clear solution. Excess potassium carbonate was removed by filtration, and the filtrate was evaporated. The residue was triturated with diethyl ether (3 times), and the solvent was removed to afford the hydrochloride salt as a pale yellow solid (13.5 g, 100% yield). ¹H NMR (400 MHz, DMSO-d₆) 1.64 (m, 2H), 1.75 (m, 2H), 1.73-1.99 (m, 4H), 2.94 (bs, 2H), 3.34-3.41 (m, 2H), 3.51-3.58 (m, 2H), 3.87 (bs, 2H), 6.11 (t, J=7.1 Hz, 1H), 7.26-7.31 (m, 2H), 7.55 (s, 1H), 7.66 (dd, J=6.4 Hz, J=3.1 Hz, 1H); MS (ES⁺) m/z 405.

### Example 43

### 2-{[(5E)-2-(1,2-Diazinan-1-yl)-4-oxo-4,5-dihydro-1,3-thiazol-5-ylidene]methyl}-5-fluorophenyl (3R)-3-(diethylamino)pyrrolidine-1-carboxylate dihydrochloride

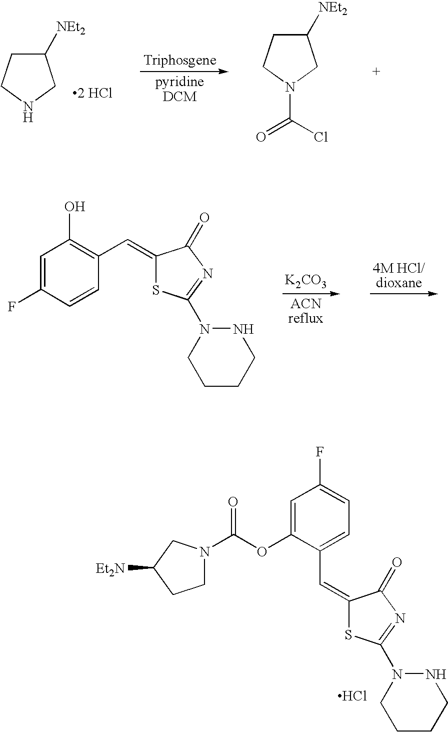

To a 0° C. solution of 3-(diethylamino)pyrrolidine dihydrochloride (2.26 g, 10.5 mmol) in anhydrous CH₂Cl₂ (100 mL) was added pyridine (4 mL, 49 mmol) followed by syringe pump addition (1 hour) of a triphosgene solution (1.1 g, 3.7 mmol) in anhydrous CH₂Cl₂ (8 mL). The mixture was stirred at room temperature overnight. The mixture was extracted with 10% NaHCO₃ (3×100 mL) and water (2×100 mL). The organic phase was dried over MgSO₄, filtered, evaporated and dried in vacuo, affording the 3-(diethylaminol)pyrrolidinecarbonyl chloride product (1.4 g, 67%). The product was used without further purification.

To a mixture of (5Z)-2-(1,2-diazinan-1-yl)-5-[(4-fluoro-2-hydroxyphenyl)methylidene]-4,5-dihydro-1,3-thiazol-4-one (1.14 g, 3.8 mmol) in anhydrous acetonitrile (10 mL) was added potassium carbonate (1.3 g, 9.2 mmol), followed by 3-(diethylaminol)pyrrolidinecarbonyl chloride (1.4 g, 6.9 mmol) in anhydrous acetonitrile (20 mL). The reaction mixture was stirred at reflux overnight. After cooling the mixture to room temperature, the solid material was removed by filtration. The filtrate was recovered and evaporated under reduced pressure. The crude product was purified by flash chromatography (Combiflash Rf, 0-15% MeOH/CH₂Cl₂) and dried in vacuo, affording the carbamate (990 mg, 57%).

To a mixture of carbamate intermediate (2.1 mmol) in methanol (5 mL) was added a solution of 4M HCl/dioxane (4 mL, 12.0 mmol). The resultant solution was filtered. The filtrate was recovered, evaporated and dried in vacuo, affording the final product (11.0 g, 91%). ¹H NMR (400 MHz, DMSO-d₆) δ 1.25 (m, 6H), 1.65 (m, 2H), 1.75 (m, 2H), 2.36 (m, 2H), 2.94 (m, 2H), 3.26 (m, 4H), 3.43 (m, 0.5H), 3.62 (m, 0.5H), 3.70 (m, 0.5H), 3.87 (m, 4H), 4.08 (m, 1.5H), 6.20 (m, 1H), 7.33 (m, 2H), 7.54 (s, 1H), 7.68 (t, 1H, J=6.3 Hz); M+476.

### Example 44

### 2-{[(5E)-2-(1,2-Diazinan-1-yl)-4-oxo-4,5-dihydro-1,3-thiazol-5-ylidene]methyl}-4-fluorophenyl 4-(dimethylamino)piperidine-1-carboxylate dihydro chloride

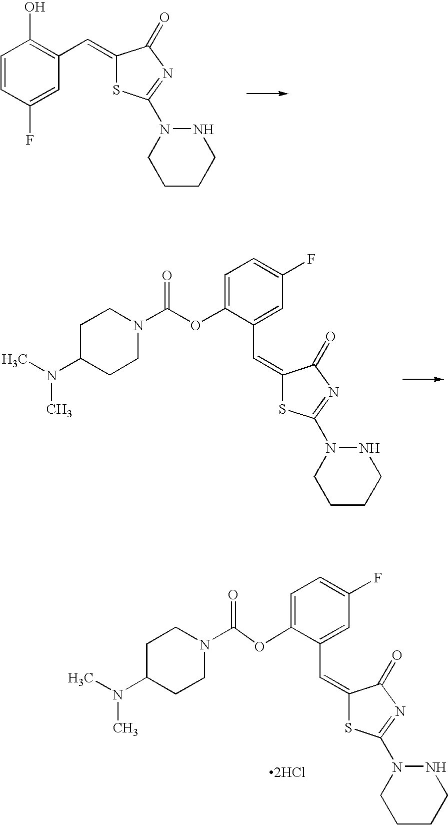

(Z)-4-fluoro-2-((4-oxo-2-(piperazin-1-yl)thiazol-5(4H)-ylidene)methyl)phenyl-4-(dimethylamino)piperidine-1-carboxylate (804 mg, 1.7 mmol) was stirred in methanol (8 mL), and a solution of 4N HCl in dioxane (1.1 mL, 4.3 mmol) was added dropwise at 0° C. The mixture was sonicated until a clear solution was obtained. The solvent was removed, and the residue was washed 2 times with diethyl ether and then dried in vacuo to afford (Z)-4-fluoro-2-((4-oxo-2-(piperazin-1-yl)thiazol-5(4H)-ylidene)methyl)phenyl-4-(dimethylamino)piperidine-1-carboxylate dihydrochloride (867 mg, 93% yield). ¹H NMR (400 MHz, DMSO-d₆) δ 1.40-1.80 (m, 4H), 2.10-2.20 (m, 2H), 2.60-2.80 (bs, 8H), 2.95 (bs, 2H), 3.13 (t, 1H), 3.43 (t, 1H), 3.88 (bs, 2H), 4.13 (d, J=12.5 Hz, 1H), 4.39 (d, J=12.5 Hz, 1H), 6.23 (bs, 1H), 7.32-7.35 (m, 3H), 7.44 (s, 1H), 11.13 (s, 1H).

### Example 45

### 2-{[(5E)-2-(1,2-Diazinan-1-yl)-4-oxo-4,5-dihydro-1,3-thiazol-5-ylidene]methyl}-4-fluorophenyl N-[3-(dimethylamino)propyl]-N-methylcarbamate dihydrochloride

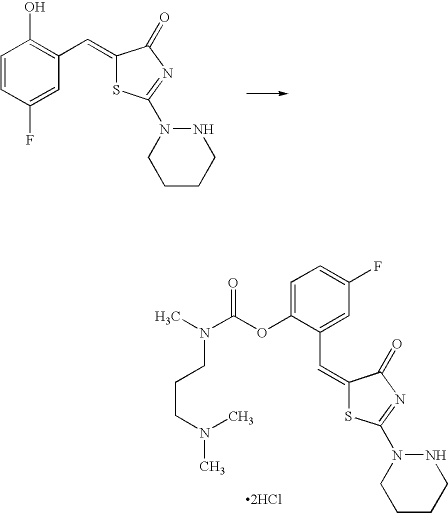

(5Z)-2-(1,2-diazinan-1-yl)-5-[(5-fluoro-2-hydroxyphenyl)methylidene]-4,5-dihydro-1,3-thiazol-4-one 1.0 g; 3.3 mmol) and potassium carbonate (585 mg, 4.3 mmol) were stirred in acetonitrile (15 mL). A solution of (3-(dimethylamino)propyl)(methyl)carbamic chloride (1.0 g, 5.8 mmol) in acetonitrile (5 mL) was then added portionwise at room temperature. The reaction mixture was heated at 75° C. overnight. The mixture was cooled at room temperature, filtered, and the solid was washed with acetone and dichloromethane. The filtrate was evaporated, and the residue was washed two times by diethyl ether and dried in vacuo to afford the product (1.22 g, 84% yield). ¹H NMR (400 MHz, DMSO-d₆) δ 1.60-1.80 (m, 4H), 1.90-2.20 (m, 4H), 2.70-2.78 (m, 6H), 2.90-3.10 (m, 2H), 3.15-3.35 (m, 2H), 3.39 (t, 1H), 3.56 (t, 1H), 3.89 (bs, 2H), 6.25 (bs, 1H), 7.32-7.36 (m, 3H), 7.45 (d, J=12.9 Hz, 1H), 10.70-10.79 (m, 1H).

### Example 46

### 2-{[(5E)-2-(1,2-diazinan-1-yl)-4-oxo-4,5-dihydro-1,3-thiazol-5-ylidene]methyl}-5-fluorophenyl thiomorpholine-4-carboxylate hydrochloride

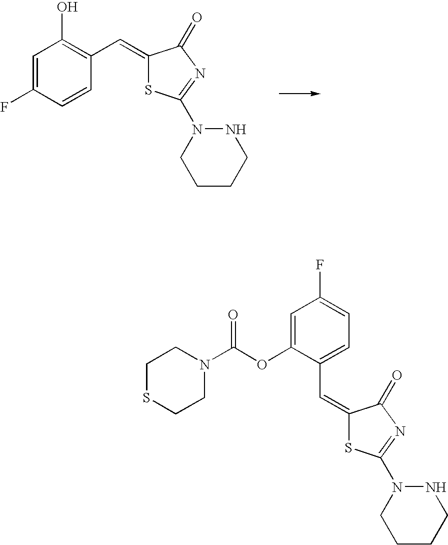

(5Z)-2-(1,2-diazinan-1-yl)-5-[(4-fluoro-2-hydroxyphenyl)methylidene]-4,5-dihydro-1,3-thiazol-4-one (1.0 g; 3.3 mmol) and potassium carbonate (585 mg, 4.2 mmol) were stirred in acetonitrile (15 mL) then a solution of thiomorpholine-4-carbonyl chloride (971 mg, 5.9 mmol, previously prepared from thiomorpholine) in acetonitrile (5 mL) was portionwise added at room temperature. The reaction mixture was heated at 80° C. overnight. The mixture was cooled at room temperature, filtered and the solid was washed with acetone. Filtrate was evaporated and the residue was crystallized in a mixture of DCM/Et₂O, Solid was filtered, washed with diethyl ether and dried in vacuo to afford the product 1.1 g, 77% yield as white solid. ¹H NMR (400 MHz, DMSO-d₆) δ 1.65 (bs, 2H), 1.75 (bs, 2H), 2.66 (bs, 2H), 2.78 (bs, 2H), 2.94 (bs, 2H), 3.70 (bs, 2H), 3.90 (bs, 4H), 6.15 (bs, 1H), 7.28-7.34 (m, 2H), 7.48 (s, 1H), 7.66 (dd, J=6.3 Hz, J=2.3 Hz, 1H).

### Example 47

### 2-{[(5E)-2-(1,2-Diazinan-1-yl)-4-oxo-4,5-dihydro-1,3-thiazol-5-ylidene]methyl}-5-fluorophenyl 1,2-oxazolidine-2-carboxylate hydrochloride

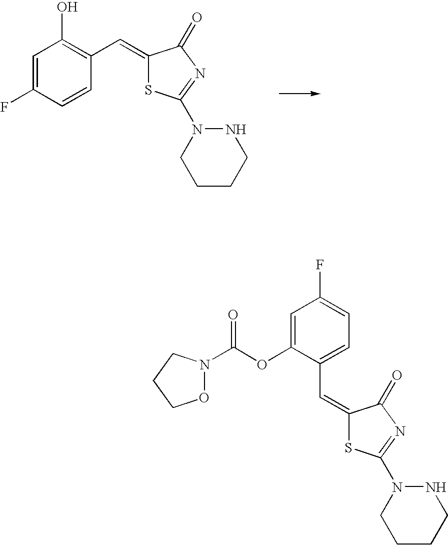

(5Z)-2-(1,2-diazinan-1-yl)-5-[(4-fluoro-2-hydroxyphenyl)methylidene]-4,5-dihydro-1,3-thiazol-4-one (1.7 g; 5.6 mmol) and potassium carbonate (1.0 g, 7.3 mmol) were stirred in acetonitrile (25 mL). A solution of isoxazolidine-2-carbonyl chloride (1.3 g, 10.0 mmol, previously prepared from isoxazolidine hydrochloride) in acetonitrile (5 mL) was then added portionwise at room temperature. The reaction mixture was heated at 80° C. for 4 hours. The mixture was cooled to room temperature, filtered, and the solid was washed with acetone and dichloromethane. The filtrate was evaporated, and the residue was triturated in DCM. The solid was filtered, washed with dichloromethane, and dried in vacuo to afford the product 776 mg, 34% yield. ¹H NMR (400 MHz, DMSO-d₆) δ 1.66 (bs, 2H), 1.76 (bs, 2H), 2.35 (m, 2H), 2.94 (bs, 2H), 3.75 (t, J=7.3 Hz, 2H), 3.87 (bs, 2H), 4.04 (t, J=6.9 Hz, 2H), 6.15 (bs, 1H), 7.31-7.42 (m, 2H), 7.45 (s, 1H), 7.69 (dd, J=6.3 Hz, J=2.5 Hz, 1H).

### Example 48

### 2-{[(5E)-2-(1,2-diazinan-1-yl)-4-oxo-4,5-dihydro-1,3-thiazol-5-ylidene]methyl}-5-fluorophenyl N-[2-(diethylamino)ethyl]-N-ethylcarbamate dihydrochloride

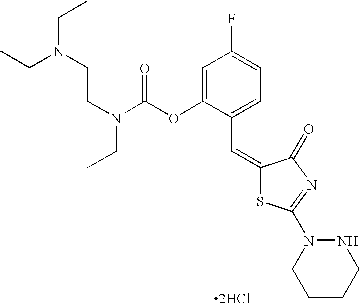

Example 48 was synthesized following the procedure described for Example 45 using (5Z)-2-(1,2-diazinan-1-yl)-5-[(4-fluoro-2-hydroxyphenyl)methylidene]-4,5-dihydro-1,3-thiazol-4-one and N,N,N′-triethylethane-1,2-diamine. ¹H-NMR (DMSO-d₆) δ 1.20 (m, 9H), 1.62-1.64 (m, 4H), 2.90 (m, 4H), 3.01-3.92 (m, 13H), 6.14 (t, J=6.6, 8.3 Hz, 1H), 7.21-7.65 (m, 3H). M⁺=514.3.

### Example 49

### 2-{[(5E)-2-(1,2-diazinan-1-yl)-4-oxo-4,5-dihydro-1,3-thiazol-5-ylidene]methyl}-5-fluorophenyl N-[3-(dimethylamino)propyl]-N-methylcarbamate dihydrochloride

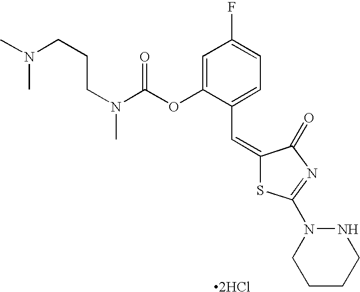

Example 49 was synthesized following the procedure described for Example 45 using (5Z)-2-(1,2-diazinan-1-yl)-5-[(4-fluoro-2-hydroxyphenyl)methylidene]-4,5-dihydro-1,3-thiazol-4-one and N,N,N′-(3-(dimethylamino)propyl)(methyl)carbamic chloride. ¹H-NMR (DMSO-d₆) δ 1.60 (m, 2H), 1.75 (m, 2H), 1.99-2.00 (m, 4H), 2.77 (3s, 9H), 3.90 (m, 3H), 3.41 (m, 1H), 3.59 (m, 1H), 3.80 (bs, 1H), 6.21 (bs, 1H), 7.25 (m, 2H), 7.40 (d, J=13.0 Hz, 1H), 7.65 (m, 1H); M⁺=450.3.

### Example 50

### 2-{[(5E)-2-(1,2-Diazinan-1-yl)-4-oxo-4,5-dihydro-1,3-thiazol-5-ylidene]methyl}-5-fluorophenyl N-[2-(diethylamino)ethyl]-N-methylcarbamate

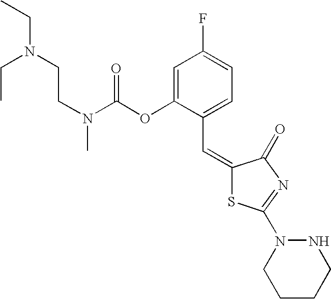

Example 50 was synthesized following an general procedure for Example 45 using (5Z)-2-(1,2-diazinan-1-yl)-5-[(4-fluoro-2-hydroxyphenyl)methylidene]-4,5-dihydro-1,3-thiazol-4-one and N,N,N′ N,N-Diethyl-N′-methyl-ethane-1,2-diamine carbamic chloride. The product was obtained in 50% yield. ¹H-NMR (DMSO-d₆) δ 1.03 (m, 6H), 1.75 (m, 4H), 2.59 (m, 3H), 2.67 and 2.76 (m, 2H), 3.05 (m, 2H), 3.43 and 3.56 (m, 2H), 3.93 (bs, 2H), 4.45 (t, NH), 6.98 (m, 2H), 7.57 (m, 1H), 7.82 (s, 1H).

### Example 51

### 2-{[(5E)-2-(1,2-diazinan-1-yl)-4-oxo-4,5-dihydro-1,3-thiazol-5-ylidene]methyl}-5-fluorophenyl piperidine-1-carboxylate hydrochloride

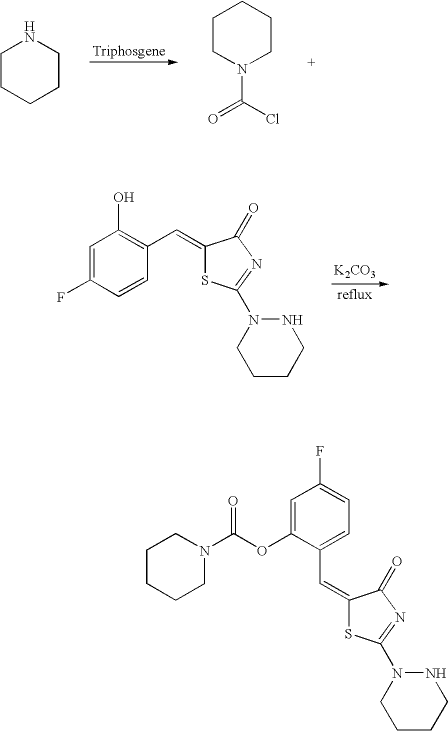

To a 0° C. solution of piperidine (2.3 mL, 23.5 mmol) in anhydrous CH₂Cl₂ (100 mL) was added by a syringe pump (1 hour) a triphosgene solution (2.6 g, 8.8 mmol) in anhydrous CH₂Cl₂ (12 mL). The mixture was stirred at room temperature overnight. The solid material was removed by filtration. The mixture was extracted with 10% NaHCO₃ (2×50 mL) and brine (1×⁻50 mL). The organic phase was dried over MgSO₄, filtered, evaporated and dried in vacuo, affording the piperidinecarbonyl chloride (1.6 g, 47%). The product was used without further purification. To a mixture of (5Z)-2-(1,2-diazinan-1-yl)-5-[(4-fluoro-2-hydroxyphenyl)methylidene]-4,5-dihydro-1,3-thiazol-4-one (1.0 g, 3.3 mmol) in anhydrous acetonitrile (15 mL) was added potassium carbonate (1.1 g, 7.8 mmol) followed by piperidinecarbonyl chloride (800 mg, 5.4 mmol). The reaction mixture was stirred at reflux overnight. After cooling the mixture to room temperature, the solid material was recovered by filtration, washed exhaustively with water (4×50 mL), diethyl ether (2×20 mL), and dried in vacuo, affording the carbamate free base (824 mg, 60%).

The hydrochloride salt was made as follows. To a mixture of carbamate free base (824 mg, 2.0 mmol) in methanol (3 mL) was added a solution of 4M HCl/dioxane (3 mL, 12.0 mmol). The resultant solution was filtered, and the filtrate was recovered and evaporated. The solid was triturated with diethyl ether (50 mL), filtered, and dried to give 1.2 g of the final product (85%). ¹H NMR (400 MHz, DMSO) δ 1.6 (m, 10H), 2.94 (m, 2H), 3.39 (m, 2H), 3.62 (m, 2H), 3.87 (m, 2H), 6.12 (t, 1H, J=7.0 Hz), 7.28 (m, 2H), 7.49 (s, 1H), 7.67 (t, 1H, J=6.4 Hz); M⁺ 419. HPLC purity: 99.3%.

### Example 52

### 2-{[(5E)-2-(1,2-diazinan-1-yl)-4-oxo-4,5-dihydro-1,3-thiazol-5-ylidene]methyl}-5-fluorophenyl morpholine-4-carboxylate hydrochloride

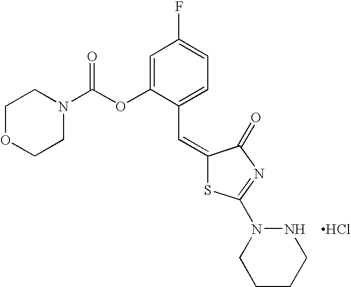

Example 52 was synthesized following the procedure described in Example 46 starting from (5Z)-2-(1,2-diazinan-1-yl)-5-[(4-fluoro-2-hydroxyphenyl)methylidene]-4,5-dihydro-1,3-thiazol-4-one and morpholine carbamoyl chloride. ¹H-NMR (DMSO-d₆) δ 1.61 (m, 2H), 1.75 (m, 2H), 2.95 (m, 2H), 3.45 (m, 2H), 3.61-3.80 (m, 6H), 3.80 (m, 2H), 6.06 (t, J=7.2, 14.4 Hz, 1H), 7.25 (m, 2H), 7.43 (s, 1H), 7.62 (m, 1H); M⁺ 421.5.

### Example 53

### 2-{[(5E)-2-(1,2-Diazinan-1-yl)-4-oxo-4,5-dihydro-1,3-thiazol-5-ylidene]methyl}-5-fluorophenyl (3S)-3-(pyrrolidin-1-yl)pyrrolidine-1-carboxylate

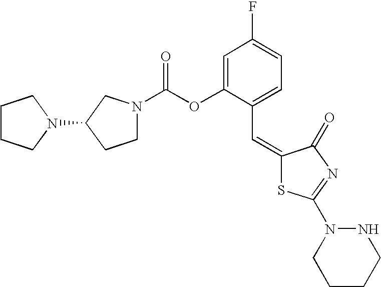

Example 53 was synthesized following the procedure described for Example 46, starting from (5Z)-2-(1,2-diazinan-1-yl)-5-[(4-fluoro-2-hydroxyphenyl)methylidene]-4,5-dihydro-1,3-thiazol-4-one and the (S)-[1,3′]bipyrrolidinyl carbamoylchloride. The product was obtained in 42% yield. ¹H-NMR (DMSO-d₆) δ 1.82 (m, 8H), 1.95-2.18 (m, 2H), 2.60 (m, 4H), 2.90 (m, 1H), 3.06 (m, 2H), 3.30-3.95 (m, 6H), 4.20 (m, NH), 6.95 (m, 1H), 7.06 (m, 1H), 7.60 (m, 1H), 7.83 (s, 1H).

### Example 54

### 2-{[(5E)-2-(1,2-Diazinan-1-yl)-4-oxo-4,5-dihydro-1,3-thiazol-5-ylidene]methyl}-5-fluorophenyl (3R)-3-(pyrrolidin-1-yl)pyrrolidine-1-carboxylate

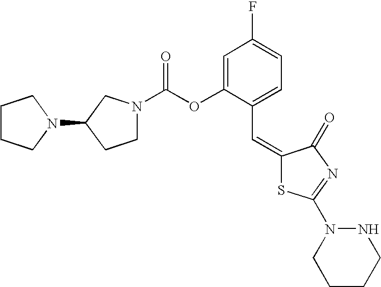

Example 53 was synthesized following the procedure described in Example 46, starting from (5Z)-2-(1,2-diazinan-1-yl)-5-[(4-fluoro-2-hydroxyphenyl)methylidene]-4,5-dihydro-1,3-thiazol-4-one and the (R)-[1,3′]Bipyrrolidinyl carbamoylchloride. The product was obtained in 75% yield. ¹H-NMR (DMSO-d₆) δ 1.80 (m, 8H), 2.00-2.20 (m, 2H), 2.60 (m, 4H), 2.82-2.95 (m, 1H), 3.10 (m, 2H), 3.35-3.95 (m, 6H), 4.42 (m, NH), 6.95 (m, 1H), 7.05 (m, 1H), 7.58 (m, 1H), 8.82 (s, 1H).

### Example 55

### 2-{[(5E)-2-(1,2-Diazinan-1-yl)-4-oxo-4,5-dihydro-1,3-thiazol-5-ylidene]methyl}-5-fluorophenyl (3S)-3-(diethylamino)pyrrolidine-1-carboxylate dihydrochloride

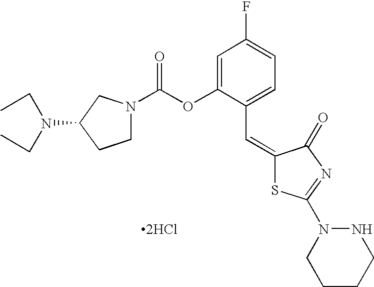

Example 55 was synthesized following the procedure described in Example 43, starting from (5Z)-2-(1,2-diazinan-1-yl)-5-[(4-fluoro-2-hydroxyphenyl)methylidene]-4,5-dihydro-1,3-thiazol-4-one and the diethyl-pyrrolidin-3-yl-amine. The product was obtained in 70% yield. ¹H-NMR (DMSO-d₆) δ 1.00 (2t, 6H) 1.60 (m, 2H), 1.80 (m, 2H), 2.12 (m, 1H), 2.60 (m, 4H), 2.90 (m, 2H), 3.40 (m, 4H), 3.55-3.99 (m, 4H), 6.00 (t, J=7.2, 14.3 Hz, 1H), 7.21 (m, 2H), 7.50 (s, 1H), 7.65 (t, J=2.5, 8.8 Hz, 1H).

### Example 56

### 2-{[(5E)-2-(1,2-Diazinan-1-yl)-4-oxo-4,5-dihydro-1,3-thiazol-5-ylidene]methyl}-5-fluorophenyl 1,3-thiazolidine-3-carboxylate hydrochloride

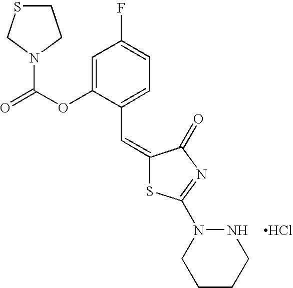

Example 56 was synthesized following the procedure described in Example 51, using (5Z)-2-(1,2-diazinan-1-yl)-5-[(4-fluoro-2-hydroxyphenyl)methylidene]-4,5-dihydro-1,3-thiazol-4-one and thiozolidinyl carbamoylchloride. The product was obtained in yields ranging from 40-60%. ¹H NMR (400 MHz, DMSO-d₆) δ 1.65 (m, 2H), 1.75 (m, 2H), 2.95 (m, 2H), 3.17 (m, 2H), 3.71 (m, 1H), 3.88 (m, 3H), 6.11 (t, 1H, J=7.0 Hz), 7.32 (m, 2H), 7.52 (s, 1H), 7.66 (t, 1H, J=6.3 Hz); M⁺ 423.

### Example 57

### 2-{[(5E)-2-(1,2-Diazinan-1-yl)-4-oxo-4,5-dihydro-1,3-thiazol-5-ylidene]methyl}-5-fluorophenyl azetidine-1-carboxylate

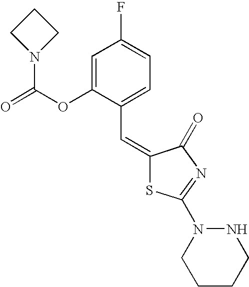

Example 57 was synthesized following the procedure described in Example 43, using (5Z)-2-(1,2-diazinan-1-yl)-5-[(4-fluoro-2-hydroxyphenyl)methylidene]-4,5-dihydro-1,3-thiazol-4-one and azetedinylcarbamoyl chloride. The product was obtained in yields ranging from 40-60%. ¹H NMR (400 MHz, DMSO-d₆) δ 1.66 (m, 2H), 1.76 (m, 2H), 2.31 (m, 2H), 2.94 (m, 2H), 3.97 (m, 2H), 4.02 (t, 2H, J=7.2 Hz), 4.24 (t, 2H, J=7.0 Hz), 6.15 (m, 1H), 7.29 (m, 2H), 7.53 (s, 1H), 7.68 (t, 1H, J=6.3 Hz); M⁺ 391.

### Example 58

### 2-{[(5E)-2-(1,2-Diazinan-1-yl)-4-oxo-4,5-dihydro-1,3-thiazol-5-ylidene]methyl}-4-fluorophenyl (3R)-3-(diethylamino)pyrrolidine-1-carboxylate dihydrochloride

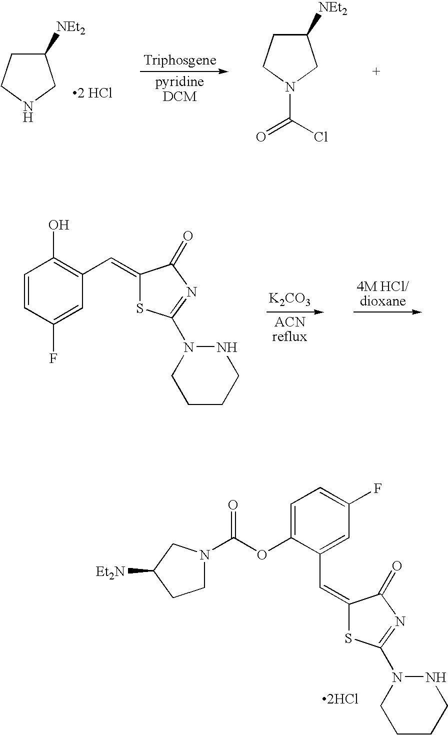

To a 0° C. solution of 3-(diethylamino)pyrrolidine dihydrochloride (2.26 g, 10.5 mmol) in anhydrous CH₂Cl₂ (100 mL) was added pyridine (4 mL, 49 mmol) followed by a syringe pump addition (1 hour) of a triphosgene solution (1.1 g, 3.7 mmol) in anhydrous CH₂Cl₂ (8 mL). The mixture was stirred at room temperature overnight. The mixture was extracted with 10% NaHCO₃ (3×100 mL) and water (2×100 mL). The organic phase was dried over MgSO₄, filtered, evaporated, and dried in vacuo, affording the 3-(diethylaminol)pyrrolidinecarbonyl chloride (1.4 g, 67%). The product was used without further purification.

To a mixture of (5Z)-2-(1,2-diazinan-1-yl)-5-[(5-fluoro-2-hydroxyphenyl)methylidene]-4,5-dihydro-1,3-thiazol-4-one (1.4 g, 4.6 mmol) in anhydrous acetonitrile (10 mL) was added potassium carbonate (1.3 g, 9.2 mmol), followed by 3-(diethylaminol)pyrrolidinecarbonyl chloride (1.4 g, 6.9 mmol) in anhydrous acetonitrile (20 mL). The reaction mixture was stirred at reflux overnight. After cooling the mixture to room temperature, the solid material was removed by filtration. The filtrate was recovered and evaporated under reduced pressure. The crude product was purified by flash chromatography (Combiflash Rf, 0-10% MeOH/CH₂Cl₂) and dried in vacuo, affording the carbamate free base (826 mg, 38%).

To a mixture of carbamate free base (826 mg, 1.7 mmol mmol) in methanol (5 mL) was added a solution of 5-6NHC1/isopropanol (10 mL). The resultant solution was filtered. The filtrate was recovered, evaporated under reduced pressure, co-evaporated with water (3 mL) and dried in vacuo, affording the final product (795 mg, 85%). ¹H NMR (400 MHz, DMSO-d₆) δ 1.25 (m, 6H), 1.65 (m, 2H), 1.75 (m, 2H), 2.35 (m, 2H), 2.95 (m, 2H), 3.23 (m, 4H), 3.37 (m, 0.5H), 3.61 (m, 0.5H), 3.68 (m, 0.5H), 3.86 (m, 4H), 4.06 (m, 1.5H), 6.21 (m, 1H), 7.38 (m, 3H), 7.51 (s, 1H); M⁺ 476. HPLC: 98.6%.

### Example 59

### 2-{[(5E)-2-(1,2-Diazinan-1-yl)-4-oxo-4,5-dihydro-1,3-thiazol-5-ylidene]methyl}-4-fluorophenyl piperidine-1-carboxylate hydrochloride

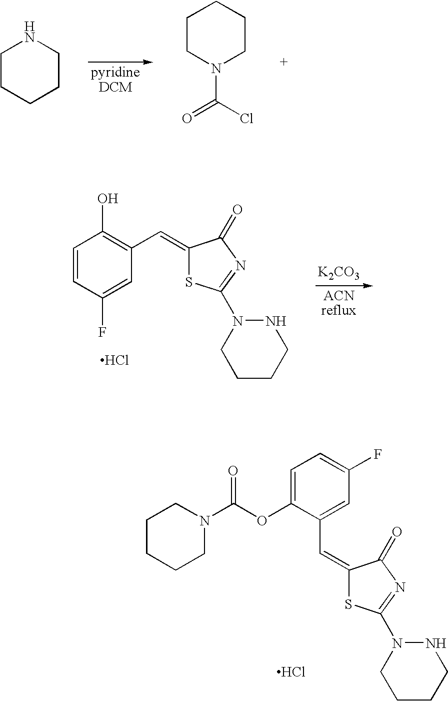

To a 0° C. solution of 4-piperidine (2.3 mL, 23.5 mmol) in anhydrous CH₂Cl₂ (100 mL) was added by syringe pump (1 hour) a triphosgene solution (2.6 g, 8.8 mmol) in anhydrous CH₂Cl₂ (12 mL). The mixture was stirred at room temperature overnight. The solid material was removed by filtration. The mixture was extracted with 10% NaHCO₃ (2×50 mL) and brine (1×50 mL). The organic phase was dried over MgSO₄, filtered, evaporated, and dried in vacuo, affording the piperidinecarbonyl chloride (1.6 g, 47%). The product was used without further purifications.

To a mixture of (5Z)-2-(1,2-diazinan-1-yl)-5-[(5-fluoro-2-hydroxyphenyl)methylidene]-4,5-dihydro-1,3-thiazol-4-one (1.0 g, 3.3 mmol) in anhydrous acetonitrile (15 mL) was added potassium carbonate (1.1 g, 7.8 mmol) followed by piperidinecarbonyl chloride (800 mg, 5.4 mmol). The reaction mixture was stirred at reflux overnight. After cooling the mixture to room temperature, the solid material was recovered by filtration, washed exhaustively with water (4×50 mL), diethyl ether (2×20 mL), and dried in vacuo, affording the carbamate free base (824 mg, 60%).

To a mixture of carbamate free base (824 mg, 2.0 mmol) in methanol (3 mL) was added a solution of 4M HCl/dioxane (12.0 mmol HCl, 3 mL). The resultant solution was filtered. The filtrate was recovered and evaporated. The solid was triturated with diethyl ether (50 mL). The solid material was recovered by filtration and dried in vacuo, affording the final product (792 mg, 87%). ¹H NMR (400 MHz, DMSO-d₆) δ 1.6 (m, 10H), 2.94 (m, 2H), 3.39 (m, 2H), 3.62 (m, 2H), 3.87 (m, 2H), 6.12 (t, 1H, J=7.0 Hz), 7.28 (m, 2H), 7.49 (s, 1H), 7.67 (t, 1H, J=6.4 Hz); M⁺ 419. HPLC purity: 99.3%

### Example 60

### 2-{[(5E)-2-(1,2-Diazinan-1-yl)-4-oxo-4,5-dihydro-1,3-thiazol-5-ylidene]methyl}-4-fluorophenyl N-[2-(diethylamino)ethyl]-N-methylcarbamate dihydrochloride

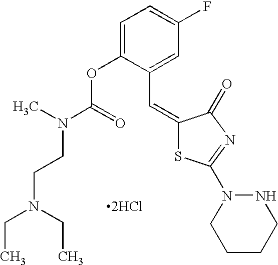

Example 60 was synthesized using the procedure described in Example 59 by combining (5Z)-2-(1,2-diazinan-1-yl)-5-[(4-fluoro-2-hydroxyphenyl)methylidene]-4,5-dihydro-1,3-thiazol-4-one with N,N,N′-(2-(diethylamino)ethyl)(methyl)carbamic chloride. The product was obtained in 40% yield. ¹H NMR (400 MHz, DMSO-d₆) δ 1.48 (m, 6H), 1.75 (m, 2H), 2.94 (m, 2H), 3.39 (m, 2H), 3.62 (m, 2H), 3.87 (m, 2H), 6.14 (t, 1H, J=7.0 Hz), 7.32 (m, 3H), 7.47 (s, 1H); M+464. HPLC purity: 98.3%.

### General Procedure for Thiorhodanine Analogues:

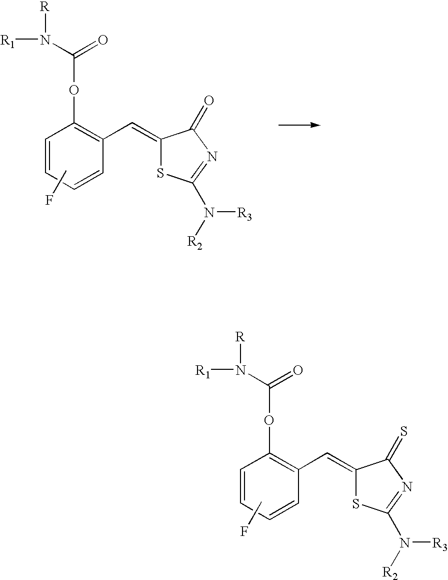

Procedure A: The mixture of rhodanine precursor (1.0 eq) and Lawesson's reagent (1.05 eq) in ACN (0.5M) was refluxed for 2 hours. After evaporation of the solvent, the crude solid was purified by CombiFlash (MeOH/dichloromethane as eluent). Yields varied from 5-50%.

Procedure B: The mixture of rhodanine precursor (1.0 eq) and P₂S₅ (1.1 eq) in THF (0.5M) was heated at 60° C. for 3 hours. After evaporation, the crude solid was purified by CombiFlash (MeOH/dichloromethane as eluent). Yields varied from 5-50%.

Procedure C: The mixture of rhodanine precursor (1.0 eq) and P₂S₅ (1.1 eq) in pyridine (0.5M) was heated at 100° C. for 2 hours. After evaporation, the crude solid was purified by CombiFlash (MeOH/dichloromethane as eluent). Yields varied from 5-50%.

### Example 61

### (5Z)-2-(1,2-Diazinan-1-yl)-5-[(4-fluoro-2-hydroxyphenyl)methylidene]-4,5-dihydro-1,3-thiazole-4-thione

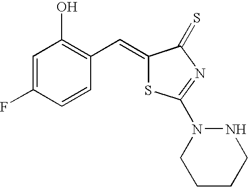

Example 61 was synthesized from Example 39 using procedure A from the general procedure for thiorhodanine analogues. ¹H NMR (400 MHz, DMSO-d₆) δ 1.62-1.80 (m, 4H), 3.00 (m, 2H), 3.95 (br, 2H), 6.28 (t, NH), 6.78 (m, 2H), 7.50 (m, 1H), 8.25 (s, 1H), 1.00 (s, 1H).

### Example 62

### 2-{[(5Z)-2-(1,2-Diazinan-1-yl)-4-sulfanylidene-4,5-dihydro-1,3-thiazol-5-ylidene]methyl}-5-fluorophenyl 4-(piperidin-1-yl)piperidine-1-carboxylate

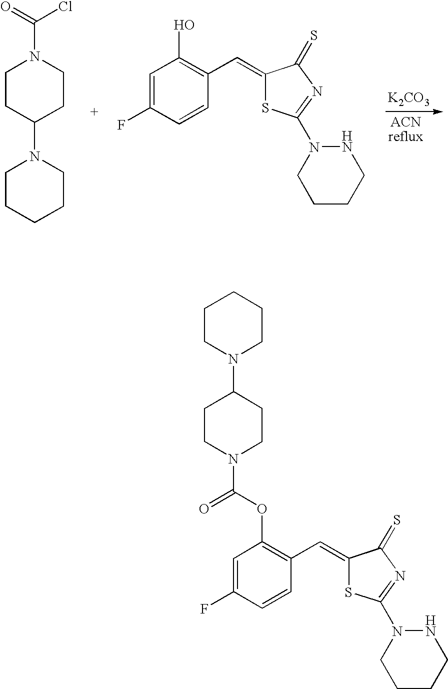

To a mixture of (5Z)-5-(4-fluoro-2-hydroxybenzylidene)-2-(tetrahydropyridazin-1(2H)-yl)-1,3-thiazole-4(5H)-thione (783 mg, 2.4 mmol) in anhydrous acetonitrile (15 mL) was added potassium carbonate (663 mg, 4.8 mmol), followed by a solution of 4-piperidinopiperidinecarbonyl chloride (3.9 mmol) in anhydrous acetonitrile (5 mL). The reaction mixture was stirred at reflux overnight. After cooling the mixture to room temperature, the solid material was removed by filtration. The filtrate was recovered and evaporated under reduced pressure. The crude product was purified by flash chromatography (Combiflash Rf, 0-30% MeOH/CH₂Cl₂), affording the title compound (43 mg, 4%). ¹H NMR (400 MHz, DMSO-d₆) δ 1.65 (m, 14H), 2.83 (m, 7H), 4.02 (m, 5H), 4.25 (m, 1H), 6.39 (t, 1H, J=7.0 Hz), 7.31 (m, 2H), 7.69 (t, 1H, J=6.5 Hz), 7.98 (s, 1H); M⁺ 518.

### Example 63

### 2-{[(5Z)-2-(1,2-Diazinan-1-yl)-4-sulfanylidene-4,5-dihydro-1,3-thiazol-5-ylidene]methyl}-5-fluorophenyl morpholine-4-carboxylate

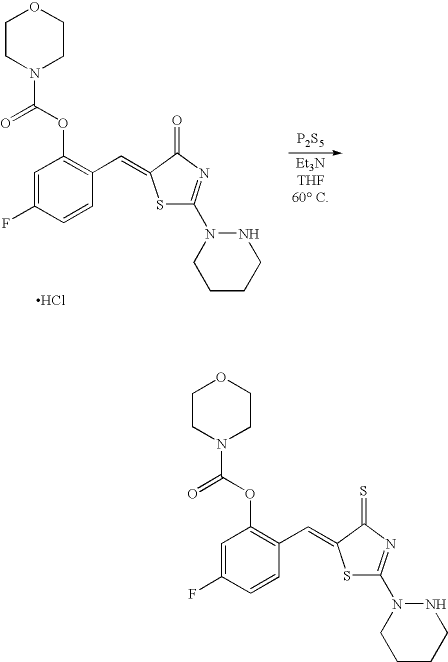

To a mixture of 4-fluoro-2-{(Z)-[4-oxo-2-(tetrahydropyridazin-1(2H)-yl)-1,3-thiazol-5(4H)-ylidene]methyl}phenyl-morpholine-4-carboxylate dihydrochloride (1.0 g, 2.2 mmol) and triethylamine (500 μL, 3.5 mmol) in anhydrous THF (20 mL) was added phosphorus pentasulfide (1.02 g, 2.3 mmol). The reaction mixture was stirred at 60° C. for 5 hours. After cooling the mixture to room temperature, the solid material was removed by filtration. The filtrate was recovered and evaporated under reduced pressure. The crude product was purified by flash chromatography (Combiflash Rf, isocratic 1% MeOH in 1:1:1 CH₂Cl₂/hexanes/EtOAc), affording the title compound (150 mg, 16%). ¹H NMR (400 MHz, DMSO-d₆) δ 1.66 (m, 2H), 1.80 (m, 2H), 3.00 (m, 2H), 3.43 (m, 2H), 3.71 (m, 6H), 3.97 (m, 2H), 6.36 (t, 1H, J=7.0 Hz), 7.32 (m, 2H), 7.70 (t, 1H, J=6.5 Hz), 7.99 (s, 1H); M⁺ 437.

### Example 64

### 2-{[(5Z)-2-(1,2-Diazinan-1-yl)-4-sulfanylidene-4,5-dihydro-1,3-thiazol-5-ylidene]methyl}-5-fluorophenyl 4-(pyrrolidin-1-yl)piperidine-1-carboxylate

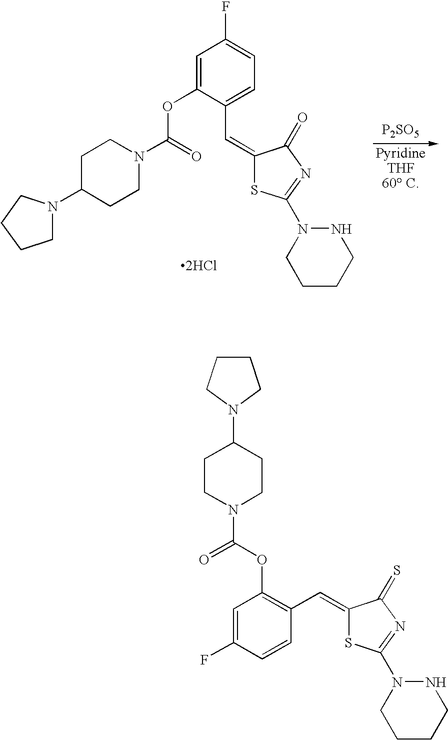

To a mixture of 2-{[(5E)-2-(1,2-diazinan-1-yl)-4-oxo-4,5-dihydro-1,3-thiazol-5-ylidene]methyl}-5-fluorophenyl 4-(pyrrolidin-1-yl)piperidine-1-carboxylate dihydrochloride (100 mg, 0.18 mmol) in pyridine (5 mL) was added phosphorus pentasulfide (85 mg, 0.19 mmol). The reaction mixture was stirred at 100° C. for 3 hours. After cooling to room temperature, the solvent was evaporated. Dichloromethane (20 mL) was added to the residue. The solid material was removed by filtration. The filtrate was recovered and extracted with water (3×20 mL). The organic phase was recovered, dried over MgSO₄, filtered, evaporated, and dried in vacuo, affording the product (25 mg, 27%). ¹H NMR (400 MHz, DMSO-d₆) δ 1.87 (m, 12H), 2.50 (m, 1H), 3.09 (m, 5H), 3.26 (m, 1H), 3.96 (m, 5H), 4.10 (m, 1H), 6.36 (t, 1H, J=6.8 Hz), 7.30 (m, 2H), 7.70 (t, 1H, J=6.3 Hz), 8.00 (s, 1H); M⁺ 504.

### Example 65

### 2-{[(5Z)-2-(1,2-Diazinan-1-yl)-4-sulfanylidene-4,5-dihydro-1,3-thiazol-5-ylidene]methyl}-5-fluorophenyl N,N-diethylcarbamate

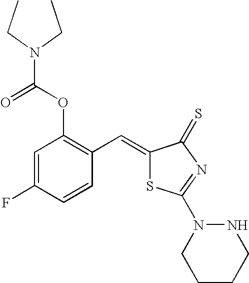

Example 65 was synthesized from 2-{[(5E)-2-(1,2-diazinan-1-yl)-4-oxo-4,5-dihydro-1,3-thiazol-5-ylidene]methyl}-5-fluorophenyl N,N-diethylcarbamate hydrochloride using the procedure described for Example 63. This provided the product in 10% yield. ¹H NMR (400 MHz, DMSO-d₆) δ 1.22 (m, 6H), 1.70-1.90 (m, 4H), 3.06 (m, 2H), 3.39 (m, 2H), 3.56 (m, 2H), 4.05 (m, 2H), 4.75 (t, NH), 6.92 (m, 1H), 7.08 (m, 1H), 7.54 (m, 1H), 8.26 (s, 1H).

### Example 66

### 2-{[(5Z)-2-(1,2-Diazinan-1-yl)-4-sulfanylidene-4,5-dihydro-1,3-thiazol-5-ylidene]methyl}-5-fluorophenyl N-ethyl-N-methylcarbamate

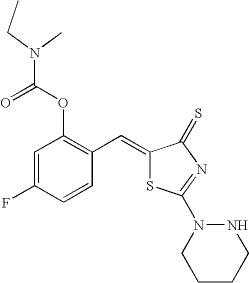

Example 66 was synthesized from 2-{[(5E)-2-(1,2-diazinan-1-yl)-4-oxo-4,5-dihydro-1,3-thiazol-5-ylidene]methyl}-5-fluorophenyl N-ethyl-N-methylcarbamate following the procedure for Example 63. The product was obtained in 10% yield. ¹H NMR δ 1.20 (m, 3H), 1.72-1.85 (m, 4H), 3.08 (m, 2H), 3.41, 3.58 (m, 2H), 4.06 (br, 2H), 4.62 (m, NH), 6.95 (m, 1H), 7.05 (m, 1H), 7.53 (m, 1H), 8.25 (s, 1H).

### Example 67

### 2-{[(5Z)-2-(1,2-Diazinan-1-yl)-4-sulfanylidene-4,5-dihydro-1,3-thiazol-5-ylidene]methyl}-5-fluorophenyl (3R)-3-(diethylamino)pyrrolidine-1-carboxylate

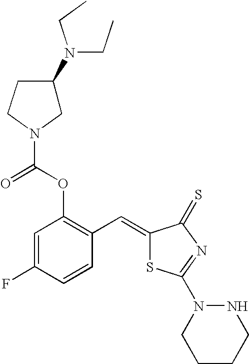

Example 67 was synthesized from 2-{[(5E)-2-(1,2-diazinan-1-yl)-4-oxo-4,5-dihydro-1,3-thiazol-5-ylidene]methyl}-5-fluorophenyl (3R)-3-(diethylamino)pyrrolidine-1-carboxylate using the procedure described for Example 64. The product was obtained in 5% yield. ¹H NMR (400 MHz, DMSO-d₆) δ 1.38 (m, 6H), 1.75-1.85 (m, 4H), 2.35 (m, 2H), 2.95-3.05 (m, 4H), 3.6-4.2 (m, 9H), 4.65 (m, NH), 6.95 (m, 1H), 7.20 (m, 1H), 7.60 (m, 1H), 8.25 (d, 1H).

### Example 68

### 2-{[(5Z)-2-(1,2-Diazinan-1-yl)-4-sulfanylidene-4,5-dihydro-1,3-thiazol-5-ylidene]methyl}-5-fluorophenyl (3R)-3-(pyrrolidin-1-yl)pyrrolidine-1-carboxylate

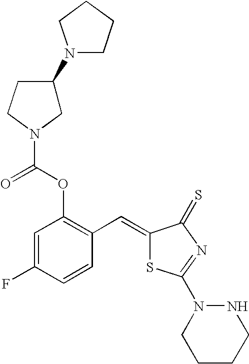

Example 68 was synthesized from 2-{[(5E)-2-(1,2-diazinan-1-yl)-4-oxo-4,5-dihydro-1,3-thiazol-5-ylidene]methyl}-5-fluorophenyl (3R)-3-(pyrrolidin-1-yl)pyrrolidine-1-carboxylate following the procedure described for Example 64. The product was obtained in 10% yield. ¹H NMR (400 MHz, DMSO-d₆) δ 1.80 (m, 8H), 2.00-2.15 (m, 2H), 2.58 (m, 4H), 2.82-2.95 (m, 1H), 3.05 (m, 2H), 3.35-3.95 (m, 4H), 4.08 (br, 2H), 4.62 (m, NH), 6.95 (m, 1H), 7.18 (m, 1H), 7.55 (m, 1H), 8.38 (d, 1H).

### Example 69

### 2-{[(5Z)-2-(1,2-diazinan-1-yl)-4-sulfanylidene-4,5-dihydro-1,3-thiazol-5-ylidene]methyl}-5-fluorophenyl (3R)-3-(diethylamino)pyrrolidine-1-carboxylate

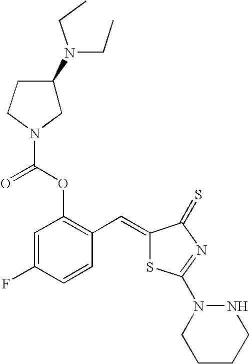

Example 69 was synthesized from 2-{[(5E)-2-(1,2-diazinan-1-yl)-4-oxo-4,5-dihydro-1,3-thiazol-5-ylidene]methyl}-5-fluorophenyl (3R)-3-(diethylamino)pyrrolidine-1-carboxylate following the general procedure described for Example 64. The product was obtained in 15% yield. ¹H-NMR (CDCl₃) δ 1.38 (m, 6H), 1.75-1.85 (m, 4H), 2.35 (m, 2H), 2.95-3.05 (m, 4H), 3.6-4.2 (m, 9H), 4.65 (m, NH), 6.95 (m, 1H), 7.20 (m, 1H), 7.60 (m, 1H), 8.25 (d, 1H).

### Example 70

### 2-{[(5Z)-2-(1,2-Diazinan-1-yl)-4-sulfanylidene-4,5-dihydro-1,3-thiazol-5-ylidene]methyl}-5-fluorophenyl (3S)-3-(diethylamino)pyrrolidine-1-carboxylate

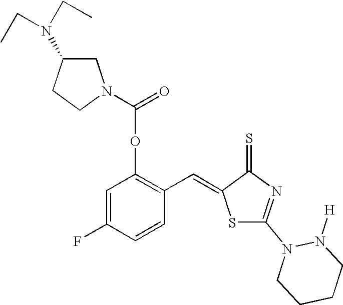

Example 71 was synthesized from the corresponding compound 2-{[(5E)-2-(1,2-diazinan-1-yl)-4-oxo-4,5-dihydro-1,3-thiazol-5-ylidene]methyl}-5-fluorophenyl (3S)-3-(diethylamino)pyrrolidine-1-carboxylate following the general procedure described for Example 64. The product was obtained in 18% yield. ¹H NMR (CDCl₃; includes cis and trans isomers) δ 1.00 (2t, 6H) 1.20 (m, 2H), 1.40 (m, 2H), 1.98 (m, 2H), 2.00 (m, 4H), 2.21 (m, 2H), 2.81 (m, 5H), 3.50 (q, 2H), 4.70 (t, J=7.6, 15.1 HZ, 1H), 6.99 (t, J=2.9, 8.1 Hz, 1H), 7.21 (m, 2H), 7.59 (m, 1H), 8.40 (2s, 2H 1H); M⁺: 492.5.

### Example 71

### 2-{[(5Z)-2-(1,2-Diazinan-1-yl)-4-sulfanylidene-4,5-dihydro-1,3-thiazol-5-ylidene]methyl}-5-fluorophenyl pyrrolidine-1-carboxylate

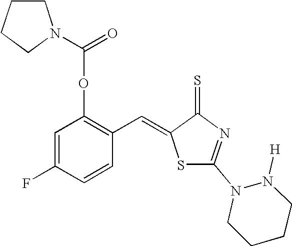

Example 71 was synthesized from Example 41, and sulfur was introduced following the procedure described in Example 63. The product was obtained in 10% yield. ¹H-NMR (DMSO-d₆) δ 1.65 (m, 2H), 1.80 (m, 2H), 1.91-2.01 (m, 2H), 2.99 (m, 2H), 3.40 (m, 4H), 3.60 (m, 2H), 4.00 (m, 2H), 6.20 (t, J=7.2, 14.4 Hz, 1H), 7.22 (m, 2H), 7.63 (m, 1H), 7.62 (m, 1H).

### Example 72

### 2-{[(5Z)-2-(1,2-Diazinan-1-yl)-4-sulfanylidene-4,5-dihydro-1,3-thiazol-5-ylidene]methyl}-5-fluorophenyl piperidine-1-carboxylate

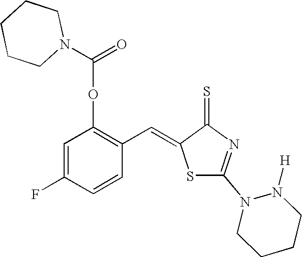

Example 72 was synthesized from Example 52 following the procedure described for the synthesis of Example 63. The product was obtained in 10% yield. ¹H-NMR (CDCl₃) δ 1.61-1.85 (m, 101H), 3.15 (m, 2H), 3.55 (m, 2H), 3.75 (m, 2H), 4.19 (m, 2H), 4.51 (t, J=7.5, 14.9 Hz, 1H), 6.93 (t, J=8.4, 14.3 Hz, 1H), 7.09 (d, J=8.4 Hz, 1H), 7.60 (m, 1H), 8.30 (s, 1H).

### Example 73

### 2-{[(5Z)-2-(1,2-Diazinan-1-yl)-4-sulfanylidene-4,5-dihydro-1,3-thiazol-5-ylidene]methyl}-5-fluorophenyl N,N-dimethylcarbamate

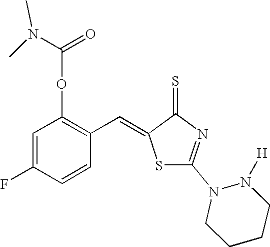

Example 73 was synthesized from Example 40 following the procedure described in Example 63. The product was obtained in 10% yield. ¹H-NMR (CDCl₃) δ 1.81 (m, 2H), 1.95 (m, 2H), 3.10 (s, 31H), 3.15 (m, 2H), 3.21 (s, 31H), 4.15 (m, 2H), 4.41 (t, J=7.5, 14.9 Hz, 1H), 6.93 (t, J=7.1. 10.9 Hz, 1H), 7.09 (d, J=10.9 Hz, 1H), 7.60 (m, 1H), 8.20 (s, 1H).

### Example 74

### 2-{[(5E)-2-(1,2-Diazinan-1-yl)-4-oxo-4,5-dihydro-1,3-thiazol-5-ylidene]methyl}-5-fluorophenyl 2-oxopyrrolidine-1-carboxylate

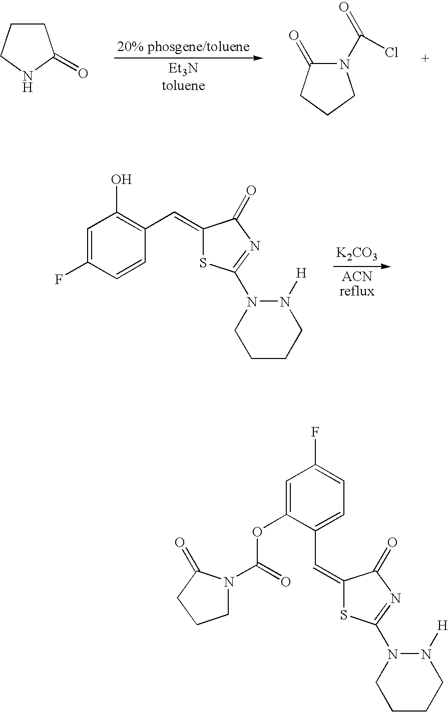

To a solution of 2-pyrrolidinone (1.8 mL, 23.5 mmol) and triethylamine (3.3 mL, 23.7 mmol) in anhydrous toluene (30 mL) at 0° C. was added slowly a solution of phosgene (20% in toluene, 12 mL). The mixture was stirred at room temperature for 36 hours. The solid material was removed by filtration. The filtrate was recovered, evaporated, and dried in vacuo, affording the 2-oxopyrrolidine-1-carbonyl chloride (2.48 g, 72%). The product was used without further purification.

To a mixture of (5Z)-2-(1,2-diazinan-1-yl)-5-[(4-fluoro-2-hydroxyphenyl)methylidene]-4,5-dihydro-1,3-thiazol-4-one (1.6 g, 5.3 mmol) and potassium carbonate (1.46 g, 10.6 mmol) in anhydrous ACN (20 mL) was added 2-oxopyrrolidine-1-carbonyl chloride (2.48 g, 16.7 mmol) in anhydrous ACN (10 mL). The reaction mixture was stirred at reflux overnight. The solid material was recovered by filtration. The filtrate was recovered and evaporated under reduced pressure. The residue was triturated with toluene (75 mL) and dichloromethane (2 mL). The solid material was recovered by filtration, washed with CH₂Cl₂/ACN (80/20, 250 mL), and dried in vacuo. The crude product was purified by flash chromatography (Combiflash Rf, 0-100% EtOAc/CH₂Cl₂), affording the final product (115 mg, 5%). ¹H NMR (400 MHz, DMSO-d₆) δ 1.65 (m, 2H), 1.75 (m, 2H), 2.04 (m, 2H), 2.57 (t, 1H, J=8.2 Hz), 2.94 (m, 2H), 3.90 (m, 4H), 6.11 (t, 1H, J=7.0 Hz), 7.38 (m, 2H), 7.52 (s, 1H), 7.73 (t, 1H, J=6.3 Hz); M⁺ 419.

### Example 75

### 2-{[(5E)-2-(1,2-Diazinan-1-yl)-4-oxo-4,5-dihydro-1,3-thiazol-5-ylidene]methyl}-5-fluorophenyl 5-chloro-2,3-dihydro-1H-pyrrole-1-carboxylate

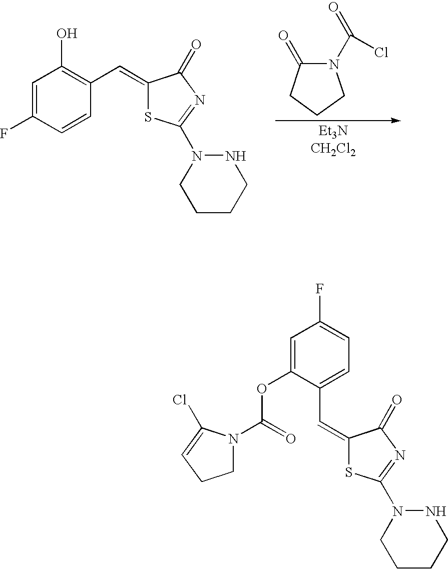

To a mixture of (5Z)-2-(1,2-diazinan-1-yl)-5-[(4-fluoro-2-hydroxyphenyl)methylidene]-4,5-dihydro-1,3-thiazol-4-one (1.4 g, 4.7 mmol) and triethylamine (1.3 mL, 9.4 mmol)) in anhydrous dichloromethane (20 mL) was added 2-oxopyrrolidine-1-carbonyl chloride (1.2 g, 8.1 mmol) in anhydrous dichloromethane (5 mL). The reaction mixture was stirred at room temperature for 3 days. The solid material was removed by filtration. The filtrate was recovered and evaporated under reduced pressure. The residue was triturated with toluene (75 mL) and dichloromethane (2 mL). The solid material was recovered by filtration, washed with water (2×25 mL), diethyl ether (3×25 mL), and dried in vacuo. The crude product was purified by flash chromatography (Combiflash Rf, 15-100% EtOAc/CH₂Cl₂), affording the final product (156 mg, 8%). ¹H NMR (400 MHz, DMSO-d₆) δ 1.64 (m, 2H), 1.74 (m, 2H), 3.00 (m, 2H), 2.62 (t, 1H, J=6.5 Hz), 2.95 (m, 2H), 3.87 (m, 2H), 4.15 (t, 1H, J=8.6 Hz), 5.55 (t, 1H, J=2.9 Hz), 6.10 (t, 1H, J=7.2 Hz), 7.32 (t, 1H, J=9.6 Hz), 7.41 (dd, 1H, J=2.5 Hz, 9.6 Hz), 7.54 (s, 1H), 7.69 (t, 1H, J=6.3 Hz); M⁺ 437.

### Example 76

### 5-Fluoro-2-{[(5E)-4-oxo-2-(1,4,5,6-tetrahydropyridazin-1-yl)-4,5-dihydro-1,3-thiazol-5-ylidene]methyl}phenyl pyrrolidine-1-carboxylate

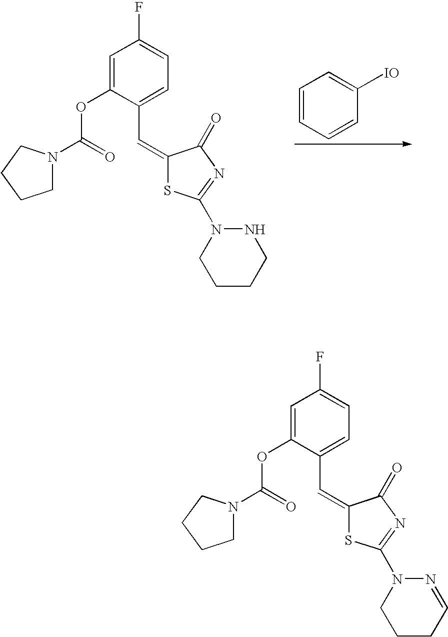

A mixture of 2-{[(5Z)-2-(1,2-diazinan-1-yl)-4-oxo-4,5-dihydro-1,3-thiazol-5-ylidene]methyl}-5-fluorophenyl pyrrolidine-1-carboxylate (2.18 g, 5.4 mmol) and iodosobenzene (2.40 g, 10.9 mmol) in anhydrous dichloromethane (30 mL) was stirred at room temperature for 4 days. The solid material was removed by filtration. The filtrate was recovered and evaporated under reduced pressure. The crude product was purified by flash chromatography (Combiflash Rf, 0-5% MeOH/CH₂Cl₂), affording the final compound (580 mg, 26%). ¹H NMR (400 MHz, DMSO-d₆) δ 1.92 (m, 6H), 2.40 (m, 2H), 2.37 (t, 2H, J=6.6 Hz), 3.58 (t, 2H, J=6.7 Hz), 4.08 (t, 2H, J=7.4 Hz), 7.30 (m, 2H), 7.46 (t, 1H, J=2.9 Hz), 7.61 (s, 1H), 7.70 (t, 1H, J=6.3 Hz); M⁺ 403.

### Example 77

### 2-{[(5E)-2-(1,2-Diazinan-1-yl)-4-oxo-4,5-dihydro-1,3-thiazol-5-ylidene]methyl}-5-fluorophenyl 2,3-dihydro-1H-pyrrole-1-carboxylate

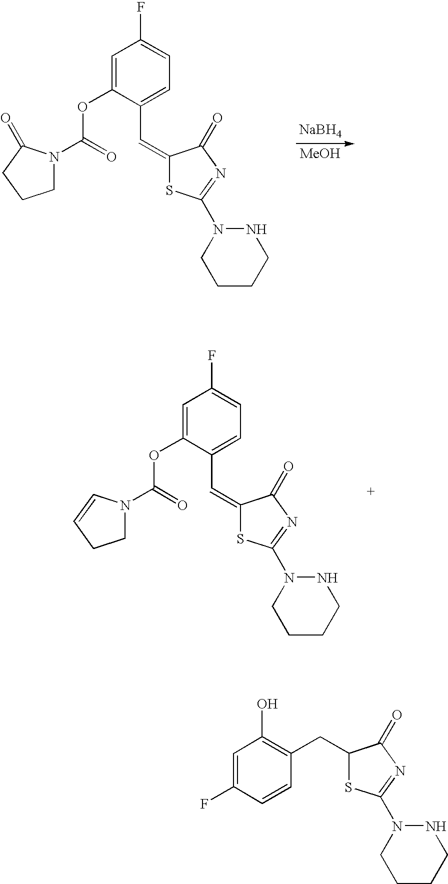

To a mixture of 2-{[(5E)-2-(1,2-diazinan-1-yl)-4-oxo-4,5-dihydro-1,3-thiazol-5-ylidene]methyl}-5-fluorophenyl 2-oxopyrrolidine-1-carboxylate (500 mg, 1.2 mmol) in anhydrous methanol (10 mL) was added portionwise sodium borohydride (250 mg, 6.6 mmol). The reaction mixture was stirred between −10° C. and −5° C. for 1.5 hours. The reaction was quenched with saturated aqueous ammonium chloride (20 mL). The mixture was extracted with CH₂Cl₂ (2×35 mL). The organic extracts were combined, dried over MgSO₄, filtered, evaporated, and dried in vacuo. The crude product was purified by flash chromatography (Combiflash Rf, 0-5% MeOH/CH₂Cl₂) affording two products.

2-{[(5E)-2-(1,2-diazinan-1-yl)-4-oxo-4,5-dihydro-1,3-thiazol-5-ylidene]methyl}-5-fluorophenyl 2,3-dihydro-1H-pyrrole-1-carboxylate (72 mg, 17%). ¹H NMR (400 MHz, CD₃OD) δ 1.68 (m, 2H), 1.75 (m, 2H), 2.70 (t, 1H, J=3.5 Hz), 2.91 (m, 2H), 3.57 (dd, 1H, J=4.5 Hz, 9.4 Hz), 3.83 (m, 2H), 4.46 (dd, 1H, J=4.5 Hz, 9.4 Hz), 6.49 (m, 2H), 7.06 (t, 1H, J=7.0 Hz); M⁺ 310.

(5Z)-2-(1,2-diazinan-1-yl)-5-[(4-fluoro-2-hydroxyphenyl)methylidene]-4,5-dihydro-1,3-thiazol-4-one. Obtained in 10 mg (2%). ¹H NMR (400 MHz, CD₃OD) δ 1.96 (m, 6H), 3.04 (t, 2H, J=2.4 Hz), 3.38 (m, 1H), 3.72 (m, 1H), 3.94 (m, 2H), 5.54 (d, 1H, J=3.7 Hz), 5.74 (t, 1H, J=5.0 Hz), 7.14 (m, 2H), 7.71 (m, 2H); M⁺ 403.

### Example 78

### Methyl 6-(2-{[(5Z)-2-(1,2-diazinan-1-yl)-4-oxo-4,5-dihydro-1,3-thiazol-5-ylidene]methyl}-5-fluorophenoxy)-3,4,5-tris(acetyloxy)oxane-2-carboxylate

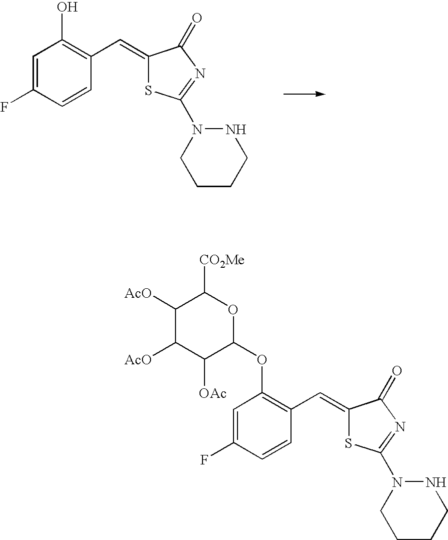

To a solution of (5Z)-2-(1,2-diazinan-1-yl)-5-[(4-fluoro-2-hydroxyphenyl)methylidene]-4,5-dihydro-1,3-thiazol-4-one (500 mg; 1.63 mmoL) in acetonitrile (20 mL) was added potassium carbonate (406 mg, 2.93 mmoL) and then methyl 3,4,5-triacetoxy-6-bromo-tetrahydro-2H-pyran-2-carboxylate (1.1 g, 2.77 mmol) in acetonitrile (5 mL). The reaction was stirred overnight at 40° C., and the solid was then filtered and washed with acetone plus dichloromethane. The filtrate was evaporated, and the residue was dissolved in DCM and purified on silica gel using 5% MeOH in DCM to afford the product (138 mg; 13%). ¹H NMR (400 MHz, DMSO-d₆) δ 1.64 (bs, 2H), 1.74 (bs, 2H), 2.00-2.02 (3×s, 9H), 2.93 (bs, 2H), 3.64 (s, 3H), 3.87 (bs, 2H), 4.73 (d, J=9.8 Hz, 1H), 5.09-5.21 (m, 2H), 5.43 (t, J=9.5 Hz, 1H), 5.70 (d, J=7.6 Hz, 1H), 6.05 (t, J=7.1 Hz, 1H), 7.12-7.19 (m, 2H), 7.58-7.63 (m, 2H).

### Example 79

### 6-(2-{[(5Z)-2-(1,2-Diazinan-1-yl)-4-oxo-4,5-dihydro-1,3-thiazol-5-ylidene]methyl}-5-fluorophenoxy)-3,4,5-trihydroxyoxane-2-carboxylic acid

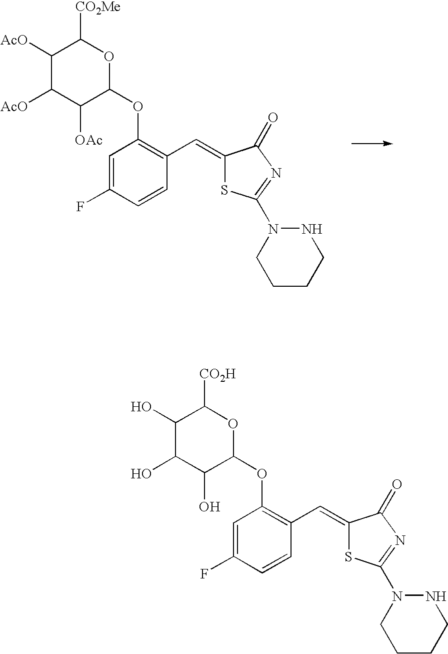

Methyl 6-(2-{[(5Z)-2-(1,2-diazinan-1-yl)-4-oxo-4,5-dihydro-1,3-thiazol-5-ylidene]methyl}-5-fluorophenoxy)-3,4,5-tris(acetyloxy)oxane-2-carboxylate (150 mg, 240 micromol) was dissolved in a mixture of THF (15 mL) and H₂O (3 mL). A solution of lithium hydroxide (81 mg, 1.93 mmol) in H₂O (2 mL) was added dropwise at 0° C. The reaction mixture was stirred at room temperature for 1.5 hours, and Amberlite IR-120 was added until neutralization. The solid was filtered and washed with methanol. The filtrate was evaporated, the residue was dissolved in MeOH, and purified on silica gel using a mixture of 7:2:1 EtOAc/MeOH/H₂O to afford the product (65 mg, 56% yield). ¹H NMR (400 MHz, DMSO-d₆) δ 1.63 (bs, 2H), 1.75 (bs, 2H), 2.94 (bs, 2H), 3.17 (s, 2H), 3.18-3.34 (m, 2H), 3.63 (d, J=9.4 Hz, 1H), 3.86 (bs, 2H), 4.12 (bs, 1H), 5.08-5.13 (m, 2H), 5.43 (d, J=4.7 Hz, 1H), 6.03 (t, J=7.1 Hz, 1H), 7.01 (dt, J=8.5 Hz, J=2.3 Hz, 1H), 7.14 (dd, J=11.2 Hz, J=2.6 Hz, 1H), 7.56 (dd, J=6.7 Hz, J=2.0 Hz, 1H), 7.86 (s, 1H).

### Example 80

### Methyl 4-[(2-{[(5E)-2-(1,2-diazinan-1-yl)-4-oxo-4,5-dihydro-1,3-thiazol-5-ylidene]methyl}-5-fluorophenoxycarbonyl)amino]butanoate

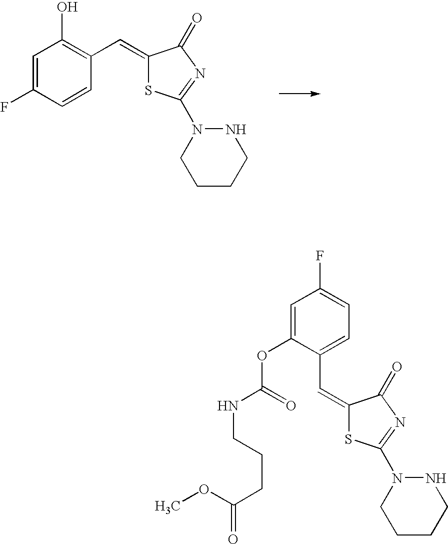

(5Z)-2-(1,2-diazinan-1-yl)-5-[(4-fluoro-2-hydroxyphenyl)methylidene]-4,5-dihydro-1,3-thiazol-4-one (488 mg; 1.6 mmol) and 4-dimethylaminopyridine (20 mg, 160 μmol) were stirred in THF (5 mL). A solution of 4-isocyanatobutyric acid methyl ester (250 mg, 1.7 mmol) in THF (2 mL) was then added portionwise at room temperature. The reaction mixture was heated at 60° C. overnight. The mixture was cooled at room temperature, and the solvent was evaporated. The residue was dissolved in DCM and purified on silica gel using 10% MeOH/DCM to afford the product (594 mg, 83% yield). ¹H NMR (400 MHz, DMSO-d₆) δ 1.62-1.78 (m, 6H), 2.39 (t, J=7.4 Hz, 2H), 2.94-2.99 (bs, 2H), 3.10 (q, J=6.7 Hz, 6.1 Hz, 2H), 3.60 (s, 3H), 3.87 (bs, 2H), 6.08 (t, J=7.2 Hz, 1H), 7.24-7.31 (m, 2H), 7.55 (s, 1H), 7.66 (dd, J=6.3 Hz, 2.3 Hz, 1H), 8.17 (t, J=5.7 Hz, 1H).

### Example 81

### 4-[(2-{[(5E)-2-(1,2-Diazinan-1-yl)-4-oxo-4,5-dihydro-1,3-thiazol-5-ylidene]methyl}-5-fluorophenoxycarbonyl)amino]butanoic acid

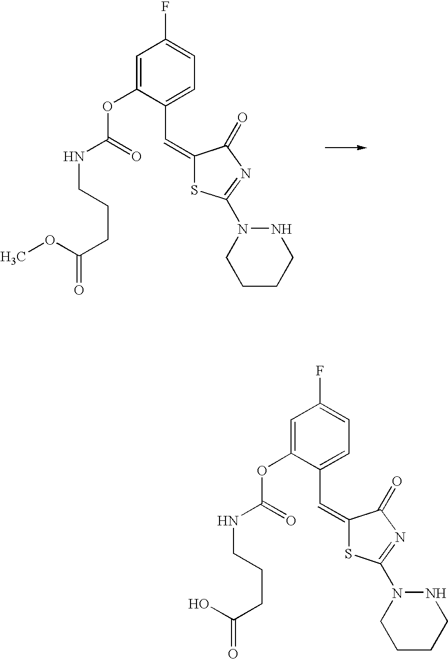

Methyl 4-[(2-{[(5Z)-2-(1,2-diazinan-1-yl)-4-oxo-4,5-dihydro-1,3-thiazol-5-ylidene]methyl}-5-fluorophenoxycarbonyl)amino]butanoate (206 mg; 417 μmol) was stirred in DCM (2 mL), and then trifluoroacetic acid (1.3 mL, 16.8 mmol) was added dropwise at 0° C. The reaction mixture was stirred overnight at room temperature. The solvent was evaporated, and the residue was triturated in MeOH and diethyl ether to afford the product (92 mg, 51% yield). HPLC-purity: 81%, with contamination by 12% of methyl ester.

### Example 82

### 5-Fluoro-2-{[(5E)-4-oxo-2-(3-oxo-1,2-diazinan-1-yl)-4,5-dihydro-1,3-thiazol-5-ylidene]methyl}phenyl pyrrolidine-1-carboxylate

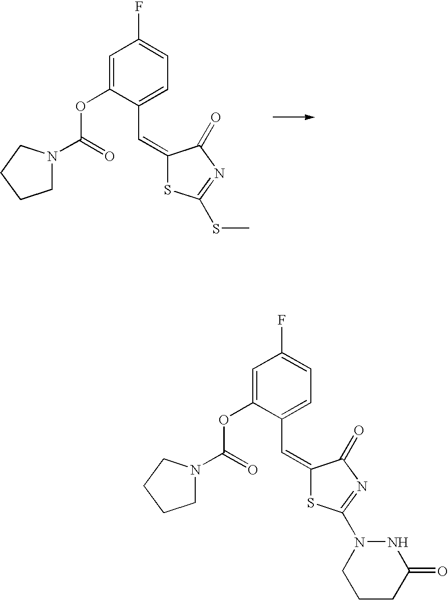

To a suspension of 5-fluoro-2-{[(5E)-4-oxo-2-(methylsulfanyl)-4,5-dihydro-1,3-thiazol-5-ylidene]methyl}phenyl pyrrolidine-1-carboxylate (700 mg, 1.91 mmol, prepared in 3 steps from 4-fluoro-2-hydroxybenzaldehyde) in absolute ethanol (15 mL) was added dropwise a solution of piperazin-3-one (268 mg, 2.68 mmol, prepared in 2 steps from 6-oxo-1,4,5,6-tetrahydropyridazine-3-carboxylic acid) in ethanol (5 mL) at room temperature. This was followed by the dropwise addition of triethylamine (665 μL, 4.78 mmol) at 0° C. The reaction mixture was stirred at 50° C. overnight. The mixture was cooled to room temperature, and the solvent was evaporated. The residue was dissolved in DCM and purified on silica gel using 15% MeOH/DCM to afford the product (344 mg, 43% yield). ¹H NMR (400 MHz, DMSO-d₆) δ 1.87-2.08 (m, 6H), 2.43 (t, J=6.7 Hz, 2H), 3.36 (t, J=6.7 Hz, 2H), 3.57 (t, J=6.7 Hz, 2H), 4.02 (t, J=6.2 Hz, 2H), 7.29-7.35 (m, 2H), 7.56 (s, 1H), 7.61-7.66 (m, 1H).

### Example 83

### (5Z)-2-(1,2-diazinan-1-yl)-5-[(4-fluoro-2-hydroxyphenyl)methylidene]-1-methyl-4,5-dihydro-1H-imidazol-4-one

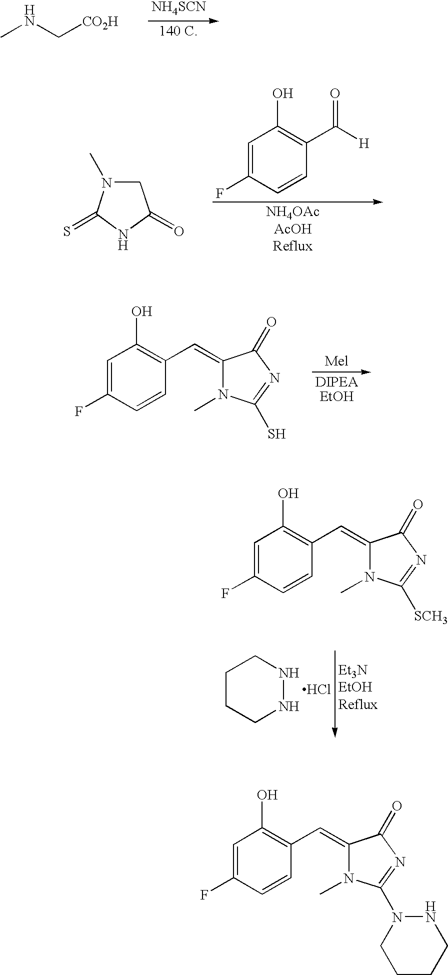

A mixture of sarcosine (1.0 g, 11.2 mmol) and ammonium thiocyanate (2.56 g, 33.6 mmol) was heated at 140° C. overnight without stirring. After cooling to room temperature, diethyl ether (25 mL) was added to the solution, and the solid residue was triturated with water (100 mL). The solid product was recovered by filtration, washed with water (3×50 mL), ethanol (1×50 mL) and diethyl ether (1×50 mL), and dried in vacuo, affording 1-methyl-2-thioxoimidazolidin-4-one (1.40 g, 96%). The product was used without further purification.

A mixture of 1-methyl-2-thioxoimidazolidin-4-one (1.40 g, 10.8 mmol), 4-fluorosalicylaldehyde (1.51 g, 10.8 mmol) and ammonium acetate (832 mg, 10.80 mmol) in acetic acid (75 mL) was stirred at reflux overnight. After cooling to room temperature, the solvent was evaporated. Water (50 mL) was then added to the residue. The mixture was extracted with EtOAc (1×50 mL). The organic phase was recovered and extracted with brine (3×50 mL), dried over MgSO₄, filtered, evaporated, and dried in vacuo, affording the thiol dimer intermediate (872 mg, 32%). The product was used without further purification.

To a mixture of thiol dimer (872 mg, 3.5 mmol) in absolute EtOH (25 mL) was added N—N-diisopropylethylamine (700 μL, 4.0 mmol) and iodomethane (435 μL, 7.0 mmol). The reaction mixture was stirred at room temperature overnight. The solid material was recovered by filtration, washed with EtOH (3×20 mL), diethyl ether (1×10 mL), and dried in vacuo, affording the methylated thiol. The product was used without further purification.

To a mixture of methylated thiol intermediate (523 mg, 1.2 mmol) in absolute ethanol (10 mL) was added hexahydropyridazine dihydrochloride (509 mg, 8.0 mmol) and triethylamine (1.1 mL, 8.0 mmol). The reaction mixture was stirred at reflux overnight. After cooling to room temperature, the solid material was recovered by filtration and washed with EtOH (2×15 mL), diethyl ether (2×10 mL), and dried in vacuo, affording the final product (335 mg, 92%). ¹H NMR (400 MHz, DMSO-d₆) δ 1.61 (m, 2H), 1.74 (m, 2H), 2.92 (m, 2H), 3.52 (s, 3H), 3.65 (m 2H), 5.29 (t, 1H, J=7.0 Hz), 6.48 (s, 1H), 6.63 (m, 2H), 8.37 (t, 1H, J=7.0 Hz); M⁺ 360.

### Example 84

### (5Z)-4-(1,2-Diazinan-1-yl)-5-[(4-fluoro-2-hydroxyphenyl)methylidene]-2,5-dihydro-1,3-thiazol-2-one

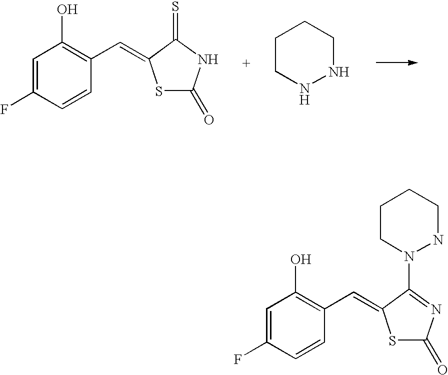

To a mixture of the 5-(4-fluoro-2-hydroxy-benzylidene)-4-thioxo-thiazolidin-2-one (1.2 g, 4.74 mmol), ethanol (5 mL), and triethylamine (1.9 g, 18.96 mmol) was added methyl iodide (0.8 g, 0.355 mmol) at room temperature. After stirring for 15 minutes, tetrahydropyridazine was added, and the reaction was heated to 50° C. for 3 hours. The solvent was evaporated, and the residue was purified by combiflash to provide the product as a yellow solid. ¹H NMR (400 MHz, DMSO-d₆) δ 1.61 (m, 2H), 1.75 (m, 2H), 2.45 (m, 1H), 3.21 (m, 1H), 3.45 (m, 1H), 4.21 (d, J=11.8 Hz, 1H), 5.21 (d, 1H), 6.65 (m, 2H), 7.61 (m, 1H), 10.51 (bs, 1H).

### Example 85

### (5Z)-5-[(5-bromo-4-fluoro-2-hydroxyphenyl)methylidene]-2-(1,2-diazinan-1-yl)-4,5-dihydro-1,3-thiazol-4-one

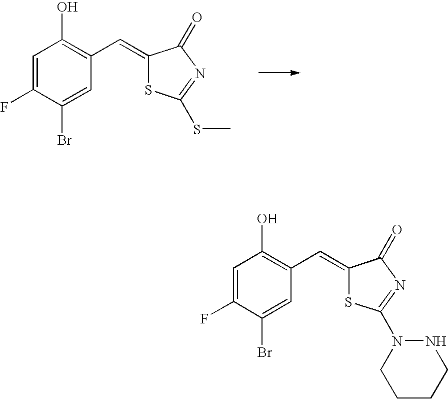

To a suspension of (5Z)-5-(5-bromo-4-fluoro-2-hydroxybenzylidene)-2-(methylthio)thiazol-4(5H)-one (467 mg, 1.34 mmol, prepared in 3 steps from 4-bromo-3-fluorophenol) in absolute ethanol (10 mL) was added dropwise a solution of hexahydropyridazine dihydrochloride (320 mg, 2.10 mmol) in ethanol (5 mL) at room temperature. Triethylamine (470 μL, 3.35 mmol) was subsequently added dropwise at 0° C. The reaction mixture was stirred at 50° C. overnight, and then the reaction was cooled in an ice-water bath. The yellow solid was recovered by filtration, washed with cold ethanol, and dried in vacuo, affording the product (122 mg, 24% yield). HPLC purity: 98.4%. ¹H NMR (400 MHz, DMSO-d₆) δ 1.65 (bs, 2H), 1.74 (bs, 2H), 2.96 (bs, 2H), 3.87 (bs, 2H), 6.06 (t, J=7.0 Hz, 1H), 6.89 (d, J=10.4 Hz, 1H), 7.56 (d, J=7.8 Hz, 1H), 7.71 (s, 1H).

### Example 86

### (5Z)-2-(1,2-Diazinan-1-yl)-5-[(3,5-dibromo-4-fluoro-2-hydroxyphenyl)methylidene]-4,5-dihydro-1,3-thiazol-4-one

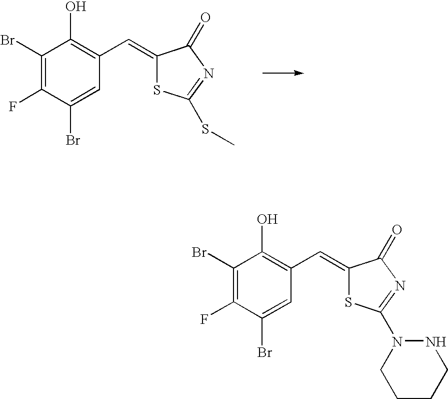

To a suspension of (5Z)-5-(3,5-dibromo-4-fluoro-2-hydroxybenzylidene)-2-(methylthio)thiazol-4(5H)-one (420 mg, 983 μmol, prepared in 3 steps from 4-fluorosalicaldehyde) in absolute ethanol (10 mL) was added dropwise a solution of hexahydropyridazine dihydrochloride (235 mg, 1.48 mmol) in ethanol (5 mL) at room temperature. Triethylamine (343 μL, 2.46 mmol) was subsequently added dropwise at 0° C. The reaction mixture was stirred at 50° C. overnight, and then solvent was removed by evaporation. Triethylammonium salts were removed by filtration, and the filtrate was evaporated. The residue was dissolved in DCM and purified on silica gel using 5-10% MeOH/DCM to afford the product (74 mg, 16% yield) as yellow solid. ¹H NMR (400 MHz, DMSO-d₆) δ 1.65 (bs, 2H), 1.74 (bs, 2H), 2.95 (bs, 2H), 3.86 (bs, 2H), 6.07 (t, J=7.1 Hz, 1H), 7.60 (d, J=7.8 Hz, 1H), 7.73 (s, 1H).

### Example 87

### (5Z)-5-[(4-Fluoro-2-hydroxyphenyl)methylidene]-2-(5-methyl-3-oxopyrazolidin-1-yl)-4,5-dihydro-1,3-thiazol-4-one

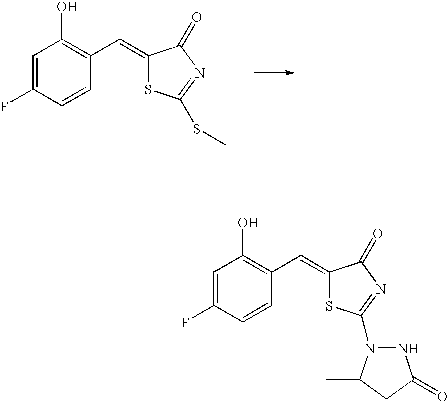

To a suspension of (5Z)-5-[(4-fluoro-2-hydroxyphenyl)methylidene]-2-(methylsulfanyl)-4,5-dihydro-1,3-thiazol-4-one (500 mg, 1.86 mmol) in absolute ethanol (15 mL) was added dropwise a solution of 5-methylpyrazolidin-3-one (322 mg, 2.78 mmol, previously prepared from ethyl trans-crotonate) in ethanol (5 mL) at room temperature. This was followed by the dropwise addition of triethylamine (650 μL, 4.64 mmol) at 0° C. The reaction mixture was stirred at 65° C. over 48 hours, and then the solvent was removed by evaporation. The residue was then dissolved in DCM and purified on silica gel using 15% MeOH/DCM to afford the product (55 mg, 9% yield) as yellow solid. ¹H NMR (400 MHz, DMSO-d₆) δ 1.34 (d, J=6.5 Hz, 3H), 2.12 (dd, J=15.8 Hz, J=3.1 Hz, 1H), 2.90 (dd, J=9.7 Hz, 6.3 Hz, 1H), 3.39 (bs, 1H), 4.56 (m, 1H), 6.72-6.80 (m, 2H), 7.43 (dd, J=6.7 Hz, 1.8 Hz, 1H), 7.62 (s, 1H), 10.80 (bs, 1H).

### General Experimental Procedure for the Synthesis of Phosphate Esters:

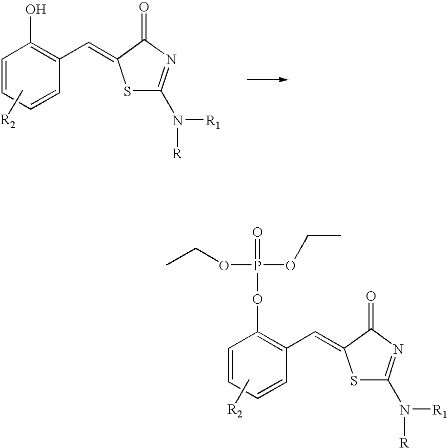

To a suspension of the phenol (1 equivalent) in acetonitrile at room temperature was added triethylamine (1.3 equiv) and diethylchloro phosphate (1.1 equiv), followed by catalytic DMAP. The reaction mixture was stirred at room temperature overnight. The solvent was evaporated, and the residue was purified by combi flash to provide the phosphate ester.

### Example 88

### Diethyl (5-fluoro-2-{[(5Z)-4-oxo-2-(pyrrolidin-1-yl)-4,5-dihydro-1,3-thiazol-5-ylidene]methyl}phenyl) phosphate

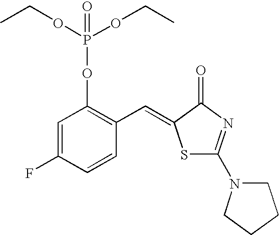

Example 88 was synthesized from (5Z)-5-[(4-fluoro-2-hydroxyphenyl)methylidene]-2-(pyrrolidin-1-yl)-4,5-dihydro-1,3-thiazol-4-one using the procedure described as above. The product was obtained as beige solid. ¹H NMR (CD₃OD, 400 MHz) δ 1.40 (t, 6H), 2.10 (m, 4H), 3.65 (t, 2H), 3.81 (t, 2H), 4.25 (q, 4H), 7.21 (m, 1H), 7.41 (m, 2H), 8.00 (s, 1H).

### Example 89

### (2-{[(5Z)-2-(1,2-Diazinan-1-yl)-4-oxo-4,5-dihydro-1,3-thiazol-5-ylidene]methyl}-5-fluorophenyl) diethyl phosphate

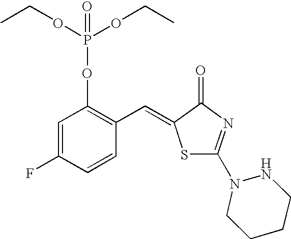

Example 89 was synthesized from (5Z)-2-(1,2-diazinan-1-yl)-5-[(4-fluoro-2-hydroxyphenyl)methylidene]-4,5-dihydro-1,3-thiazol-4-one using the procedure described as above. The product was obtained as yellow oil; M⁺ 444.5.

### Example 90

### (2-{[(5Z)-2-(1,2-Diazinan-1-yl)-4-oxo-4,5-dihydro-1,3-thiazol-5-ylidene]methyl}-5-fluorophenoxymethyl)diethyl phosphonate

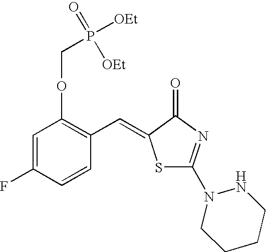

To a suspension in anhydrous acetonitrile of the phenol, (5Z)-2-(1,2-diazinan-1-yl)-5-[(4-fluoro-2-hydroxyphenyl)methylidene]-4,5-dihydro-1,3-thiazol-4-one, K₂CO₃ (1.5 equiv), trifluoro-methanesulfonic acid, and diethoxy-phosphorylmethyl ester (1.2 equiv, prepared per J. Org. Chem. 61:7697, 1996) were added and the mixture was refluxed overnight. The reaction mixture was then filtered and evaporated to provide the product as a semi solid; M⁺ 458.3.

### General Procedure for Phosphate Prodrugs

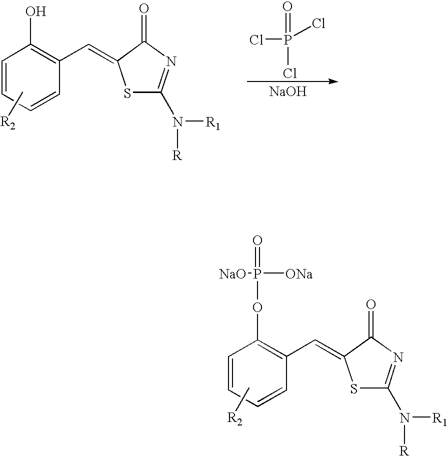

In a 250 mL round bottom flask, the phenolic analogue (10 mmol) and triethylamine (3.08 mL, 22 mmol) were combined in THF (100 mL). POCl₃ (1.0 mL, 11 mmol) was added slowly at 0° C. and stirred 2 hours. The resulting mixture was then stirred at room temperature for another 5 hours. The mixture was filtered to remove triethylamine salt and unreacted phenol. To the clear filtrate, water (0.72 mL, 40 mmol) was added, and the mixture stirred for 3 hours. The yellow solid was collected and washed with THF to provide the phosphate product (yield 80%).

The sodium salt was prepared in the following manner. To the slurry of 10% by weight of phosphoric acid in water was added aqueous NaOH (1.0 eq, 2N). The clear solution was freeze-dried to provide pure sodium salt.

### Example 91

### 2-{[(5Z)-2-(1,2-Diazinan-1-yl)-4-oxo-4,5-dihydro-1,3-thiazol-5-ylidene]methyl}-5-fluorophenoxyphosphonic acid

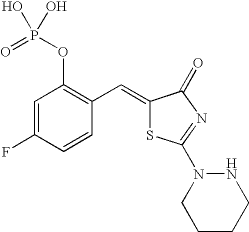

Example 91 was synthesized using the general procedure for phosphate prodrugs as described herein from (5Z)-2-(1,2-diazinan-1-yl)-5-[(4-fluoro-2-hydroxyphenyl)methylidene]-4,5-dihydro-1,3-thiazol-4-one. The product was obtained in 80% yield. ¹H-NMR (400 MHz, DMSO-d₆) δ 1.65-1.85 (m, 4H), 2.95 (m, 2H), 3.88 (m, 2H), 6.15 (br, NH), 7.19 (m, 1H), 7.31 (m, 1H), 7.65 (m, 1H), 7.78 (s, 1H), 13.02 (br, 2H).

### Example 92

### (2-{[(5Z)-2-(1,2-Diazinan-1-yl)-4-oxo-4,5-dihydro-1,3-thiazol-5-ylidene]methyl}-5-fluorophenyl) disodium phosphate

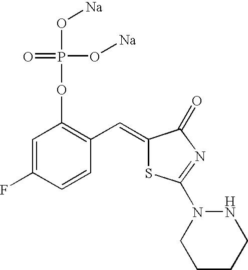

Example 92 was synthesized using the general procedure for phosphate prodrugs as described in Example 91. The product was obtained as an off white powder after NaOH treatment and lyophilization.

### Example 93

### 5-Fluoro-2-{[(5Z)-4-oxo-2-(pyrrolidin-1-yl)-4,5-dihydro-1,3-thiazol-5-ylidene]methyl}phenoxy phosphoric acid

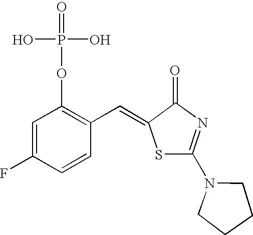

Example 93 was synthesized using the general procedure for phosphate prodrugs as described above from (5Z)-5-[(4-fluoro-2-hydroxyphenyl)methylidene]-2-(pyrrolidin-1-yl)-4,5-dihydro-1,3-thiazol-4-one. The product was obtained as an off white powder after NaOH treatment and lyophilization. ¹H-NMR (400 MHz, DMSO-d₆) δ 2.11 (m, 4H), 3.61 (t, 2H), 3.88 (t, 2H), 7.11 (t, 1H), 7.41-7.61 (m, 2H), 7.90 (s, 1H).

### Example 94

### 4-Fluoro-2-{[(5Z)-4-oxo-2-(pyrrolidin-1-yl)-4,5-dihydro-1,3-thiazol-5-ylidene]methyl}phenoxy phosphoric acid

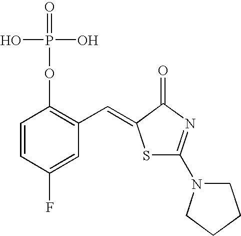

Example 94 was synthesized using the general procedure for phosphate prodrugs as described above for (5Z)-5-[(5-fluoro-2-hydroxyphenyl)methylidene]-2-(pyrrolidin-1-yl)-4,5-dihydro-1,3-thiazol-4-one. ¹H-NMR (400 MHz, DMSO-d₆) δ 1.99 (m, 4H), 3.344-3.88 (m, 4H), 7.19 (m, 2H), 7.40 (t, 1H), 7.95 (s, 1H).

### Example 95

### Disodium (4-fluoro-2-{[(5Z)-4-oxo-2-(pyrrolidin-1-yl)-4,5-dihydro-1,3-thiazol-5-ylidene]methyl}phenyl) phosphate

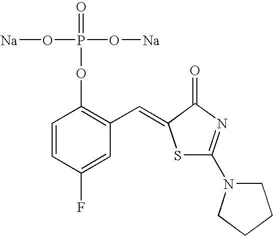

Example 95 was synthesized from Example 94 using the general procedure for phosphate prodrugs as described above. ¹H NMR (400 MHz, D₂O) δ 2.10 (m, 4H), 3.44-3.78 (m, 4H), 7.19 (t, 1H), 7.35 (d, 1H), 7.40 (t, 1H), 8.00 (s, 1H).

### Example 96

### (5Z)-5-{[2-(Dimethylamino)phenyl]methylidene}-2-(piperidin-1-yl)-4,5-dihydro-1,3-thiazol-4-one

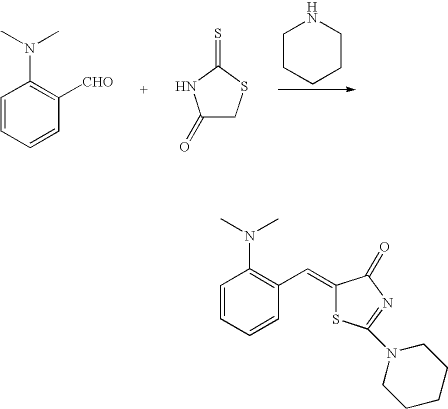

Example 96 was prepared as described in Example 1 using dimethylamino benzaldehyde, rhodanine, and piperidine. Yield 75%. ¹H NMR (400 MHz, DMSO-d₆) δ 1.97 (m, 4H), 2.81 (s, 6H), 3.58 (t, 2H, J=6.5 Hz), 3.87 (t, 2H, J=6.7 Hz), 7.24 (t, 1H), 7.11 (d, 1H, 8.0 Hz), 7.21 (t, 1H), 7.11 (d, 1H, 8.0 Hz), 7.81 (s, 1H).

### Example 97

### (5Z)-5-[(5-Fluoro-2-hydroxyphenyl)methylidene]-2-(piperidin-1-yl)-4,5-dihydro-1,3-thiazol-4-one

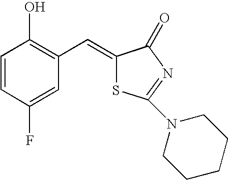

Example 97 was synthesized as described in Example 1, using 5-fluoro-2-hydroxy-benzadehyde, rhodanine, and piperidine. Yield 72%. ¹H NMR (400 MHz, DMSO-d₆) δ 1.63 (m, 6H), 3.63 (m, 2H), 3.94 (m, 2H), 6.95 (m, 1H), 7.18 (m, 2H), 7.83 (s, 1H), 10.38 (s, 1H).

### Example 98

### (5Z)-5-[(2-Hydroxyphenyl)methylidene]-2-(morpholin-4-yl)-4,5-dihydro-1,3-thiazol-4-one

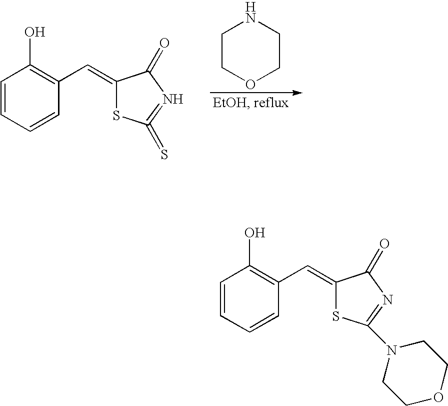

A solution of (5Z)-5-(2-hydroxybenzylidene)-2-thioxo-1,3-thiazolidin-4-one (500 mg, 2.1 mmol) and morpholine (252 μL, 2.9 mmol) in absolute ethanol (30 mL) was stirred at reflux overnight. After cooling to room temperature, the solid material was recovered by filtration, washed with EtOH (2×15 mL), and dried in vacuo, affording the product (202 mg, 33%). ¹H NMR (400 MHz, DMSO-d₆) δ 3.70 (m, 6H), 3.92 (t, 2H, J=4.8 Hz), 6.94 (m, 2H), 7.27 (td, 1H, J=1.6 Hz, 8.5 Hz), 7.44 (dd, 1H, J=1.4 Hz, 7.8 Hz), 7.94 (s, 1H), 10.40 (s, 1H); M⁺ 291.

### Example 99

### (5Z)-5-[(3-Hydroxyphenyl)methylidene]-2-(piperidin-1-yl)-4,5-dihydro-1,3-thiazol-4-one

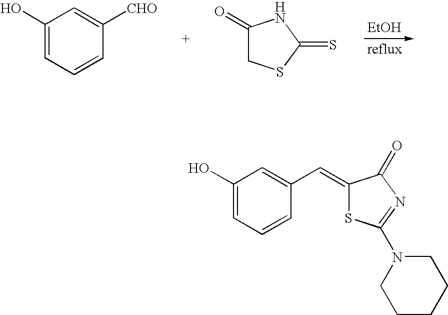

To a solution of rhodanine (500 mg, 3.8 mmol) in absolute ethanol (25 mL) was added 3-hydroxyaldehyde (500 mg, 4.1 mmol) and piperidine (750 uL, 7.6 mmol). The reaction mixture was stirred at reflux for 60 hours. After cooling to room temperature, the solid material was recovered by filtration, washed with EtOH (2×20 mL), and dried in vacuo, affording the desired compound (381 mg, 35%). ¹H NMR (400 MHz, DMSO-d₆) 1.63 (m, 6H), 3.59 (m, 2H), 3.88 (t, 2H, J=5.1 Hz), 6.82 (dd, 1H, J=1.6 Hz, 7.8 Hz), 7.00 (s, 1H), 7.03 (d, 1H, J=7.6 Hz), 7.28 (t, 1H, J=7.8 Hz), 7.50 (s, 1H), 9.75 (s(br), 1H); M⁺ 289.

### Example 100

### 2-{[(5E)-2-(1,2-Diazinan-1-yl)-4-oxo-4,5-dihydro-1,3-thiazol-5-ylidene]methyl}-5-fluorophenyl N,N-diethylcarbamate hydrochloride

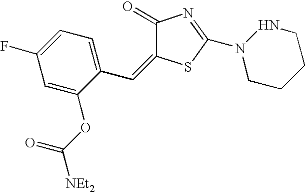

Example 100 was synthesized as described in Example 40, using (5Z)-2-(1,2-diazinan-1-yl)-5-[(4-fluoro-2-hydroxyphenyl)methylidene]-4,5-dihydro-1,3-thiazol-4-one and diethyl carbamoyl chloride (55% yield). ¹H NMR (400 MHz, DMSO-d₆) δ 1.20 (t, 3H), 1.34 (t, 3H), 1.75-1.85 (m, 4H), 3.01 (m, 2H), 3.40 (m, 2H), 3.55 (m, 2H), 3.92 (m, 2H), 4.89 (HCl), 7.08 (m, 1H), 7.15 (m, 1H), 7.70 (m, 2H).

### Example 101

### 2-{[(5E)-2-(1,2-diazinan-1-yl)-4-oxo-4,5-dihydro-1,3-thiazol-5-ylidene]methyl}-5-fluorophenyl 4-(piperidin-1-yl)piperidine-1-carboxylate dihydrochloride

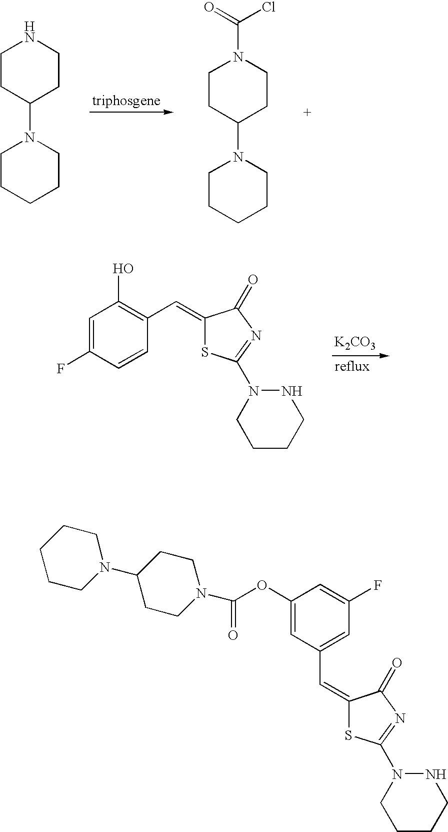

To a solution of 4-piperidinepiperidine (2.0 g, 11.9 mmol) in anhydrous CH₂Cl₂ (50 mL) at 0° C. was added a triphosgene solution (1.3 g, 4.3 mmol) in anhydrous CH₂Cl₂ (10 mL) by syringe pump addition (1 hour). The mixture was stirred at room temperature overnight. The solid material was removed by filtration. The mixture was extracted with 10% NaHCO₃ (2×50 mL) and brine (1×50 mL). The organic phase was dried over MgSO₄, filtered, evaporated, and dried in vacuo, affording the 4-piperidinopiperidinecarbonyl chloride (1.6 g, 59%). The product was used without further purification.

To a mixture of (5Z)-2-(1,2-diazinan-1-yl)-5-[(4-fluoro-2-hydroxyphenyl)methylidene]-4,5-dihydro-1,3-thiazol-4-one (1.0 g, 3.3 mmol) in anhydrous acetonitrile (15 mL) was added potassium carbonate (900 mg, 6.6 mmol), followed by a solution of 4-piperidinopiperidinecarbonyl chloride (1.07 g, 4.6 mmol) in anhydrous acetonitrile (5 mL). The reaction mixture was stirred at reflux for 60 hours. After cooling the mixture to room temperature, the solid material was removed by filtration. The filtrate was recovered and evaporated under reduced pressure. The crude product was purified by flash chromatography (Combiflash Rf, 0-20% MeOH/CH₂Cl₂). The residue was triturated with diethyl ether (50 mL×2). The solid material was recovered by filtration and dried in vacuo, affording 2-{[(5E)-2-(1,2-diazinan-1-yl)-4-oxo-4,5-dihydro-1,3-thiazol-5-ylidene]methyl}-5-fluorophenyl 4-(piperidin-1-yl)piperidine-1-carboxylate (954 mg, 57%).

To a mixture of 2-{[(5E)-2-(1,2-diazinan-1-yl)-4-oxo-4,5-dihydro-1,3-thiazol-5-ylidene]methyl}-5-fluorophenyl 4-(piperidin-1-yl)piperidine-1-carboxylate (954 mg, 1.9 mmol) in methanol (5 mL) was added a solution of 4M HCl/dioxane (3 mL, 12.0 mmol). The resultant solution was filtered, and the filtrate was recovered and evaporated. The solid was triturated with diethyl ether (50 mL). The solid material was recovered by filtration and dried in vacuo, affording the final compound (931 mg, 91%). ¹H NMR (400 MHz, DMSO-d₆) 1.45 (m, 1H), 1.81 (m, 1H), 2.16 (m, 2H), 2.96 (m, 5H), 3.15 (m, 1H), 3.44 (m, 3H), 3.88 (m, 2H), 4.11 (m, 1H), 4.35 (m, 1H) 6.17 (t, 1H, J=6.7 Hz), 7.32 (m, 2H), 7.47 (s, 1H), 7.67 (t, 1H, J=6.3 Hz);

M+502. HPLC purity: 99.1%.

### Example 102

### 2-{[(5E)-2-(1,2-diazinan-1-yl)-4-oxo-4,5-dihydro-1,3-thiazol-5-ylidene]methyl}-4-fluorophenyl 4-(pyrrolidin-1-yl)piperidine-1-carboxylate dihydrochloride

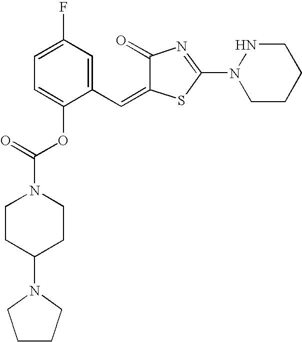

Example 102 was synthesized per Example 101, using (5Z)-2-(1,2-diazinan-1-yl)-5-[(5-fluoro-2-hydroxyphenyl)methylidene]-4,5-dihydro-1,3-thiazol-4-one and 4-pyrrolidin-1-yl-piperidine carbamoylchloride (65% yield). ¹H NMR (400 MHz, DMSO-d₆) δ 1.62-2.08 (m, 12H), 2.5-3.2 (m, 9H), 4.00 (br, 2H), 4.19, 4.36 (dd, 2H), 4.60 (t, NH), 7.06 (m, 1H), 7.15 (m, 1H), 7.32 (m, 1H), 7.72 (s, 1H).

### Example 103

### 2-{[(5E)-2-(1,2-Diazinan-1-yl)-4-oxo-4,5-dihydro-1,3-thiazol-5-ylidene]methyl}-4-fluorophenyl 4-(piperidin-1-yl)piperidine-1-carboxylate dihydrochloride

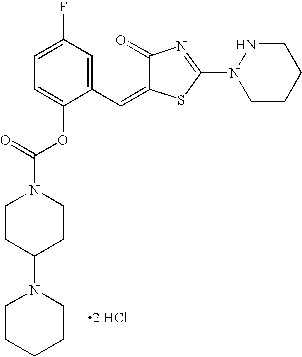

Example 103 was synthesized per Example 101, using (5Z)-5-(5-fluoro-2-hydroxybenzylidene)-2-(hexahydropyridazin-1 (2H)-yl)-1,3-thiazol-4(5H)-one and 4-piperidinopiperidinecarbonyl chloride. ¹H NMR (400 MHz, DMSO-d₆) δ 1.43 (m, 1H), 1.81 (m, 11H), 2.18 (m, 2H), 2.95 (m, 5H), 3.13 (m, 1H), 3.43 (m, 3H), 3.88 (m, 2H), 4.11 (m, 1H), 4.36 (m, 1H) 6.13 (m, 1H), 7.33 (m, 3H), 7.44 (s, 1H), 10.64 (s, br), 1H); M⁺ 502. HPLC purity: 98.7%.

### Example 104

### 2-{[(5E)-2-(1,2-Diazinan-1-yl)-4-oxo-4,5-dihydro-1,3-thiazol-5-ylidene]methyl}-4-fluorophenyl (3R)-3-(dimethylamino)pyrrolidine-1-carboxylate dihydrochloride

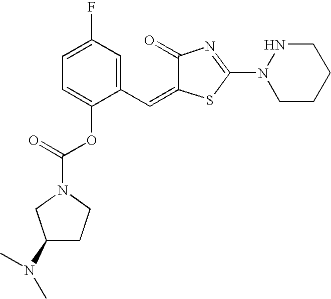

Example 104 was synthesized per Example 43, using (5Z)-5-(5-fluoro-2-hydroxybenzylidene)-2-(hexahydropyridazin-1(2H)-yl)-1,3-thiazol-4(5H)-one and (R)-3-dimethylamino pyrrolidine carbamoyl chloride (yield 55%). ¹H NMR (400 MHz, DMSO-d₆) δ 1.65-1.75 (m, 4H), 2.10 (m, 8H), 2.79 (m, 1H), 2.90 (m, 2H), 3.50 (m 2H), 3.71-3.87 (m, 4H), 6.11 (m, 1H), 7.30 (m, 3H), 7.49 (s, 1H).

### Example 105

### 2-{[(5E)-2-(1,2-diazinan-1-yl)-4-oxo-4,5-dihydro-1,3-thiazol-5-ylidene]methyl}-5-fluorophenyl (3S)-3-(dimethylamino)pyrrolidine-1-carboxylate dihydrochloride

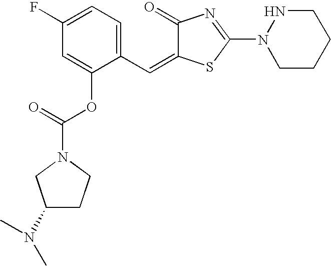

Example 105 was synthesized per Example 43, using (5Z)-2-(1,2-diazinan-1-yl)-5-[(4-fluoro-2-hydroxyphenyl)methylidene]-4,5-dihydro-1,3-thiazol-4-one and (S)-3-dimethylamino pyrrolidine carbamoyl chloride (60% yield). ¹H NMR (400 MHz, DMSO-d₆) δ 1.60-1.70 (m, 4H), 2.30 (m, 2H), 2.75 (m, 6H), 2.90 (m, 2H), 3.60 (m, 1H), 3.80-4.00 (m, 4H), 7.30 (m, 2H), 7.49 (s, 1H), 7.61 (m, 1H).

### Example 106

### (5Z)-5-[(4-aminopyridin-3-yl)methylidene]-2-(piperidin-1-yl)-4,5-dihydro-1,3-thiazol-4-one hydrochloride

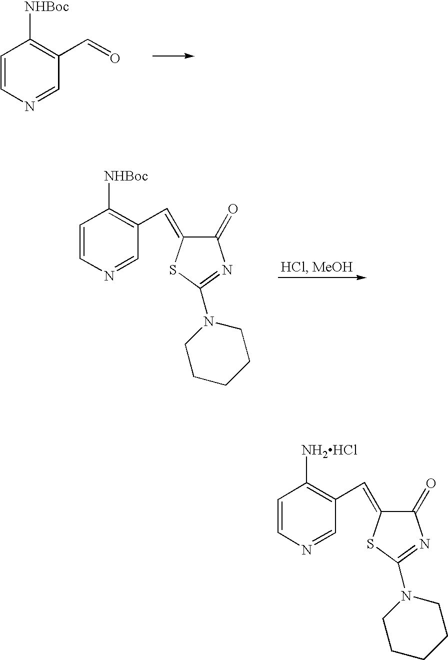

(3-Formyl-pyridin-4-yl)-carbamic acid tert-butyl ester (490 mg, 1.5 mmol), piperidine (0.30 mL, 3.0 mmol), and rhodanine (203 mg, 1.5 mmol) were added into 10 mL of ethanol in 50 mL flask. The resulting mixture was heated at 75° C. overnight. The solvent was evaporated, and the crude was purified by flash chromatography (0 to 10% MeOH in DCM gradient). 200 mg of [3-(4-Oxo-2-piperidin-1-yl-4H-thiazol-5-ylidenemethyl)-pyridin-4-yl]-carbamic acid tert-butyl ester yellow solid was collected as pure product (yield 34%).

[3-(4-Oxo-2-piperidin-1-yl-4H-thiazol-5-ylidenemethyl)-pyridin-4-yl]-carbamic acid tert-butyl ester (200 mg) was dissolved in 5 mL of methanolic HCl (4M). The resulting mixture was stirred at room temperature for 2 hours. The solvent was evaporated, and the remaining solid was dried under vacuum to provide desired product (yield 99%). ¹H NMR (400 MHz, DMSO-d₆) δ 1.67 (m, 6H), 3.5 (m, 2H), 3.90 (m, 2H), 6.97 (d, 1H), 7.50 (s, 1H), 8.13 (d, 1H), 8.30 (s, 1H).

### Example 107

### 2-{[(5E)-2-(1,2-Diazinan-1-yl)-4-oxo-4,5-dihydro-1,3-thiazol-5-ylidene]methyl}-5-fluorophenyl pyrrolidine-1-carboxylate; methanesulfonic acid

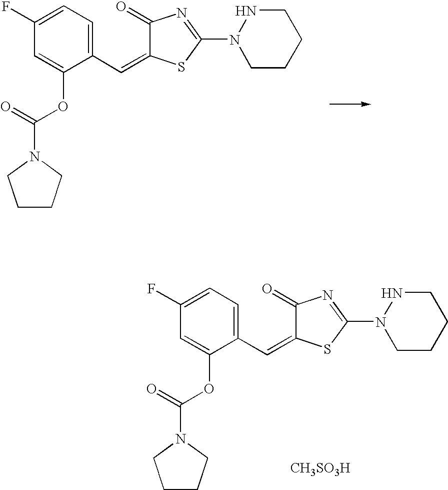

2-{[(5E)-2-(1,2-diazinan-1-yl)-4-oxo-4,5-dihydro-1,3-thiazol-5-ylidene]methyl}-5-fluorophenyl pyrrolidine-1-carboxylate hydrochloride (40.4 g, 0.100 mol) and 120 mL of DCM were combined in a 500 mL round bottom flask. Methanesulfonic acid (12.0 g, 0.125 mol) was then added. The resulting mixture was stirred at room temperature for 2 hours and filtered. The filtrate was precipitated with MTBE (120 mL×3). The resulting solid was filtered and dried under vacuum to provide the salt in 98% yield. ¹H NMR (400 MHz, DMSO-d₆) δ 1.65 (m, 2H), 1.75 (m, 2H), 1.95 (m, 4H), 2.46 (s, 3H), 2.94 (br, 2H), 3.36 (m, 2H), 3.57 (m, 2H), 3.87 (br, 2H), 6.11 (br, 1H), 7.26 (m, 2H), 7.55 (s, 1H), 7.66 (m, 1H).

### Example 108

### 5-Fluoro-2-{[(5E)-2-[4-(2-hydroxyethyl)piperazin-1-yl]-4-oxo-4,5-dihydro-1,3-thiazol-5-ylidene]methyl}phenyl pyrrolidine-1-carboxylate; methanesulfonic acid

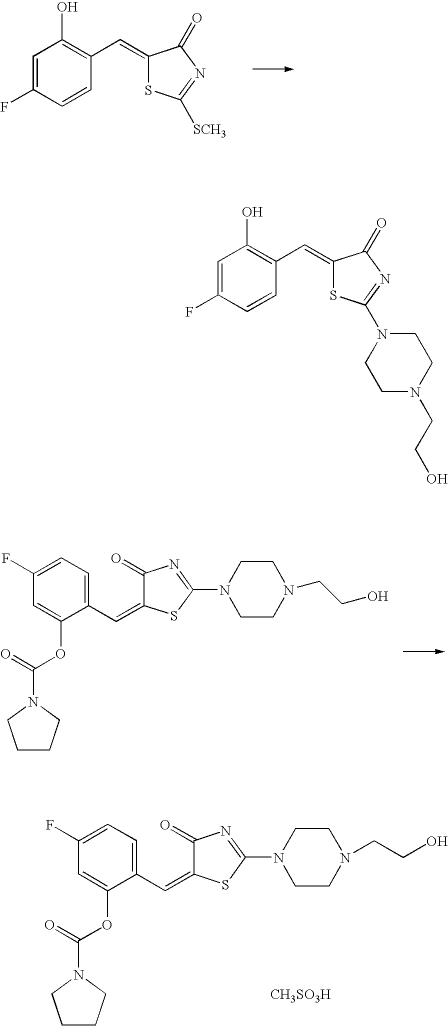

To a suspension of (5Z)-5-[(4-fluoro-2-hydroxyphenyl)methylidene]-2-(methylsulfanyl)-4,5-dihydro-1,3-thiazol-4-one (40.0 g, 149 mmol) in absolute ethanol (350 mL) was added dropwise a solution of 1-piperazine-ethanol (27.1 g, 209 mmol) in ethanol (50 mL) at room temperature. The reaction mixture was stirred at 80° C. for 3 hours, and then the mixture was cooled in an ice-water bath. The yellow solid was recovered by filtration, washed with cold ethanol, and dried in vacuo, affording (5Z)-5-(4-fluoro-2-hydroxybenzylidene)-2-[4-(2-hydroxyethyl)piperazin-1-yl]-1,3-thiazol-4(5H)-one, 31.2 g, 60% yield. ¹H NMR (400 MHz, DMSO-d₆) δ 2.44-2.60 (m, 6H), 3.53 (bs, 2H), 3.62 (bs, 2H), 3.90 (bs, 2H), 4.50 (bs, 1H), 6.71-6.82 (m, 2H), 7.46 (t, J=6.7 Hz, 1H), 7.84 (s, 1H), 10.94 (bs, 1H).

To a mixture of (5Z)-5-(4-fluoro-2-hydroxybenzylidene)-2-[4-(2-hydroxyethyl)piperazin-1-yl]-1,3-thiazol-4(5H)-one (11.5 g, 32.8 mmol) in anhydrous acetonitrile (200 mL) was added potassium carbonate (9.0 g, 65.7 mmol) followed by pyrrolidine carbamoyl chloride (5.5 mL, 49.8 mmol). The reaction mixture was stirred at reflux overnight. The solid material was removed by filtration while warm. The filtrate was recovered and evaporated under reduced pressure. The solid residue was dissolved in dichloromethane (10 mL), and diethyl ether (150 mL) was then added slowly. The solid product was recovered by filtration, washed with diethyl ether (2×10 mL) and dried in vacuo, affording 4-fluoro-2-{(E)-[4-oxo-2-(tetrahydropyridazin-1(2H)-yl)-1,3-thiazol-5(4H)-ylidene]-methyl}phenyl)-(pyrrolidinyl)carbamate (11.1 g, 76%). The product was used without further purification.

To a solution of 4-fluoro-2-{(E)-[4-oxo-2-(tetrahydropyridazin-1(2H)-yl)-1,3-thiazol-5(4H)-ylidene]-methyl}phenyl)-(pyrrolidinyl)carbamate (5.0 g, 11.15 mmol) in methanol (50 mL) was added methanesulfonic acid (723 up, 11.15 mmol). The solution was stirred for 0.5 hours, evaporated to dryness, and dried in vacuo, affording the final compound (5.81 g, 96%). ¹H NMR (400 MHz, D₂O) δ 1.81 (m, 4H), 2.63 (s, 3H), 3.22 (t, 2H, J=6.5 Hz), 3.28 (t, 2H, J=5.1 Hz), 3.43 (m, 6H), 3.82 (m, 4H), 4.05 (m (br), 2H), 6.86 (m, 2H), 7.20 (td, 1H, J=2.7 Hz, 6.1 Hz), 7.30 (s, 1H); M⁺ 449. HPLC purity: 98.8%.

### Example 109

### 2-{[(5E)-2-(1,2-Diazinan-1-yl)-4-oxo-4,5-dihydro-1,3-thiazol-5-ylidene]methyl}-5-fluorophenyl 4-(diethylamino)piperidine-1-carboxylate dihydrochloride

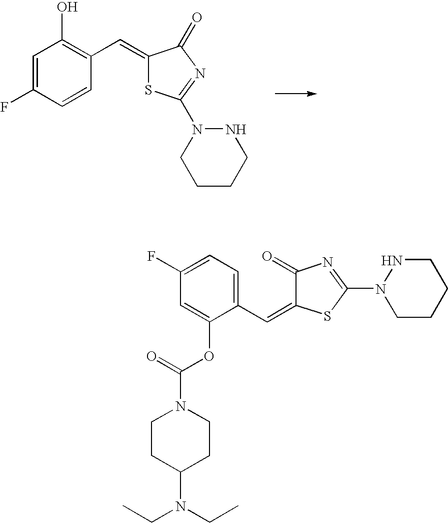

Example 109 was synthesized from (5Z)-2-(1,2-diazinan-1-yl)-5-[(4-fluoro-2-hydroxyphenyl)methylidene]-4,5-dihydro-1,3-thiazol-4-one and diethyl-piperidin-4-yl-amine carbamoyl chloride following the procedure described in Example 44. Yield was 21%. LRMS (ES⁺) m/z 490.

### Example 110

### 2-{[(5E)-2-(1,2-Diazinan-1-yl)-4-oxo-4,5-dihydro-1,3-thiazol-5-ylidene]methyl}-4-fluorophenyl-4-(diethylamino)piperidine-1-carboxylate dihydrochloride

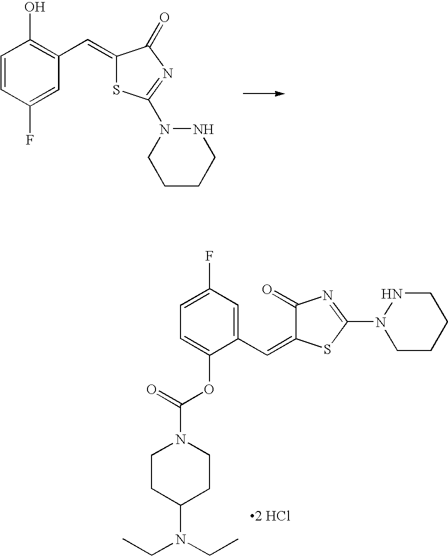

Example 110 was synthesized from (5Z)-2-(1,2-diazinan-1-yl)-5-[(5-fluoro-2-hydroxyphenyl)methylidene]-4,5-dihydro-1,3-thiazol-4-one and diethyl-piperidin-4-yl-amine carbamoyl chloride using the procedure described for Example 4. Yield 22%. LRMS (ES⁺) m/z 490. ¹H NMR (400 MHz, DMSO) δ 1.31 (t, J=7.2 Hz, 6H), 1.65-1.85 (m, 3×2H), 2.01-2.20 (m, 2H), 2.95-3.41 (m, 4×2H), 3.58 (m, 1H), 3.88 (bs, 2H), 4.09 (d, J=12.7 Hz, 1H), 4.35 (d, J=12.7 Hz, 1H), 6.21 (bs, 1H), 7.31-7.38 (m, 3H), 7.44 (s, 1H), 10.50 (s, 1H).

### Example 111

### 2-{[(5Z)-2-(1,2-Diazinan-1-yl)-4-sulfanylidene-4,5-dihydro-1,3-thiazol-5-ylidene]methyl}-5-fluorophenyl-4-(diethylamino)piperidine-1-carboxylate dihydrochloride

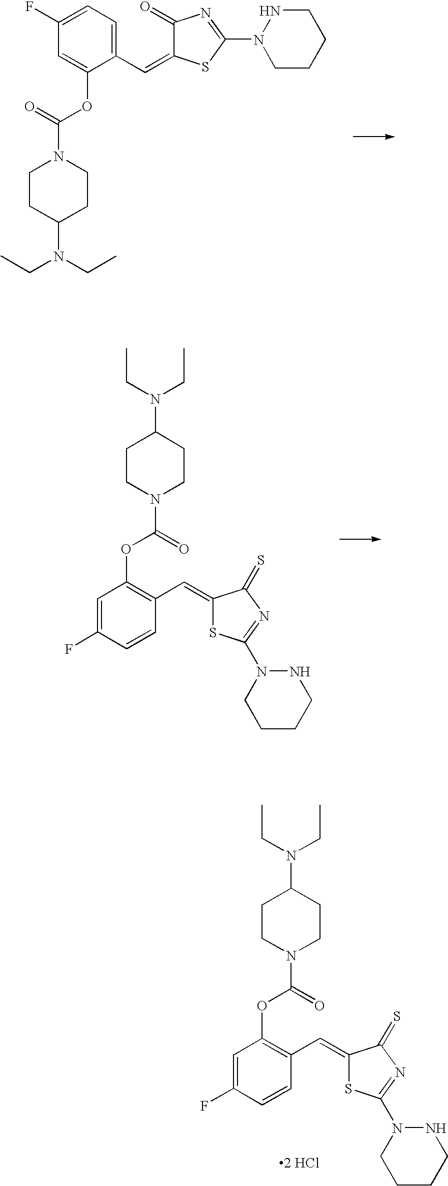

2-{[(5E)-2-(1,2-diazinan-1-yl)-4-oxo-4,5-dihydro-1,3-thiazol-5-ylidene]methyl}-5-fluorophenyl 4-(diethylamino)piperidine-1-carboxylate dihydrochloride (800 mg, 1.63 mmol) was dissolved in pyridine (24 mL), and phosphorus pentasulfide (800 mg, 1.80 mmol) was then added. The reaction mixture was stirred at 90° C. for 3 hours. The solvent was evaporated, and the residue triturated in DCM, and the solid removed by filtration. The filtrate was evaporated and coevaporated with toluene 3 times. The residue was dissolved in DCM and purified on silica gel using 10% MeOH/DCM to afford 2-{[(5Z)-2-(1,2-diazinan-1-yl)-4-sulfanylidene-4,5-dihydro-1,3-thiazol-5-ylidene]methyl}-5-fluorophenyl 4-(diethylamino)piperidine-1-carboxylate 124 mg, 15% yield. LRMS (ES⁺) m/z 405 (M⁺, 100). λ max=358 nm.

2-{[(5Z)-2-(1,2-diazinan-1-yl)-4-sulfanylidene-4,5-dihydro-1,3-thiazol-5-ylidene]methyl}-5-fluorophenyl 4-(diethylamino)piperidine-1-carboxylate (340 mg, 671 micromol) was stirred in methanol (5 mL), and a solution of HCl 4N in dioxane (420 microL, 1.68 mmol) was added dropwise at 0° C. The mixture was sonicated to afford a clear solution. The solvent was evaporated to provide a residue, and this residue was then washed 3 times with diethyl ether and dried in vacuo to afford to (Z)-5-fluoro-2-((2-(piperazin-1-yl)-4-thioxothiazol-5(4H)-ylidene)methyl)phenyl-4-(diethylamino)piperidine-1-carboxylate dihydrochloride (346 mg, 89% yield). LRMS (ES⁺) m/z 405 (M⁺, 100).

### Example 112

### (5Z)-5-[(4-Fluoro-2-hydroxyphenyl)methylidene]-2-(3-oxo-1,2-diazinan-1-yl)-4,5-dihydro-1,3-thiazol-4-one

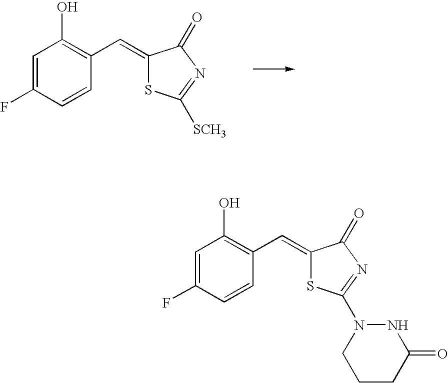

To a suspension of (5Z)-5-[(4-fluoro-2-hydroxyphenyl)methylidene]-2-(methylsulfanyl)-4,5-dihydro-1,3-thiazol-4-one (456 mg, 1.70 mmol) in absolute ethanol (15 mL) was added dropwise a solution of piperazin-3-one (254 mg, 2.54 mmol, prepared in 2 steps from 6-oxo-1,4,5,6-tetrahydropyridazine-3-carboxylic acid) in ethanol (5 mL) at room temperature. Triethylamine (592 μL, 4.25 mmol) was then added dropwise at 0° C. The reaction mixture was stirred at 50° C. overnight then cooled in an ice-water bath. The solid was recovered by filtration, washed with cold ethanol, and dried in vacuo, affording (5Z)-5-[(4-fluoro-2-hydroxyphenyl)methylidene]-2-(3-oxo-1,2-diazinan-1-yl)-4,5-dihydro-1,3-thiazol-4-one (346 mg, 63% yield). ¹H NMR (400 MHz, DMSO-d₆) δ 1.92-1.99 (m, 2H), 2.37 (t, J=6.7 Hz, 2H), 3.97 (t, J=6.3 Hz, 2H), 6.72-6.82 (m, 2H), 7.41-7.46 (m, 1H), 7.78 (s, 1H).

### Example 113

### (5Z)-5-{(4-Fluoro-2-hydroxyphenyl)[(3S)-pyrrolidin-3-ylamino]methylene}-2-(tetra-hydropyridazin-1(2H)-yl)-1,3-thiazol-4(5H)-one trihydrochloride

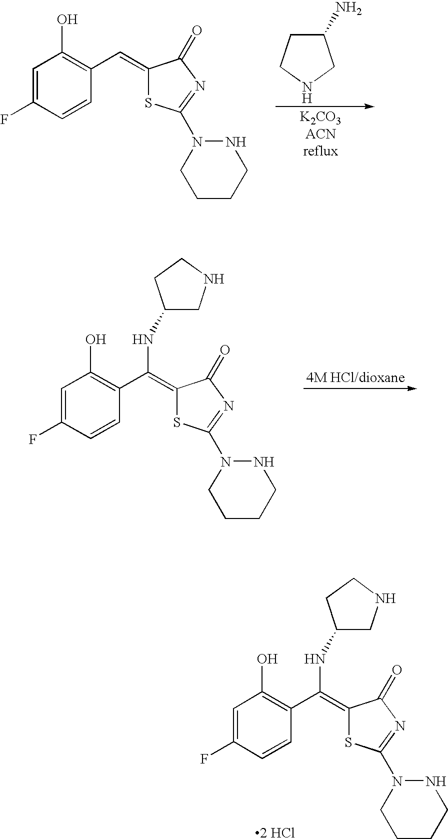

To a mixture of (5Z)-5-(4-fluoro-2-hydroxybenzylidene)-2-[4-(2-hydroxyethyl)piperazin-1-yl]-1,3-thiazol-4(5H)-one (1.3 g, 4.2 mmol) in anhydrous acetonitrile (10 mL) was added potassium carbonate (1.3 g, 9.4 mmol), followed by (3S)-3-aminopyrrolidine-1-carbonyl chloride (1.3 g, 5.5 mmol). The reaction mixture was stirred at reflux for 48 hours. After cooling to room temperature, the solid material was removed by filtration, and the filtrate was recovered and evaporated under reduced pressure. The crude product was purified by flash chromatography (0-10% MeOH/CH₂Cl₂ and 0-5% MeOH/CH₂Cl₂), affording (5Z)-5-{(4-fluoro-2-hydroxyphenyl)[(3S)-pyrrolidin-3-ylamino]methylene}-2-(tetra-hydropyridazin-1(2H)-yl)-1,3-thiazol-4(5H)-one (943 mg, 59%). The product was used without further purification.

To a solution of (5Z)-5-{(4-fluoro-2-hydroxyphenyl)[(3S)-pyrrolidin-3-ylamino]methylene}-2-(tetra-hydropyridazin-1(2H)-yl)-1,3-thiazol-4(5H)-one (900 mg, 2.3 mmol) in methanol (3 mL) was added 4M HCl solution in 1,4-dioxane (4 mL, 16 mmol). The solution was stirred for 0.5 hours, evaporated to dryness, and lyophilized, affording the title compound (780 mg, 68%). ¹H NMR (400 MHz, DMSO-d₆) δ 1.43 (m, 1H), 1.16 (m, 2H), 1.61 (m, 2H), 1.72 (m, 2H), 2.25 (m, 2H), 2.92 (m, 2H), 3.37 (m, 1H), 3.58 (m, 1H), 3.84 (m, 3H), 6.06 (t, 1H, J=6.8 Hz), 7.30 (m, 2H), 7.51 (m, 1H), 7.65 (t, 1H, J=2.3 Hz, 8.8 Hz); M⁺ 392.

### Example 114

### 2-{[(5E)-2-(1,2-Diazinan-1-yl)-4-oxo-4,5-dihydro-1,3-thiazol-5-ylidene]methyl}-5-fluorophenyl 2-amino-2-methylpropanoate dihydrochloride

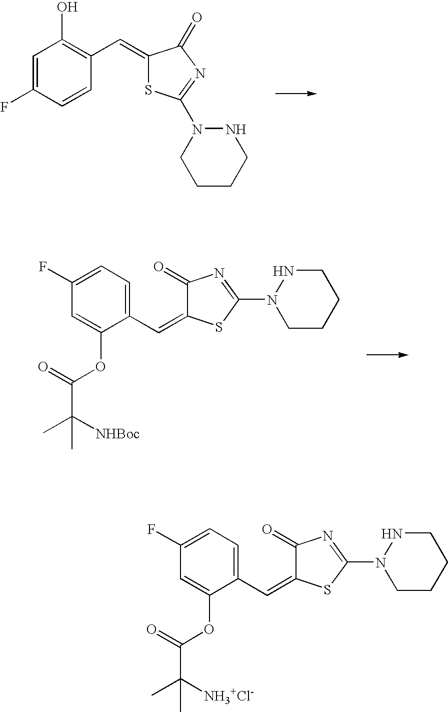

In a solution of (5Z)-5-(4-fluoro-2-hydroxybenzylidene)-2-[1,2-diazinan-1-yl]-1,3-thiazol-4(5H)-one (1.5 g; 4.9 mmol) in DCM (40 mL) was added at 0° C., DIEA (1.73 mL, 9.6 mmol), DMAP (catalytic), HOBt (822 mg, 5.4 mmol), Boc-Aib-OH (1.1 g, 5.4 mmol), and EDC (1.4 g, 7.3 mmol). The mixture was stirred at room temperature overnight, and water was added. The mixture was then extracted with DCM (3 times), washed with brine (2 times) and dried over MgSO₄. After the evaporation of solvent, the solid was triturated with DCM/Et₂O and then filtered to afford (E)-5-fluoro-2-((4-oxo-2-(1,2-diazinan-1-yl)thiazol-5(4H)-ylidene)methyl)phenyl-2-(tert-butoxycarbonyl amino)-2-methylpropanoate (1.83 g, 76% yield) as white solid. ¹H NMR (400 MHz, DMSO-d₆) δ 1.41 (s, 6H), 1.49 (s, 9H), 1.65 (bs, 2H), 1.74 (bs, 2H), 2.95 (bs, 2H), 3.86 (bs, 2H), 6.09 (t, J=7.2, 1H), 6.95 (dd, J=9.2 Hz, 2.5 Hz, 1H), 7.34 (dt, 8.5 Hz, 2.6 Hz, 1H), 7.46 (s, 1H), 7.68-7.72 (m, 1H), 7.79 (bs, 1H).

To a solution of (E)-5-fluoro-2-((4-oxo-2-(1,2-diazinan-1-yl)thiazol-5(4H)-ylidene)methyl)phenyl 2-(tert-butoxycarbonyl amino)-2-methylpropanoate (900 mg; 1.8 mmol) in dioxane (25 mL) was added dropwise at 0° C. a solution of HCl (4N in dioxane; 23 mL, 91 mmol). The reaction was stirred at room temperature overnight and evaporated under vacuum until solidification of the residue. This solid was recrystallized in MeOH/Et₂O to afford the final product (721 mg, 85% yield). ¹H NMR (400 MHz, DMSO-d₆) δ 1.65 (bs, 2H), 1.73 (bs, 2H and 6H), 2.94 (bs, 2H), 3.87 (bs, 2H), 6.17 (t, J=7.0 Hz, 1H), 7.39-7.49 (m, 3H), 7.71-7.76 (m, 1H), 8.99 (bs, 2H).

### Example 115

### 2-{[(5E)-2-(1,2-Diazinan-1-yl)-4-oxo-4,5-dihydro-1,3-thiazol-5-ylidene]methyl}-5-fluorophenyl (2R)-2-amino-3-methylbutanoate dihydrochloride

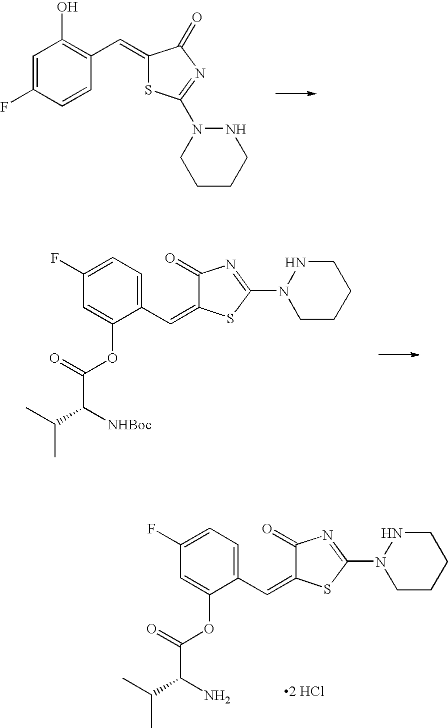

To a solution of (5Z)-2-(1,2-diazinan-1-yl)-5-[(4-fluoro-2-hydroxyphenyl)methylidene]-4,5-dihydro-1,3-thiazol-4-one (1.5 g; 4.9 mmol) in DCM (40 mL) was added at 0° C., DIEA (1.75 mL, 9.8 mmol), DMAP (catalytic), HOBt (822 mg, 5.4 mmol), Boc-L-valine (1.2 g, 5.4 mmol), and EDC (1.4 g, 7.3 mmol). The mixture was stirred at room temperature overnight, and water was added. The mixture was then extracted with DCM, washed with brine, and dried over MgSO₄. Evaporation of solvent afforded crude (R,E)-5-fluoro-2-((4-oxo-2-(1,2-diazinan-1-yl)thiazol-5(4H)-ylidene)methyl)phenyl-2-(tert-butoxycarbonyl amino)-3-methylbutanoate (1.6 g) as pale yellow solid that was used without further purification.

To a solution of (R,E)-5-fluoro-2-((4-oxo-2-(1,2-diazinan-1-yl)thiazol-5(4H)-ylidene)methyl)phenyl 2-(tert-butoxycarbonyl amino)-3-methylbutanoate (920 mg; 1.8 mmol) in dioxane (25 mL) was added dropwise at 0° C. a solution of HCl (4N in dioxane; 23 mL, 91 mmol). The reaction was stirred at room temperature overnight and evaporated under vacuum to afford a solid. This solid was recrystallized in MeOH/Et₂O to afford the final product (663 mg, 76% yield). ¹H NMR (400 MHz, DMSO-d₆) δ 1.11 (t, J=6.9, 6H), 1.65 (bs, 2H), 1.74 (bs, 2H), 2.37-2.43 (m, 1H), 2.93 (bs, 2H), 3.87 (bs, 2H), 4.20 (t, J=5.4 Hz, 1H), 6.17 (bs, 1H), 7.38-7.44 (m, 1H), 7.48-7.51 (m, 2H), 7.72-7.76 (m, 1H), 8.96 (bs, 2H).

### Example 116

### 2-{[(5E)-2-(1,2-Diazinan-1-yl)-4-oxo-4,5-dihydro-1,3-thiazol-5-ylidene]methyl}-5-fluorophenyl (2S)-2-amino-3-methylbutanoate dihydrochloride

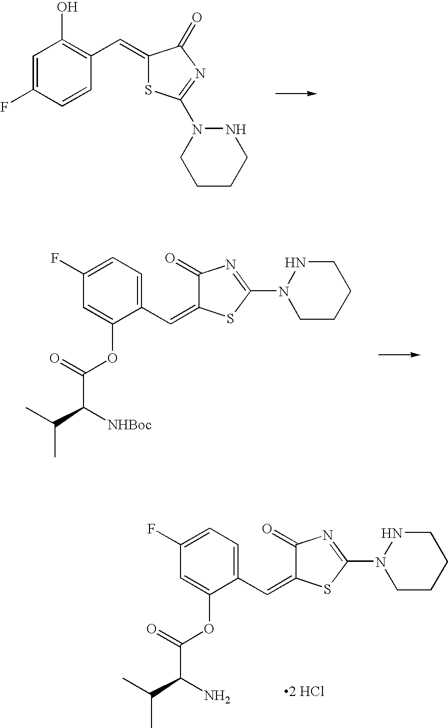

To a solution of (5Z)-2-(1,2-diazinan-1-yl)-5-[(4-fluoro-2-hydroxyphenyl)methylidene]-4,5-dihydro-1,3-thiazol-4-one (2.0 g; 6.5 mmol) in DCM (55 mL) was added DIEA (2.30 mL, 13.0 mmol), DMAP (catalytic), HOBt (1.1 g, 7.2 mmol), Boc-D-valine (1.6 g, 7.2 mmol), and EDC (1.9 g, 9.8 mmol) at 0° C. The mixture was stirred at room temperature overnight. Water was then added, and the mixture was then extracted with DCM (3 times), washed with brine (2 times), and dried over MgSO₄. Evaporation of solvent afforded crude (S,E)-5-fluoro-2-((4-oxo-2-(1,2-diazinan-1-yl)thiazol-5(4H)-ylidene)methyl)phenyl-2-(tert-butoxycarbonyl amino)-3-methylbutanoate (3.3 g) as a pale yellow solid that was used in the next step without further purification.

To a solution of (S,E)-5-fluoro-2-((4-oxo-2-(1,2-diazinan-1-yl)thiazol-5(4H)-ylidene)methyl)phenyl 2-(tert-butoxycarbonyl amino)-3-methylbutanoate (1 g; 2.0 mmol) in dioxane (30 mL) was added dropwise a solution of HCl (4N in dioxane; 25 mL, 99 mmol) at 0° C. The reaction was stirred at room temperature overnight and evaporated under vacuum to afford a solid. This solid was recrystallized in MeOH/Et₂O to afford the final compound (784 mg, 83% yield). ¹H NMR (400 MHz, DMSO-d₆) δ 1.09 (t, J=7.0, 6H), 1.63 (bs, 2H), 1.72 (bs, 2H), 2.35-2.40 (m, 1H), 2.91 (bs, 2H), 3.84 (bs, 2H), 4.19 (t, J=5.3 Hz, 1H), 6.13 (t, J=7.0 Hz, 1H), 7.36-7.46 (m, 2H), 7.47 (s, 1H), 7.72 (m, 1H), 8.87 (bs, 2H).

### Example 117

### (R,E)-tert-butyl 2-((5-fluoro-2-((4-oxo-2-(1,2-diazinan-1-yl)thiazol-5(4H)-ylidene)methyl)phenoxy)carbonyl amino)-3-methylbutanoic acid

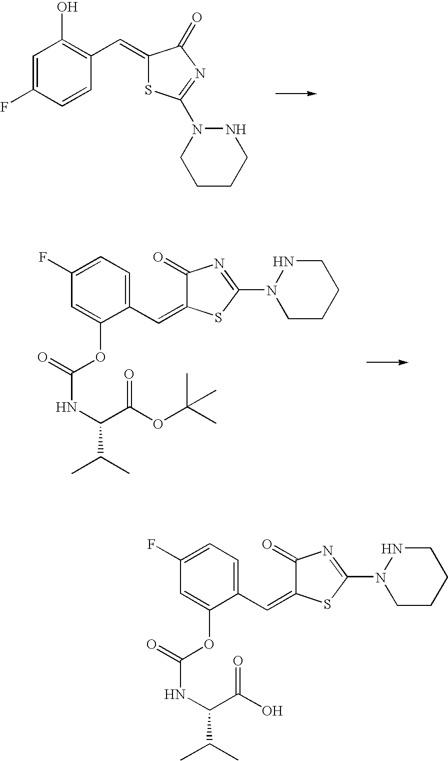

To a solution of (5Z)-2-(1,2-diazinan-1-yl)-5-[(4-fluoro-2-hydroxyphenyl)methylidene]-4,5-dihydro-1,3-thiazol-4-one (1.0 g; 3.2 mmol) in THF (16 mL) was added at 0° C., TEA (544 μL, 3.9 mmol) and a solution of (R)-tert-butyl 2-isocyanato-3-methylbutanoate (712 mg; 1.6 mmol) in THF (4 mL). The mixture was stirred at 60° C. overnight and then evaporated under vacuum. The residue was dissolved in DCM and purified on silica gel using 10% MeOH/DCM to afford (R,E)-tert-butyl-2-((5-fluoro-2-((4-oxo-2-(1,2-diazinan-1-yl)thiazol-5(4H)-ylidene)methyl)phenoxy)carbonyl amino)-3-methylbutanoate (435 mg, 27% yield) as yellow solid.

To a solution of (R,E)-tert-butyl 2-((5-fluoro-2-((4-oxo-2-(1,2-diazinan-1-yl)thiazol-5(4H)-ylidene)methyl)phenoxy)carbonyl amino)-3-methylbutanoate (200 mg; 396 μmol) in DCM (3 mL) was added dropwise TFA (1.2 mL, 15.8 mmol) at 0° C. The reaction was stirred at room temperature overnight and then evaporated under vacuum. The residue was dissolved in DCM and purified on silica gel using 5% MeOH/DCM to afford the final compound (103 mg, 58% yield, 90% pure by HPLC). ¹H NMR (400 MHz, DMSO-d₆) δ 0.93 (t, J=6.8 Hz, 6H), 1.62 (bs, 2H), 1.73 (bs, 2H), 2.09-2.16 (m, 1H), 2.92 (bs, 2H), 3.70-4.00 (m, 2H+1H), 6.05 (t, J=7.1 Hz, 1H), 7.17 (d, J=9.6 Hz, 1H), 7.27 (t, J=6.2 Hz, 1H), 7.58 (s, 1H), 7.65 (t, J=6.4 Hz, 1H), 8.42 (d, J=8.6 Hz, 1H).

### Example 118

### 5-Fluoro-2-{(E)-[4-oxo-2-(tetrahydropyridazin-1(2H)-yl)-1,3-thiazol-5(4H)-ylidene]-methyl}phenyl(2S)-2-amino-4-methylpentanoate dihydrochloride

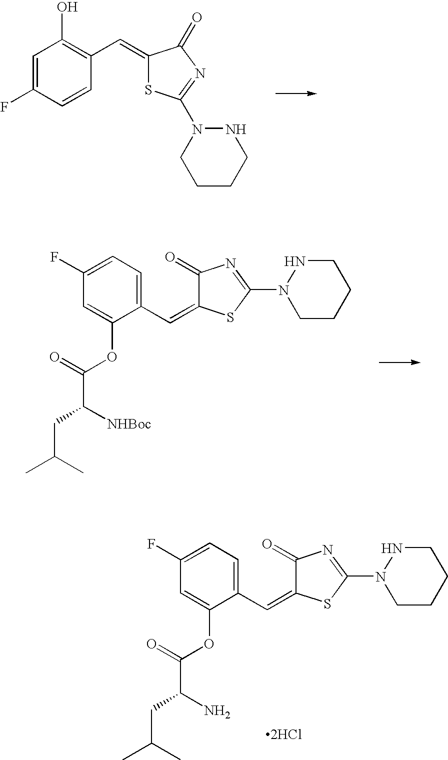

To a mixture of (5Z)-2-(1,2-diazinan-1-yl)-5-[(4-fluoro-2-hydroxyphenyl)methylidene]-4,5-dihydro-1,3-thiazol-4-one, 2.46 g, 8.0 mmol) and triethylamine (2.3 mL, 16.5 mmol) in anhydrous dichloromethane (30 mL) and DMF (7 mL) was added N-Boc-Leu-OH (2.0 g, 8.0 mmol), followed by hydroxybenzotriazole (1.84 g, 12.0 mmol) and EDC hydrochloride (2.29 g, 12.0 mmol). The reaction mixture was stirred at room temperature for 5 hours. The mixture was extracted with saturated NaHCO₃ (2×50 mL), 10% KHSO₄ (2×50 mL) and brine (2×50 mL). The organic phase was dried over MgSO₄, filtered, evaporated, and dried in vacuo. The oil was dissolved in diethyl ether (20 mL). The solution was extracted with brine (1×40 mL). The organic phase was dried over MgSO₄, filtered, evaporated, and dried in vacuo, affording (S,E)-5-fluoro-2-((4-oxo-2-(1,2-diazinan-1-yl)thiazol-5(4H)-ylidene)methyl)phenyl 2-(tert-butoxycarbonyl amino)-4-methylpentanoate (3.34 g, 80%), which was used without further purification.

To a mixture of (S,E)-5-fluoro-2-((4-oxo-2-(1,2-diazinan-1-yl)thiazol-5(4H)-ylidene)methyl)phenyl-2-(tert-butoxycarbonyl amino)-4-methylpentanoate (3.3 mg, 6.4 mmol) in 1,4-dioxanel (35 mL) was added a solution of 4M HCl/dioxane (8 mL, 32.0 mmol). The mixture was stirred at room temperature overnight. The solid material was recovered by filtration, washed with dioxane (1×20 mL) and diethyl ether (1×20 mL), and dried in vacuo. The solid material (1.69 g) was dissolved in methanol (5 mL), and then diethyl ether (10 mL) was added slowly. A solid precipitated slowly. More diethyl ether (50 mL) was added. The solid material was recovered by filtration, washed with diethyl ether (1×10 mL), and dried in vacuo, affording the desired compound (1.1 g, 65%). ¹H NMR (400 MHz, DMSO-d₆) δ 0.99 (m, 6H), 1.69 (m, 2H), 1.78 (m, 2H), 1.88 (m, 3H), 2.94 (m, 2H), 3.87 (m, 2H), 4.29 (m, 1H), 6.16 (t, 1H, J=7.0 Hz), 7.43 (m, 3H), 7.67 (td, 1H, J=2.7 Hz, 8.6 Hz), 8.86 (d, 1H, J=4.1 Hz); M+421.

### Example 119

### 5-fluoro-2-{(E)-[4-oxo-2-(tetrahydropyridazin-1(2H)-yl)-1,3-thiazol-5(4H)-ylidene]-methyl}phenyl-3-aminopropanoate dihydrochloride

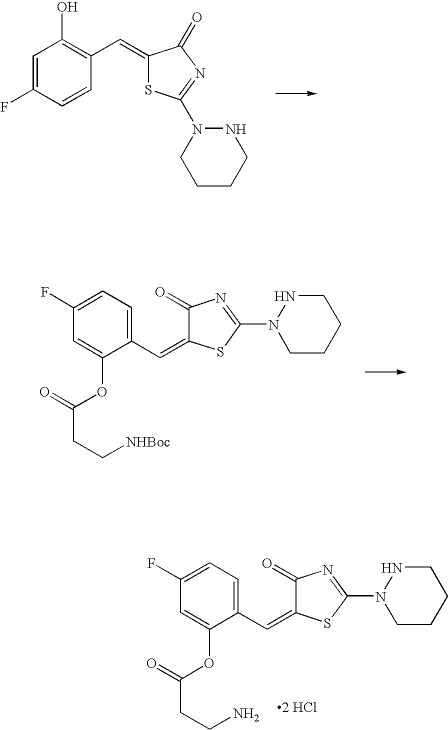

Example 119 was prepared from (5Z)-2-(1,2-diazinan-1-yl)-5-[(4-fluoro-2-hydroxyphenyl)methylidene]-4,5-dihydro-1,3-thiazol-4-one and 3-tert-butoxycarbonylamino-propionic acid following the procedure described for Example 118.1H NMR (400 MHz, DMSO-d₆) δ 1.66 (m, 2H), 1.75 (m, 2H), 2.94 (m, 2H), 3.12 (m, 4H), 3.88 (m, 2H), 6.20 (m, 1H), 7.36 (td, 1H, J=2.7 Hz, 8.6 Hz), 7.45 (m, 2H), 7.71 (td, 1H, J=6.3 Hz, 8.6 Hz), 8.27 (s, 1H); M⁺ 379.

### Example 120

### 2-{[(5E)-2-(1,2-diazinan-1-yl)-4-oxo-4,5-dihydro-1,3-thiazol-5-ylidene]methyl}-5-fluorophenyl (2S)-pyrrolidine-2-carboxylate dihydrochloride

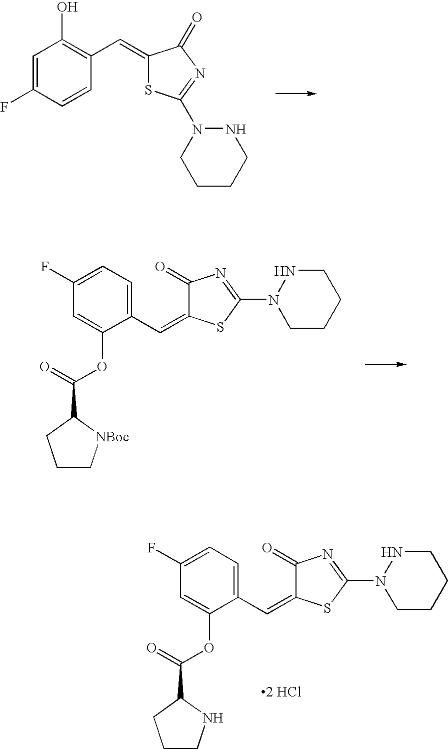

Example 120 was prepared from (5Z)-2-(1,2-diazinan-1-yl)-5-[(4-fluoro-2-hydroxyphenyl)methylidene]-4,5-dihydro-1,3-thiazol-4-one and 3-tert-butoxycarbony-L-proline following the procedure as described in Example 118. ¹H NMR (400 MHz, DMSO-d₆) δ 1.65 (m, 2H), 1.75 (m, 2H), 2.04 (m, 2H), 2.29 (m, 1H), 2.45 (m, 1H), 2.94 (m, 2H), 3.30 (m, 2H), 3.87 (m, 2H), 4.78 (m, 1H), 6.18 (t, 1H, J=6.1 Hz), 7.41 (td, 1H, J=2.3 Hz, 8.4 Hz), 7.48 (s, 1H), 7.52 (dd, 1H, J=2.5 Hz, 6.8 Hz), 7.74 (td, 1H, J=2.3, Hz, 8.6 Hz), 9.45 (s(br), 1H), 10.37 (s(br), 1H); M⁺ 405.

### Example 121

### (5Z)-5-[(2-hydroxyphenyl)methylidene]-2-[(3R)-3-hydroxypyrrolidin-1-yl]-4,5-dihydro-1,3-thiazol-4-one

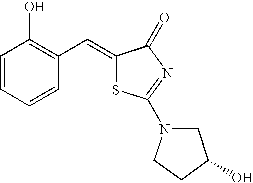

This compound was synthesized as per the procedure described for Example 1 by using 2-hydroxybenzaldehyde, rhodanine and (R)-3-pyrrolidinol to provide yellow solid in 76% yield. NH-NMR (400 MHz, DMSO): 1.90-2.15 (m, 2H), 3.41-3.80 (m, 4H), 4.42 (br, 1H), 5.24 (dd, 1H), 6.95 (m, 2H), 7.28 (m, 1H), 7.42 (m, 1H), 7.92 (s, 1H). LRMS: M+291.

### Example 122

### (5Z)-5-[(5-fluoro-2-hydroxyphenyl)methylidene]-2-[(3R)-3-hydroxypyrrolidin-1-yl]-4,5-dihydro-1,3-thiazol-4-one

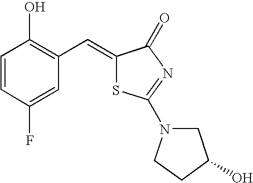

This compound was synthesized as per the procedure described for Example 1 by using 5-fluoro-2-hydroxybenzaldehyde, rhodanine, and (R)-3-pyrrolidinol to provide a yellow solid in 28% yield. ¹H-NMR (400 MHz, DMSO): 1.95-2.11 (m, 2H), 3.44-3.82 (m, 4H), 4.42 (br, 1H), 5.24 (dd, 1H), 6.95 (m, 1H), 7.16 (m, 2H), 7.82 (s, 1H).

### Example 123

### (5Z)-5-[(2-hydroxyphenyl)methylidene]-2-[(3S)-3-hydroxypyrrolidin-1-yl]-4,5-dihydro-1,3-thiazol-4-one

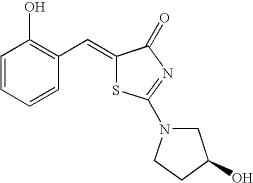

This compound was synthesized by following the procedure for Example 1 using 2-hydroxy-benzaldehyde, rhodanine, and (S)-pyrrolidin-3-ol. Yield 66%. ¹H NMR (400 MHz, DMSO) 2.05 (m, 2H), 3.44 (d, 1H, J=10.9 Hz), 3.75 (m, 4H), 4.42 (m, 1H), 5.24 (m, 1H), 6.93 (m, 2H), 7.26 (t, 1H, J=7.0 Hz), 7.42 dt, IH, J=8.0 Hz), 7.91 (s, 1H); M+291.

### Example 124

### (5Z)-5-[(5-fluoro-2-hydroxyphenyl)methylidene]-2-[(3S)-3-hydroxypyrrolidin-1-yl]-4,5-dihydro-1,3-thiazol-4-one

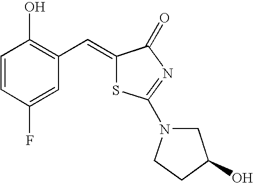

This compound was synthesized by following the procedure for Example 1 using 5-fluoro-2-hydroxy-benzaldehyde, rhodanine, and (S)-pyrrolidin-3-ol. Yield 55%. ¹H NMR (400 MHz, DMSO) 2.03 (m, 2H), 3.44 (d, 1H, J=10.9 Hz), 3.79 (m, 4H), 4.42 (m, 1H), 5.24 (m, 1H), 6.92 (m, 1H), 7.12 (m, 2H), 7.80 (s, 1H), 10.37 (s, 1H); M+ 309.

### Example 125

### (5Z)-5-[(2-hydroxyphenyl)methylidene]-2-(1,3-thiazolidin-3-yl)-4,5-dihydro-1,3-thiazol-4-one

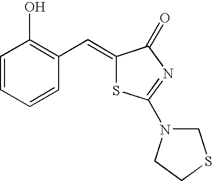

This compound was synthesized by following the procedure for Example 1 using 5-fluoro-2-hydroxy-benzaldehyde, rhodanine, and isothiazolidine. Yield 65%. ¹H NMR (400 MHz, DMSO) 3.21 (m, 2H), 2.38 (m, 1H), 3.93 (m, 1H), 4.01 (m, 1H), 4.74 (s, 1H), 4.81 (s, 1H), 6.93 (m, 1H), 7.15 (m, 2H), 7.84 (s, 1H), 10.43 (s, 1H), 13.20 (s, 1H); M+ 311.

### Example 126

### (5Z)-5-[(2-hydroxy-5-methanesulfonylphenyl)methylidene]-2-(pyrrolidin-1-yl)-4,5-dihydro-1,3-thiazol-4-one

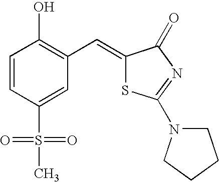

This compound was synthesized as per the procedure described for Example 1 by using 2-hydroxy-5-methanesulfonylbenzaldehyde, rhodanine, and pyrrolidine to provide a solid in 83% yield. ¹H-NMR (400 MHz, DMSO): 2.00 (m, 4H), 3.08 (s, 3H), 3.60 (m, 4H), 6.70 (m, 1H), 7.55 (m, 1H), 7.72 (s, 1H), 7.87 (s, 1H). LRMS: M+ 353.

### Example 127

### 1-[(5Z)-5-[(5-fluoro-2-hydroxyphenyl)methylidene]-4-oxo-4,5-dihydro-1,3-thiazol-2-yl]pyrrolidine-2-carboxamide

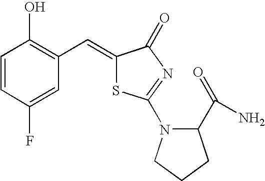

This compound was prepared from Example 38 and pyrrolidine-2-carboxylic acid amide by following the procedure for Example 39. Yield: 2%; ¹H NMR (400 MHz, DMSO) 2.01 (m, 2H), 2.37 (m, 1H), 2.71 (m, 1H), 3.76 (m, 2H), 4.46 (m, 0.4H), 4.57 (m, 0.6H), 6.93 (m, 1H), 7.03 (m, 0.4H), 7.13 (m, 2.6H), 7.37 (s, 0.4H), 7.60 (s, 0.6H), 7.81 (s, 1H), 10.45 (s(br), 1H); M+ 336.

### Example 128

### (2R)-1-[(5Z)-5-[(5-fluoro-2-hydroxyphenyl)methylidene]-4-oxo-4,5-dihydro-1,3-thiazol-2-yl]pyrrolidine-2-carboxylic acid

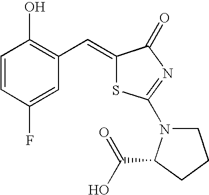

This compound was prepared from Example 38 and (R) proline by following the procedure for Example 39. Yield: 21%, ¹H NMR (400 MHz, DMSO) 2.04 (m, 3H), 2.41 (m, 1H), 3.77 (m, 2H), 4.65 (m, 2H), 6.95 (m, 1H), 7.15 (m, 2H), 7.85 (s, 1H), 10.39 (s, 1H), 13.09 (s(br), 1H), M+ 337.

### Example 129

### (2S)-1-[(5Z)-5-[(5-fluoro-2-hydroxyphenyl)methylidene]-4-oxo-4,5-dihydro-1,3-thiazol-2-yl]pyrrolidine-2-carboxylic acid

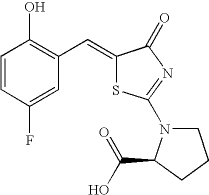

This compound was prepared from Example 38 using and (S) proline by following the procedure for Example 39. Yield: 13%, ¹H NMR (400 MHz, DMSO) 2.07 (m, 3H), 2.38 (m, 1H), 3.74 (m, 2H), 4.66 (m, 2H), 6.96 (m, 1H), 7.165 (m, 2H), 7.84 (s, 1H), 10.46 (s, 1H), 13.20 (s(br), 1H), M+ 337.

### Example 130

### (5Z)-5-[(5-Fluoro-2-hydroxyphenyl)methylidene]-2-[(2R)-2-(hydroxymethyl)pyrrolidin-1-yl]-4,5-dihydro-1,3-thiazol-4-one

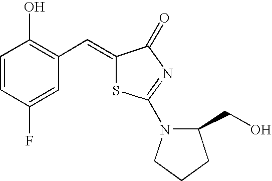

This compound was synthesized from Example 38 and D-prolinol by following the procedure for Example 39. A yellow solid was obtained in 56% yield. ¹H-NMR (400 MHz, DMSO): 1.95-2.11 (m, 4H), 3.44-3.68 (m, 4H), 3.95, 4.20 (m, 1H), 6.70 (m, 2H), 7.38 (m, 1H), 7.82 (s, 1H)

### Example 131

### tert-Butyl N-[(3S)-1-[(5Z)-5-[(5-fluoro-2-hydroxyphenyl)methylidene]-4-oxo-4,5-dihydro-1,3-thiazol-2-yl]pyrrolidin-3-yl]carbamate

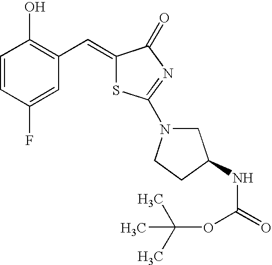

This compound was synthesized by following the procedure for Example 1 using 5-fluoro-2-hydroxy-benzaldehyde, rhodanine, and pyrrolidin-3-yl-carbamic acid tert-butyl ester. Yield 66%. ¹H NMR (400 MHz, DMSO) 1.94 (m, 1H), 2.29 (m, 1H), 3.45 (m, 1H), 3.60 (m, 1H), 3.78 (m, 3H), 4.15 (m, 1H), 6.95 (m, 1H), 7.14 (m, 2H), 7.38 (m, 1H), 7.83 (s, 1H), 10.41 (s (br), 1H); M+408.

### Example 132

### (5Z)-5-[(4,5-difluoro-2-hydroxyphenyl)methylidene]-2-(pyrrolidin-1-yl)-4,5-dihydro-1,3-thiazol-4-one

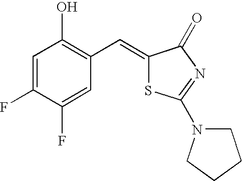

This compound was prepared from 4,5-difluoro-2-hydroxy-benzaldehyde and 2-pyrrolidin-1-yl-1,3-thiazol-4-one following the procedure for example 28. A yellow solid was obtained in 86% yield. ¹H NMR (400 MHz, DMSO-d₆): 1.90 (m, 4H), 2.95 (m, 1H), 3.70 (m, 2H), 3.80 (m, 2H), 6.90 (m, 1H), 7.30 (m, 1H), 7.80 (s, 1H).

### Example 133

### (5Z)-2-[(8aS)-octahydropyrrolo[1,2-a]piperazin-2-yl]-5-[(5-fluoro-2-hydroxyphenyl)methylidene]-4,5-dihydro-1,3-thiazol-4-one

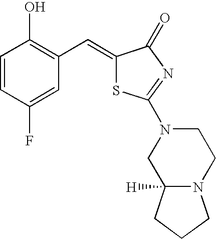

This compound was synthesized from Example 38 and (S) octahydro-pyrrolo[1,2-c]piperazine by following the procedure for Example 39. The product was obtained as yellow solid in 82% yield. ¹H NMR (400 MHz, DMSO-d₆): 1.40 (m, 1H), 1.60-2.30 (m, 3H), 2.90-3.60 (m, 3H), 3.90 (m, 3H), 3.80 (2d, J=13.1, 11.6 Hz, 1H), 4.60 (2d, J=13.1, 11.6 Hz, 1H), 6.95 (m, 1H), 7.20 (m, 2H), 7.81 (s, 1H), 10.4 ((s, 1H). M+347.4.

### Example 134

### (5Z)-2-[(8aR)-octahydropyrrolo[1,2-a]piperazin-2-yl]-5-[(5-fluoro-2-hydroxyphenyl)methylidene]-4,5-dihydro-1,3-thiazol-4-one

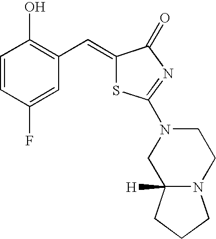

This compound was synthesized from Example 38 and (R) octahydro-pyrrolo[1,2-c]piperazine by following the procedure for Example 39. The product was obtained as yellow solid in 75% yield. ¹H NMR (400 MHz, DMSO-d₆): 1.40 (m, 1H), 1.60-2.30 (m, 3H), 2.90-3.60 (m, 3H), 3.90 (m, 3H), 3.80 (2d, J=13.1, 11.6 Hz, 1H), 4.60 (2d, J=13.1, 11.6 Hz, 1H), 6.95 (m, 1H), 7.20 (m, 2H), 7.81 (s, 1H), 10.4 ((s, 1H). M+347.4.

### Example 135

### (5Z)-5-[(5-fluoro-2-hydroxyphenyl)methylidene]-2-[(2S)-2-methylpiperazin-1-yl]-4,5-dihydro-1,3-thiazol-4-one hydrochloride

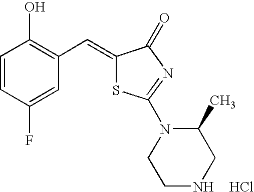

This compound was prepared from Example 38 and N-Boc-(S)-methylpiperazine as described in Example 149. Yield: 68%. ¹H NMR (400 MHz, DMSO) 1.47 (m, 3H), 3.19 (m, 2H), 3.38 (m, 2H), 3.59 (m, 0.5H), 3.81 (m, 0.5H), 3.95 (m, 0.5H), 4.38 (m, 0.5H), 4.65 (m, 0.5H), 5.02 (m, 0.5H), 7.04 (m, 1H), 7.17 (m, 2H), 7.94 (s, 1H), 9.50 (s(br), 1H), 9.85 (s(br), 1H), 10.56 (s(br), 1H); M+322.

### Example 136

### tert-butyl (3S)-4-[(5Z)-5-[(5-fluoro-2-hydroxyphenyl)methylidene]-4-oxo-4,5-dihydro-1,3-thiazol-2-yl]-3-methylpiperazine-1-carboxylate

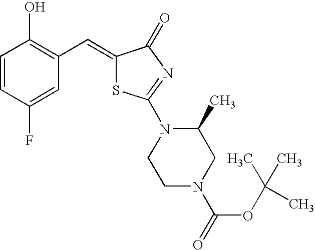

This compound was prepared from Example 38 and N-Boc-(S)-methylpiperazine as described in Example 149. Yield: 48%; ¹H NMR (400 MHz, DMSO) 1.26 (m, 3H), 1.39 (s, 9H), 3.59 (m, 5.5H), 4.44 (m, 1H), 4.85 (m, 0.5H), 6.95 (m, 1H), 7.16 (m, 2H), 7.86 (s, 1H); M+422.

### Example 137

### (5Z)-5-[(5-fluoro-2-hydroxyphenyl)methylidene]-2-(pyrazolidin-1-yl)-4,5-dihydro-1,3-thiazol-4-one

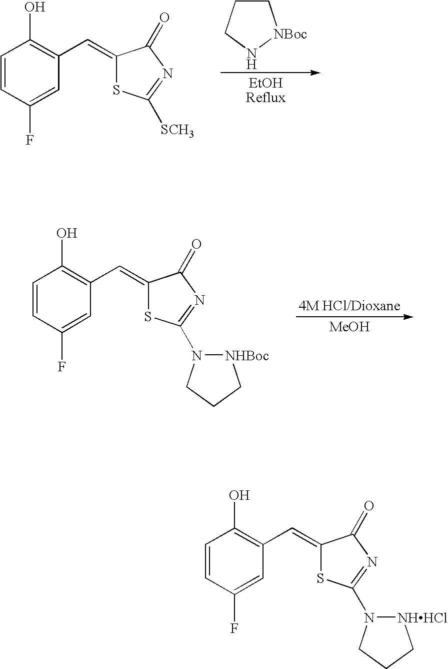

To a solution of (5Z)-5-[(5-fluoro-2-hydroxyphenyl)methylidene]-2-(methylsulfanyl)-4,5-dihydro-1,3-thiazol-4-one (710 mg, 2.6 mmol) in absolute ethanol (5 mL) was added N′-Boc-pyrazolidine (500 mg, 2.9 mmol). The reaction mixture was stirred at reflux overnight. After cooling to room temperature, the solid material was recovered by filtration and washed with EtOH (1×10 mL) and diethyl ether (2×10 ml) and dried in vacuo, affording the boc protected compound. The product was used without further purification. To a mixture of boc protected compound (300 mg, 0.76 mmol) in MeOH (2 mL) was added 4M HCl in dioxane (3 mL, 12.0 mmol). The reaction mixture was stirred at room temperature overnight. The solid material was recovered by filtration and washed with diethyl ether (2×10 ml) and dried in vacuo, affording the title compound 200 mg (80%); ¹H NMR (400 MHz, DMSO) 2.16 (m, 2H), 3.04 (t, 2H, J=6.7 Hz), 3.73 (t, 2H, J=7.5 Hz), 6.98 (m, 1H), 7.13 (m, 2H), 7.80 (d, 1H, J=1.2 Hz); M+294.

### Example 138

### (5Z)-5-[(5-fluoro-2-hydroxyphenyl)methylidene]-2-[(2R)-4-(2-hydroxyethyl)-2-methylpiperazin-1-yl]-4,5-dihydro-1,3-thiazol-4-one

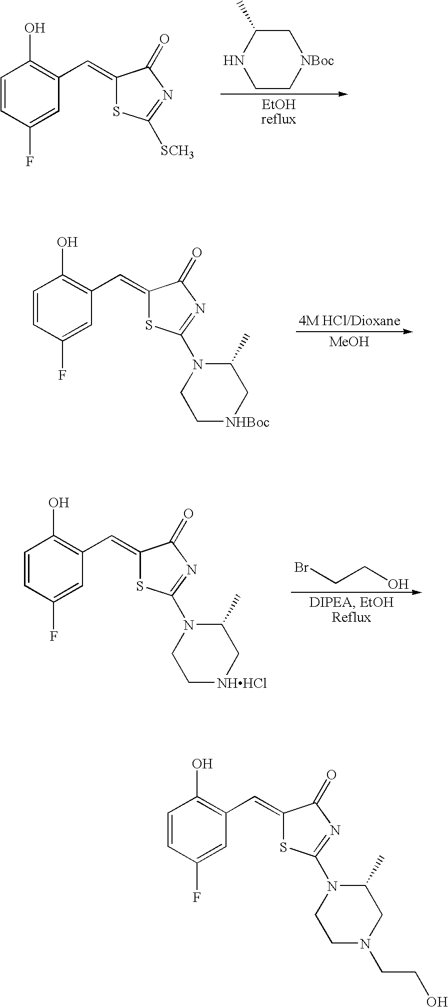

To a solution of (5Z)-5-[(5-fluoro-2-hydroxyphenyl)methylidene]-2-(methylsulfanyl)-4,5-dihydro-1,3-thiazol-4-one (1.35 g, 5.0 mmol) in absolute ethanol (10 mL) was added N-Boc-(R)-methylpiperazine (2.0 g, 10.0 mmol). The reaction mixture was stirred at 90° C. overnight. After cooling to room temperature, the solvent was evaporated under reduced pressure. The crude product was purified by flash chromatography (normal phase, 0-10% MeOH in CH₂Cl₂) to afford, tert-butyl 4-(5Z)-5-(5-fluoro-2-hydroxybenzylidene)-[(3R)-3-methyl-4-(4-oxo-4,5-dihydro-1,3-thiazol-2-yl)piperazine]-1-carboxylate (3.37 g, yield 80%).

To tert-butyl 4-(5Z)-5-(5-fluoro-2-hydroxybenzylidene)-[(3R)-3-methyl-4-(4-oxo-4,5-dihydro-1,3-thiazol-2-yl)piperazine]-1-carboxylate (3.37 g, 8.0 mmol) in MeOH (10 mL) was added 4M HCl in dioxane (10 mL). The reaction mixture was stirred at room temperature overnight. The solid material was recovered by filtration and washed with diethyl ether (2×20 ml) and dried under vacuum, affording (5Z)-5-(5-fluoro-2-hydroxybenzylidene)-2-[(2R)-2-methylpiperazin-1-yl]-1,3-thiazol-4(5H)-one (2.85 g, 99%). The product was used without further purification.

To a solution of (5Z)-5-(5-fluoro-2-hydroxybenzylidene)-2-[(2R)-2-methylpiperazin-1-yl]-1,3-thiazol-4(5H)-one (536 g, 1.5 mmol) in THF (10 mL) was added N—N-diisopropylethylamine (485 mg, 3.8 mmol) and bromoethanol (225 mg, 1.8 mmol). The resulting mixture was stirred at room temperature overnight. After cooling to room temperature, the solvent was evaporated under reduced pressure. The crude product was purified by flash chromatography (0-10% MeOH in DCM) and dried under vacuum to afford the final compound (75 mg, 14%). ¹H-NMR (400 MHz, CD₃OD): 1.44 (m, 3H), 2.20-2.51 (m, 4H), 2.95-3.05 (m, 2H), 3.50-3.76 (m, 4H), 4.10, 4.52 (m, 1H), 6.88 (m, 1H), 7.02 (m, 1H), 7.18 (m, 1H), 8.06 (s, 1H). LRMS: M⁺ 366

### Example 139

### (5Z)-5-[(5-fluoro-2-hydroxyphenyl)methylidene]-2-[(2S)-4-(2-hydroxyethyl)-2-methylpiperazin-1-yl]-4,5-dihydro-1,3-thiazol-4-one

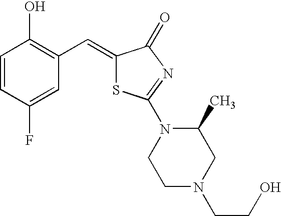

This compound was prepared from Example 38 and N-Boc-(S)-methylpiperazine as described in Example 138. Yield: 22%. ¹H NMR (400 MHz, DMSO) 1.40 (m, 3H), 2.09 (m, 1H), 2.29 (m, 1H), 2.44 (m, 2H), 2.86 (m, 1H), 3.00 (m, 1H), 3.60 (m, 4H), 4.42 (m, 0.5H), 4.50 (m, 1H), 4.79 (m, 0.5H), 6.95 (t, 1H, J=8.8 Hz), 7.16 (m, 2H), 7.84 (s, 1H), 10.40 (s, 1H); M+366.

### Example 140

### (5Z)-2-[(8aR)-octahydropyrrolo[1,2-a]piperazin-2-yl]-5-[(4-fluoro-2-hydroxyphenyl)methylidene]-4,5-dihydro-1,3-thiazol-4-one

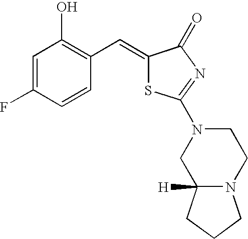

This compound was synthesized from Example 37 and (R)-1,4-diazabicyclo[4.3.0]nonane by following the procedure for Example 39. A yellow solid was obtained in 74% yield. ¹H-NMR (400 MHz, DMSO): 1.40 (m, 1H), 1.65-2.20 (m, 6H), 2.98-3.45 (m, 4H), 3.80-3.93 (m, 1H), 4.57-4.71 (m, 1H), 6.75 (m, 2H), 7.46 (m, 1H), 7.85 (m, 1H).

LRMS: M⁺ 348.

### Example 141

### (5Z)-2-(4-cyclopropylpiperazin-1-yl)-5-[(4-fluoro-2-hydroxyphenyl)methylidene]-4,5-dihydro-1,3-thiazol-4-one

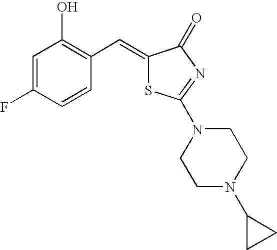

This compound was synthesized from Example 37 and 1-cyclopropylpiperazine by following the procedure for Example 39. A yellow solid was obtained in 82% yield. ¹H NMR (400 MHz, DMSO-d₆): 0.65-1.20 (m, 4H), 2.80 (m, 1H), 2.80 (m, 2H), 3.02-4.01 (m, 8H), 1H), 6.80 (m, 2H), 7.51 (m, 1H), (m, 2H), 7.95 (s, 1H), 11.01 (bs, 1H). M+347.4.

### Example 142

### (5Z)-2-(4-cyclopropylpiperazin-1-yl)-5-[(5-fluoro-2-hydroxyphenyl)methylidene]-4,5-dihydro-1,3-thiazol-4-one

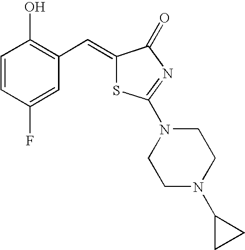

This compound was synthesized from Example 38 and 1-cyclopropyl-piperazine by following the procedure for Example 39. A yellow solid was obtained in 83% yield. ¹H NMR (400 MHz, DMSO-d₆)-0.8-0.20 (m, 4H), 1.59 (bs, 1H), 2.89 (m, 1H) 3.21-3.81 (m, 6H), 6.61 (m, 2H), 6.81 (m, 2H), 7.61 (s, 1H), 10.01 (bs, 1H).

### Example 143

### (5Z)-5-[(4-fluoro-2-hydroxyphenyl)methylidene]-2-[(2S)-2-methylpiperazin-1-yl]-4,5-dihydro-1,3-thiazol-4-one hydrochloride

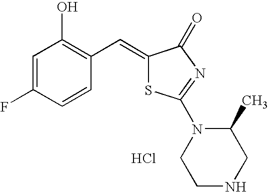

### Described in Example 149.

### Example 144

### (5Z)-5-[(4-fluoro-2-hydroxyphenyl)methylidene]-2-[(2R)-4-(2-hydroxyethyl)-2-methylpiperazin-1-yl]-4,5-dihydro-1,3-thiazol-4-one

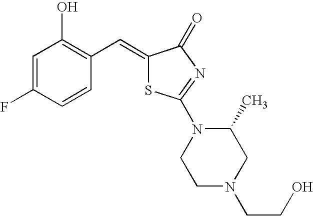

This compound was prepared by following the same procedure described for Example 138 by using (5Z)-5-[(4-fluoro-2-hydroxyphenyl)methylidene]-2-(methylsulfanyl)-4,5-dihydro-1,3-thiazol-4-one as starting material. ¹H-NMR (400 MHz, CD₃OD): 1.36 (m, 3H), 2.05-2.41 (m, 4H), 2.90-3.00 (m, 2H), 3.50-3.60 (m, 4H), 4.02, 4.42 (m, 1H), 6.77 (m, 2H), 7.43 (m, 1H), 7.82 (s, 1H). LRMS: M⁺ 366.

### Example 145

### (5Z)-5-[(4-fluoro-2-hydroxyphenyl)methylidene]-2-[(2R)-2-(hydroxymethyl)pyrrolidin-1-yl]-4,5-dihydro-1,3-thiazol-4-one

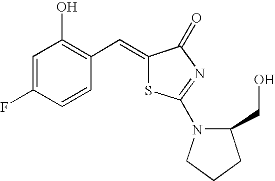

This compound was synthesized from Example 37 and D-prolinol by following the procedure for Example 39. A yellow solid was obtained in 56% yield. ¹H-NMR (400 MHz, DMSO): 1.95-2.11 (m, 4H), 3.44-3.68 (m, 4H), 3.95, 4.20 (m, 1H), 6.70 (m, 2H), 7.38 (m, 1H), 7.82 (s, 1H)

### Example 146

### (5Z)-5-[(4-fluoro-2-hydroxyphenyl)methylidene]-2-[(2S)-2-(hydroxymethyl)pyrrolidin-1-yl]-4,5-dihydro-1,3-thiazol-4-one

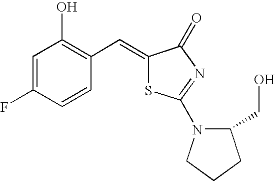

This compound was synthesized from Example 37 and L-prolinol by following the procedure for Example 39. A yellow solid was obtained in 56% yield. ¹H-NMR (400 MHz, DMSO): 1.95-2.11 (m, 4H), 3.44-3.68 (m, 4H), 3.95, 4.20 (m, 1H), 6.72 (m, 2H), 7.41 (m, 1H), 7.82 (s, 1H). LRMS: M+323.

### Example 147

### (5Z)-2-[(2S)-4-(cyclopropylmethyl)-2-methylpiperazin-1-yl]-5-[(4-fluoro-2-hydroxyphenyl)methylidene]-4,5-dihydro-1,3-thiazol-4-one

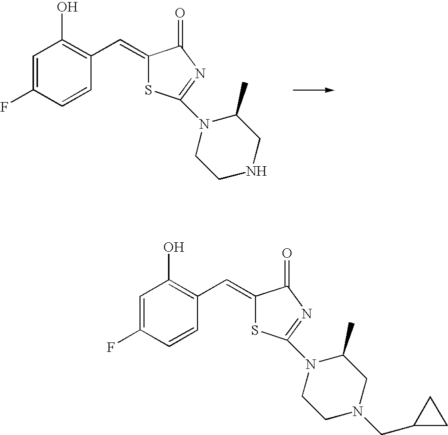

To a solution of (5Z)-5-[(4-fluoro-2-hydroxyphenyl)methylidene]-2-[(2S)-2-methylpiperazin-1-yl]-4,5-dihydro-1,3-thiazol-4-one (0.2 g, 0.56 mmol) in ethanol was added diisopropylethylamine (250 uL, 1.44 mmol) followed by bromomethylcyclopropane. The mixture was stirred at reflux overnight. After cooling to room temperature, the solvent was evaporated, and the residue was purified using dichloromethane and methanol to provide the product in 29% yield. ¹H NMR (400 MHz, DMSO): 0.09 (m, 2H), 0.47 (m, 2H), 0.83 (m, 1H), 1.38 (m, 3H), 2.04 (m, 1H), 2.22 (m, 3H), 2.90 (m, 1H), 3.09 (m, 1H), 3.38 (m, 1H), 3.63, 4.46 (m, 1H), 4.07, 4.83 (m, 1H), 6.80 (m, 2H), 7.46 (m, 1H), 7.85 (s, 1H). LRMS: M+376.

### Example 148

### (5Z)-5-[(5-fluoro-2-hydroxyphenyl)methylidene]-2-[4-(propan-2-yl)piperazin-1-yl]-4,5-dihydro-1,3-thiazol-4-one

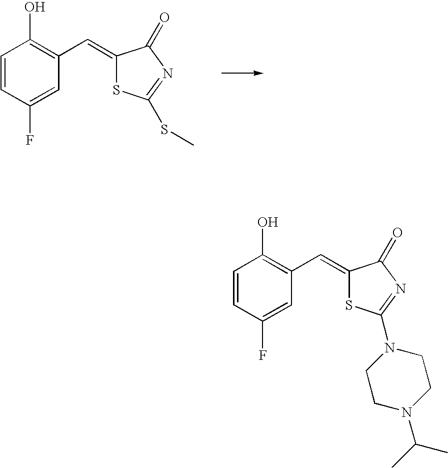

To a suspension of (5Z)-5-[(5-fluoro-2-hydroxyphenyl)methylidene]-2-(methylsulfanyl)-4,5-dihydro-1,3-thiazol-4-one (200 mg, 743 micromol) in absolute ethanol (8 mL) was added dropwise a solution of 1-isopropyl piperazine (159 microL, 1.11 mmol) in ethanol (2 mL) at 0° C. The reaction mixture was stirred at reflux overnight then cooled in an ice-water bath. After addition of water, the yellow solid was recovered by filtration, washed with water and dried in vacuo, affording the product, 108 mg, 42% yield. ¹H NMR (400 MHz, DMSO) δ 0.98 (d, J=6.5 Hz, 6H), 2.51-2.60 (m, 4H), 2.71-2.78 (m, 1H), 3.60 (t, J=4.9 Hz, 2H), 3.89 (t, J=4.9 Hz, 2H), 6.71-6.75 (m, 1H), 6.77-6.83 (m, 1H), 7.44-7.48 (m, 1H), 7.84 (s, 1H), 10.96 (bs, 1H).

### Example 149

### (5Z)-5-[(4-fluoro-2-hydroxyphenyl)methylidene]-2-[(2S)-4-(2-hydroxyethyl)-2-methylpiperazin-1-yl]-4,5-dihydro-1,3-thiazol-4-one

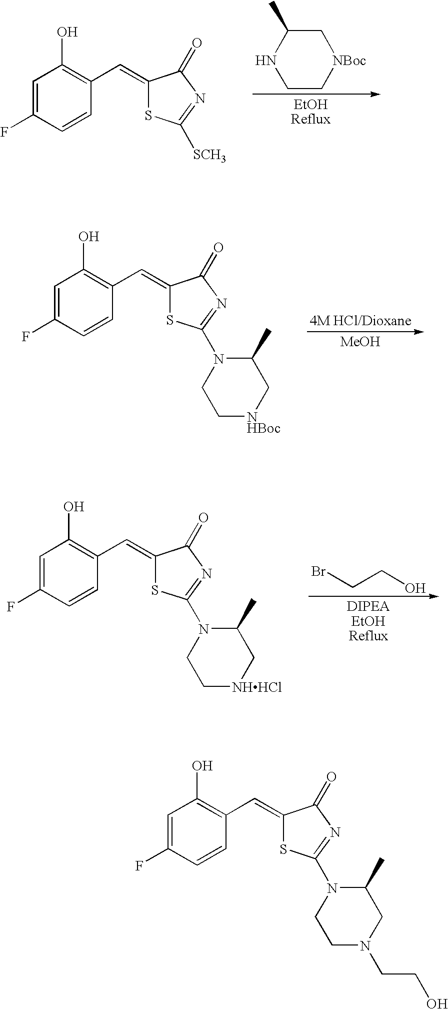

To a solution of (5Z)-5-[(4-fluoro-2-hydroxyphenyl)methylidene]-2-(methylsulfanyl)-4,5-dihydro-1,3-thiazol-4-one (1 g, 3.7 mmol) in absolute ethanol (5 mL) was added N-Boc-(S)-methylpiperazine (800 mg, 4.0 mmol). The reaction mixture was stirred at reflux overnight. After cooling to room temperature, the solvent was evaporated under reduced pressure. The crude product was purified by flash chromatography (Normal phase, 0-10% MeOH/CH₂Cl₂). The residue was triturated with diethyl ether (100 mL). The solid material was recovered by filtration and dried in vacuo, affording, tert-butyl 4-(5Z)-5-(4-fluoro-2-hydroxybenzylidene)-[(3S)-3-methyl-4-(4-oxo-4,5-dihydro-1,3-thiazol-2-yl)piperazine]-1-carboxylate (689 mg, 44%).

To a mixture of tert-butyl 4-(5Z)-5-(4-fluoro-2-hydroxybenzylidene)-[(3S)-3-methyl-4-(4-oxo-4,5-dihydro-1,3-thiazol-2-yl)piperazine]-1-carboxylate (685 mg, 1.6 mmol) in MeOH (3 mL) was added 4M HCl in dioxane (4 mL, 16.0 mmol). The reaction mixture was stirred at room temperature overnight. The solid material was recovered by filtration and washed with diethyl ether (2×10 ml) and dried in vacuo, affording (5Z)-5-[(4-fluoro-2-hydroxyphenyl)methylidene]-2-[(2S)-2-methylpiperazin-1-yl]-4,5-dihydro-1,3-thiazol-4-one hydrochloride (428 mg, 75%). The product was used without further purification.

To a solution of (5Z)-5-[(4-fluoro-2-hydroxyphenyl)methylidene]-2-[(2S)-2-methylpiperazin-1-yl]-4,5-dihydro-1,3-thiazol-4-one hydrochloride (428 g, 1.1 mmol) in absolute EtOH (5 mL) was added N—N-diisopropylethylamine (505 uL, 2.9 mmol) and bromoethanol (120 uL, 1.7 mmol). The reaction mixture was stirred at reflux overnight. After cooling to room temperature, the solvent was evaporated under reduced pressure. The crude product was purified by flash chromatography (0-10% MeOH/CH₂Cl₂ and 0-40% ACN/0.5% TFA_{(aq)}) and dried in vacuo, affording the final compound (77 mg, 19%); ¹H NMR (400 MHz, DMSO) 1.45 (m, 3H), 3.57 (m, 10.5H), 4.42 (m, 0.5H), 4.69 (m, 0.5H), 5.08 (m, 0.5H), 6.76 (dd, 1H, J=2.5 Hz, 10.8 Hz), 6.83 (td, 1H, J=2.5 Hz, 8.4 Hz), 7.46 (t, 1H, J=6.3 Hz), 7.90 (s, 1H), 11.10 (s, 1H); M+366.

### Example 150

### (5Z)-5-[(4-fluoro-2-hydroxyphenyl)methylidene]-2-[(2S)-2-(pyrrolidin-1-ylmethyl)pyrrolidin-1-yl]-4,5-dihydro-1,3-thiazol-4-one

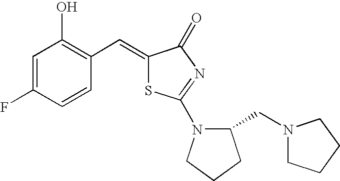

This compound was synthesized from Example 37 and (2S)-2-[pyrrolidin-1-ylmethyl]pyrrolidine by following the procedure for Example 39. The product was obtained in 55% yield as a red solid. ¹H NMR (400 MHz, DMSO-d₆): 1.65 (m, 4H), 2.04 (m, 4H), 2.85 (m, 1H), 3.40 (m, 4H), 3.62 (m, 4H), 6.80 (m, 1H), 7.40 (m, 1H), 7.80 (s, 1H). M=357.4.

### Example 151

### (5Z)-5-[(5-fluoro-2-hydroxyphenyl)methylidene]-2-[(2S)-2-(pyrrolidin-1-ylmethyl)pyrrolidin-1-yl]-4,5-dihydro-1,3-thiazol-4-one

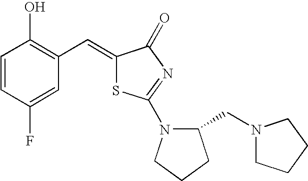

This compound was synthesized from Example 38 and (2S)-2-[pyrrolidin-1-ylmethyl]pyrrolidine by following the procedure for Example 39. The product was obtained as a red solid in 83% yield. ¹H NMR (400 MHz, DMSO-d₆): 1.60 (m, 4H), 2.04 (m, 4H), 2.60 (m, 6H), 3.40 (m, 3H), 3.69 (m, 1H), 4.00-4.40 (m, 1H), 6.99 (m, 1H), 7.20 (m, 2H), 8.00 (s, 1H), 10.20 (bs, 1H).

### Example 152

### (5Z)-5-[(4-fluoro-2-hydroxyphenyl)methylidene]-2-(pyrazolidin-1-yl)-4,5-dihydro-1,3-thiazol-4-one

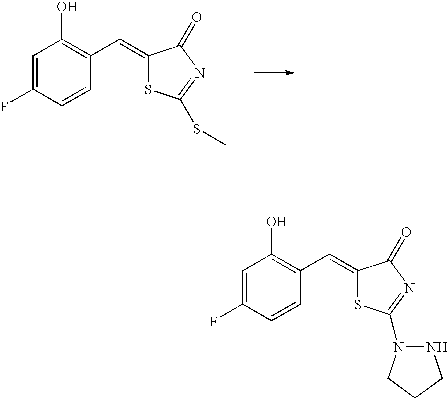

To a suspension of (5Z)-5-[(4-fluoro-2-hydroxyphenyl)methylidene]-2-(methylsulfanyl)-4,5-dihydro-1,3-thiazol-4-one (100 mg, 371 micromol) in absolute ethanol (4 mL) was added dropwise a solution of pyrazolidinone dihydrochloride (70 mg, 483 micromol) in ethanol (1 mL) at room temperature followed by dropwise addition of diisopropylethylamine (136 mL, 779 micromol) at 0° C. The reaction mixture was stirred at reflux overnight then solvent is evaporated. Solid was triturated in a mixture of H₂O/MeOH and recovered by filtration, washed with water and dried in vacuo, affording the compound, 55 mg, 50% yield. ¹H NMR (400 MHz, DMSO) δ 3.03 (bs, 2H), 3.17 (bs, 2H), 3.71 (bs, 2H), 6.22 (bs, 1H), 6.73 (bs, 1H), 6.80 (bs, 1H), 7.45 (bs, 1H), 7.80 (bs, 1H), 10.87 (bs, 1H). LRMS (ES⁺) m/z 294 (M⁺, 100).

### Example 153

### (5Z)-5-[(5-fluoro-2-hydroxyphenyl)methylidene]-2-[2-(2-hydroxyethyl)-1,2-diazinan-1-yl]-4,5-dihydro-1,3-thiazol-4-one

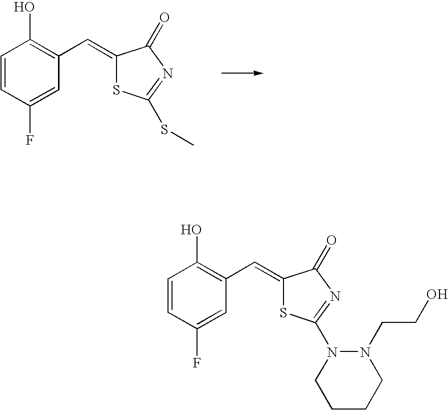

To a suspension of (5Z)-5-[(5-fluoro-2-hydroxyphenyl)methylidene]-2-(methylsulfanyl)-4,5-dihydro-1,3-thiazol-4-one (217 mg, 805 μmol) in absolute ethanol (6 mL) was added dropwise a solution of 2-(piperazin-1-yl)ethanol (1.05 mmol) in ethanol (2 mL) followed by dropwise addition of triethylamine (123 μL, 886 μmol) at 0° C. The reaction mixture was stirred at reflux overnight then cooled in an ice-water bath. Solid was removed by filtration and washed with methanol. Filtrate was evaporated, and the residue was dissolved in DCM and purified on silica gel using DCM/MeOH to afford the product, 102 mg, 36% yield. LRMS (ES⁺) m/z 352 (M⁺, 100).

### Example 154

### (5Z)-5-[(4-fluoro-2-hydroxyphenyl)methylidene]-2-[(3R)-3-(pyrrolidin-1-yl)pyrrolidin-1-yl]-4,5-dihydro-1,3-thiazol-4-one

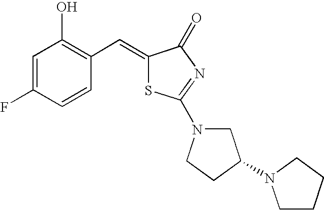

This compound was synthesized from Example 37 and (R) [1,3′]bipyrrolidine by following the procedure for Example 39. A yellow solid was obtained in 82% yield. ¹H NMR (400 MHz, DMSO-d6): 1.80 (m, 2H), 2.01 (m, 1H), 2.20 (m, 1H), 3.00 (m, 1H), 3.45-4.00 (m, 8H), 6.75 (d, J=8.0 Hz, 1H), 6.80 (m, 1H), 7.40 (m, 1H), 7.50 (m, 1H), 7.80 (s, 1H).

### Example 155

### (5Z)-5-[(4-fluoro-2-hydroxyphenyl)methylidene]-2-[(3S)-3-(pyrrolidin-1-yl)pyrrolidin-1-yl]-4,5-dihydro-1,3-thiazol-4-one

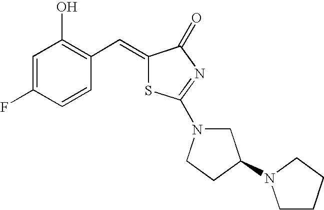

This compound was synthesized from Example 37 and (S) [1,3′]bipyrrolidine by following the procedure for Example 39. A yellow solid was obtained in 78% yield. ¹H NMR (400 MHz, DMSO-d6): 1.80 (m, 2H), 2.01 (m, 1H), 2.20 (m, 1H), 3.00 (m, 1H), 3.45-4.00 (m, 8H), 6.75 (d, J=8.0 Hz, 1H), 6.80 (m, 1H), 7.40 (m, 1H), 7.50 (m, 1H), 7.80 (s, 1H).

### Example 156

### (5Z)-2-(1,2-diazinan-1-yl)-5-[(5-fluoro-2-hydroxyphenyl)methylidene]-4,5-dihydro-1,3-thiazol-4-one

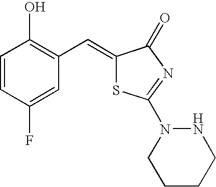

This compound was synthesized from Example 38 and hexahydropyridazine by following the procedure for Example 39. A yellow solid was obtained in 40% yield. ¹H-NMR (400 MHz, DMSO): 1.64-1.77 (m, 4H), 2.95 (m, 2H), 3.86 (br, 2H), 6.05 (m, 1H), 6.95 (m, 1H), 7.12 (m, 2H), 7.80 (s, 1H). LRMS: M⁺ 308

The following compounds were synthesized but found to be chemically unstable:

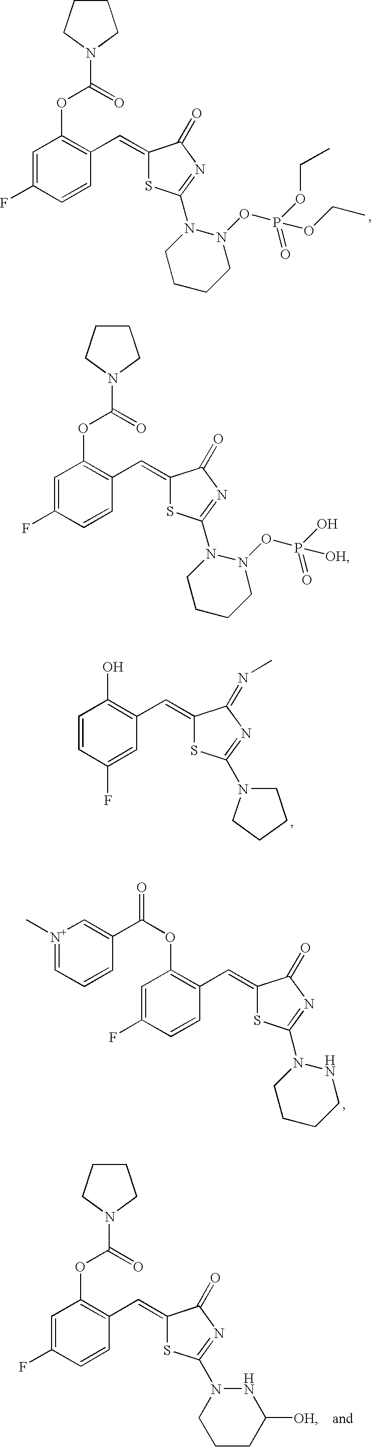

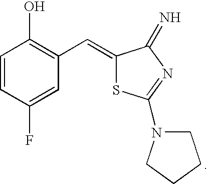

certain embodiments, these compounds (in their acid, base, or salt forms) are specifically excluded from the compositions and methods described herein.

### Example 157

The analgesic effect of a representative number of the compounds of the invention was determined using the procedures described hereafter.

### Determination of Analgesic Effect in an Experimental Model of Neuropathic Pain

Adult, male Sprague-Dawley rats were obtained from Charles River Laboratories (Wilmington, Mass.) and housed under standard conditions at the Institut Armand-Frappier (Laval, QC). Food and water were provided to experimental animals ad libitum, and rats weighed 175-200 grams at the time of assessment.

Compounds were prepared for intrathecal administration by dissolving them in a vehicle of Captisol® (CyDex, Lenexa, Kans.); total volume of solution administered to rats was 20 μL.

Neuropathic pain was induced in rats via chronic constriction injury (CCl) of the left sciatic nerve in accordance with the procedure described by Bennett & Xie (Pain, 1988). Briefly, under ketamine/xylazine anaesthesia, the sciatic nerve was exposed by dissection at the level of mid-thigh, and four loose ligatures (USP 4/0, Braun Melsungen, FRG) were implanted around the nerve—with due attention not to interrupt the epineural circulation. The incision was closed-up using simple suturing, and the rats allowed to recover.

After approximately two weeks, a stable allodynia to blunt mechanical stimuli was identified in the hind paw ipsilateral to the CCl, manifested as a reduction of 50% withdrawal threshold, and identified using the Von Frey technique, as described by Chaplan et al. (Journal of Neuroscience Methods, 1994). Rats were considered to be fully neuropathic upon displaying a 50% withdrawal threshold of <3.5 grams consistently over the course of 72 hours.

Under brief isoflurane analgesia, compounds were administered to neuropathic rats via acute local delivery in the intrathecal space surrounding the lumbar enlargement of the spinal cord.

Thirty minutes following intrathecal administration of representative compounds to neuropathic rats, the 50% withdrawal threshold rose from a mean 3.3±0.5 g to a mean 6.7±2.1 g (significantly higher than that evoked by vehicle, p<0.05, as assessed by repeated-measures ANOVA). Sixty minutes post-administration of compounds, the mean 50% withdrawal threshold was 5.8±1.4 g (p<0.05 compared to vehicle).

Compounds of the invention that demonstrated efficacy in this assay include:

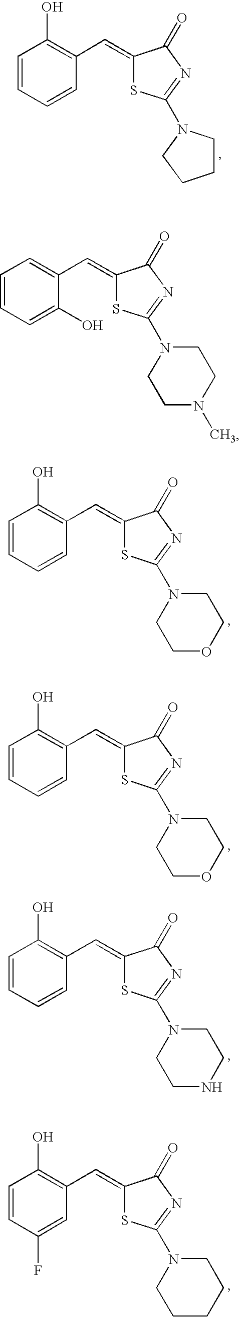

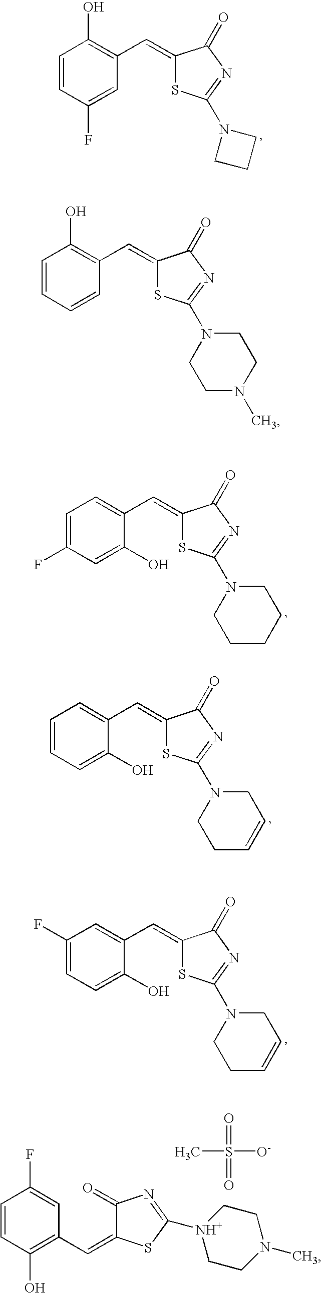

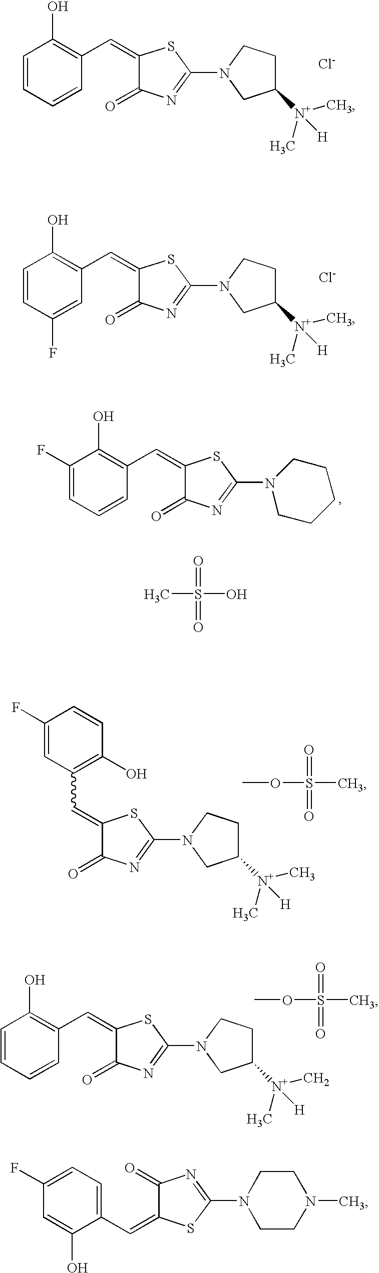

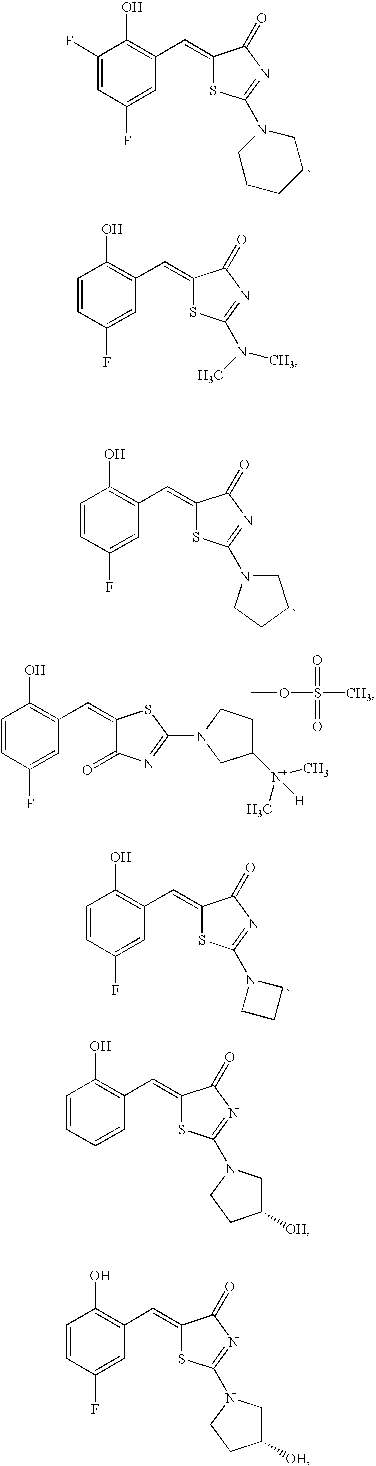

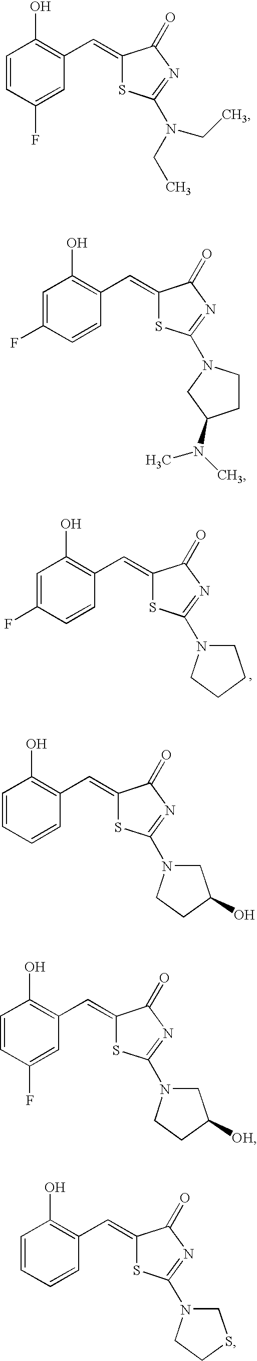

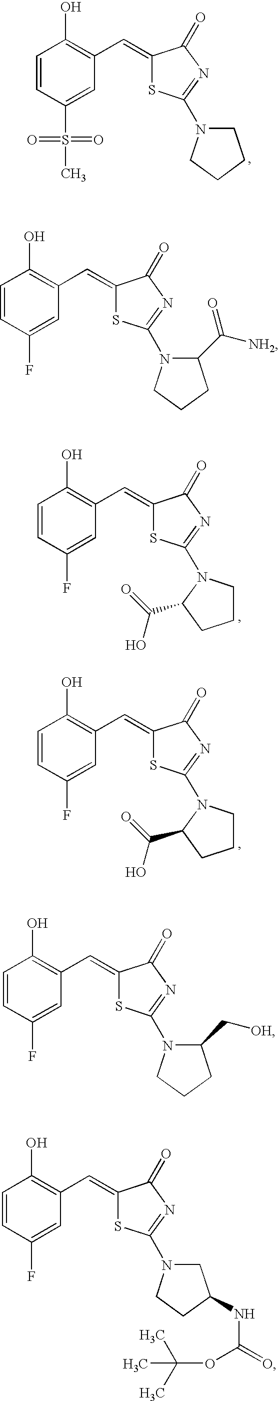

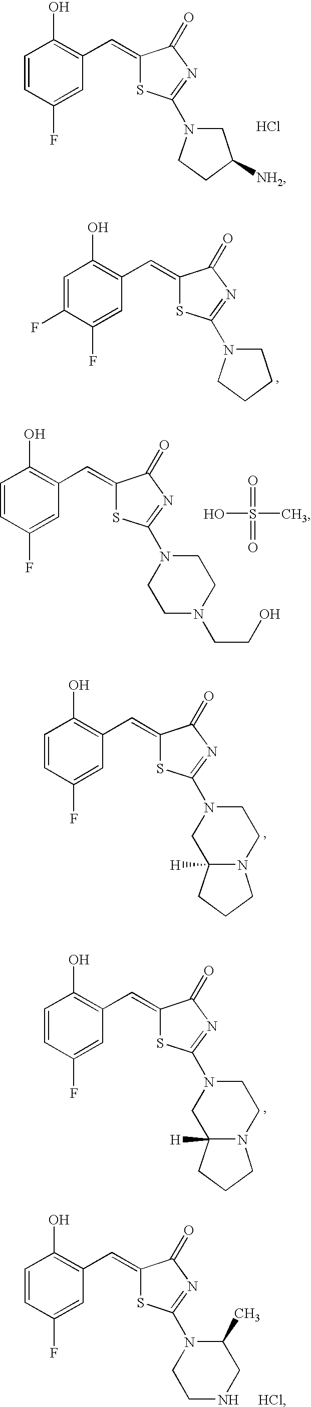

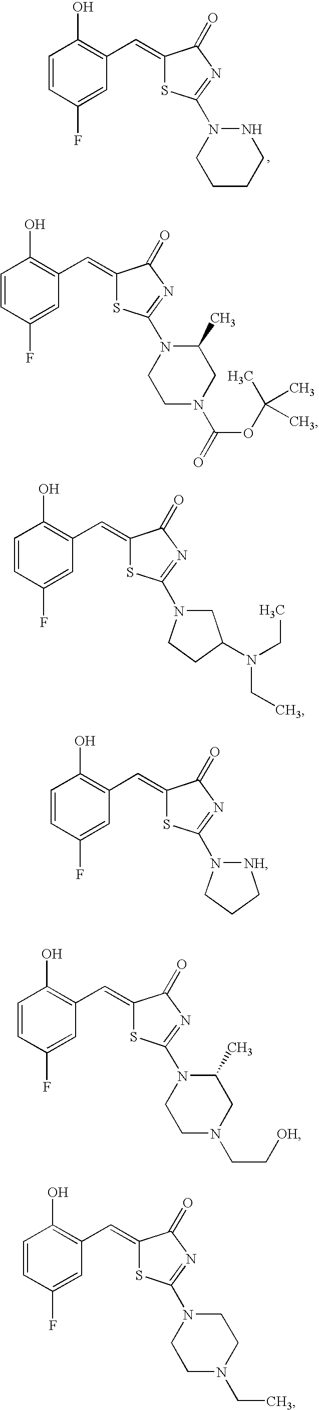

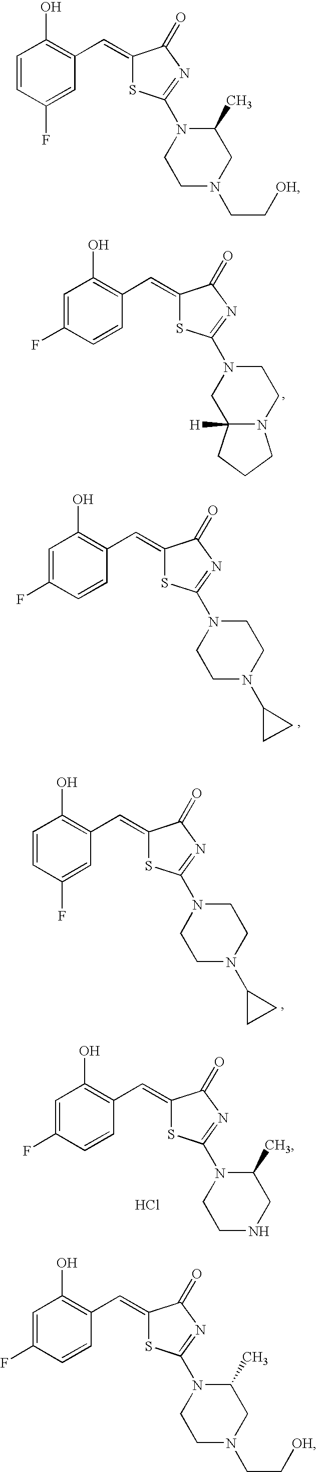

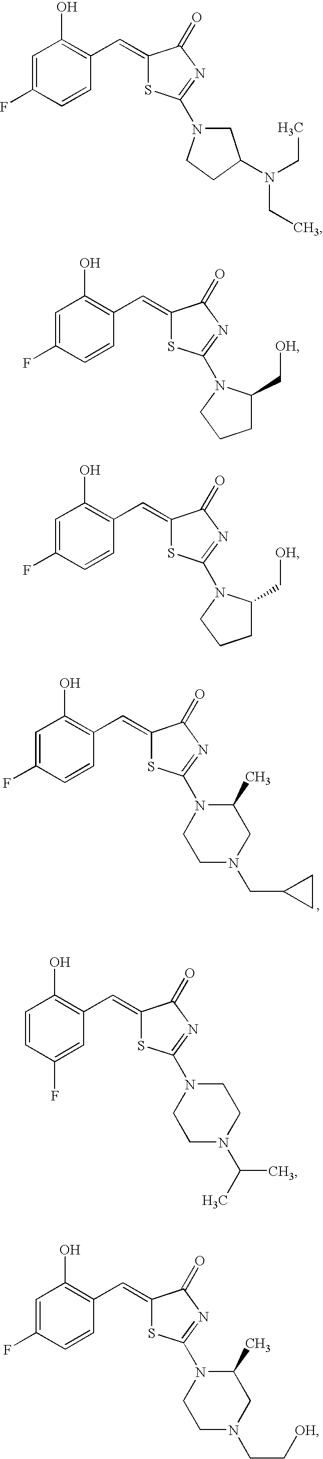

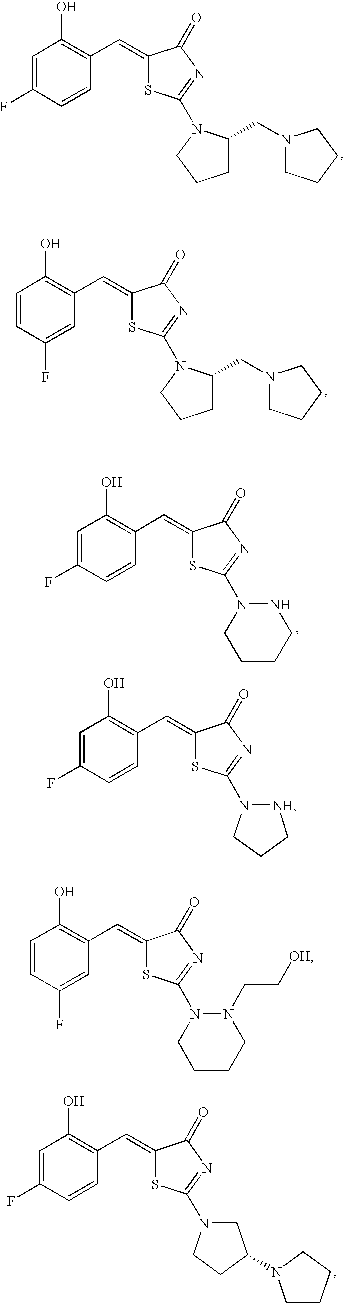

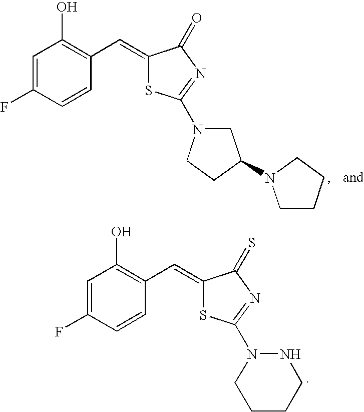

Table 3 below presents the peak efficacy of several representative compounds in rats rendered neuropathic via the Bennett Model, in terms of 50% withdrawal threshold. Data are presented as mean efficacy standard error of the mean. Note that in all cases the peak efficacy shown for the compounds was significantly different from the 50% withdrawal threshold of neuropathic rats administered vehicle control (p<0.05, as assessed by repeated-measures ANOVA).

TABLE 3

| | Peak 50% Withdrawal |
|---|---|
| IUPAC name | Threshold (g) |
| (5Z)-5-[(2-hydroxyphenyl)methylidene]-2-(piperidin-1-yl)-4,5- | 6.74 ± 2.13 |
| dihydro-1,3-thiazol-4-one | |
| (3R)-1-[(5Z)-5-[(5-fluoro-2-hydroxyphenyl)methylidene]-4,5- | 6.44 ± 1.33 |
| dihydro-1,3-thiazol-2-yl]-N,N-dimethylpyrrolidin-3-aminium | |
| chloride | |
| (3S)-1-[(5Z)-5-[(5-fluoro-2-hydroxyphenyl)methylidene]-4-oxo-4,5- | 9.72 ± 2.04 |
| dihydro-1,3-thiazol-2-yl]-N,N-dimethylpyrrolidin-3-aminium; | |
| methanesulfonate | |
| (5Z)-2-[(3R)-3-(dimethylamino)pyrrolidin-1-yl]-5-[(4-fluoro-2- | 5.6 ± 1.94 |
| hydroxyphenyl)methylidene]-4,5-dihydro-1,3-thiazol-4-one | |
| (5Z)-2-[(3S)-3-(dimethylamino)pyrrolidin-1-yl]-5-[(4-fluoro-2- | 7.3 ± 1.37 |
| hydroxyphenyl)methylidene]-4,5-dihydro-1,3-thiazol-4-one; | |
| methanesulfonic acid | |
| (5Z)-5-[(5-fluoro-2-hydroxyphenyl)methylidene]-2-[4-(2- | 6.40 ± 2.45 |
| hydroxyethyl)piperazin-1-yl]-4,5-dihydro-1,3-thiazol-4-one; | |
| methanesulfonic acid | |
| (5Z)-2-(1,2-diazinan-1-yl)-5-[(5-fluoro-2- | 9.03 ± 2.00 |
| hydroxyphenyl)methylidene]-4,5-dihydro-1,3-thiazol-4-one | |
| (5Z)-5-[(4-fluoro-2-hydroxyphenyl)methylidene]-2-[4-(2- | 8.23 ± 2.40 |
| hydroxyethyl)piperazin-1-yl]-4,5-dihydro-1,3-thiazol-4-one | |
| (5Z)-2-(1,2-diazinan-1-yl)-5-[(4-fluoro-2- | 7.37 ± 2.02 |
| hydroxyphenyl)methylidene]-4,5-dihydro-1,3-thiazol-4-one | |
| 2-{[(5E)-2-[3-(diethylamino)pyrrolidin-1-yl]-4-oxo-4,5-dihydro-1,3- | 11.84 ± 1.58 |
| thiazol-5-ylidene]methyl}-4-fluorophenyl N,N-dimethylcarbamate | |
| 2-{[(5E)-2-(1,2-diazinan-1-yl)-4-oxo-4,5-dihydro-1,3-thiazol-5- | 6.18 ± 1.77 |
| ylidene]methyl}-5-fluorophenyl N,N-dimethylcarbamate | |
| hydrochloride | |
| 2-{[(5E)-2-(1,2-diazinan-1-yl)-4-oxo-4,5-dihydro-1,3-thiazol-5- | 8.18 ± 1.98 |
| ylidene]methyl}-4-fluorophenylpyrrolidine-1-carboxylate | |
| hydrochloride | |
| 2-{[(5E)-2-(1,2-diazinan-1-yl)-4-oxo-4,5-dihydro-1,3-thiazol-5- | 7.42 ± 1.21 |
| ylidene]methyl}-5-fluorophenyl pyrrolidine-1-carboxylate | |
| hydrochloride | |

The following compounds were found to be inactive in the assay. In certain embodiments, these compounds (in their acid, base, or salt forms) are specifically excluded from the compositions and methods described herein.

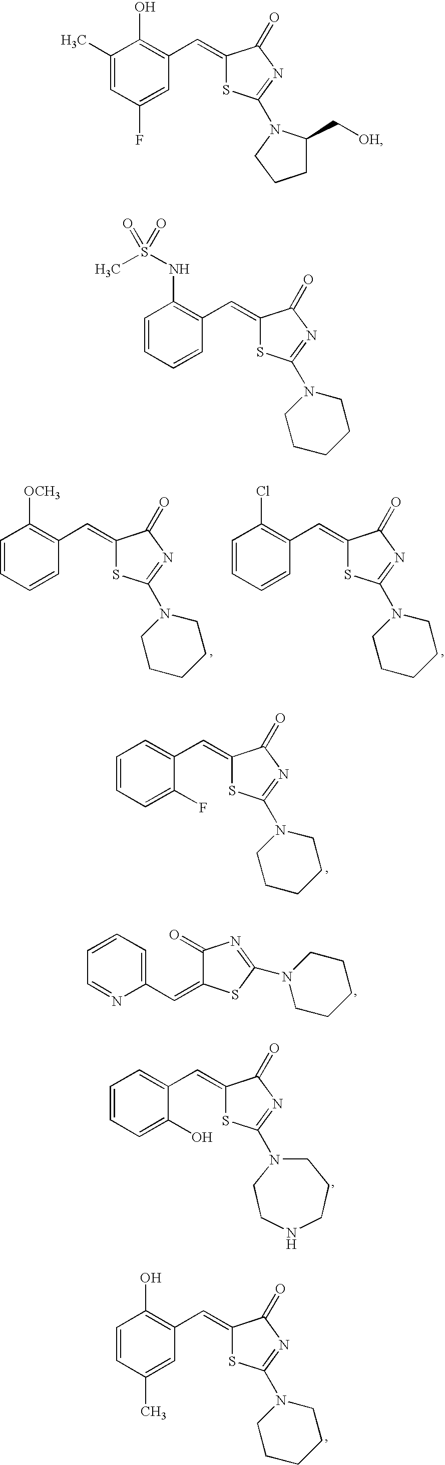

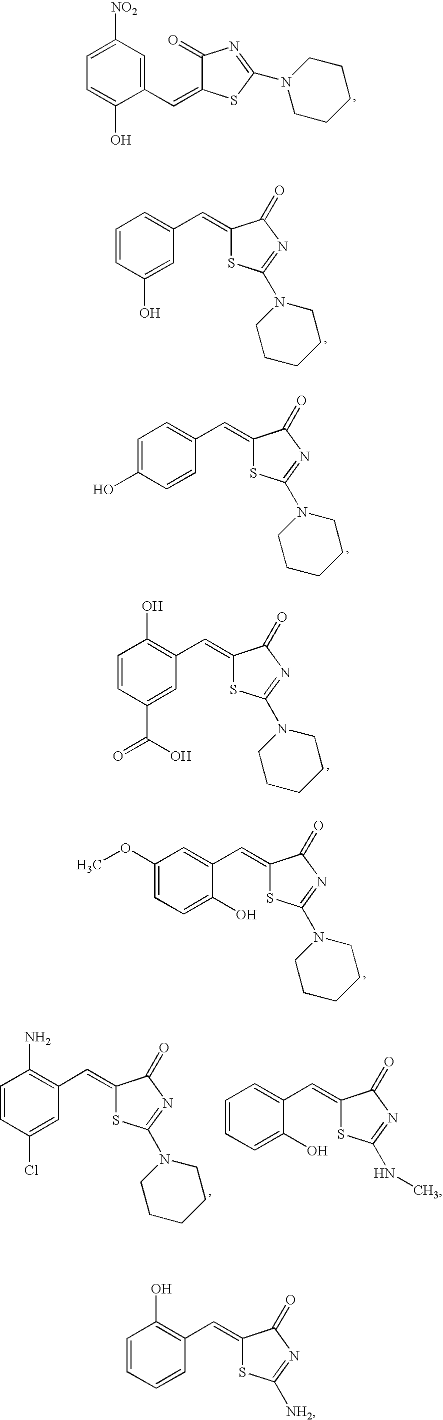

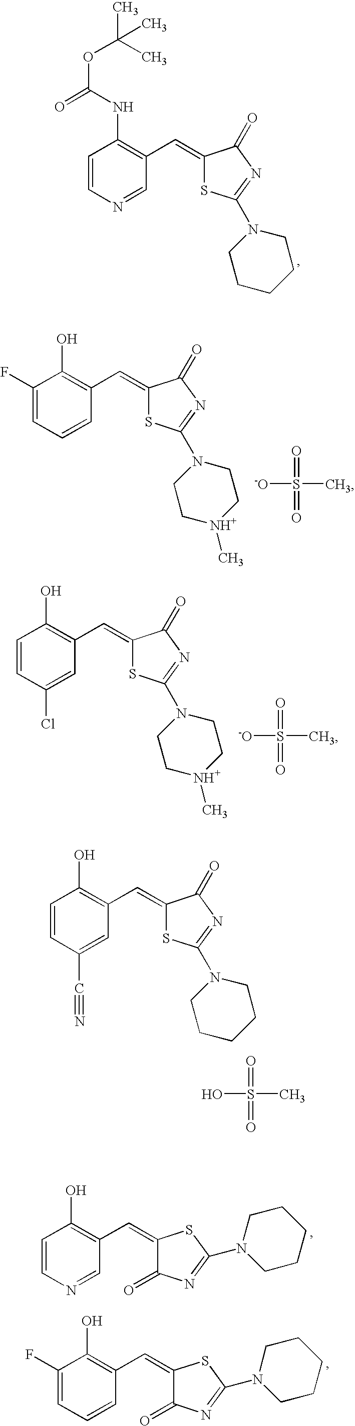

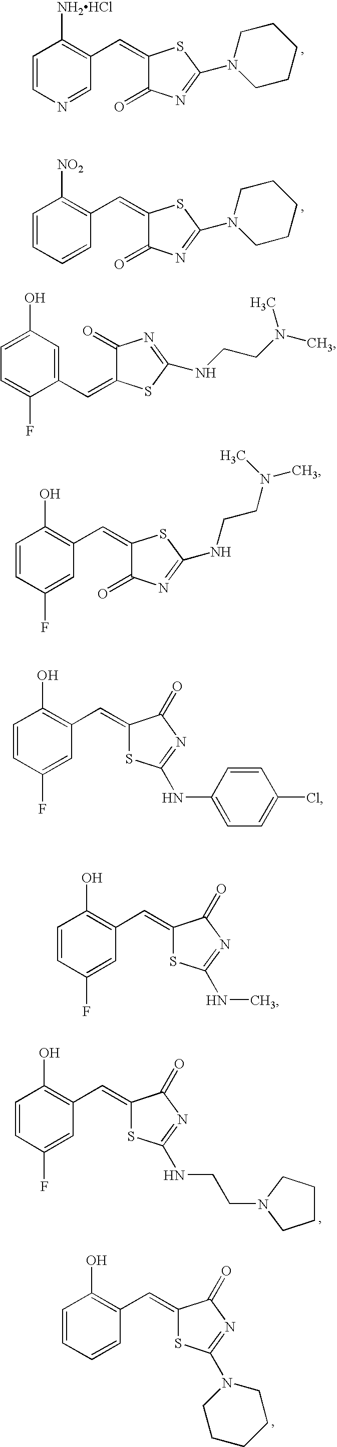

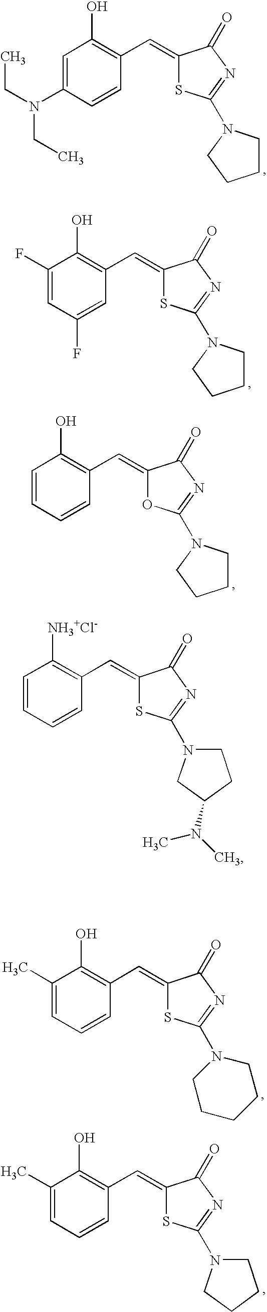

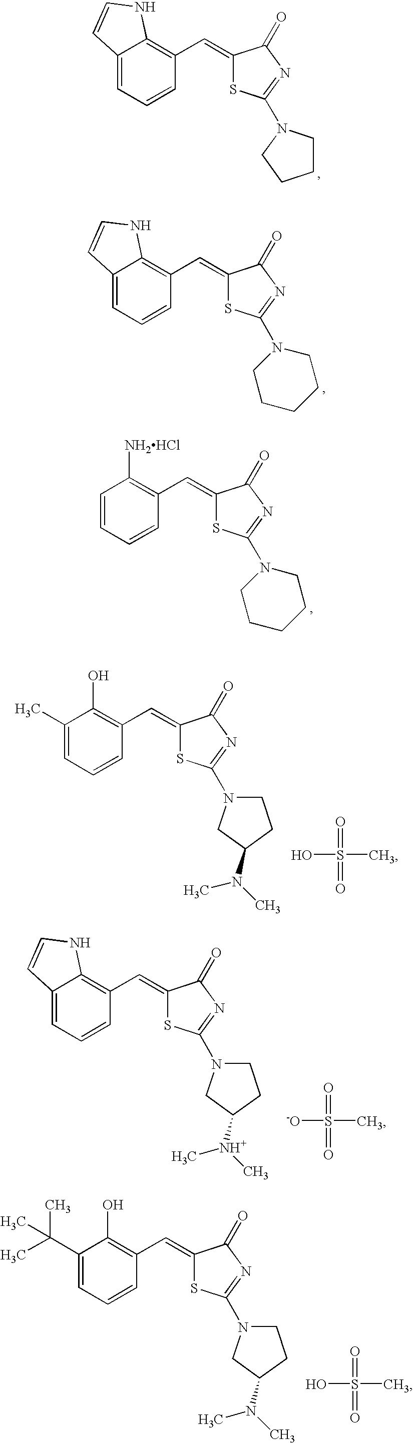

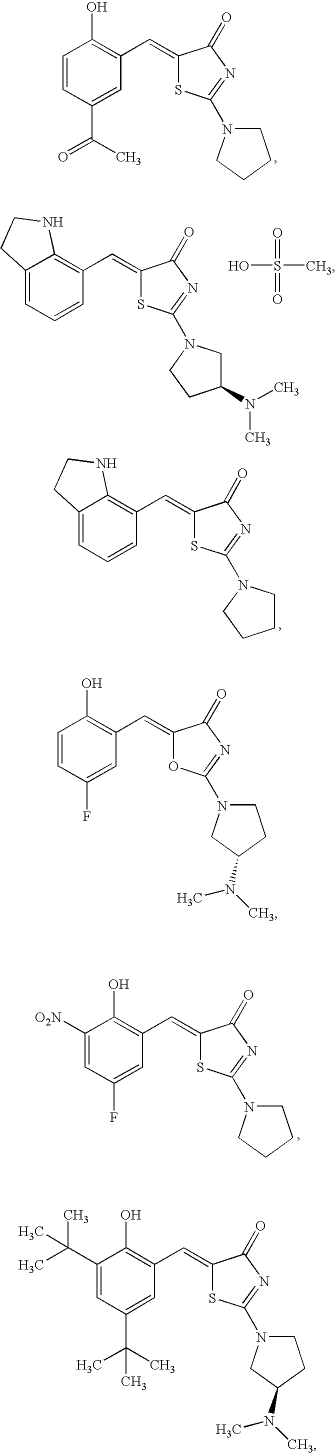

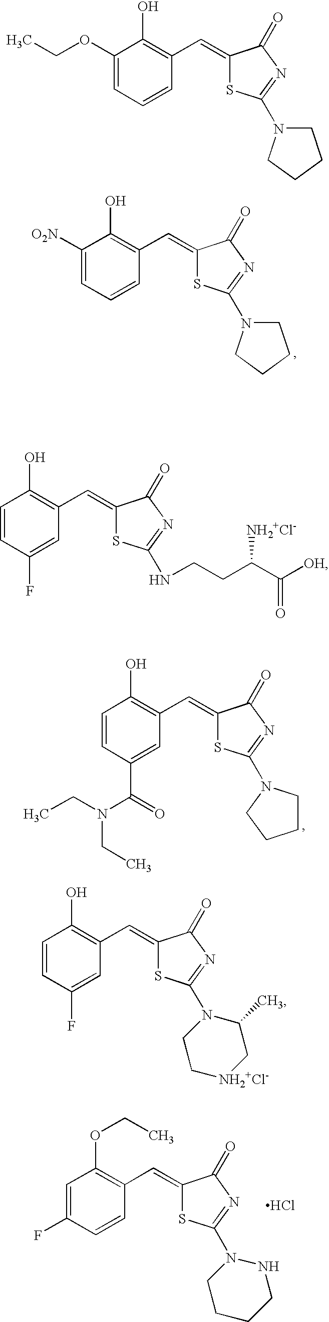

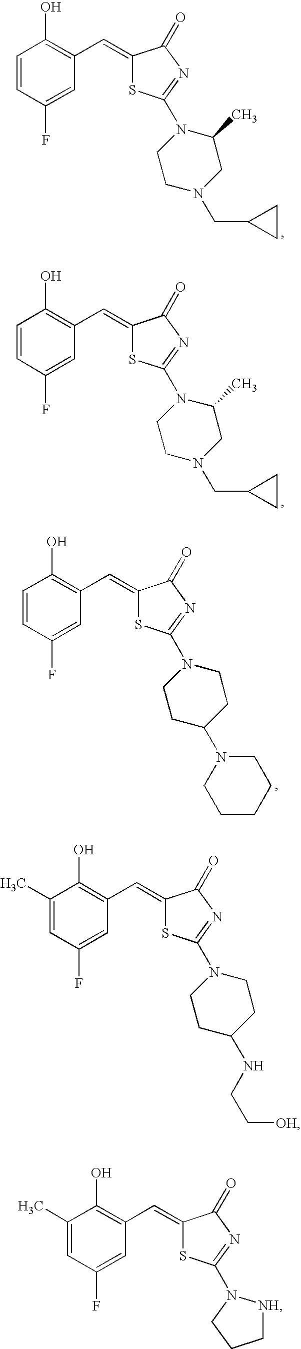

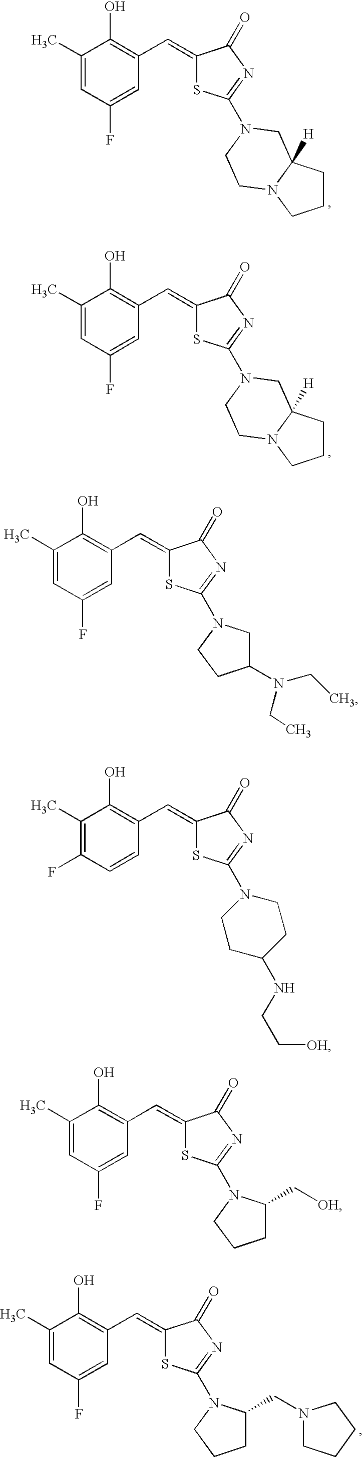

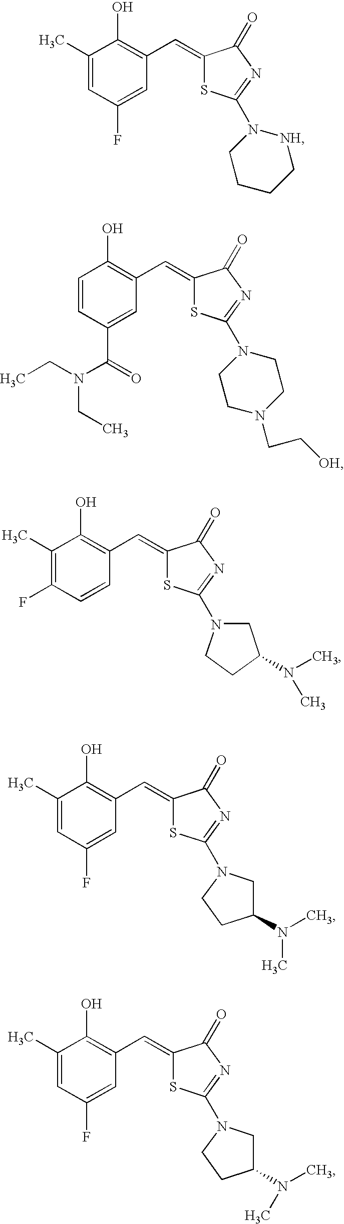

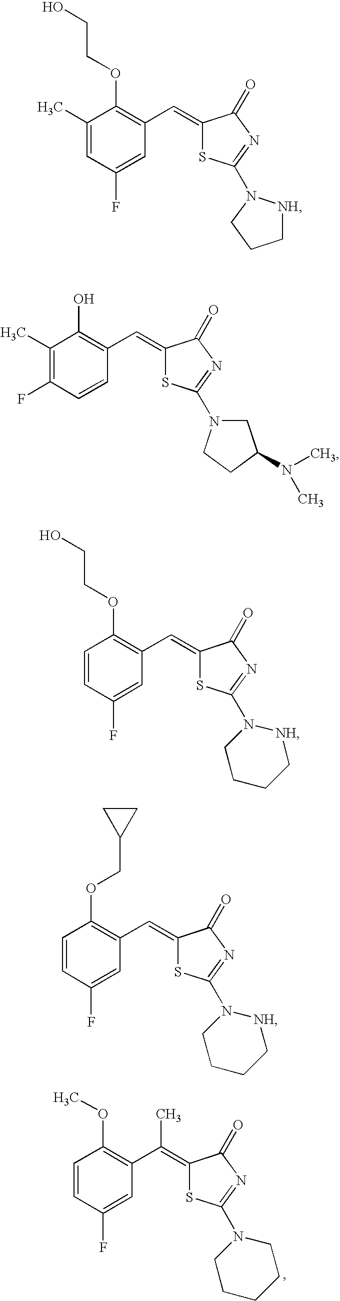

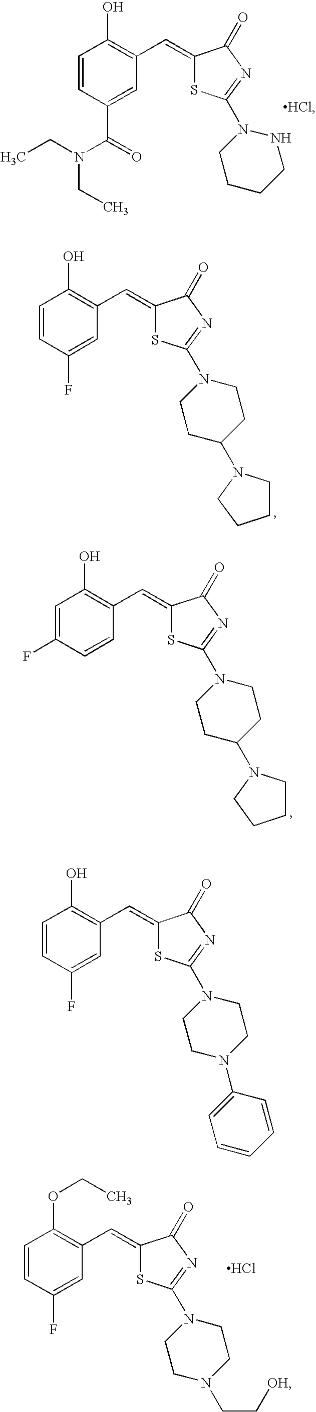

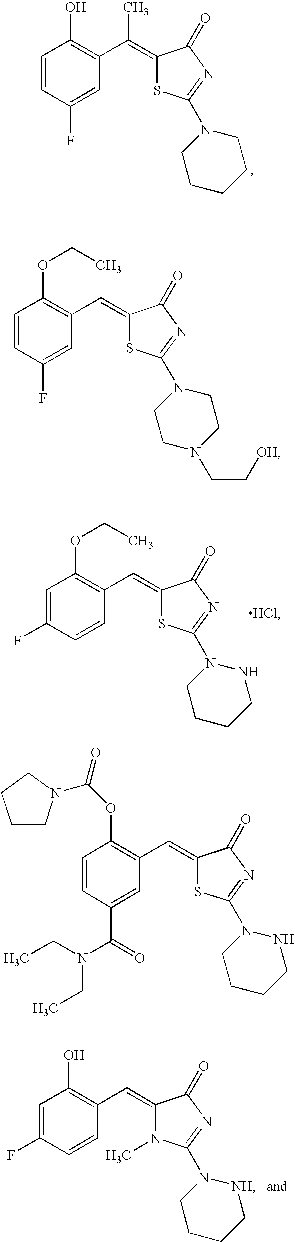

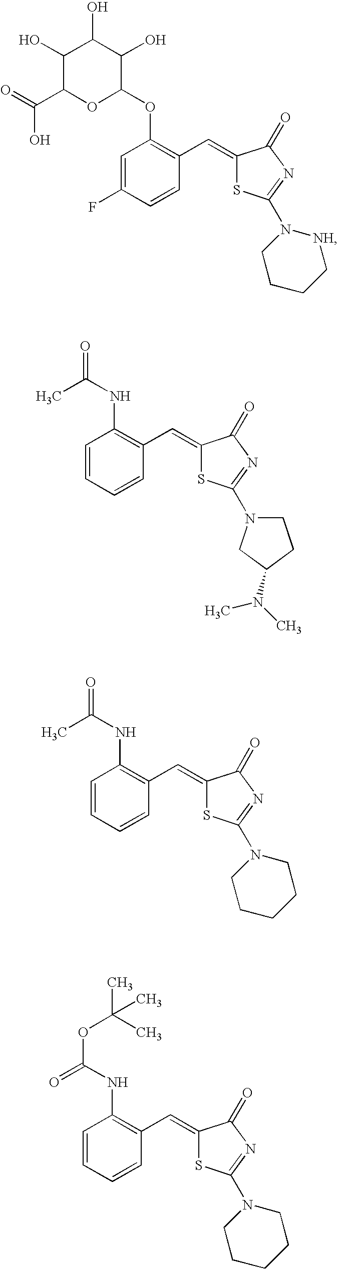

The following compounds are prodrugs that are expected to hydrolyze in vivo to produce active compounds.

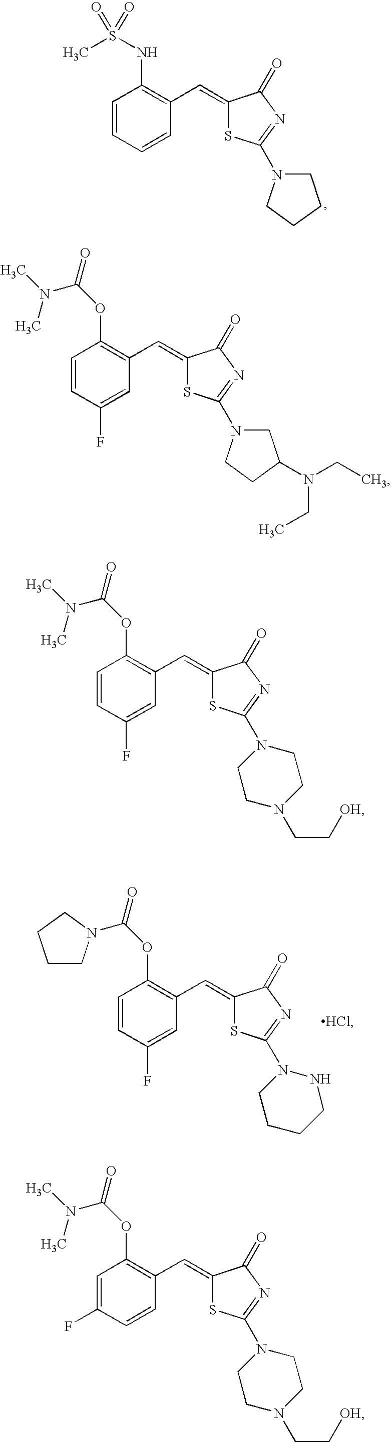

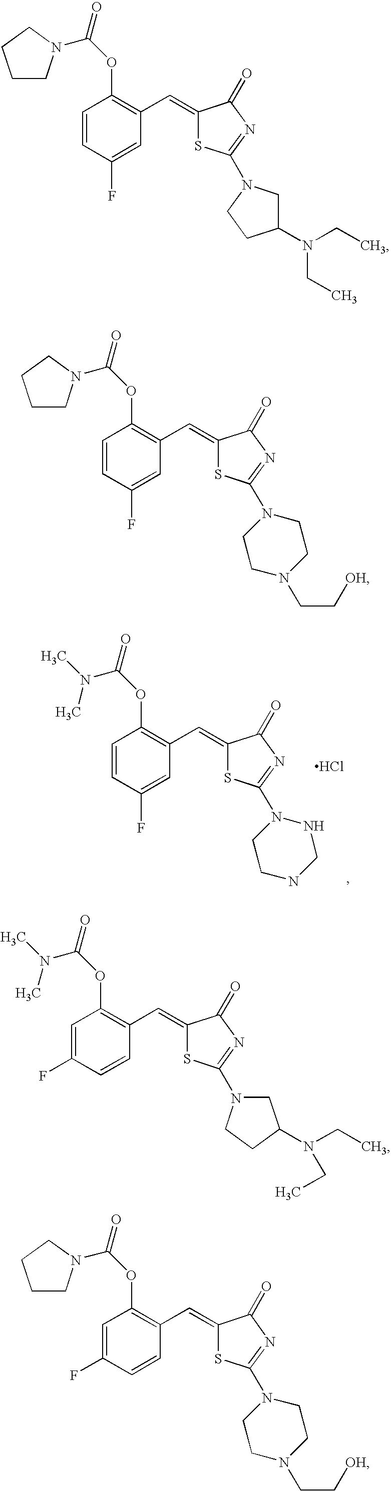

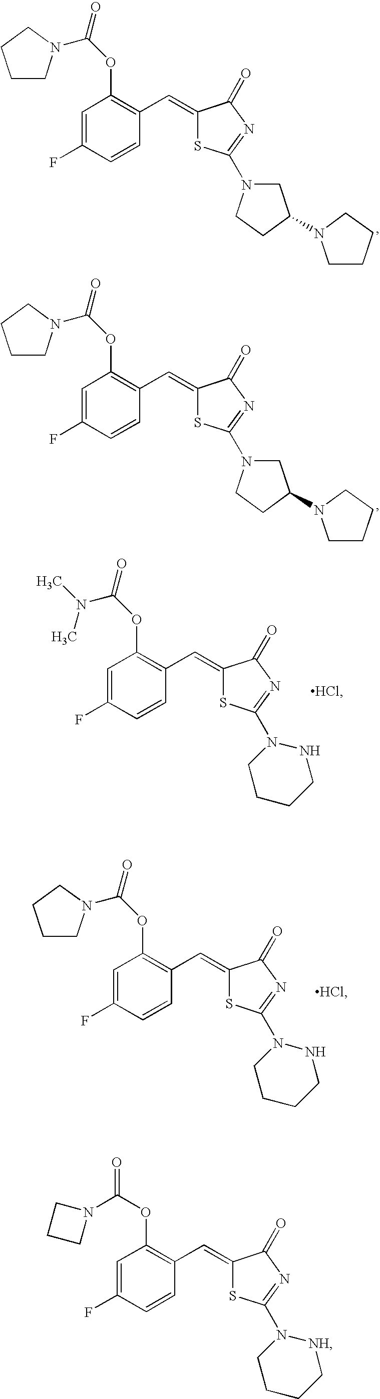

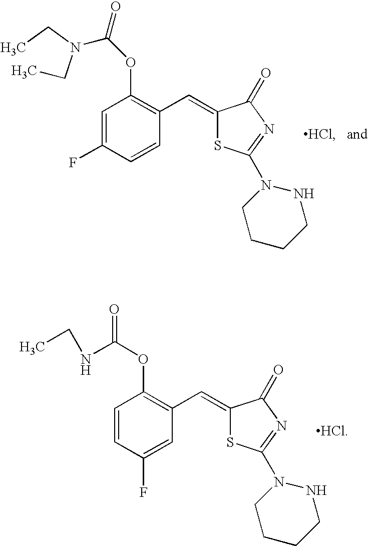

It should also be noted that for in vivo medicinal uses, potency is not the only factor to be considered to estimate the suitability of a compound as a pharmaceutical agent. Other factors such as toxicity and bioavailability also determine the suitability of a compound as a pharmaceutical agent. Toxicity and bioavailability can also be tested in any assay system known to the skilled artisan.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

What is claimed is:

1. A compound having the following structure: 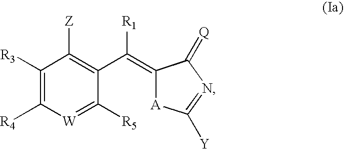
including stereoisomers, E/Z stereoisomers, prodrugs, and pharmaceutically acceptable salts thereof, wherein:
A is —O—, —S—, —SO—, —SO₂—, >NR₆, or >NC(O)R₆;
Q is O, S, or NR₆;
Z is —F, —Cl, —NO₂, —OR₂, —C(O)R₆, —C(O)(CR₆R₆)ₒNH₂, —N(R₆)₂, or —NHC(O)R₆;
W is CX or N;
X is —H, —F, —Cl, —CN, —OH, —C₂-C₈ alkyl, —C₂-C₈ alkenyl, —C₂-C₈ alkynyl, —C₃-C₁₂ cycloalkyl, —OC₂-C₄alkyl, —OC₂-C₄ alkenyl, —OC₂-C₄ alkynyl, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —S(O)R₆, —S(O)₂R₆, —S(O)₂N(R₆)₂, —NHC(O)R₆, —NHS(O)₂R₆, —NHC(NH)N(R₆)₂, —NR₆C(NH)N(R₆)₂, —NR₆C(NCN)N(R₆)₂, N-terminal linked amino acid, or C-terminal linked amino acid;
Y is —C₃-C₈ cycloalkyl, 3 to 8-membered aromatic or non aromatic heterocycle, —SR₆, —S(O)R₆, —S(O)₂R₆, —N(R₆)₂, —NHC(O)R₆, —NHS(O)₂Rr, —NHC(NH)N(R₆)₂, —NR₆C(NH)N(R₆)₂, or —NR₆C(NCN)N(R₆)₂;
R₁ is —H, halogen, —C₁-C₈ alkyl, —C₂-C₈ alkenyl, or —C₂-C₈ alkynyl;
R₂ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, —(CH₂)ₙOR₆, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid;
each R_{2A} and R_{2B} is, independently, H or C₁₋₅ alkyl;
R₃, R₄, and R₅ are each, independently, —H, —OH, halogen, —CN, —NO₂, —SH, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3 to 9-membered aromatic or non aromatic heterocycle, —OR₆, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —SOR₆, —S(O)₂R&, —NHC(O)R&, —NHS(O)₂R₆, —NHC(NH)N(R₆)₂, —NR₆C(NH)N(R₆)₂, —NHC(NCN)N(R₆)₂, —NR₆C(NCN)N(R₆)₂, or —PO(OR₆)₂, or R₃ and R₄, together with the carbon atoms to which each is attached, join to form a 5- to 6-membered aromatic or non aromatic carbocycle or heterocycle;
each R₆ is, independently, —H, —C₁-C₈ alkyl, alkcycloalkyl, alkheterocyclyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic carbocycle or heterocycle;
n is 1 or 2;
o is an integer between 0-3;
each r2 is an integer between 1-3;
each r3 is an integer between 0-2;
wherein R₃ is not —Br, when R₅ is —OH;
wherein, when W ix CX, one of X and R₄ is not —H; and
wherein Formula (Ia) excludes compounds having the structure

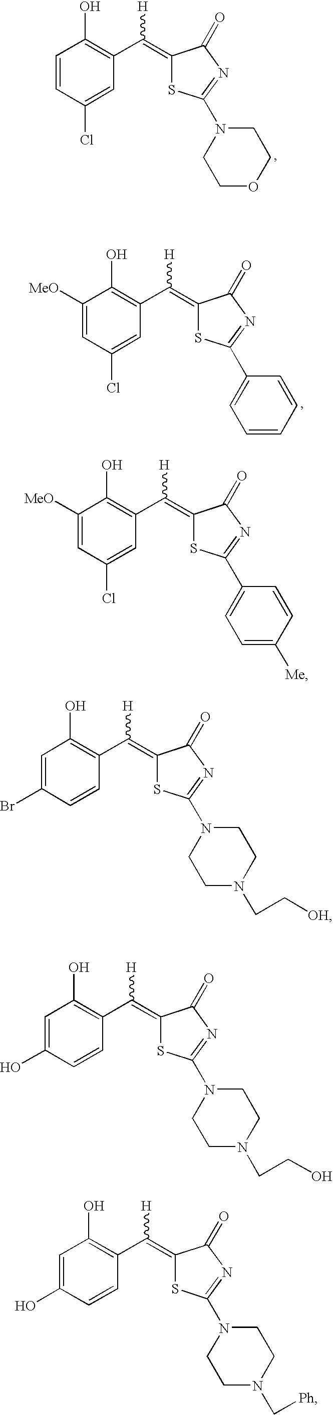

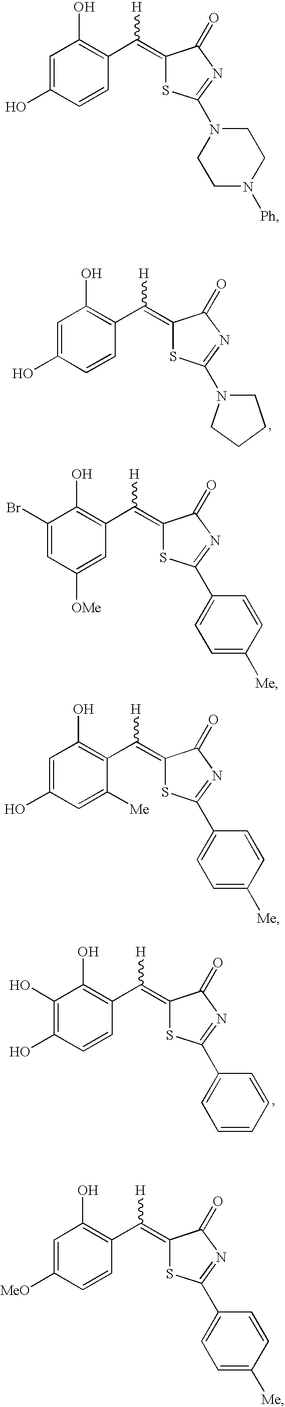

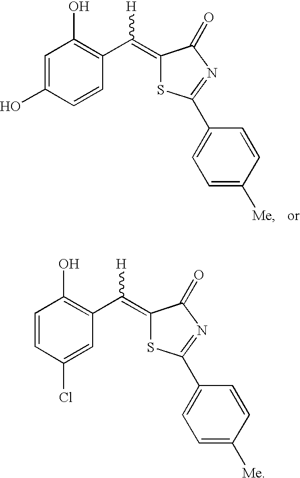

2-5. (canceled)

6. The compound of claim 1, wherein said compound of Formula (Ia) has the following structure

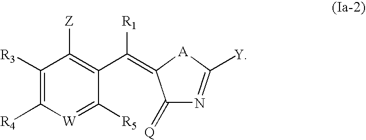

7. The compound of claim 1, wherein said compound of Formula (Ia) has the following structure: 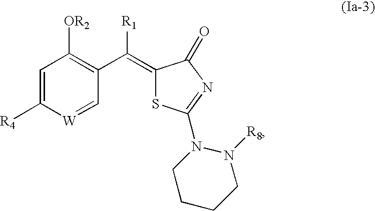
wherein
R₈ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3 to 9-membered aromatic or non aromatic heterocycle, —(CH₂)ₙOR₆, —C(O)R₆, —C(O)OR₆, —C(O)NHR₆, —C(O)N(R₆)₂, —C(O)N(R₆)₂, —(CR_{Y1}R_{Y2})_{y2}PO(OR_{Y3})(OR_{Y4}); —C(NH)N(R₆)₂, or —S(O)₂R₆;
each R_{Y1}, R_{Y2}, R_{Y3}, and R_{Y4} is, independently, H or C₁₋₅ alkyl; and
y₂ is 0 or 2.

8. The compound of claim 1, wherein said compound of Formula (Ia) has the following structure: 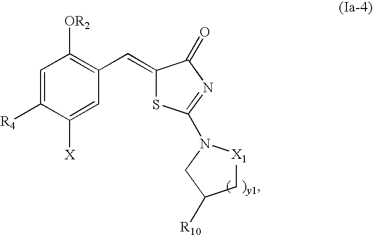
wherein
X is H or F;
R₂ is —H, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid;
R₄ is H or F;
R₁₀ is H or N(CH₃)₂;
X₁ is CH₂ or NR₈;
R₈ is H or —(CR_{Y1}R_{Y2})_{y2}PO(ORy₃)(OR_{Y4});
each R_{Y1}, R_{Y2}, R_{Y3}, and R_{Y4} is, independently, H, C₁₋₅ alkyl, or R_{Y3} and R_{Y4} combine to form a 5 to 7 membered ring;
each y1 and y2 is, independently, 0, 1, or 2.

9-13. (canceled)

14. The compound of claim 1, wherein Y is a 5 to 6-membered non aromatic heterocycle.

15. The compound of claim 14, wherein Y is 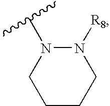
wherein
R₈ is H, —(CR_{Y1}R_{Y2})_{y2}PO(ORy₃)(ORy₄), or —C(O)R_{Y5};
each R_{Y1}, R_{Y2}, R_{Y3}, and R_{Y4} is, independently, H, C₁₋₅ alkyl, or R_{Y3} and R_{Y4} combine to form a 5 to 7 membered ring;
each R_{Y5} is aryl; and
y₂ is 0, 1, or 2.

16. The compound of claim 15, wherein R₈ is H.

17. The compound of claim 15, wherein R₈ is —(CH₂)_{y2}PO(OR_{Y4})(OR_{Y5}).

18. The compound of claim 14, wherein Y is optionally substituted azetidinyl, optionally substituted pyrrolidinyl, optionally substituted piperidinyl, optionally substituted piperazinyl, optionally substituted morpholinyl, optionally substituted tetrahydropyridinyl, or optionally substituted hexamethyleneiminyl.

19. The compound of claim 18, wherein Y is selected from the group consisting of:

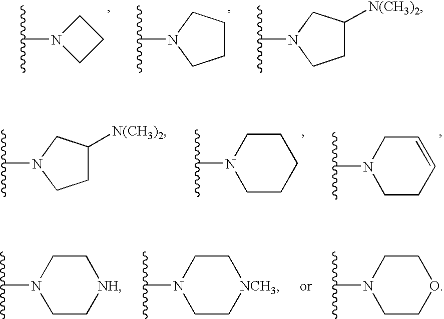

20. The compound of claim 1, wherein R₆ is either H or CH₃.

21. The compound of claim 1, wherein two R₆, together with the atom to which each is attached, join to form a 5-, 6-, or 7-membered non aromatic heterocycle.

22. The compound of claim 1, wherein R₃ and R₄, together with the atom to which each is attached, join to form a 5- or 6-membered aromatic or non aromatic carbocycle or heterocycle.

23. The compound of claim 1, wherein W is CX.

24. The compound of claim 1, wherein R₆ is H and Z is OR₂.

25. The compound of claim 24, wherein R₂ is H, —C(O)N(R₆)₂, —C(O)R₆, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid.

26-38. (canceled)

39. The compound of claim 1, wherein said compound is selected from the group consisting of:

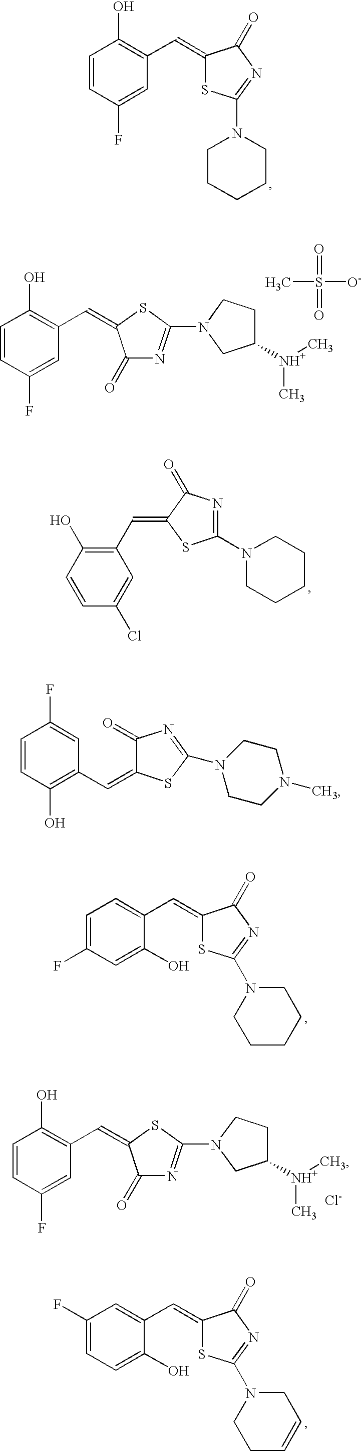

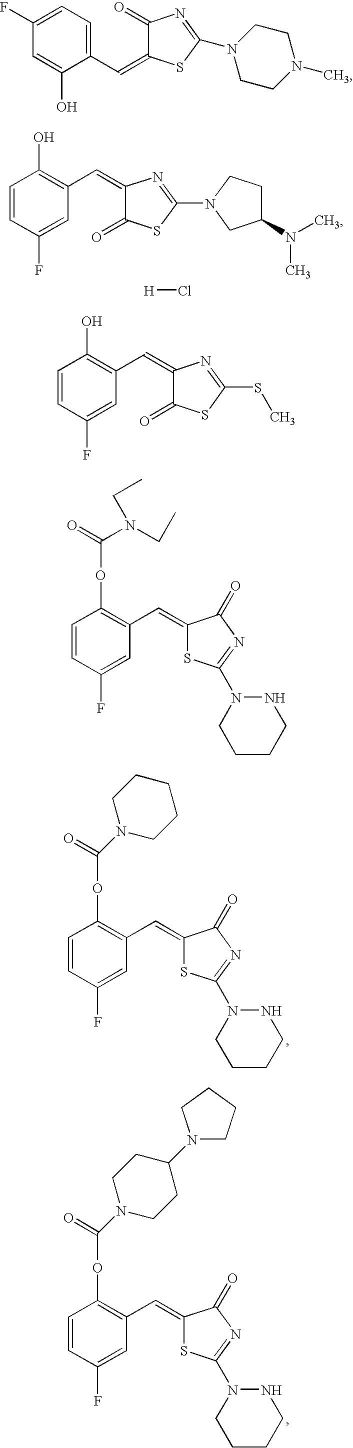

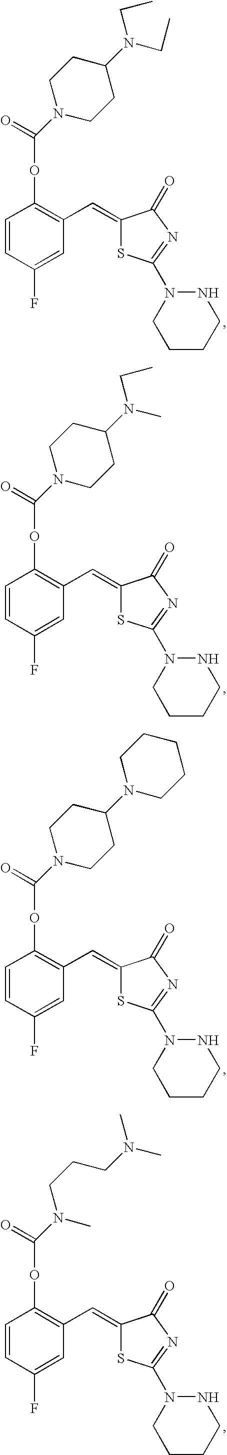

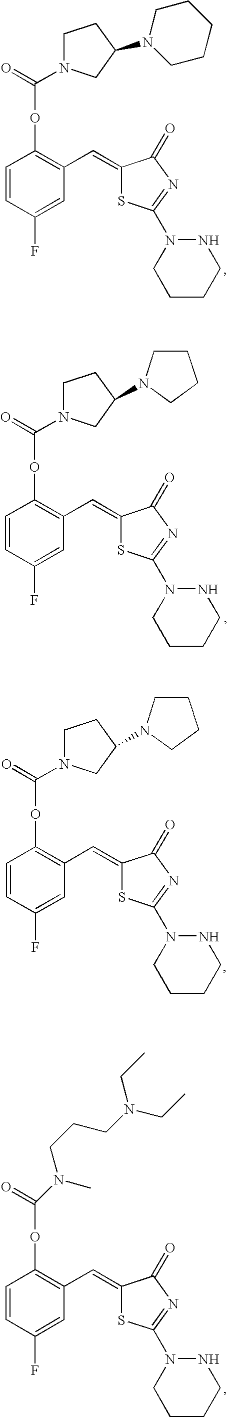

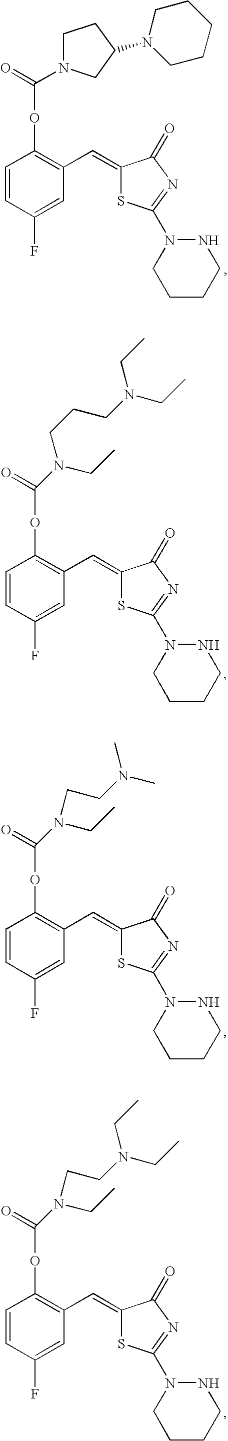

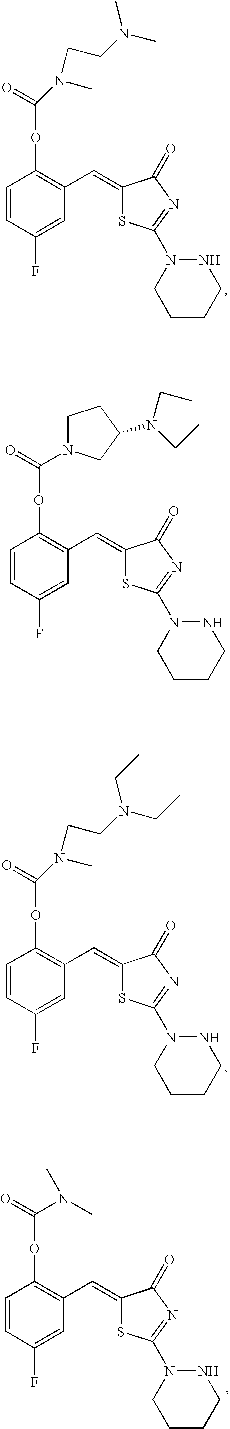

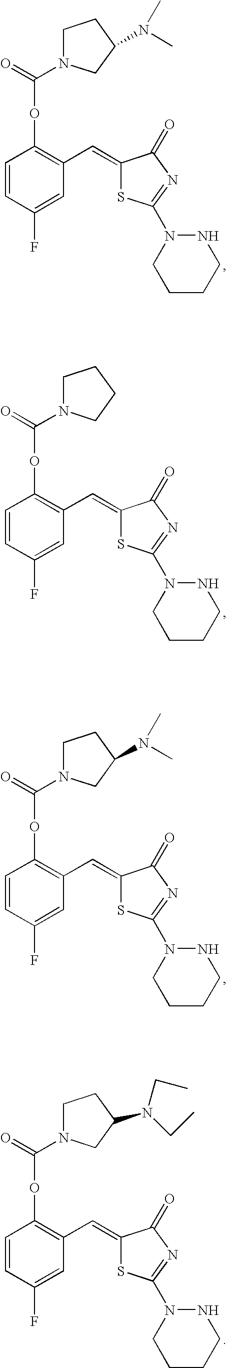

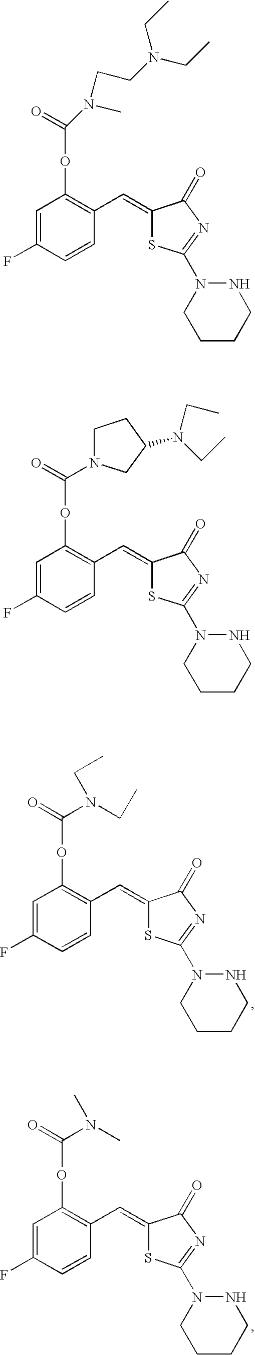

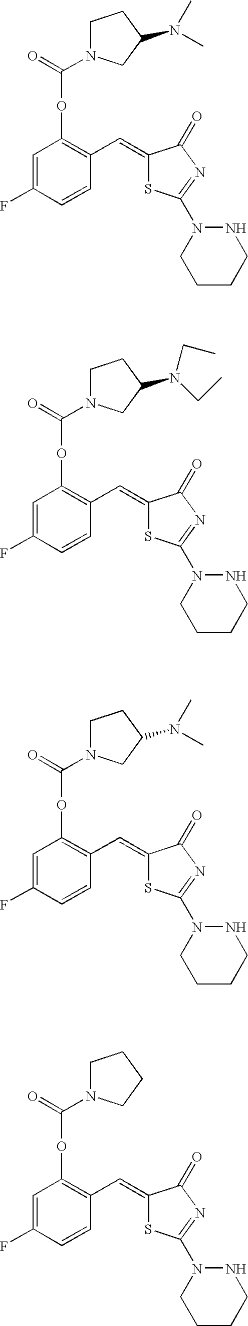

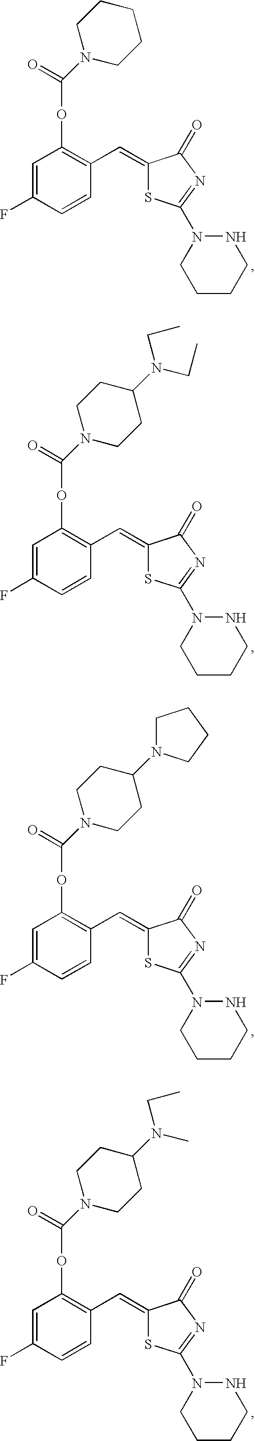

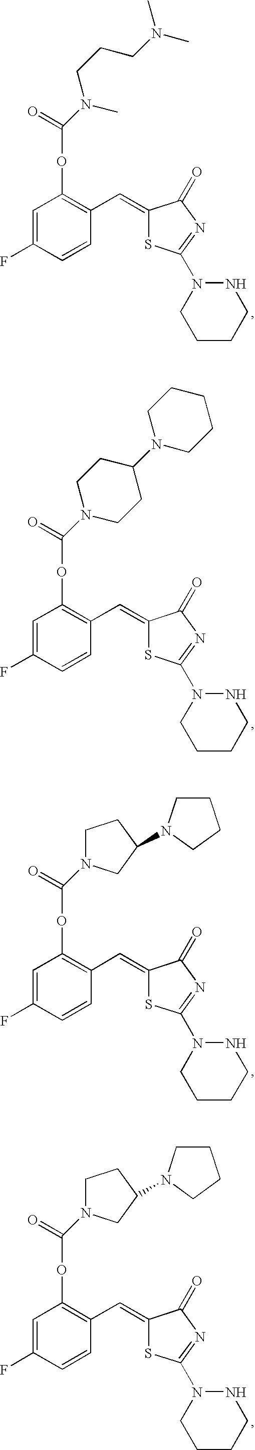

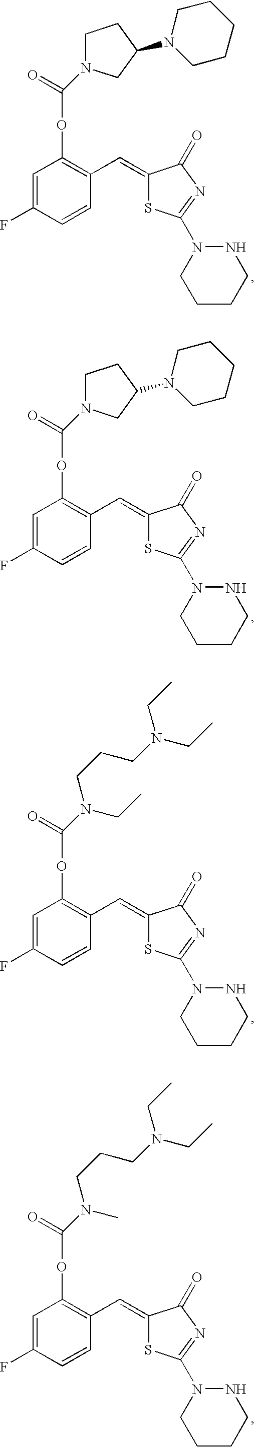

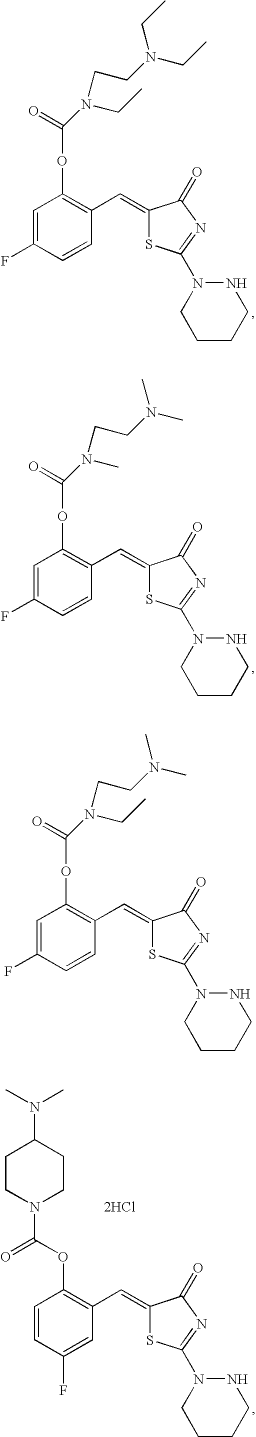

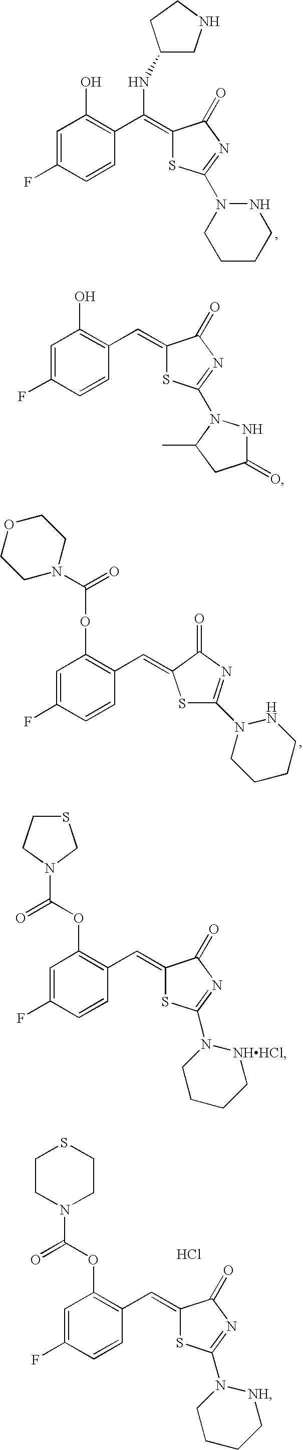

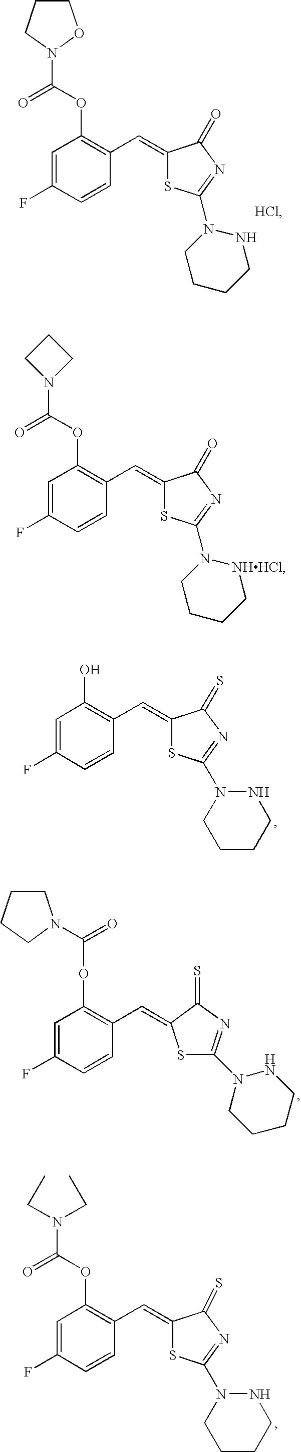

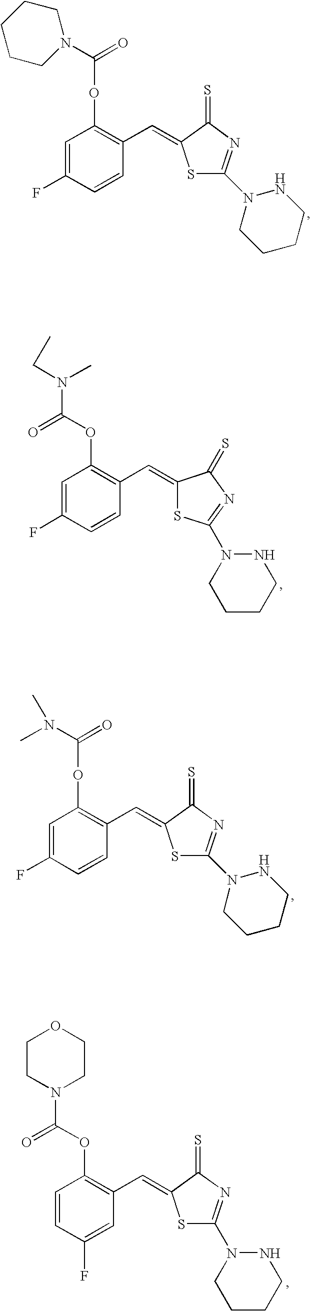

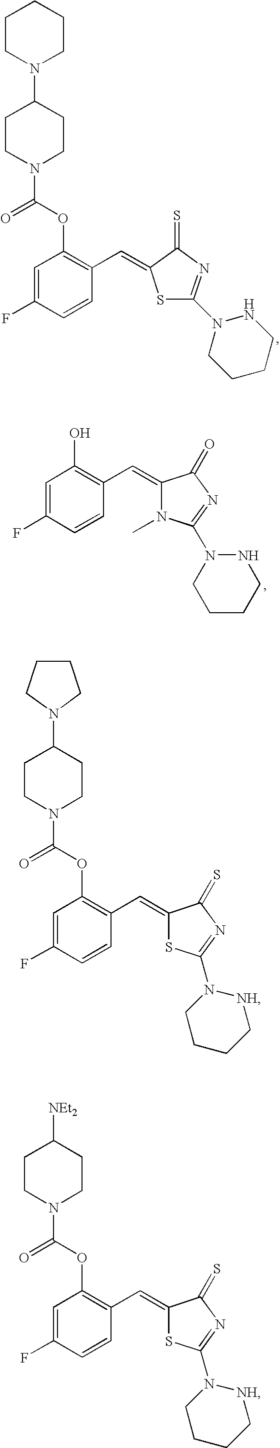

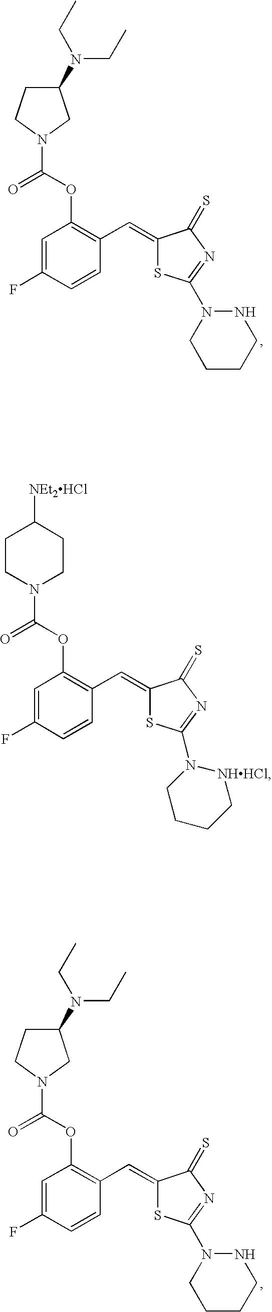

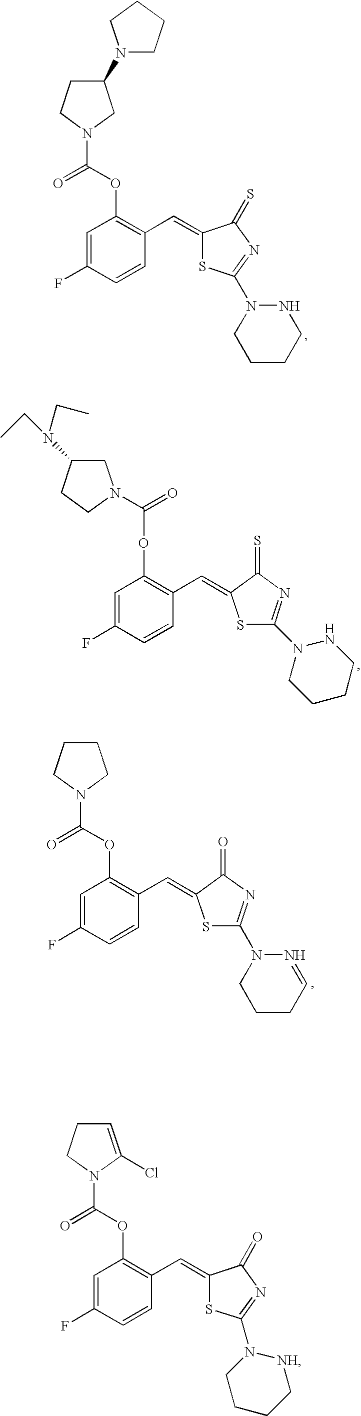

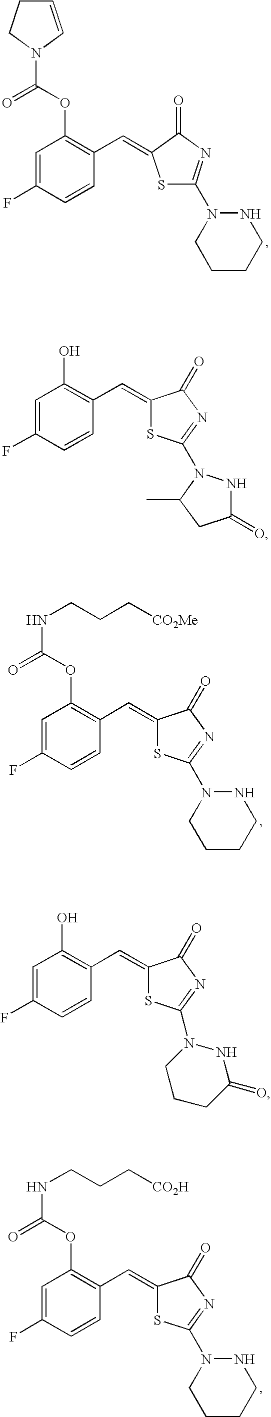

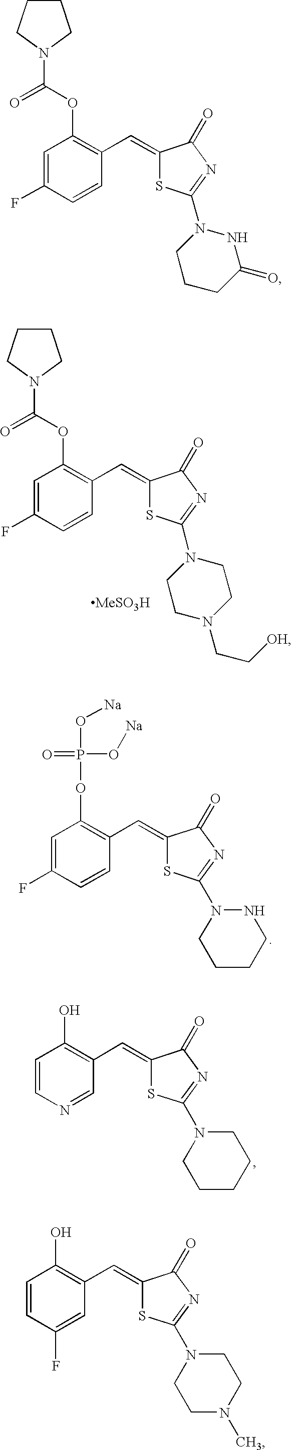

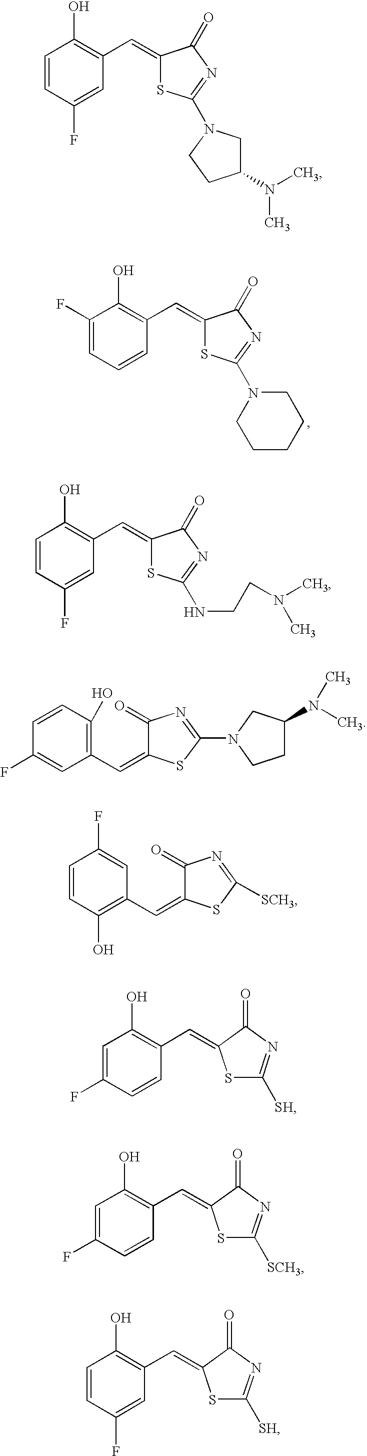

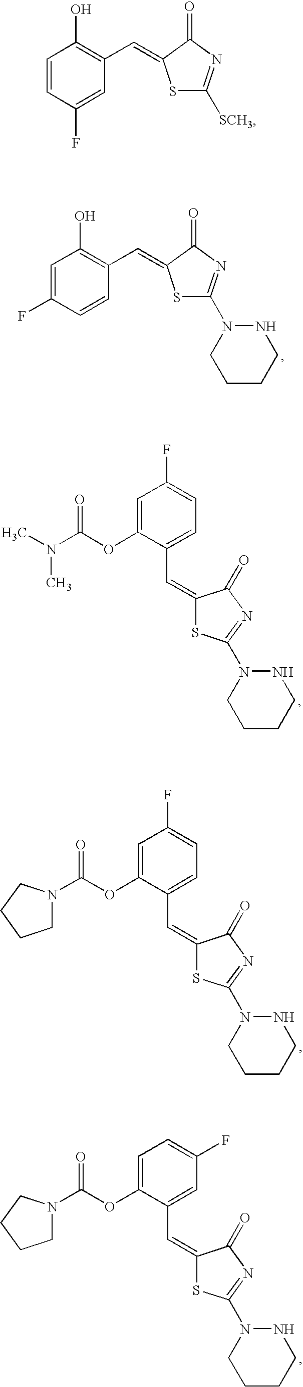

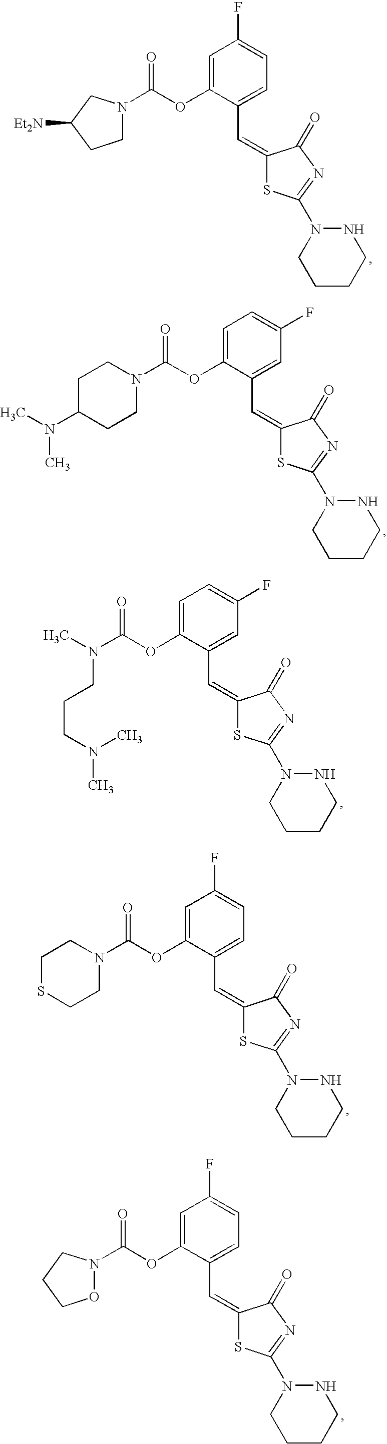

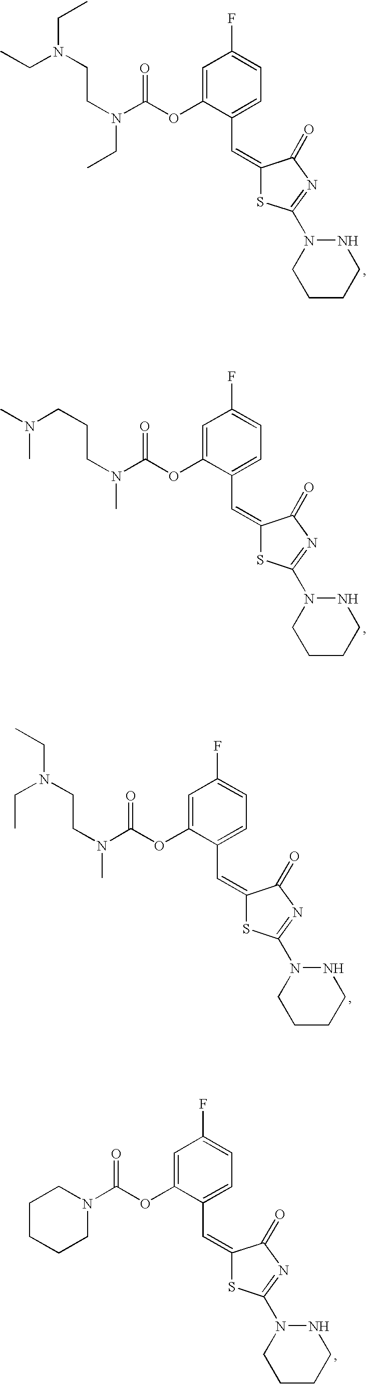

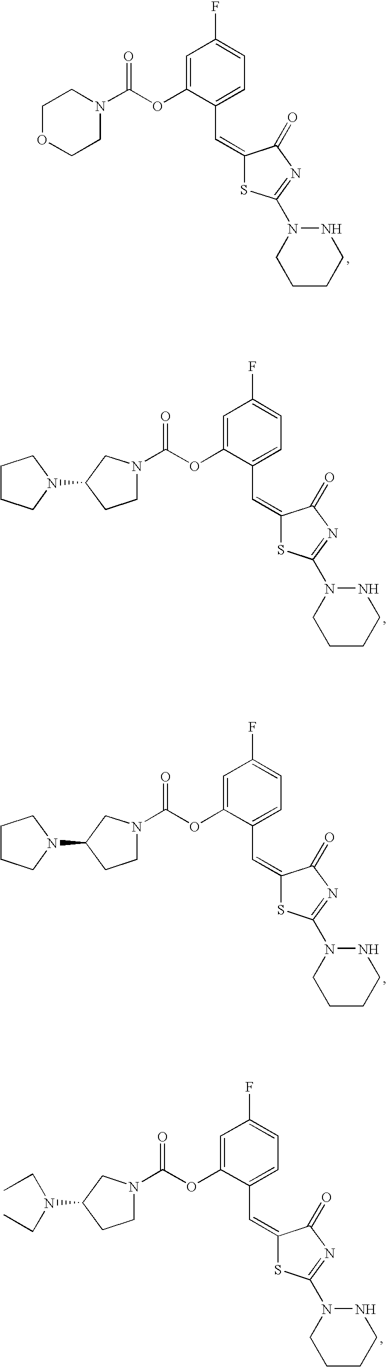

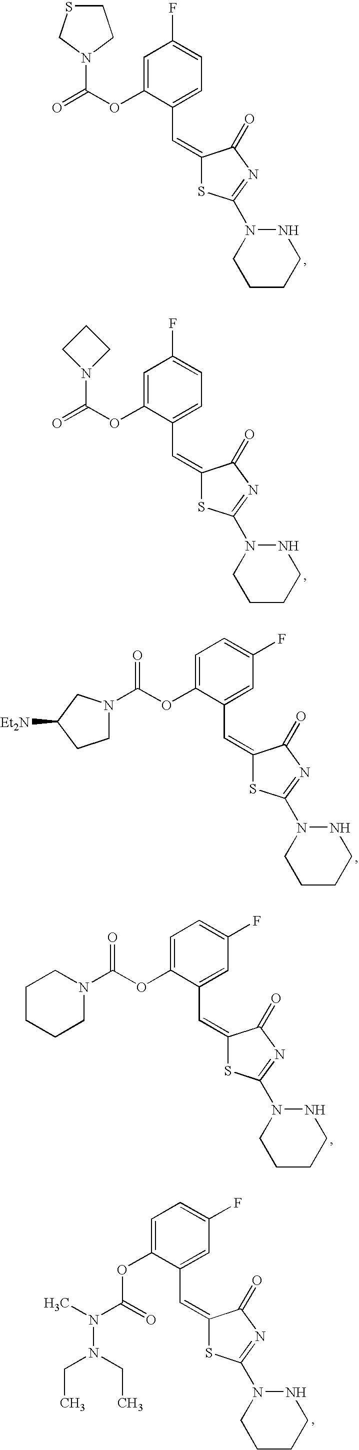

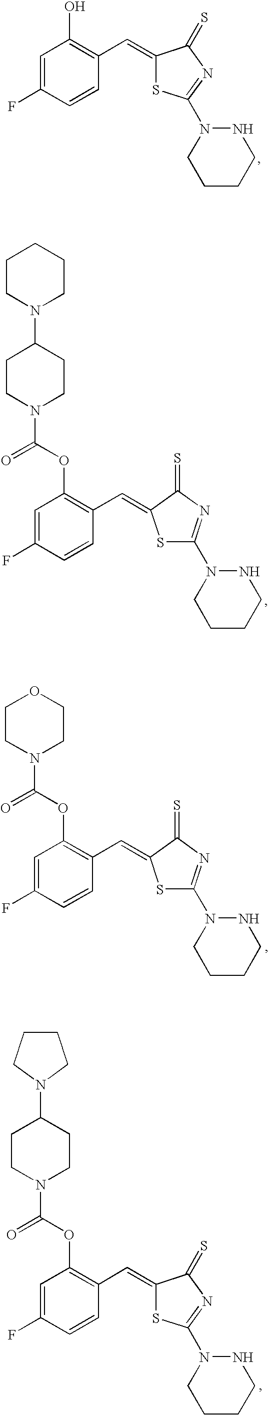

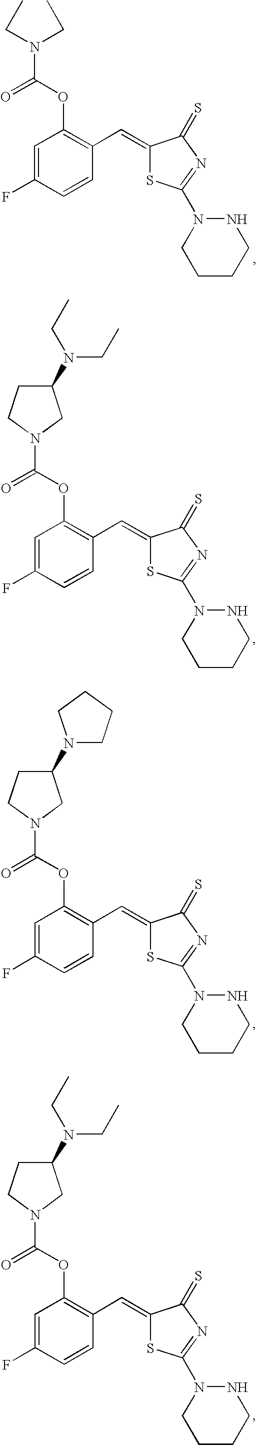

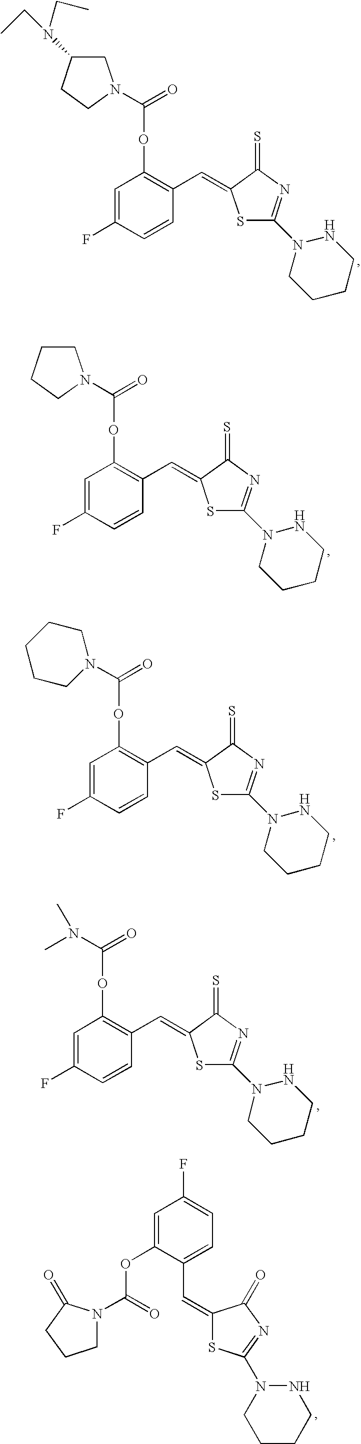

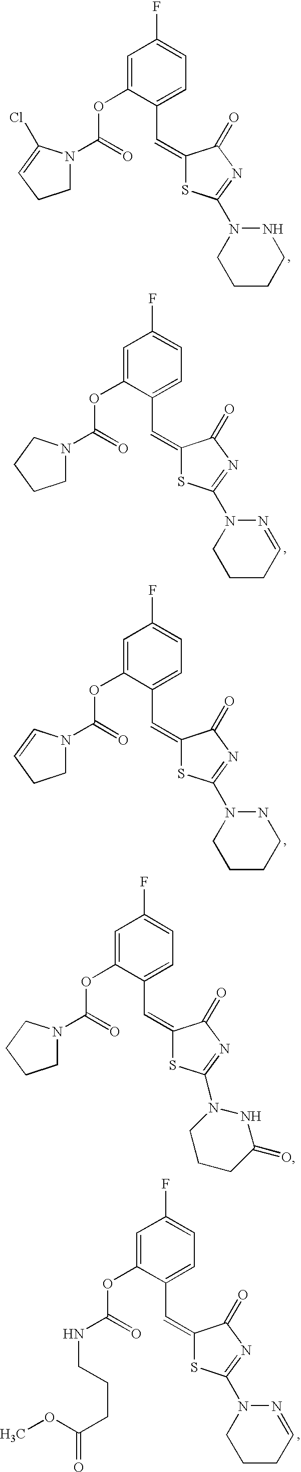

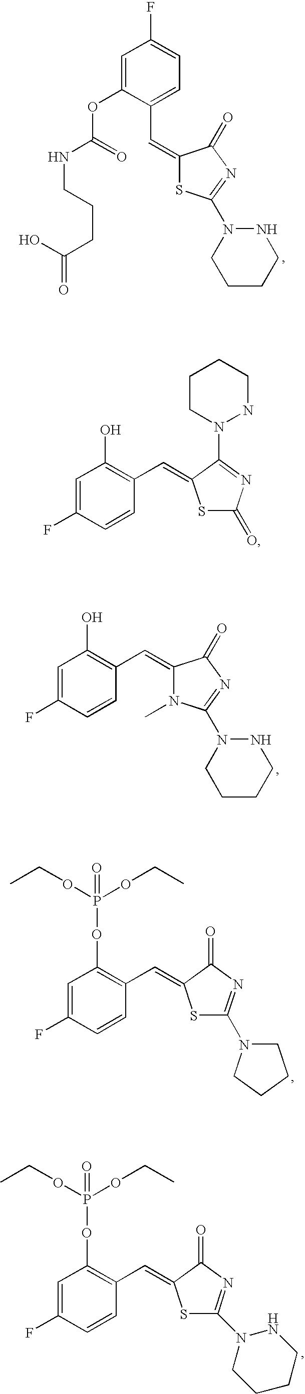

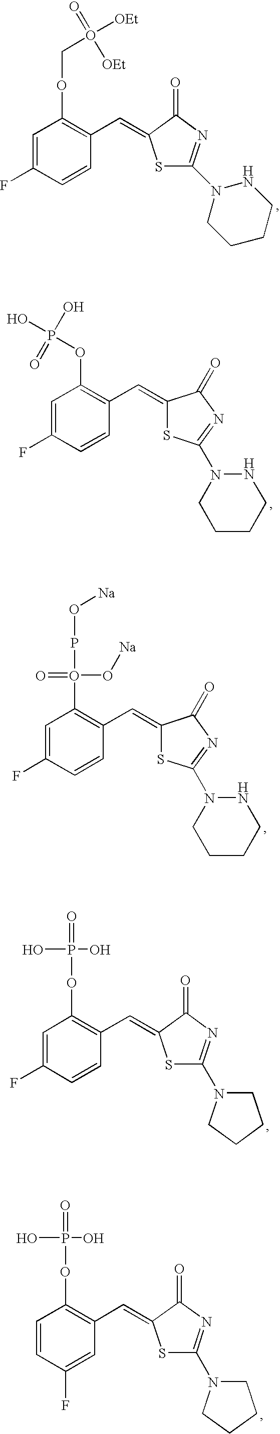

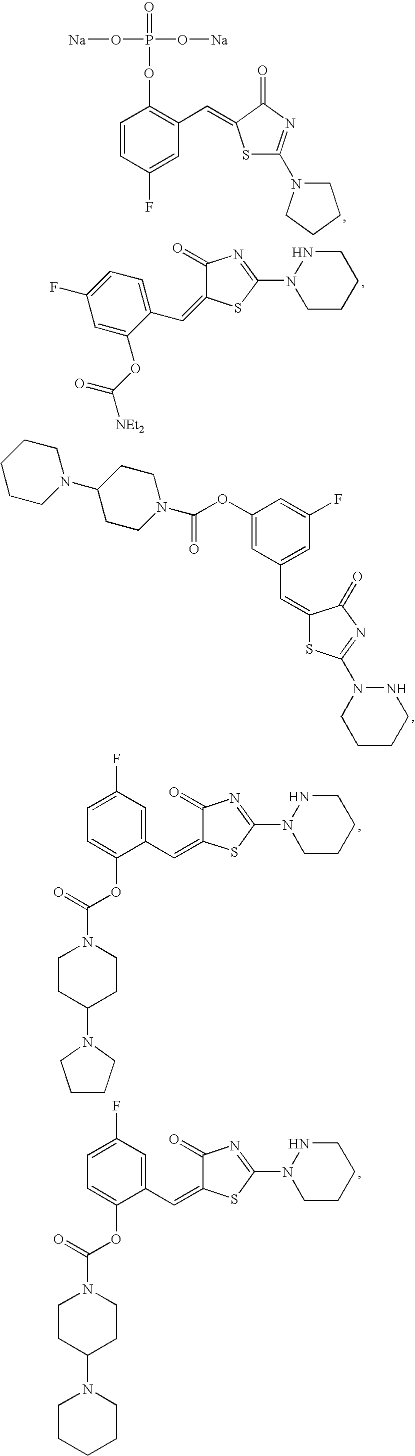

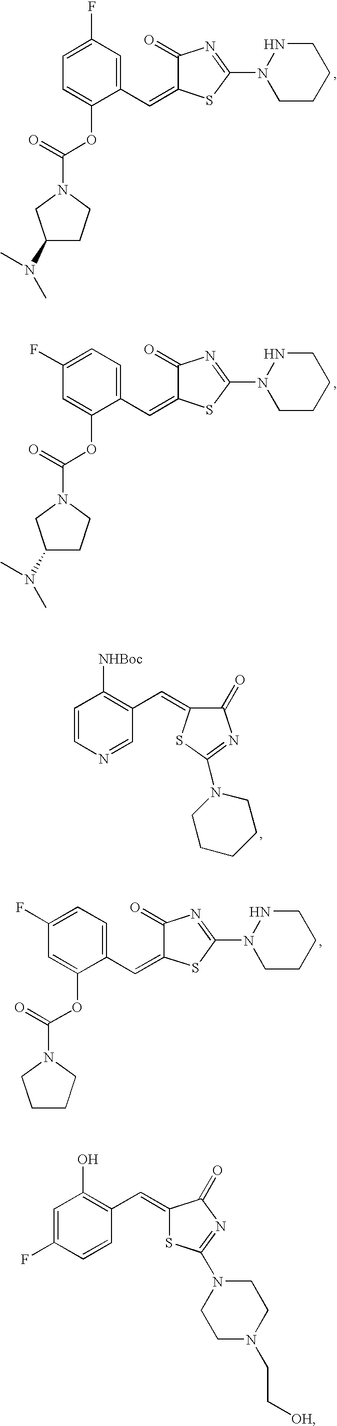

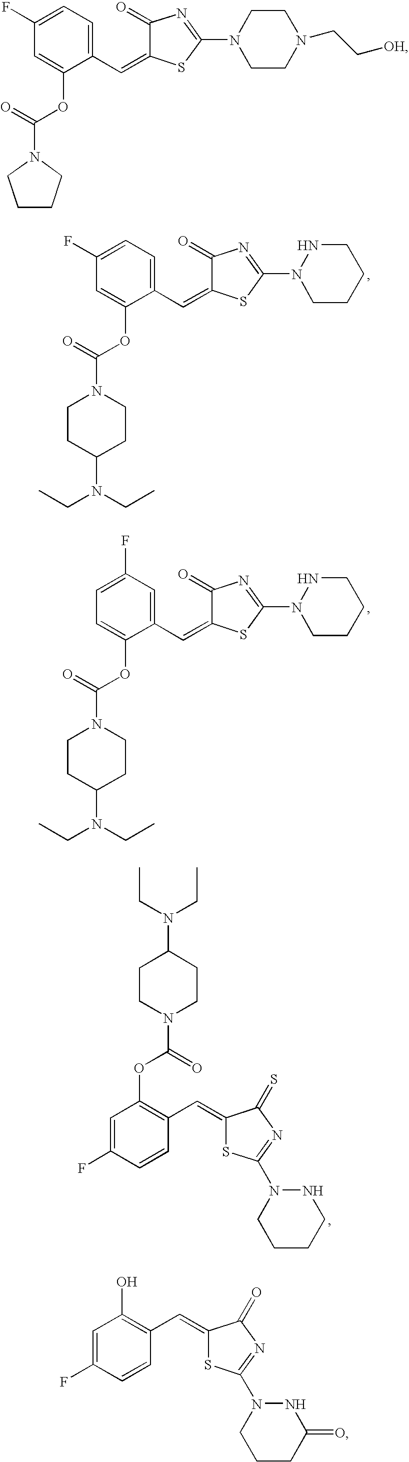

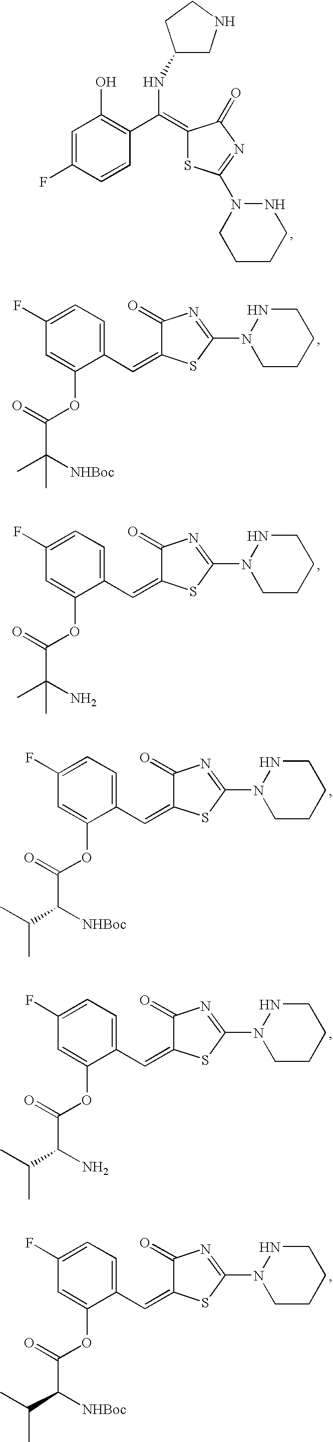

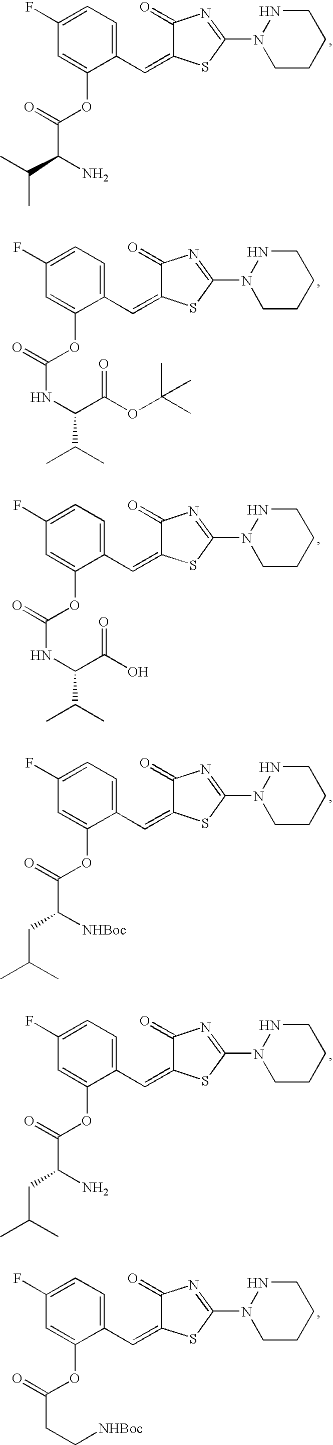

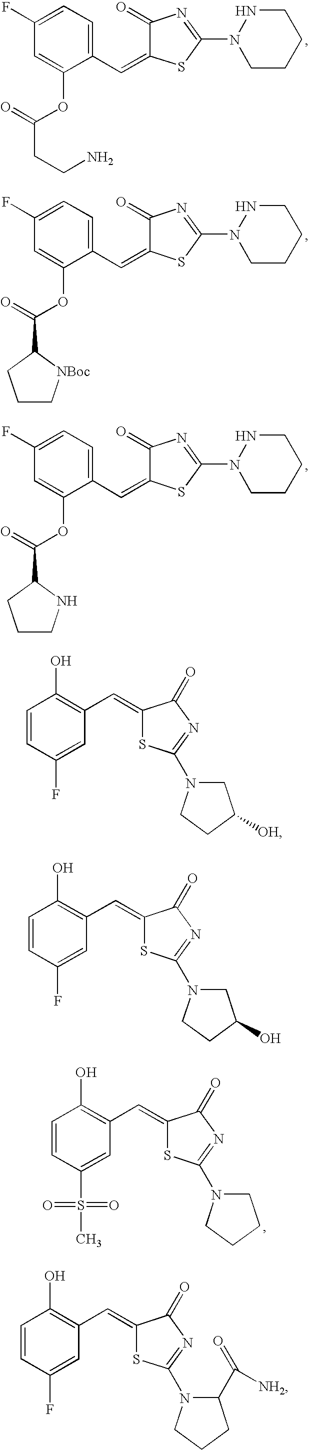

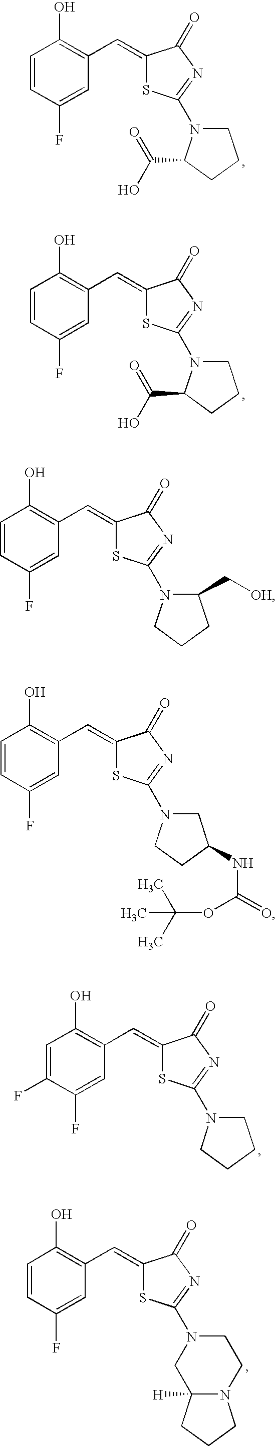

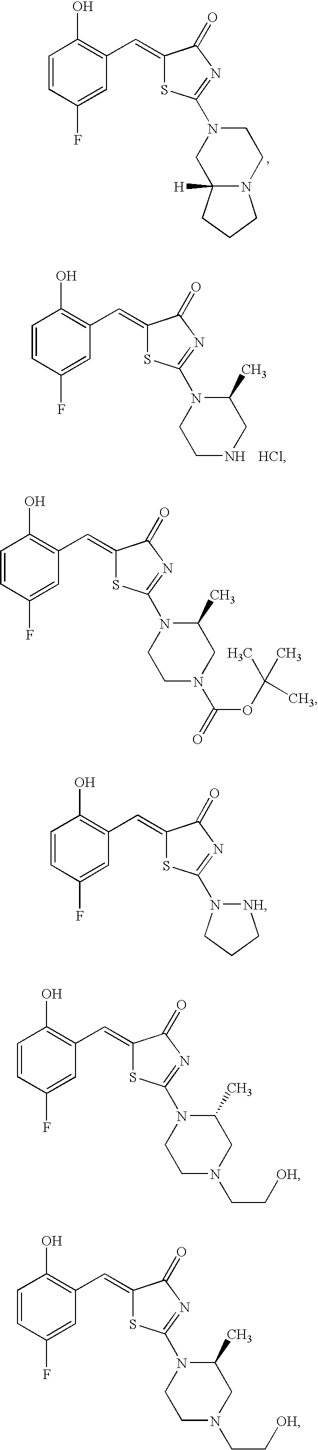

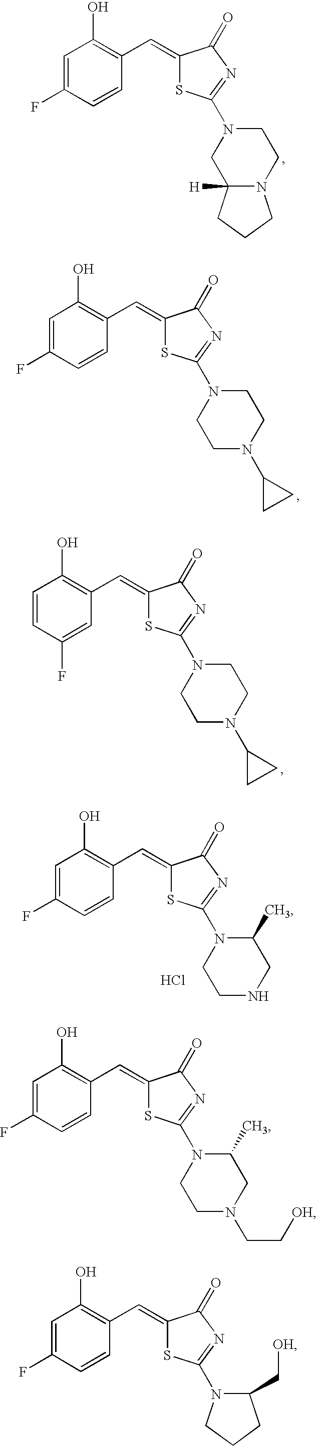

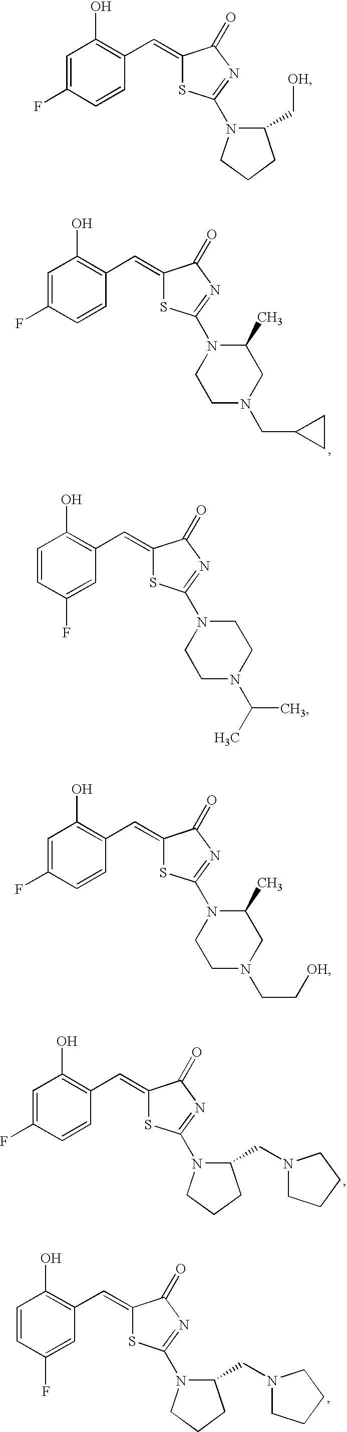

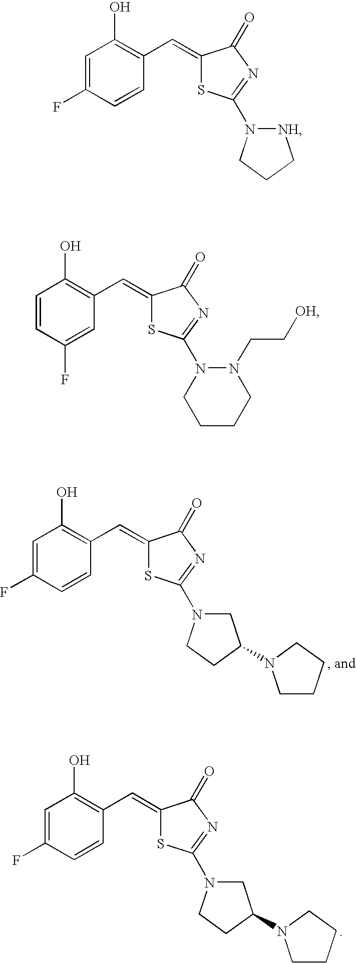

40. The compound of claim 1, wherein said compound has a structure selected from the group consisting of: 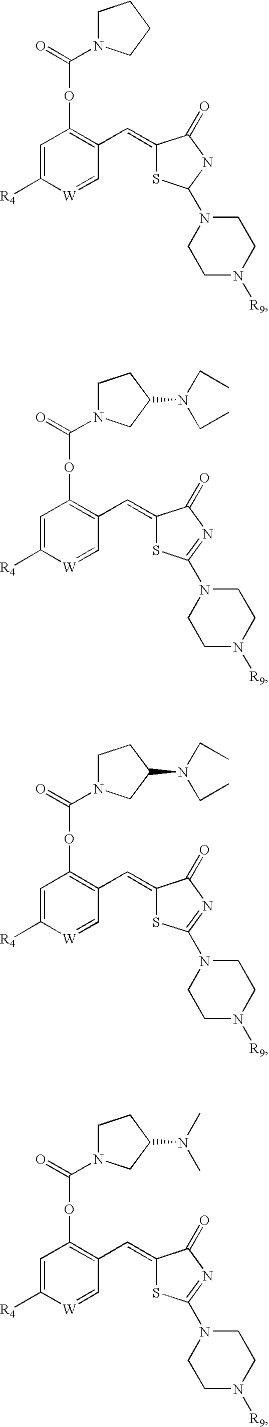 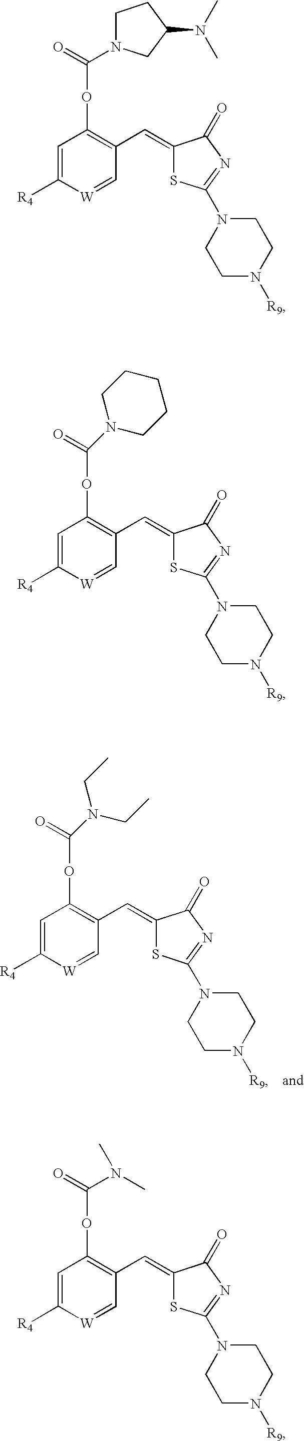
wherein, independently, W is CH or CF, R₄ is —H or —F, and R₉ is —C₁-C₃ alkyl that is optionally substituted with one —OH group.

41-42. (canceled)

43. A method for treating or preventing pain or inflammation in a patient, comprising administering to a patient in need thereof an effective amount of a compound of claim 1.

44. The method of claim 43, wherein said patient has neuropathic pain.

45-48. (canceled)

49. A composition comprising a pharmaceutically acceptable carrier or vehicle and an effective amount of a compound of claim 1.

50. A method for treating pain or inflammation in a patient, comprising administering to a patient in need thereof an effective amount of a compound having the Formula (Ib), 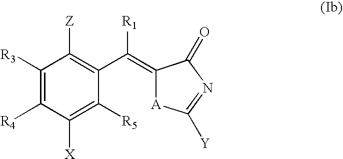
including stereoisomers, E/Z stereoisomers, prodrugs and pharmaceutically acceptable salts thereof, wherein:
A is —O—, —S—, —SO—, —SO₂—, >NR₆, or >NC(O)R₆;
Q is O, S, or NR₆;
Z is halogen, —NO₂, —OR₂, —N(R₆)₂, —C(O)R₆, or —C(O)(C(R₆)₂)ₒNH₂;
X is H, Br, I, OCH₃, NO₂, —C₆-C₁₂ aryl, —C₇-C₁₄ arylalkyl, N-terminal linked amino acid, or C-terminal linked amino acid;
Y is —C₃-C₈ cycloalkyl, —C₆-C₁₂ aryl, —C₇-C₁₄ arylalkyl, 3 to 9-membered heterocycle, —N(R₆)₂, —NHC(O)R₆, —NHS(O)₂R₆, —NHC(NH)N(R₆)₂, —NR₆C(NH)N(R₆)₂, —NHC(NCN)N(R₆)₂, or —NR₆C(NCN)N(R₆)₂;
R₁ is —H, halogen, —C₁-C₈ alkyl, —C₂-C₈ alkenyl, or —C₂-C₈ alkynyl;
R₂ is —H, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, —(CH₂)ₙOR₆, —C(O)R₆, —C(O)OR₆, —C(O)NHR₆, —C(O)N(R₆)₂, —(CR_{2A}R_{2B})ᵣ₂OPO(OR₆)₂, —(CR_{2A}R_{2B})ᵣ₃PO(OR₆)₂, N-terminal linked amino acid, or C-terminal linked amino acid;
R₃, R₄, and R₅ are each, independently, —H, —OH, halogen, —CN, —NO₂, —SH, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3 to 9-membered aromatic or non aromatic heterocycle, —OR₆, —N(R₆)₂, —C(NH)N(R₆)₂, —O(CH₂)ₙOR₆, —C(O)R₆, —OC(O)R₆, —OC(O)OR₆, —OC(O)N(R₆)₂, —C(O)N(R₆)₂, —C(O)OR₆, —SR₆, —SOR₅, —S(O)₂R₆, —NHC(O)R₆, —NHS(O)₂R₆, —NHC(NH)N(R₆)₂, —NR₆C(NH)N(R₆)₂, —NHC(NCN)N(R₆)₂, —NR₆C(NCN)N(R₆)₂, or —PO(OR₆)₂, or R₃ and R₄, together with the carbon atom to which each is attached, join to form a 5- to 6-membered aromatic or non aromatic carbocycle or heterocycle;
each R₆ is, independently, —H, —C₁-C₈ alkyl, —C₃-C₁₂ cycloalkyl, —C₆-C₁₂ aryl, —C₇-C₁₄ arylalkyl, 3 to 9-membered aromatic or non aromatic heterocycle, —C₂-C₈ alkenyl, or —C₂-C₈ alkynyl, or two R₆, together with the atom to which each is attached, join to form a 3- to 7-membered aromatic or non aromatic carbocycle or heterocycle;
n is 1 or 2;
o is an integer between 0-3;
r2 is an integer between 1-3; and
r3 is an integer between 0-2.

51-52. (canceled)

53. The method of claim 50, wherein said patient has neuropathic pain.

54-55. (canceled)

56. The method of claim 50, wherein the compound of Formula (Ib) has the structure selected from the group consisting of: 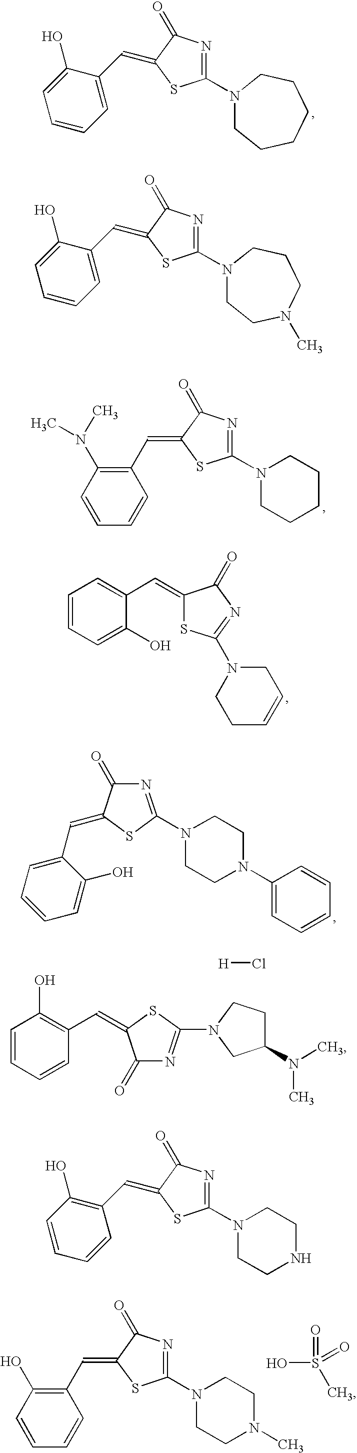 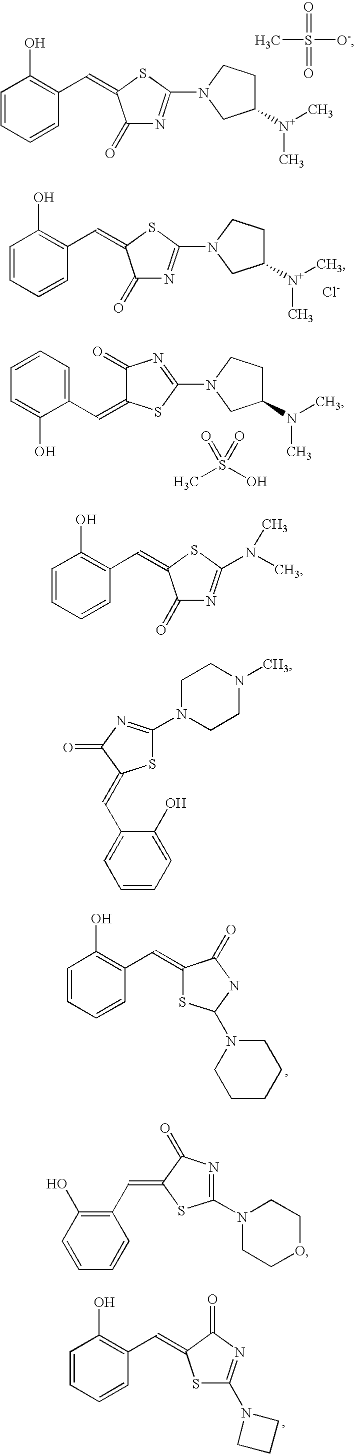 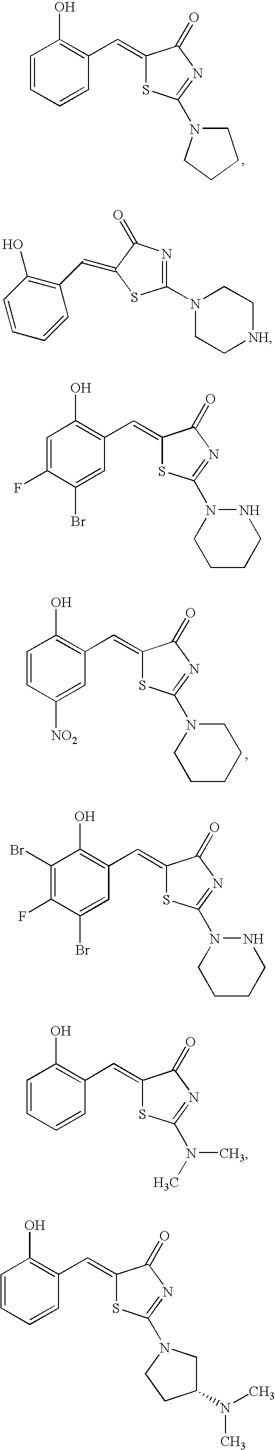 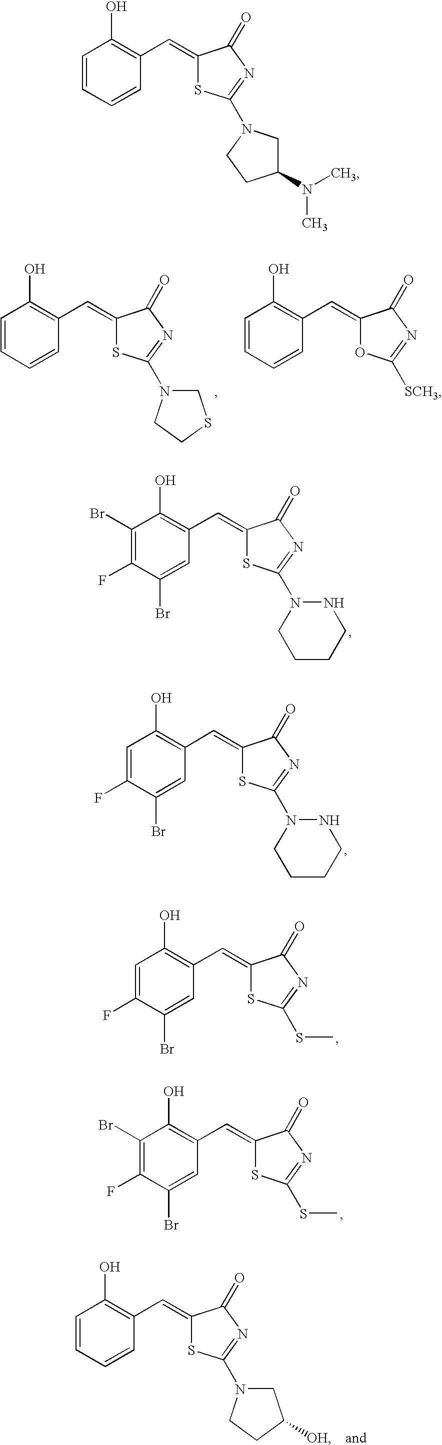 [chemical structure]
